SEQUENCE LISTING (16 sequences)

SEQ ID NO: 1 - protein, 4 aa, synthetic construct
GFLG

SEQ ID NO: 2 - protein, 4 aa, synthetic construct
ALAL

SEQ ID NO: 3 - protein, 218 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 4 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 224 aa, synthetic construct
EVQLLESGGGLVQPGGSLRLSCAASGFTFSSFSMSWVRQAPGKGLEWVSSISGSSGTTYYADSVKGRFTISRDNSKNTLYLQMNSLRAEDTAVYYCAKPFPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHT

SEQ ID NO: 6 - protein, 215 aa, synthetic construct
EIVLTQSPGTLSLSPGERATLSCRASQSVSSSFLAWYQQKPGQAPRLLIYYASSRATGIPDRFSGSGSGTDFTLTISRLEPEDFAVYYCQQTGRIPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPCTKSFNRGEC

SEQ ID NO: 7 - protein, 224 aa, synthetic construct
EVQLLESGGGLVQPGGSLRLSCAASGFTFSRYGMSWVRQAPGKGLEWVSAISGSGGSTYYADSVKGRFTISRDNSKNTLYLQMNSLRAEDTAVYYCAKAHNAFDYWGQGTLVTVSSCSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSSDKTHT

SEQ ID NO: 8 - protein, 214 aa, synthetic construct
SSELTQDPAVSVALGQTVRITCQGDSLRSYYASWYQQKPGQAPVLVIYGKNNRPSGIPDRFSGSSSGNTASLTITGAQAEDEADYYCNSSVYTMPPVVFGGGTKLTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 9 - protein, 132 aa, synthetic construct
QVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGGHHHHHH

SEQ ID NO: 10 - protein, 133 aa, synthetic construct
QVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGGHHHHHHC

SEQ ID NO: 11 - protein, 280 aa, synthetic construct
QVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGGGSGGGSGGGSGGGSGGGSGGGSQVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGGHHHHHH

SEQ ID NO: 12 - protein, 283 aa, synthetic construct
QVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGGHHHHHHCGGGSGGGSGGGSGGGSGGGSGGGSQVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGG

SEQ ID NO: 13 - protein, 283 aa, synthetic construct
QVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGGGGGSGGGSGGGSGGGSGGGSGGGSCHHHHHHQVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGG

SEQ ID NO: 14 - protein, 316 aa, synthetic construct
QVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGSGSGSGSGSGSGSGSGSGSGSGSGSGSGSGSGSGSGSGSGSGSGSGSGSGSGSGSGSGSQVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGGHHHHHH

SEQ ID NO: 15 - protein, 319 aa, synthetic construct
QVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGGHHHHHHCGSGSGSGSGSGSGSGSGSGSGSGSGSGSGSGSGSGSGSGSGSGSGSGSGSGSGSGSGSGSQVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGG

SEQ ID NO: 16 - protein, 317 aa, synthetic construct
QVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGSGSGSGSGSGSGSGSGSGSGSGSGSGSGSGSGSGSGSGSGSGSGSGSGSGSGSGSGSGSCHHHHHHQVQLVETGGGLVQAGGSLRLSCAASGSTFSHNAGGWYRQAPEKQRELVAGISSDGNINYADSVKDRFTISRDNASNTMYLQMNLKPEDTAVYVCNIRGSYGNTYYSRWGQGTQVTVSSGGLPETGG

(19) United States
(12) Patent Application Publication
(10) Pub. No.: US 2025/0186592 A1
(43) Pub. Date: Jun. 12, 2025

(54) TETRAZINE CONJUGATES FOR IN VIVO TARGETED DELIVERY OF A PAYLOAD

(71) Applicant: Tambo, Inc., San Francisco, CA (US)

(72) Inventors: José Manuel Mejía Oneto, San Francisco, CA (US); Michael ZAKHARIAN, Los Angeles, CA (US); Jesse M. MCFARLAND, Berkeley, CA (US); Amir MAHMOODI, Fairfield, CA (US); George CORICOR, San Francisco, CA (US); Jaime CABRERA-PARDO, Salt Lake City, UT (US)

(21) Appl. No.: 18/705,504

(22) PCT Filed: Oct. 31, 2022
(86) PCT No.: PCT/US2022/078995

§ 371 Date: Apr. 26, 2024

Related U.S. Application Data
(60) Provisional application No. 63/391,136, filed on Jul. 21, 2022.
(60) Provisional application No. 63/315,482, filed on Mar. 1, 2022.
(60) Provisional application No. 63/273,792, filed on Oct. 29, 2021.
(60) Provisional application No. 63/273,785, filed on Oct. 29, 2021.

(51) Int. Cl. A61K 47/54; A61K 47/60; A61K 47/65; A61K 47/68; A61P 35/00

(52) U.S. Cl. CPC A61K 47/545; A61K 47/542; A61K 47/60; A61K 47/65; A61K 47/6851; A61K 47/6889; A61P 35/00

(57) ABSTRACT

The present disclosure relates generally to targeting moieties for bioorthogonal delivery of a payload to a targeted location in a subject. The compositions and methods have applications in the treatment of cancer, tumor growths, and immunotherapy.

### CROSS REFERENCE TO RELATED APPLICATIONS

This application claims the benefit under 35 U.S.C. § 119 (e) to U.S. Provisional Application Nos. 63/273,792, filed Oct. 29, 2021, 63/273,785, filed Oct. 29, 2021, 63/315,482, filed Mar. 1, 2022, and 63/391,136, filed Jul. 21, 2022, each of which is incorporated by reference in its entirety.

### REFERENCE TO AN ELECTRONIC SEQUENCE LISTING

The contents of the electronic sequence listing (2022-10-31_Sequence_Listing_63XT-342805-WO.xml; Size: 18,193 bytes; and Date of Creation: Oct. 31, 2022) are herein incorporated by reference in their entireties.

### FIELD

The present disclosure relates generally to tetrazine conjugates, including antibody-tetrazine conjugates, for bioorthogonal delivery of a payload to a targeted location in a subject, which conjugates have applications, e.g., in the treatment of cancer, tumor growth, and immunotherapy.

### BACKGROUND

Bioorthogonal conjugation or click reactions are selective and orthogonal (non-interacting with) functionalities found in biological systems, and have found use in various applications in the fields of chemistry, chemical biology, molecular diagnostics, and medicine, where they can be used to facilitate the selective manipulation of molecules, cells, particles and surfaces, and the tagging and tracking of biomolecules in vitro and in vivo. These reactions include the Staudinger ligation, the azide-cyclooctyne cycloaddition, and the inverse-electron-demand Diels-Alder reaction.

### SUMMARY

Provided herein are targeting moieties which comprise an antibody or antibody fragment moiety covalently bonded to one or more tetrazine moieties. The targeting moieties described herein are designed to, once administered to a subject, localize at a target site within the subject. The targeting moieties can be administered locally or systemically. Once administered, a prodrug comprising a payload or therapeutic agent and one or more complimentary bioorthogonal components (i.e., a trans-cyclooctene moiety) can be administered, which when in contact with the targeting moiety in vivo, allows for targeted delivery of the payload or therapeutic agent. In some embodiments, the targeting moiety is a therapeutic targeting moiety. In some embodiments, the targeting moieties described herein comprise a diagnostic agent such that the targeting moieties described herein can be used in diagnosing conditions or diseases, with or without administering a payload or therapeutic agent.

In some embodiments, provided is a method for treating cancer, comprising administering to a subject in need thereof, a support composition as described herein to a target location, and administering to the subject a conjugate, or the pharmaceutically acceptable salt or composition thereof, as described herein.

In some embodiments, the cancer is metastatic. In some embodiments the cancer is melanoma, renal cancer, prostate cancer, ovarian cancer, endometrial carcinoma, breast cancer, glioblastoma, lung cancer, soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, Non-Hodgkin's lymphoma, Hodgkin's lymphoma Wilm's tumor/neuroblastoma, bladder cancer, thyroid adenocarcinoma, pancreatic neuroendocrine tumors, prostatic adenocarcinoma, nasopharyngeal carcinoma, or cutaneous T-cell lymphoma.

In some embodiments, the cancer is a melanoma, renal cancer, prostate cancer, ovarian cancer, breast cancer, glioma, lung cancer, soft tissue carcinoma, soft tissue sarcoma, osteosarcoma, or pancreatic cancer. In some embodiments, the cancer is a solid tumor. In some embodiments, the cancer is a lymphoma or leukemia. In some embodiments, the cancer is a hematologic malignancy.

### BRIEF DESCRIPTION OF THE FIGURES

FIG. 1 shows SDS-Page of a methyltetrazine-trastuzumab targeting moiety.

FIG. 2 shows LCMS of a methyltetrazine-trastuzumab targeting moiety.

FIG. 3 shows SDS-PAGE of a methyltetrazine-Fab targeting moiety.

FIG. 4 shows LCMS of a methyltetrazine-Fab targeting moiety.

FIG. 5 shows efficacy of a methyltetrazine-Fab targeting moiety with a doxorubicin-TCO prodrug (doxorubicin drug modified with TCO) in a mouse HCC1954 xenograft model.

FIG. 6 shows LC-MS analysis of conjugated trastuzumab-Me-Tet-PEG9 ADC.

FIG. 7 shows FACS analysis of binding effect of trastuzumab Fab, Fab-Me-Tet-PEG9 ADC, and IgG (as negative control) to NCI-N87 cell line.

FIG. 8 shows tumor volume in the NCI-N87 model.

FIG. 9 shows tumor growth inhibition curves in the NCI-N87 model.

FIGS. 10A and 10B shows xenograft results and % body weight loss (BWL) for the Ab-Tz conjugate prepared in Example 6.

FIG. 11 shows a schematic of the tetrazine-antigen-binding protein targeting moiety prepared in Example 6.

### DETAILED DESCRIPTION

The following description sets forth exemplary embodiments of the present technology. It should be recognized, however, that such description is not intended as a limitation on the scope of the present disclosure but is instead provided as a description of exemplary embodiments.

### 1. Definitions

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed, to the extent that such combinations embrace subject matter that are, for example, compounds that are stable compounds (i.e., compounds that can be made, isolated, characterized, and tested for biological activity). In addition, all sub-combinations of the various embodiments and elements thereof (e.g., elements of the chemical groups listed in the embodiments describing such variables) are also specifically embraced by the present disclosure and are disclosed herein just as if each and every such sub-combination was individually and explicitly disclosed herein.

### A. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

The conjunctive term “or” includes any and all combinations of one or more listed elements associated by the conjunctive term. For example, the phrase “an apparatus comprising A or B” may refer to an apparatus including A where B is not present, an apparatus including B where A is not present, or an apparatus where both A and B are present. The phrases “at least one of A, B, . . . and N” or “at least one of A, B, . . . . N, or combinations thereof” are defined in the broadest sense to mean one or more elements selected from the group comprising A, B, . . . and N, that is to say, any combination of one or more of the elements A, B, . . . or N including any one element alone or in combination with one or more of the other elements which may also include, in combination, additional elements not listed.

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; Carruthers, Some Modern Methods of Organic Synthesis, 3^{rd} Edition, Cambridge University Press, Cambridge, 1987; the entire contents of each of which are incorporated herein by reference.

The term “alkyl” as used herein, means a straight or branched, saturated hydrocarbon chain containing from 1 to 30 carbon atoms. The term “lower alkyl” or “C₁-C₆-alkyl” means a straight or branched chain hydrocarbon containing from 1 to 6 carbon atoms. The term “C₁-C₃-alkyl” means a straight or branched chain hydrocarbon containing from 1 to 3 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkoxy” as used herein, refers to an alkyl group, as defined herein, appended to the parent molecular moiety through an oxygen atom. Representative examples of alkoxy include, but are not limited to, methoxy, ethoxy, propoxy, 2-propoxy, butoxy, and tert-butoxy.

The term “alkenyl” as used herein, means a hydrocarbon chain containing from 2 to 30 carbon atoms with at least one carbon-carbon double bond. The alkenyl group may be substituted or unsubstituted. For example, the alkenyl group may be substituted with an aryl group, such as a phenyl.

The term “alkynyl,” as used herein, refers to straight or branched monovalent hydrocarbyl groups having from 2 to 30 carbon atoms, such as 2 to 20, or 2 to 10 carbon atoms and having at least 1 site of triple bond unsaturation. The term “alkyne” also includes non-aromatic cycloalkyl groups of from 5 to 20 carbon atoms, such as from 5 to 10 carbon atoms, having single or multiple rings and having at least one triple bond. Examples of such alkynyl groups include, but are not limited to acetylenyl (—C≡CH), and propargyl (—CH₂C≡CH), and cycloalkynyl moieties, such as, but not limited to, substituted or unsubstituted cyclooctyne moieties.

The term “alkoxyalkyl” as used herein, refers to an alkoxy group, as defined herein, appended to the parent molecular moiety through an alkyl group, as defined herein.

The term “alkylene” as used herein, refers to a divalent group derived from a straight or branched chain hydrocarbon of 1 to 30 carbon atoms, for example, of 2 to 10 carbon atoms. Representative examples of alkylene include, but are not limited to, —CH₂—, —CH(CH₃)—, —C(CH₃)₂—, —CH₂CH₂—, —CH(CH₃)CH₂—, —C(CH₃)₂CH₂—, —CH₂CH₂CH₂—, —CH(CH₃)CH₂CH₂—, —C(CH₃)₂CH₂CH₂—, —CH₂C(CH₃)₂CH₂—, —CH₂CH₂CH₂CH₂—, and —CH₂CH₂CH₂CH₂CH₂—.

The term “amino acid” refers to both natural and unnatural amino acids, protected natural and unnatural amino acids, as well as amino acid analogs and amino acid mimetics that function in a manner similar to the naturally occurring amino acids. Naturally encoded amino acids include 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine) and pyrrolidine and selenocysteine. Non-natural amino acids refer to amino acid analogs having the same basic chemical structure as a naturally occurring amino acid, i.e., by way of example only, an α-carbon attached to a hydrogen, carboxyl group, amino group, and R group. Such analogs can have a modified R group (e.g., norleucine as an example) or retain a modified peptide backbone while retaining the same basic chemical structure as a natural amino acid. Non-limiting examples of non-natural amino acids or amino acid analogs include citrulline, homoserine, norleucine, methionine sulfoxide, methionine methylsulfonium, homophenylalanine, ornithine, formyl glycine, phenyl glycine, para-azidophenyl glycine, para-azidophenylalanine, para-acetophenylalanine, 4-(3-methyl-(1,2,4,5-tetrazine))-phenylglyine, and 4-(3-methyl-(1,2,4,5-tetrazine))-phenylalanine.

The term “aryl” as used herein, refers to an aromatic carbocyclic group having a single ring (e.g. monocyclic) or multiple rings (e.g. bicyclic or tricyclic) including fused systems. Representative examples of aryls include, but are not limited to, phenyl, naphthyl, and anthracenyl. The monocyclic, bicyclic, and tricyclic aryls are connected to the parent molecular moiety through any carbon atom contained within the rings, and can be unsubstituted or substituted. The aromatic bicyclic ring system or aromatic tricyclic ring system does not contain non-aromatic rings. Thus, if a bicyclic ring system or tricyclic ring system contains a non-aromatic ring, the ring system is a cycloalkyl or heterocyclyl, depending on whether a heteroatom is present in the non-aromatic ring, regardless of the point of attachment to the remainder of the molecule.

In some embodiments, the term “aryl” as used herein, refers to a phenyl group, or bicyclic aryl or tricyclic aryl fused ring systems. Bicyclic fused ring systems are exemplified by a phenyl group appended to the parent molecular moiety and fused to a phenyl group. Tricyclic fused ring systems are exemplified by a phenyl group appended to the parent molecular moiety and fused to two other phenyl groups. Representative examples of bicyclic aryls include, but are not limited to, naphthyl. Representative examples of tricyclic aryls include, but are not limited to, anthracenyl. The monocyclic, bicyclic, and tricyclic aryls are connected to the parent molecular moiety through any carbon atom contained within the rings, and can be unsubstituted or substituted.

The term “azide” as used herein, refers to the functional group —N₃.

The term “cycloalkyl” as used herein, refers to a non-aromatic carbocyclic ring system containing 3 to 10, or 3 to 8, or 3 to 6, or 5 to 10, carbon atoms and zero heteroatoms. Cycloalkyl ring systems may contain one or more double bonds, so long as the ring is not aromatic; and thus, the term cycloalkyl includes cycloalkenyl ring systems. Representative examples of cycloalkyl include, but are not limited to, cyclopropyl, cyclobutyl, cyclopentyl, cyclohexyl, cycloheptyl, cyclooctyl, cyclononyl, and cyclodecyl. Exemplary monocyclic cycloalkenyl rings include cyclopentenyl, cyclohexenyl, or cycloheptenyl. “Cycloalkyl” also includes carbocyclic ring systems in which a cycloalkyl group is fused to an aryl or heteroaryl as defined herein, regardless of the point of attachment to the remainder of the molecule.

In some embodiments, the term “cycloalkyl” as used herein, refers to a carbocyclic ring system containing three to ten carbon atoms, zero heteroatoms and zero double bonds. Representative examples of cycloalkyl include, but are not limited to, cyclopropyl, cyclobutyl, cyclopentyl, cyclohexyl, cycloheptyl, cyclooctyl, cyclononyl and cyclodecyl. “Cycloalkyl” also includes carbocyclic ring systems in which a cycloalkyl group is appended to the parent molecular moiety and is fused to an aryl group as defined herein, a heteroaryl group as defined herein, or a heterocycle as defined herein.

The term “cycloalkenyl” as used herein, means a non-aromatic monocyclic or multicyclic ring system containing at least one carbon-carbon double bond and preferably having from 5-10 carbon atoms per ring. Exemplary monocyclic cycloalkenyl rings include cyclopentenyl, cyclohexenyl or cycloheptenyl.

The term “cyclooctene” as used herein, refers to a substituted or unsubstituted non-aromatic cyclic alkyl group of 8 carbon atoms, having a single ring with a double bond. Examples of such cyclooctene groups include, but are not limited to, substituted or unsubstituted trans-cyclooctene (TCO).

The term “fluoroalkyl” as used herein, means an alkyl group, as defined herein, in which one, two, three, four, five, six, seven or eight hydrogen atoms are replaced by fluorine. Representative examples of fluoroalkyl include, but are not limited to, 2-fluoroethyl, 2,2,2-trifluoroethyl, trifluoromethyl, difluoromethyl, pentafluoroethyl, and trifluoropropyl such as 3,3,3-trifluoropropyl.

The term “alkoxyfluoroalkyl” as used herein, refers to an alkoxy group, as defined herein, appended to the parent molecular moiety through a fluoroalkyl group, as defined herein.

The term “fluoroalkoxy” as used herein, means at least one fluoroalkyl group, as defined herein, is appended to the parent molecular moiety through an oxygen atom. Representative examples of fluoroalkyloxy include, but are not limited to, difluoromethoxy, trifluoromethoxy and 2,2,2-trifluoroethoxy.

The term “halogen” or “halo” as used herein, means Cl, Br, I, or F.

The term “haloalkyl” as used herein, means an alkyl group, as defined herein, in which one, two, three, four, five, six, seven or eight hydrogen atoms are replaced by a halogen.

The term “haloalkoxy” as used herein, means at least one haloalkyl group, as defined herein, is appended to the parent molecular moiety through an oxygen atom.

The term “heteroalkyl” as used herein, means an alkyl group, as defined herein, in which one or more of the carbon atoms has been replaced by a heteroatom selected from S, Si, O, P and N. The heteroatom may be oxidized. Representative examples of heteroalkyls include, but are not limited to, alkyl ethers, secondary and tertiary alkyl amines, and alkyl sulfides.

The term “heteroaryl” as used herein, refers to an aromatic group having a single ring, multiple rings or multiple fused rings, with one or more ring heteroatoms independently selected from nitrogen, oxygen and sulfur. In some embodiments, the term “heteroaryl” as used herein, refers to an aromatic monocyclic ring or an aromatic bicyclic ring system or an aromatic tricyclic ring system. The aromatic monocyclic rings are five or six membered rings containing at least one heteroatom independently selected from the group consisting of N, O and S (e.g. 1, 2, 3, or 4 heteroatoms independently selected from O, S, and N). The five membered aromatic monocyclic rings have two double bonds and the six membered aromatic monocyclic rings have three double bonds. Representative examples of monocyclic heteroaryl include, but are not limited to, pyridinyl (including pyridin-2-yl, pyridin-3-yl, pyridin-4-yl), pyrimidinyl, pyrazinyl, thienyl, furyl, thiazolyl, thiadiazolyl, isoxazolyl, pyrazolyl, and 2-oxo-1,2-dihydropyridinyl. Representative examples of bicyclic heteroaryl include, but are not limited to, chromenyl, benzothienyl, benzodioxolyl, benzotriazolyl, quinolinyl, thienopyrrolyl, thienothienyl, imidazothiazolyl, benzothiazolyl, benzofuranyl, indolyl, quinolinyl, imidazopyridine, benzooxadiazolyl, and benzopyrazolyl. Representative examples of tricyclic heteroaryl include, but are not limited to, dibenzofuranyl and dibenzothienyl. The monocyclic, bicyclic, and tricyclic heteroaryls are connected to the parent molecular moiety through any carbon atom or any nitrogen atom contained within the rings, and can be unsubstituted or substituted. In some embodiments, the aromatic bicyclic ring system or aromatic tricyclic ring system does not contain non-aromatic rings. Thus, if a bicyclic ring system or tricyclic ring system contains a non-aromatic ring, the ring system is a cycloalkyl or heterocyclyl, depending on whether a heteroatom is present in the non-aromatic ring, regardless of the point of attachment to the remainder of the molecule.

In some embodiments, the five membered aromatic monocyclic rings have two double bonds and the six membered aromatic monocyclic rings have three double bonds. In some embodiments, exemplary bicyclic heteroaryl groups are exemplified by a monocyclic heteroaryl ring appended to the parent molecular moiety and fused to a monocyclic cycloalkyl group, as defined herein, a monocyclic aryl group, as defined herein, a monocyclic heteroaryl group, as defined herein, or a monocyclic heterocycle, as defined herein. In some embodiments, the tricyclic heteroaryl groups are exemplified by a monocyclic heteroaryl ring appended to the parent molecular moiety and fused to two of a monocyclic cycloalkyl group, as defined herein, a monocyclic aryl group, as defined herein, a monocyclic heteroaryl group, as defined herein, or a monocyclic heterocycle, as defined herein.

The terms “heterocyclyl,” “heterocycle,” or “heterocyclic” as used herein, refers to a non-aromatic ring system containing 3 to 10, or 3 to 8, or 3 to 6, or 5 to 10, carbon atoms and at least one (e.g., 1-5, 1-4, 1-3, 1-2, or 1) heteroatom, and optionally one or more oxo and/or double bonds. The terms “heterocyclyl”, “heterocycle” or “heterocyclic” include monocyclic, bicyclic, tricyclic, fused, spirocyclic, or bridged ring systems, provided that at least one non-aromatic ring system containing at least one heteroatom is present. In some embodiments, the monocyclic heterocycle is a three-, four-, five-, six-, seven-, or eight-membered ring containing at least one heteroatom independently selected from the group consisting of O, N, and S. In some embodiments, the three- or four-membered ring contains zero or one double bond, and one heteroatom selected from the group consisting of O, N, and S. In some embodiments, the five-membered ring contains zero or one double bond and one, two or three heteroatoms selected from the group consisting of O, N and S. In some embodiments, the six-membered ring contains zero, one or two double bonds and one, two, or three heteroatoms selected from the group consisting of O, N, and S. In some embodiments, the seven- and eight-membered rings contains zero, one, two, or three double bonds and one, two, or three heteroatoms selected from the group consisting of O, N, and S. Representative examples of monocyclic heterocycles include, but are not limited to, azetidinyl, azepanyl, aziridinyl, diazepanyl, 1,3-dioxanyl, 1,3-dioxolanyl, 1,3-dithiolanyl, 1,3-dithianyl, 1,3-dimethylpyrimidine-2,4(1H,3H)-dione, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, oxetanyl, piperazinyl, piperidinyl, pyranyl, pyrazolinyl, pyrazolidinyl, pyrrolinyl, pyrrolidinyl, tetrahydrofuranyl, tetrahydropyranyl, tetrahydropyridinyl, tetrahydrothienyl, thiadiazolinyl, thiadiazolidinyl, 1,2-thiazinanyl, 1,3-thiazinanyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to a phenyl group, or a monocyclic heterocycle fused to a monocyclic cycloalkyl, or a monocyclic heterocycle fused to a monocyclic cycloalkenyl, or a monocyclic heterocycle fused to a monocyclic heterocycle, or a spiro heterocycle group, or a bridged monocyclic heterocycle ring system in which two non-adjacent atoms of the ring are linked by an alkylene bridge of 1, 2, 3, or 4 carbon atoms, or an alkenylene bridge of two, three, or four carbon atoms. Representative examples of bicyclic heterocycles include, but are not limited to, benzopyranyl, benzothiopyranyl, chromanyl, 2,3-dihydrobenzofuranyl, 2,3-dihydrobenzothienyl, 2,3-dihydroisoquinoline, 2-azaspiro[3.3]heptan-2-yl, azabicyclo[2.2.1]heptyl (including 2-azabicyclo[2.2.1]hept-2-yl), 2,3-dihydro-1H-indolyl, isoindolinyl, octahydrocyclopenta[c]pyrrolyl, octahydropyrrolopyridinyl, and tetrahydroisoquinolinyl. Tricyclic heterocycles are exemplified by a bicyclic heterocycle fused to a phenyl group, or a bicyclic heterocycle fused to a monocyclic cycloalkyl, or a bicyclic heterocycle fused to a monocyclic cycloalkenyl, or a bicyclic heterocycle fused to a monocyclic heterocycle, or a bicyclic heterocycle in which two non-adjacent atoms of the bicyclic ring are linked by an alkylene bridge of 1, 2, 3, or 4 carbon atoms, or an alkenylene bridge of two, three, or four carbon atoms. Examples of tricyclic heterocycles include, but are not limited to, octahydro-2,5-epoxypentalene, hexahydro-2H-2,5-methanocyclopenta[b]furan, hexahydro-1H-1,4-methanocyclopenta[c]furan, aza-adamantane (1-azatricyclo[3.3.1.1^{3,7}]decane), and oxa-adamantane (2-oxatricyclo[3.3.1.1^{3,7}]decane). The monocyclic, bicyclic, and tricyclic heterocycles are connected to the parent molecular moiety through any carbon atom or any nitrogen atom contained within the rings, and can be unsubstituted or substituted.

The term “hydroxyl” as used herein, means an —OH group.

The term “hydroxyalkyl” as used herein, means an alkyl group, as defined herein, in which one, two, three, four, five, six, seven or eight hydrogen atoms are replaced by a hydroxyl group.

In some instances, the number of carbon atoms in a hydrocarbyl substituent (e.g., alkyl or cycloalkyl) is indicated by the prefix “Cₓ-C_{y}-” or “C_{x-y},” wherein x is the minimum and y is the maximum number of carbon atoms in the substituent. Thus, for example, “C₁-C₃-alkyl” and “C₁₋₃alkyl” refer to an alkyl substituent containing from 1 to 3 carbon atoms. The two conventions “Cₓ-C_{y}-” and “C_{x-y}” are used interchangeably and have the same meaning.

The term “substituted” refers to a group that may be further substituted with one or more non-hydrogen substituent groups. Substituent groups include, but are not limited to, halogen, ═O, ═S, cyano, nitro, fluoroalkyl, alkoxyfluoroalkyl, fluoroalkoxy, alkyl, alkenyl, alkynyl, haloalkyl, haloalkoxy, heteroalkyl, cycloalkyl, cycloalkenyl, aryl, heteroaryl, heterocycle, cycloalkylalkyl, heteroarylalkyl, arylalkyl, hydroxy, hydroxyalkyl, alkoxy, alkoxyalkyl, alkylene, aryloxy, phenoxy, benzyloxy, amino, alkylamino, acylamino, aminoalkyl, arylamino, sulfonylamino, sulfinylamino, sulfonyl, alkylsulfonyl, arylsulfonyl, aminosulfonyl, sulfinyl, —COOH, ketone, amide, carbamate, and acyl.

The term “tetrazine” refers to a substituted or unsubstituted aromatic cyclic group of 2 carbon atoms and 4 nitrogen atoms, having a single ring with three double bonds. Examples of tetrazine groups include 1,2,3,4-tetrazine and 1,2,4,5-tetrazine. As used herein, 1,2,4,5-tetrazine is referred to as a “Tz” group.

The term “selectively delivering” refers to delivering an agent (e.g., a payload) to an organ or tissue (or portion thereof) in need of treatment or diagnosis, without significant binding to other non-target organs or tissues (or portions thereof). In some embodiments, the targeting moieties, or therapeutic targeting moiety, described herein do not themselves have a therapeutic effect, but rather are designed to allow the selective or targeted delivery of a therapeutic agent. However, it may be that the targeting moiety does have a therapeutic effect, and thus, such constructs are not excluded by the present disclosure.

The term “payload” refers to an agent for delivery to a target site in a subject. Payloads include therapeutic agents.

The term “therapeutic agent” refers to an agent capable of treating and/or ameliorating a condition or disease, or one or more symptoms thereof, in a subject. Therapeutic agents of the present disclosure also include prodrug forms of therapeutic agents.

The term “diagnostic agent” refers to agents that assist in diagnosing conditions or diseases. Representative diagnostic agents include imaging agents such as paramagnetic agents, optical probes, radionuclides, and the like. Paramagnetic agents are imaging agents that are magnetic under an externally applied field. Examples of paramagnetic agents include, but are not limited to, iron particles including iron nanoparticles and iron microparticles. Optical probes are fluorescent compounds that can be detected by excitation at one wavelength of radiation and detection at a second, different, wavelength of radiation. Optical probes of the present disclosure include, but are not limited to, Cy5.5, Alexa 680, Cy5, DiD (1,1′-dioctadecyl-3,3,3′,3′-tetramethylindodicarbocyanine perchlorate) and DiR (1,1′-dioctadecyl-3,3,3′,3′-tetramethylindotricarbocyanine iodide). Other optical probes include quantum dots. Radionuclides are elements that undergo detectable radioactive decay. Radionuclides useful in embodiments of the present disclosure include, but are not limited to, ³H, ¹¹C, ¹³N, ¹⁸F, ¹⁹F, ⁶⁰Co, ⁶⁴Cu, ⁶⁷Cu, ⁶⁸Ga, ⁸²Rb, ⁸⁹Zr, ⁹⁰Sr, ⁹⁰Y, ⁹⁹Tc, ^{99m}Tc, ¹¹¹ In, ¹²³I, ¹²⁴I, ¹²⁵I, ¹²⁹I, ¹³¹I, ¹³⁷Cs, ¹⁷⁷Lu, ¹⁸⁶Re, ¹⁸⁸Re, ²¹¹At, Rn, Ra, Th, U, Pu, and ²⁴¹Am.

The term “targeting agent” refers to a chemical or biological agent that specifically binds to a target (e.g., a targeted organ or tissue), thereby forming a stable association between the targeting agent and the specific target. By “stably associated” or “stable association” is meant that a moiety is bound to or otherwise associated with another moiety or structure under standard physiological conditions. Bonds may include covalent bonds and non-covalent interactions, such as, but not limited to, ionic bonds, hydrophobic interactions, hydrogen bonds, van der Waals forces (e.g., London dispersion forces), dipole-dipole interactions, and the like. A targeting agent may be a member of a specific binding pair, such as, but are not limited to: a member of a receptor/ligand pair; a ligand-binding portion of a receptor; a member of an antibody/antigen pair; an antigen-binding fragment of an antibody; a hapten; a member of a lectin/carbohydrate pair; a member of an enzyme/substrate pair; biotin/avidin; biotin/streptavidin; digoxin/antidigoxin; a member of a DNA or RNA aptamer binding pair; a member of a peptide aptamer binding pair; and the like. Targeting agents include ligands that specifically bind (or substantially specifically bind) a particular clinically-relevant target receptor or cell surface target. The ligand can be an antibody, peptide, nucleic acid, phage, bacteria, virus, or other molecule with a specific affinity for a target receptor or cell surface target. Examples of receptors and cell surface targets include, but are not limited to, PD-1, CTLA-4, HER2/neu, HER1/EGFR, VEGFR, 4-1BB, GITR, LT4-human mAb directed against the inhibitory immune checkpoint receptor immunoglobulin-like transcript 4 (ILT4; leukocyte immunoglobulin-like receptor subfamily B member 2, LILRB2, lymphocyte immunoglobulin-like receptor 2, LIR2, monocyte/macrophage immunoglobulin-like receptor 10, MIR-10, CD85d, or other cellular receptors or cell surface targets. Additional examples are included in various embodiments disclosed herein.

The term “targeted organ or tissue” refers to an organ or tissue that is being targeted for delivery of the payload. Representative organs and tissues for targeting include those that can be targeted by chemical or biological targeting agents, as well as those organs and tissues that cannot be targeted by chemical or biological targeting agents.

The term “implanting” refers to surgical implantation into a subject's body.

The term “contacting” or “contact” refers to the process of bringing into contact at least two distinct species such that they can interact with each other, such as in a non-covalent or covalent binding interaction or binding reaction. It should be appreciated, however, the resulting complex or reaction product can be produced directly from an interaction or a reaction between the added reagents or from an intermediate from one or more of the added reagents or moieties, which can be produced in the contacting mixture.

The term “binding agent” refers to an agent having a functional group capable of forming a covalent bond to a complementary functional group of another binding agent in a biological environment. Binding between binding agents in a biological environment may also be referred to as bioconjugation. Binding agents include bioorthogonal binding agents, which are binding agents having bioorthogonal functional groups. Bioorthogonal functional groups of bioorthogonal binding agents selectively react with a complementary bioorthogonal functional group of another bioorthogonal binding partner. Selective reaction between bioorthogonal binding partners can minimize side reactions with other binding agents, biological compounds, or other non-complementary bioorthogonal binding agents or non-complementary bioorthogonal functional groups. Bioorthogonal moieties or functional groups of bioorthogonal binding agents include, but are not limited to, an azide and alkyne for formation of a triazole via Click-chemistry reactions, trans-cyclooctene (TCO) and tetrazine (Tz) (e.g., 1,2,4,5-tetrazine), and others. The binding agents useful in the present disclosure may have a high reactivity with the corresponding binding agent so that the reaction is rapid.

The term “functionalized” refers to a moiety having a functional group attached to the moiety, such as for example a moiety having a binding agent functional group (e.g., a bioorthogonal functional group) attached thereto.

The term “administering” refers to any suitable route of administration to a subject, such as, but not limited to, oral administration, administration as a suppository, topical contact, parenteral, intravenous, intraperitoneal, intramuscular, intralesional, intranasal or subcutaneous administration, intrathecal administration, or the implantation of a slow-release device, e.g., a mini-osmotic pump, to the subject.

The term “parenterally,” as used herein, refers to modes of administration which include intravenous, intramuscular, intraperitoneal, intrasternal, subcutaneous and intraarticular injection and infusion.

The term “leaving group” refers to an atom (or a group of atoms) with electron withdrawing ability that can be displaced as a stable species, taking with it the bonding electrons. Examples of suitable leaving groups include halides (e.g., Br, Cl, I), sulfonate esters (e.g., triflate, mesylate, tosylate, and brosylate), and nitrophenols.

The term “pharmaceutically effective amount” and “therapeutically effective amount” refer to an amount of a compound sufficient to treat a specified disorder or disease or one or more of its symptoms and/or to prevent or reduce the risk of the occurrence or reoccurrence of the disease or disorder or symptom(s) thereof. In reference to tumorigenic proliferative disorders, a pharmaceutically or therapeutically effective amount comprises an amount sufficient to, among other things, cause the tumor to shrink or decrease the growth rate of the tumor.

As used herein, the term “subject,” “patient,” or “organism” includes humans and mammals (e.g., mice, rats, pigs, cats, dogs, and horses). Typical subjects to which an agent(s) of the present disclosure may be administered may include mammals, particularly primates, especially humans. For veterinary applications, suitable subjects may include, for example, livestock such as cattle, sheep, goats, cows, swine, and the like; poultry such as chickens, ducks, geese, turkeys, and the like; and domesticated animals particularly pets such as dogs and cats. For diagnostic or research applications, suitable subjects may include mammals, such as rodents (e.g., mice, rats, hamsters), rabbits, primates, and swine such as inbred pigs and the like.

The term “treating” or “treatment” as used herein means the treating or treatment of a disease or medical condition or symptom(s) thereof in a patient, such as a mammal (particularly a human) that includes: (a) ameliorating the disease or medical condition or symptom(s) thereof, such as, eliminating or causing regression of the disease or medical condition or symptom(s) thereof in a patient; (b) suppressing the disease or medical condition or symptom(s) thereof, for example by, slowing or arresting the development of the disease or medical condition or symptom(s) thereof in a patient; or (c) alleviating a symptom of the disease or medical condition or symptom(s) thereof in a patient.

The term “physiological conditions” is meant to encompass those conditions compatible with living cells, e.g., predominantly aqueous conditions of a temperature, pH, salinity, etc. that are compatible with living cells.

For compounds described herein, groups and substituents thereof may be selected in accordance with permitted valence of the atoms and the substituents, such that the selections and substitutions result in a stable compound, e.g., which does not spontaneously undergo transformation such as by rearrangement, cyclization, elimination, etc.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The compounds may exist as stereoisomers wherein asymmetric or chiral centers are present. The stereoisomers are “R” or “S” depending on the configuration of substituents around the chiral carbon atom. The terms “R” and “S” used herein are configurations as defined in IUPAC 1974 Recommendations for Section E, Fundamental Stereochemistry, in Pure Appl. Chem., 1976, 45:13-30. The disclosure contemplates various stereoisomers and mixtures thereof, and these are specifically included within the scope of this disclosure. Stereoisomers include enantiomers and diastereomers and mixtures of enantiomers or diastereomers. Individual stereoisomers of the compounds may be prepared synthetically from commercially available starting materials, which contain asymmetric or chiral centers or by preparation of racemic mixtures followed by methods of resolution well-known to those of ordinary skill in the art. These methods of resolution are exemplified by (1) attachment of a mixture of enantiomers to a chiral auxiliary, separation of the resulting mixture of diastereomers by recrystallization or chromatography, and optional liberation of the optically pure product from the auxiliary as described in Furniss, Hannaford, Smith, and Tatchell, “Vogel's Textbook of Practical Organic Chemistry,” 5^{th} edition (1989), Longman Scientific & Technical, Essex CM20 2JE, England, or (2) direct separation of the mixture of optical enantiomers on chiral chromatographic columns, or (3) fractional recrystallization methods.

It should be understood that the compounds may possess tautomeric forms as well as geometric isomers, and that these also constitute an aspect of the disclosure.

The present disclosure also includes isotopically-labeled compounds, which are identical to those recited herein, but for the fact that one or more atoms are replaced by an atom having an atomic mass or mass number different from the atomic mass or mass number usually found in nature. Examples of isotopes suitable for inclusion in the compounds of the disclosure are hydrogen, carbon, nitrogen, oxygen, phosphorus, sulfur, fluorine, and chlorine, such as, but not limited to, ²H, ³H, ¹³C, ¹⁴C, ¹⁵N, ¹⁸O, ¹⁷O, ³¹P, ³²P, ³⁵S, ¹⁸F, and ³⁶Cl, respectively. Substitution with heavier isotopes such as deuterium, i.e., ²H, can afford certain therapeutic advantages resulting from greater metabolic stability, for example increased in vivo half-life or reduced dosage requirements, and, hence, may be preferred in some circumstances. The compound may incorporate positron-emitting isotopes for medical imaging and positron-emitting tomography (PET) studies for determining the distribution of receptors. Suitable positron-emitting isotopes that can be incorporated are ¹¹C, ¹³N, ¹⁵O, and ¹⁸F. Isotopically-labeled compounds disclosed herein can generally be prepared by conventional techniques known to those skilled in the art or by processes analogous to those described in the accompanying Examples using appropriate isotopically-labeled reagent in place of non-isotopically-labeled reagent.

### B. Targeting Moieties

Provided herein are targeting moieties which comprise a biocompatible support, an antibody or antibody fragment moiety, or in certain embodiments an antibody or antibody fragment moiety, covalently bonded to one or more tetrazine moieties. The targeting moieties described herein are designed to, once administered to a subject, localize at a target site within the subject. The targeting moieties can be administered locally or systemically. In some embodiments, the targeting moiety is a therapeutic targeting moiety. Once administered, a prodrug comprising a complimentary bioorthogonal component (i.e., a trans-cyclooctene moiety) can be administered, which when in contact with the targeting moiety in vivo, allows for targeted drug delivery of a payload or therapeutic agent. In some embodiments, the targeting moieties described herein comprise a diagnostic agent such that the targeting moieties described herein can be used in diagnosing conditions or diseases, with or without administering a payload or therapeutic agent.

Provided herein is a targeting moiety of Formula I, Formula II, or Formula V:

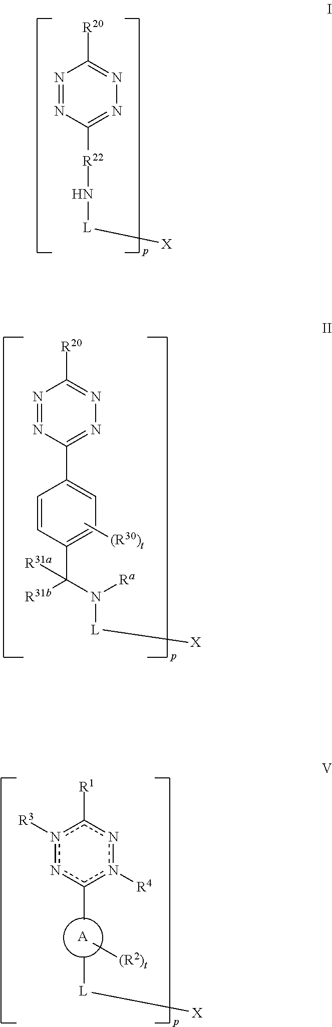

wherein:
ring A is aryl, cycloalkyl, heterocyclyl, or heteroaryl;
the dotted lines represent additional bonds to form a tetrazine when R³ and R⁴ are both absent, or a dihydrotetrazine when R³ and R⁴ are both present; provided that when ring A is aryl, then R³ and R⁴ are both present;
X is a biocompatible support, antibody, or antibody fragment moiety; provided that for Formula I and Formula II, X is not a biocompatible support;
p is 1-150;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R′″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or R³ and R⁴ are each independently hydrogen or a group capable of being removed after a triggering event;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R²², at each occurrence, is independently a linker of 1 to 100 linking atoms optionally comprising one or more ethylene-oxy, amine, ester, amide, carbamate, carbonate, or ketone functional group;
R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl;
R^{a}, R^{31a} and R^{31b} are each independently hydrogen, C₁-C₆-alkyl, or C₁-C₆-haloalkyl;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl; and
t, at each occurrence, is independently is 0, 1, 2, 3, or 4.

### Compounds of Formula I, II, IIA, IIB, IIC, and III:

In one embodiment, provided is a targeting moiety of Formula I:

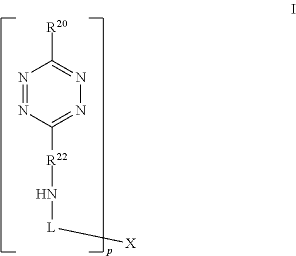

wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R²², at each occurrence, is independently a linker of 1 to 100 linking atoms optionally comprising one or more ethylene-oxy, amine, ester, amide, carbamate, carbonate, or ketone functional group;
R′ and R″, at each occurrence, are independently selected from hydrogen, aryl, and alkyl; and
R′″ at each occurrence is independently selected from aryl and alkyl.

In one embodiment, provided is a targeting moiety of Formula II:

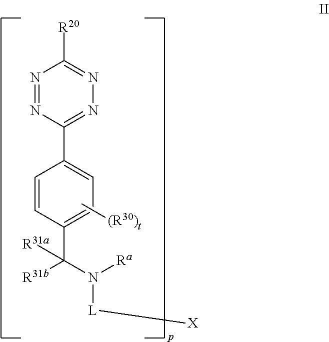

wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl;
R^{a}, R^{31a} and R^{31b} are each independently hydrogen, C₁-C₆-alkyl, or C₁-C₆-haloalkyl;
R′ and R″, at each occurrence, are independently selected from hydrogen, aryl, and alkyl;
R″, at each occurrence, is independently selected from aryl and alkyl; and
t is independently is 0, 1, 2, 3, or 4.

In one embodiment, R²², at each occurrence, is independently a linker of 1 to 100 linking atoms, and can include ethylene-oxy groups, amines, esters, amides, carbamates, carbonates, and ketone functional groups.

In one embodiment, provided is a targeting moiety of Formula IIA:

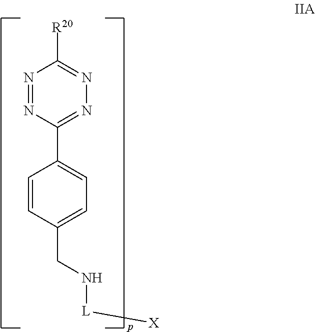

wherein L, p, X, and R²⁰ are each independently as defined herein.

In one embodiment, provided is a targeting moiety of Formula IIB:

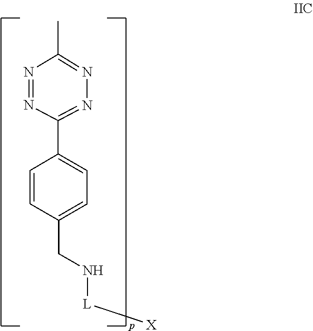

wherein L, p, and X are each independently as defined herein.

In one embodiment, provided is a targeting moiety of Formula IIC:

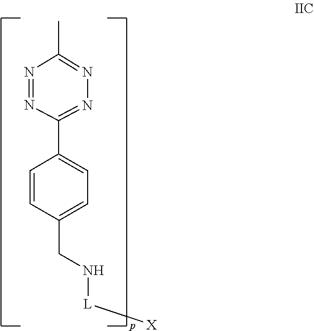

wherein L, p, and X are each independently as defined herein.

In one embodiment, provided is a targeting moiety of Formula III:

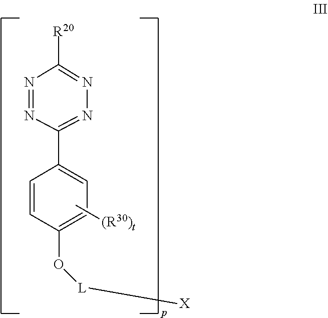

wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;

R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl;
R′ and R″, at each occurrence, are independently selected from hydrogen, aryl, and alkyl;
R′″, at each occurrence, is independently selected from aryl and alkyl; and
t is independently is 0, 1, 2, 3, or 4.

In some embodiments, provided is a targeting moiety represented by Formula IID:

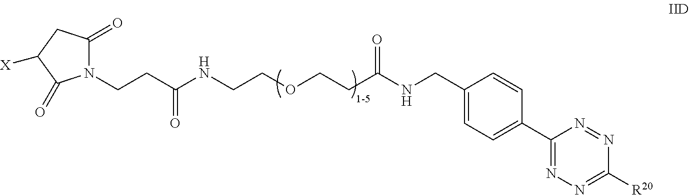

wherein X and R²⁰ are each independently as defined herein. In some embodiments, R²⁰ is methyl. In some embodiments, X is an antigen-binding protein. In some embodiments, X is an antigen-binding protein which targets HER2.

Also provided is a therapeutic moiety of Formula IIE:

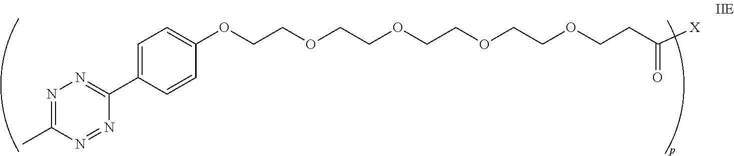

wherein p and X are each independently as defined herein.

Also provided is a targeting moiety of Formula IIF:

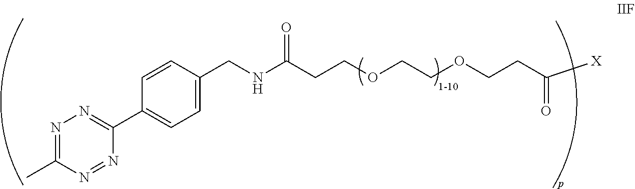

wherein p and X are each independently as defined herein.

Also provided is a targeting moiety of Formula IIG:

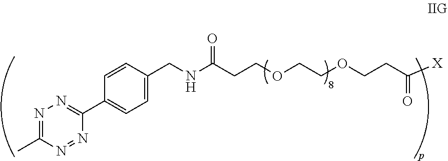

wherein p and X are each independently as defined herein.

In some embodiments of Formula IIA, at least one of:

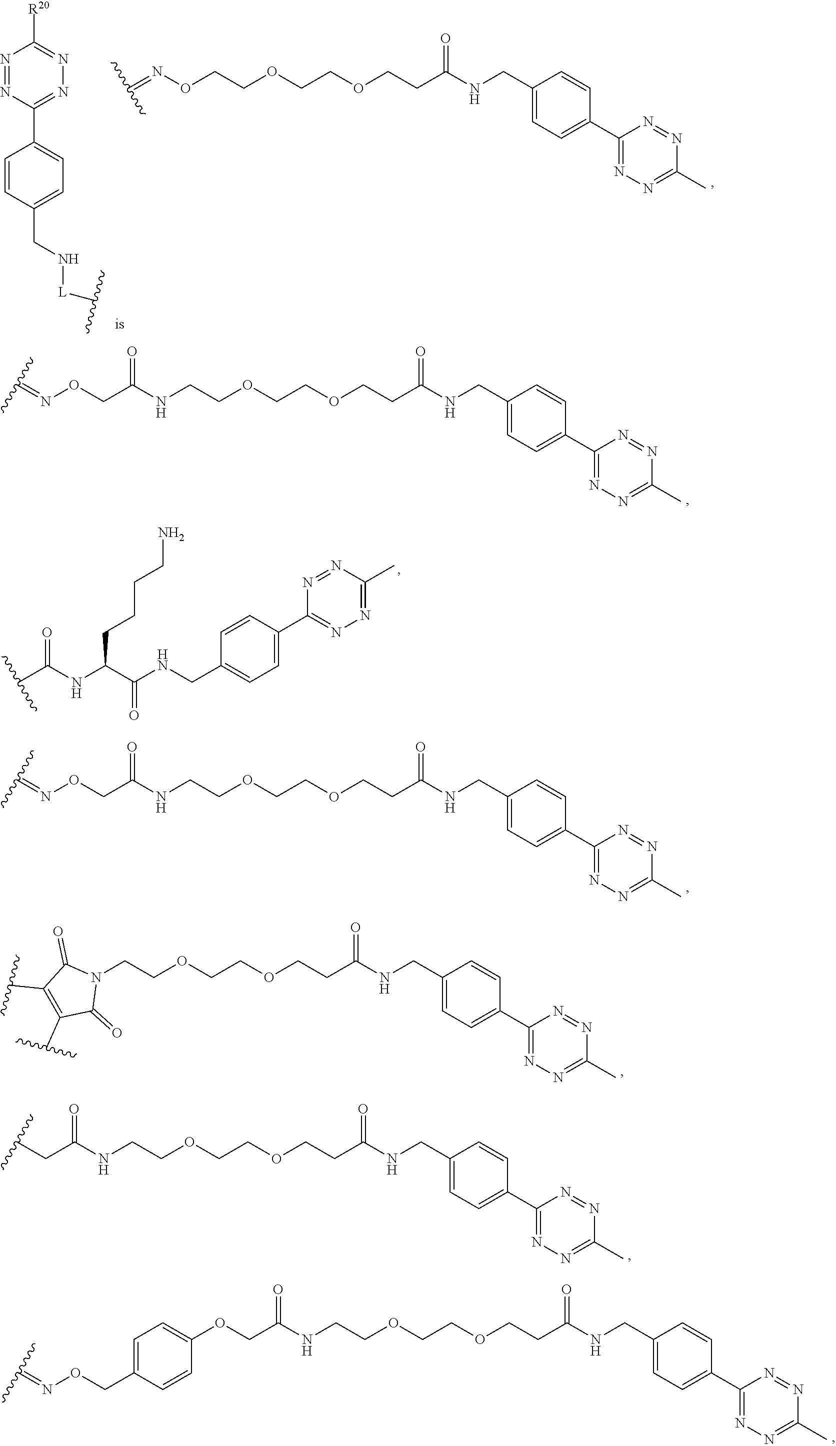

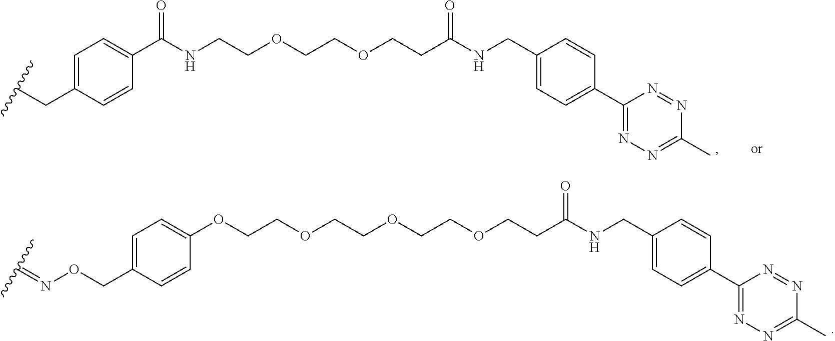 [SMILES: *Cc1ccc(C(=O)NCCOCCOCCC(=O)NCc2ccc(-c3nnc(C)nn3)cc2)cc1 ; *=NOCc1ccc(OCCOCCOCCOCCC(=O)NCc2ccc(-c3nnc(C)nn3)cc2)cc1]

In some embodiments of Formula IIA, at least one of:

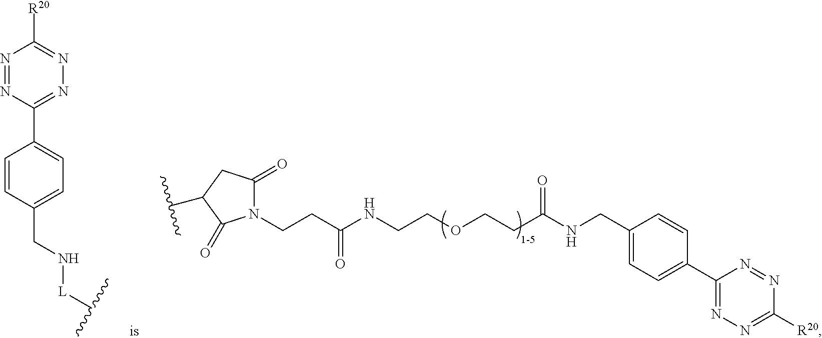

where R²⁰ is as defined herein.

In some embodiments of Formula IIA, at least one of:

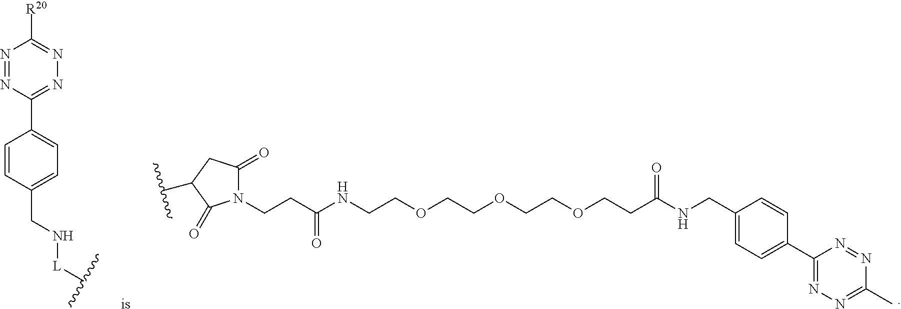

In some embodiments of Formula IIA, at least one of:

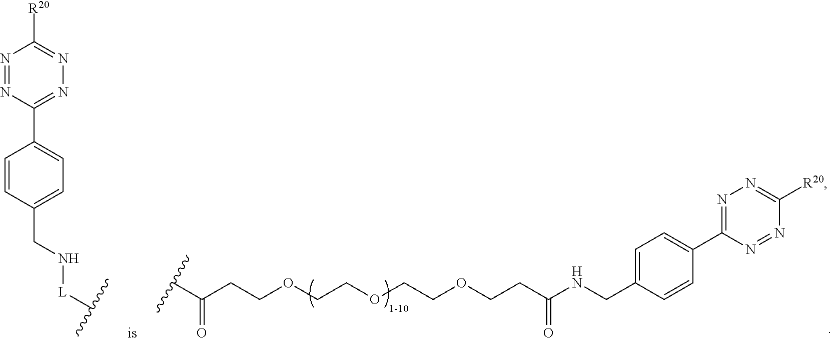

where R²⁰ is as defined herein.

In some embodiments of Formula IIA, at least one of:

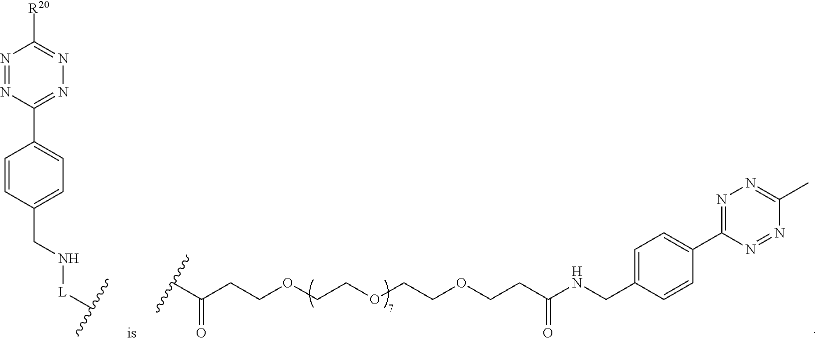

In some embodiments, p is 1-12. In some embodiments, X is an antibody. In some embodiments, p is 1-6, or 5-6. 2. In some embodiments, p is 1-16, or 1-8, or 1-7, or 1-6, or 1-5, or 1-4, or 1-3, or 1-2. In some embodiments, X is an antibody fragment moiety (e.g., Fab).

### Compounds of Formula V, VI, and VII:

In one embodiment, Provided herein is a targeting moiety of Formula V:

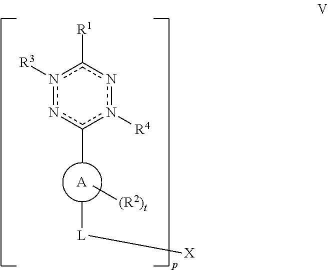

wherein:
ring A is aryl, cycloalkyl, heterocyclyl, or heteroaryl;
the dotted lines represent additional bonds to form a tetrazine when R³ and R⁴ are both absent, or a dihydrotetrazine when R³ and R⁴ are both present; provided that when ring A is aryl, then R³ and R⁴ are both present;
X is a biocompatible support, antibody, or antibody fragment moiety;
p is 1-150;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or R³ and R⁴ are each independently hydrogen or a group capable of being removed after a triggering event;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl; and
t, at each occurrence, is independently is 0, 1, 2, 3, or 4.

Provided herein is a targeting moiety of Formula V:

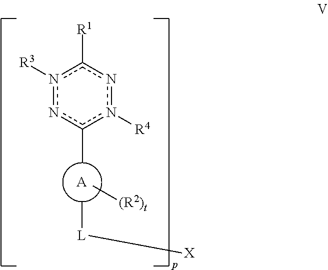

wherein:
ring A is cycloalkyl, heterocyclyl, or heteroaryl;
the dotted lines represent additional bonds to form a tetrazine when R³ and R⁴ are both absent, or a dihydrotetrazine when R³ and R⁴ are both present;
X is a biocompatible support, antibody, or antibody fragment moiety;
p is 1-150;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or
R³ is a group capable of being removed after a triggering event;
R⁴ is hydrogen or R³;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl; and
t, at each occurrence, is independently is 0, 1, 2, 3, or 4.

In some embodiments, provided is a targeting moiety of Formula VI:

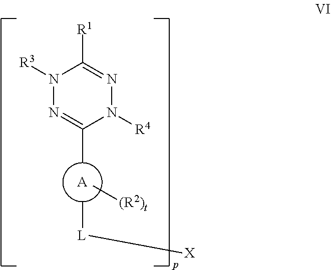

wherein each of R¹, R², R³, R⁴, ring A, L, p, t, and X are independently as defined herein.

In some embodiments, R⁴ is hydrogen.

In the targeting moieties described herein, R³ is a group capable of being removed after a triggering event. In some embodiments, the triggering event occurs in vivo. Once the triggering event occurs and R³ is removed, the dihydrotetrazine moiety is oxidized to provide a tetrazine as in Formula VII:

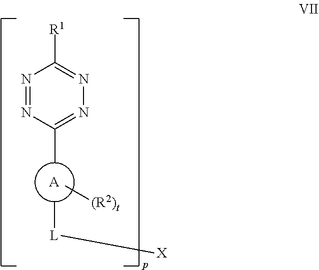

wherein each of R¹, R², ring A, L, p, t, and X are independently as defined herein.

The triggering event is initiated after administration of the targeting moiety to the subject, and can be initiated by any means, such as internal means (e.g., via enzymatic cleavage of a functional group, optionally followed by a decomposition) or by external means (e.g., photocleavable linkers). In some embodiments, R³ comprises a targeting moiety, such as an antibody or antibody fragment as described herein.

In some embodiments, R³ comprises an amino acid sequence specific for cleavage by a protease or esterase.

In some embodiments, R³ comprises an amino acid sequence specific for cleavage by a protease as shown in Table 1A.

TABLE 1A

| Class of Proteases | Protease |
|---|---|
| Metalloproteinases | Meprin |
| | Neprilysin (CD10) |
| | PSMA |
| | BMP-1 |
| | A disintegrin and metalloproteinases (ADAMs) |
| | ADAM8 |
| | ADAM9 |
| | ADAM10 |
| | ADAM12 |
| | ADAM15 |
| | ADAM17 (TACE) |
| | ADAM19 |
| | ADAM28 (MDC-L) |
| | ADAM with thrombospondin motifs (ADAMTS) |
| | ADAMTS1 |
| | ADAMTS4 |
| | ADAMTS5 |
| | Matric Metalloproteinases (MMPs) |
| | MMP-1 (Collagenase 1) |
| | MMP-2 (Gelatinase A) |
| | MMP-3 (Stromelysin 1) |
| | MMP-7 (Matrilysin 1) |
| | MMP-8 (Collagenase 2) |
| | MMP-9 (Gelatinase B) |
| | MMP-10 (Stromelysin 2) |
| | MMP-11 (Stromelysin 3) |
| | MMP-12 (Macrophage elastase) |
| | MMP-13 (Collagenase 3) |
| | MMP-14 (MT1-MMP) |
| | MMP-15 (MT2-MMP) |
| | MMP-19 |
| | MMP-23 (CA-MMP) |
| | MMP-24 (MT5-MMP) |
| | MMP-26 (Matrilysin 2) |
| | MMP-27 (CMMP) |
| Cysteine Proteases | Legumain |
| | Cysteine Cathepsins |
| | Cathepsin B |
| | Cathepsin C |
| | Cathepsin K |
| | Cathepsin L |
| | Cathepsin S |
| | Cathepsin X |
| Aspartate Proteases | Cathepsin D |
| | Cathepsin E |
| | Secretase |
| Serine Proteases | Urokinase (uPA) |
| | Tissue-type plasminogen activator (tPA) |
| | Plasmin |
| | Thrombin |
| | Prostate-specific antigen (PSA, KLK3) |
| | Human neutrophil elastase (HNE) |
| | Elastase |
| | Tryptase |
| | Type II transmembrane serine proteases (TTSPs) |
| | DESC1 |
| | Hepsin (HPN) |
| | Matriptase |
| | Matriptase-2 |
| | TMPRSS2 |
| | TMPRSS3 |
| | TMPRSS4 (CAP2) |
| | Fibroblast Activation Protein (FAP) |
| | kallikrein-related peptidase (KLK family) |
| | KLK4 |
| | KLK5 |
| | KLK6 |
| | KLK7 |
| | KLK8 |
| | KLK10 |
| | KLK11 |
| | KLK13 |
| | KLK14 |

In some embodiments, R³ comprises an amino acid sequence specific for cleavage by a cathepsin, matrix metalloprotease (MMP), or PSMA. For example, in some embodiments, R³ comprises Val-Ala, Val-Cit, Ala-Ala, Phe-Lys, Lys-Lys, Phe-Arg, or Gly-Gly-Gly for cleavage by cathepsins. In some embodiments, R³ comprises Ac-γE-PLG-S(OBn)YL, or Ac-PLG-HofOrnL, where Hof is homophenylalanine and Orn is ornithine for cleavage by MMPs. In some embodiments, R³ comprises an amino acid sequence as shown Table 1B.

TABLE 1B

| PCM | Protease Acting | Exemplary | |
|---|---|---|---|
| Designation | Upon Sequence | Cleavage Sequence | Cleavage Sequences* |
| BSRS1 | MMP-2, 7, 9, 14, | LSGR ↓ SDN ↓ HSPLG ↓ | |
| | matriptase, uPA, | LAGS | |
| | legumain | | |
| BSRS2 | MMP-2, 7, 9, 14, | SPLG ↓ LAGSLSGR ↓ SDN ↓ | |
| | matriptase, uPA, | H | |
| | legumain | | |
| BSRS3 | MMP-2, 7, 9, 14, | SPLG ↓ LSGR ↓ SDN ↓ H | |
| | matriptase, uPA, | | |
| | legumain | | |
| BSRS4 | MMP-2, 7, 9, 14, | LAGR ↓ SDN ↓ HSPLG ↓ | |
| | matriptase, uPA, | LAGS | |
| | legumain | | |
| BSRS5 | MMP-2, 7, 9, 14, | LAGR ↓ SDN ↓ HVPLS ↓ | |
| | matriptase, uPA, | LSMG | |
| | legumain | | |
| BSRS6 | MMP-2, 7, 9, 14, | LAGR ↓ SDN ↓ HEPLE ↓ | |
| | matriptase, uPA, | LVAG | |
| | legumain | | |
| RS1 | MMP-2, 7, 9, 14 | SPLG ↓ LAGS | |
| RS2 | MMP-2, 7, 9, 14, | GPLG ↓ LAR ↓ G | |
| | matriptase, uPA, | | |
| | legumain | | |
| RS3 | Matriptase, uPA, | LSGR ↓ SDN ↓ H | |
| | legumain | | |
| RS4 | MMP-2, 14 | GTAH ↓ LMGG | |
| RS5 | MMP-14 | RIGS ↓ LRTA | |
| RS6 | MMP-14 | RIGA ↓ LRTA | |
| RS7 | MMP-14 | RIGW ↓ LRTA | |
| RS8 | MMP-14 | RIGN ↓ LRTA | |
| RS9 | MMP-14 | RIGF ↓ LRTA | |
| RS10 | MMP-14 | RIFF ↓ LRTA | |
| RS11 | MMP-14 | RILF ↓ LRTA | |
| RS12 | MMP-14 | RIYF ↓ LRTA | |
| RS13 | MMP-14 | RIQF ↓ LRTA | |
| RS14 | MMP-14 | EPAA ↓ LMAG | |
| RS15 | MMP-14 | EPAN ↓ LMAG | |
| RS16 | MMP-14 | EPAS ↓ LMAG | |
| RS17 | MMP-14 | EPFH ↓ LMAG | |
| RS18 | MMP-14 | EPWH ↓ LMAG | |
| RS19 | MMP-14 | EPRH ↓ LMAG | |
| RS20 | MMP-7 | VPLS ↓ LFMG | |
| RS21 | MMP-7 | VPLS ↓ LHMG | |
| RS22 | MMP-7 | VPLS ↓ LQAG | |
| RS23 | MMP-2, 7, 9, 14 | VPLS ↓ LTMG | |
| RS24 | MMP-2, 7, 9, 14, | VPLS ↓ LKMG | |
| | matriptase | | |
| RS25 | MMP-2, 7, 9, 14 | VPLS ↓ LSMG | |
| RS26 | MMP-7 | VPLS ↓ LNAG | |
| RS27 | MMP-7 | VPLS ↓ LLMG | |
| RS28 | MMP-7 | EPLE ↓ LPAG | |
| RS29 | MMP-2, 7, 9, 14 | EPLE ↓ LAAG | |
| RS30 | MMP-2, 7, 9 | EPLE ↓ LVAG | |
| RS31 | MMP-7 | EPLE ↓ LSAG | |
| RS32 | MMP-7 | EPLE ↓ LDAG | |
| RS33 | MMP-7 | EPLE ↓ LQAG | |
| RS34 | MMP-2, 7, 9, 14, | EPLE ↓ LRAG | |
| | matriptase | | |
| RS35 | MMP-7 | EPLE ↓ LKAG | |
| RS36 | MMP-2, 7, 9, 14 | EPLE ↓ LIAG | |
| RS37 | Elastase-2 | LGPV ↓ SGVP | -/-/-/VIAT/-/-/-/- |
| RS38 | Granzyme-B | VAGD ↓ SLEE | V/-/-/D/-/-/-/- |
| RS39 | MMP-12 | GPAG ↓ LGGA | G/PA/-/G/L/-/G/- |
| RS40 | MMP-13 | GPAG ↓ LRGA | G/P/-/G/L/-/GA/- |
| RS41 | MMP-17 | APLG ↓ LRLR | -/PS/-/-/LQ/-/LT/- |
| RS42 | MMP-20 | PALP ↓ LVAQ | |
| RS43 | TEV | ENLYFQ ↓ G | ENLYFQ/G/S |
| RS44 | Enterokinase | DDDK ↓ IVGG | DDDK/IVGG |
| RS45 | Protease 3C | LEVLFQ ↓ GP | LEVLFQ/GP |
| | (PreScission ™) | | |
| RS46 | Sortase A | LPKT ↓ GSES | L/P/KEAD/T/G/-/EKS/S |
| RS47 | Trypsin | K ↓ X ** or R ↓ X | K/X or R/X |
| RS48 | Trypsin | R ↓ X ** | SASRSA |
| RS49 | uPA | SGR ↓ SA | S/G/R/SRKA/AGSVR |
| RS50 | tPA | YGR ↓ SA | RYFLI/GA/R/RVAS/AG |
| RS51 | PSA | SSYY ↓ SG | S/S/FY/Y/S/G |
| RS52 | DESC1 | RRAR ↓ VVGG | R/RAL/ALY/R/AV/V/G/G |
| RS53 | Hepsin | RQLR ↓ VVGG | R/RQ/YL/R/V/V/G/G |
| RS54 | Matriptase-2 | RRAR ↓ VVGG | R/R/A/R/AV/V/G/G |
| RS55 | MT-SP1/Matriptase | RQAR ↓ VVGG | R/QR/A/R/AVY/V/G/G |
| RS56 | PSMA | N ↓ γN | NYN |
| RS57 | Cathepsin C | GF ↓ FY | GP/FWR/**/- |
| RS58 | Cathepsin D | F ↓ IK | FL/IV/KE |
| RS59 | Cathepsin E | F ↓ IK | FL/IV/KE |
| RS60 | Cathepsin F | WLR ↓ | WYRNle/L/RKQ |
| RS61 | Cathepsin K | KPR ↓ | KMGH/ILPNle/RKQ |
| RS62 | Cathepsin L | KFR ↓ | RKLnL/FYW/RKQ |
| RS63 | Cathepsin S | RVK ↓ | RPI/VLMnL/RKQ |
| RS64 | Cathepsin V/L2 | PWR ↓ | PNleR/WYF/RKQ |
| RS65 | MMP | PLG ↓ HofOrnL | |
| RS66 | MMP | EPCitF ↓ HofYL | |
| RS67 | MMP-2 | PQG ↓ IAGQ | |
| RS68 | MMP-2 | PQG ↓ IMelG | |
| RS69 | MMP-9 | AALG ↓ NvaP | |
| RS70 | MMP-9 | GPQG ↓ IAGQR | |
| RS71 | MMP-9 | SGKIPRT ↓ ATA | P/R/PSTRA/Hy/ST |
| RS72 | MMP-9 | SGPLF↓ YSVTA | |
| RS73 | MMP-9 | PLR ↓ LSW | |
| RS74 | MMP-9 | GKGPRQ ↓ ITA | |
| RS75 | MMP-9 | SGRR ↓ LIHHT | S/G/R/R/L/IL |
| RS76 | MMP-9 | SGQPHY ↓ LTTA | |
| RS77 | MMP-9 | SG ↓ LKALM | |
| RS78 | MMP-9 | SGFGSRY ↓ LTA | |
| RS79 | MMP-9 | SGLRPAK ↓ STA | |
| RS80 | MMP-9 | LGP ↓ STST | |
| RS81 | MMP-9 | PQG ↓ VR | |
| RS82 | MMP-9 | PSG ↓ LP | P/S/G/L/HyP |
| RS83 | MMP-9 | PAG ↓ VQ | |
| RS84 | MMP-9 | PSG ↓ RD | |
| RS85 | MMP-9 | PPG ↓ IV | P/PG/G/Hy/HyR |
| RS86 | MMP-9 | PEN ↓ FF | |
| RS87 | MMP-9 | PLK ↓ LM | |
| RS88 | MMP-9 | PGA ↓ YH | |
| RS89 | MMP-9 | AIH ↓ IQ | |
| RS90 | MMP-9 | HFF ↓ KN | |
| RS91 | MMP-9 | GLS ↓ LS | |
| RS92 | MMP-9 | ASD ↓ YK | |
| RS93 | MMP-2, MMP-9 | GPLG ↓ MLSQ | P/Hy/G/Hy/HyWR |
| RS94 | MMP-2, MMP-9 | CG ↓ LDD | |
| RS95 | MMP-2, MMP-9, | GPQG ↓ IWGQ | |
| | MT1-MMP | | |
| RS96 | MMP-7 | RPLA ↓ LWRS | |
| RS97 | MMP-7 | GPLG ↓ LARK | |
| RS98 | Hk2 | GKAFR ↓ RL | |
| RS99 | MMP-9, uPA | RPSA ↓ SRSA | |
| RS100 | MMP-2 | PLGLDpaAR | |
| RS101 | MMP-9 | | P/LMVQChaHofNva/G/LI |
| | | | YSFC/ST |
| RS102 | MMP-9 | PChaG ↓ SmcHA | P/LCha/G/LSmc/HW/A |
| RS103 | MMP-13, MMP-8 | PChaGNvaHAdF | |
| RS104 | ADAM10 | PTASA ↓ LKG | P/T/A/AS/A/LFYQ/KRTI/ |
| | | | GAS |
| RS105 | ADAM17 | PRPAA ↓ VKGT | P/HR/P/AS/A/VIL/KRTVI/ |
| | | | GST/TP |
| RS106 | Cathepsin B | V ↓ Cit | |
| RS107 | Cathepsin B | F ↓ K | |
| RS108 | Elastase | AA ↓ PV | |
| RS109 | Cathepsin D | GPIC ↓ FRLG | |
| RS110 | Plasmin | A ↓ FK | |
| RS111 | Legumain | AAN ↓ L | |
| RS112 | Legumain | PTN ↓ | PTAWS/TPASI/N |
| RS113 | Meprin | | ED/GTAV/- |
| RS114 | Meprin A | F ↓ SPFR | SFAMTY/ |
| | | | /SFAMTY/P/PVIGA/- |
| RS115 | Meprin B | E ↓ EEAY | DE/DE/YEFDG/PVIGA/- |
| RS116 | Neprilysin | _{β}-AIA ↓ L | _{β}-A/LI/A/L |
| RS117 | ADAMTS4 | E ↓ VQRKTGT | E/AFVLMY/(-)/RK/--(-)/ST |
| RS118 | ADAMTS4 | DVQE ↓ FRGVTAVIR | |
| RS119 | ADAMTS4 | HNE ↓ FRQRETYMVF | |
| RS120 | ADAMTS4 | KEEE ↓ GLGS | |
| RS121 | ADAMTS4 | GELE ↓ GRGT | |
| RS122 | ADAMTS4 | NITEGE ↓ ARGS | |
| RS123 | ADAMTS4 | TAQE ↓ AGEG | |
| RS124 | ADAMTS4 | VSQE ↓ LGQR | |
| RS125 | ADAMTS4 | PTAQE ↓ AGE | |

↓ indicates cleavage site
Special amino acid abbreviation:
Cit: Citrilline; Cha: β-cyclohexylalanine; Hof: homophenylalanine; Nva: aminosuberic acid; Dpa: D- phenylalanine; Nle: Norleucine; Smc: S-methylcysteine
*the listing of multiple amino acids before, between, or after a slash indicate alternative amino acids that can be substituted at the position; “-“ indicates that any amino acid may be substituted for the corresponding amino acid indicated in the middle column
**x is any L-amino acid other than proline
Hy is any hydrophobic L-amino acid
γ indicates that bond is a gamma carboxy linkage

Additional cleavable groups are described in Choi, et al., Theranostics. 2012; 2 (2): 156-178, in which Table 2 is hereby incorporated by reference.

In some embodiments, R³ is photolabile. In some embodiments, the photolabile group is labile, or decomposes, with exposure to light at a wavelength matched to the absorbance profile of the photolabile group.

In some embodiments, R³ is

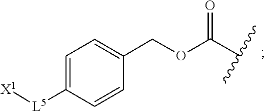

L⁵ is a direct bond or linker; and
X¹ is —NO₂, an optionally substituted sugar moiety, or an optionally substituted peptide unit comprising one or more natural or unnatural amino acids.

In some embodiments, at least one of the moiety:

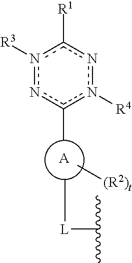

is represented by a formula selected from:

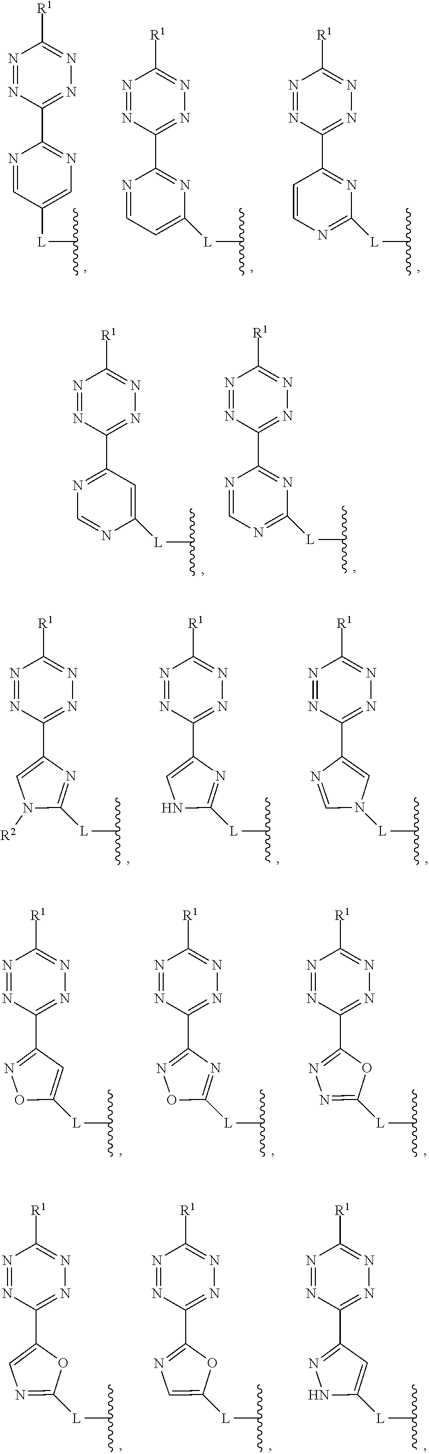

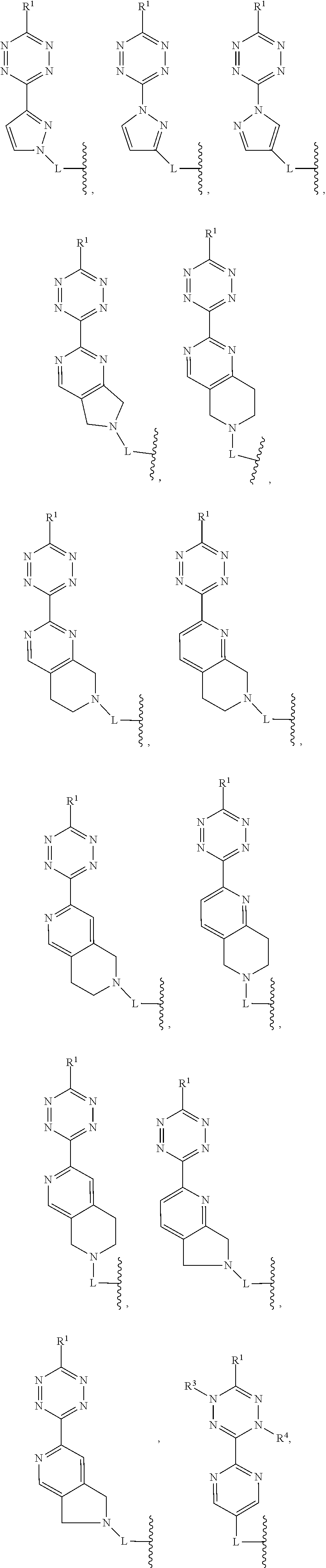

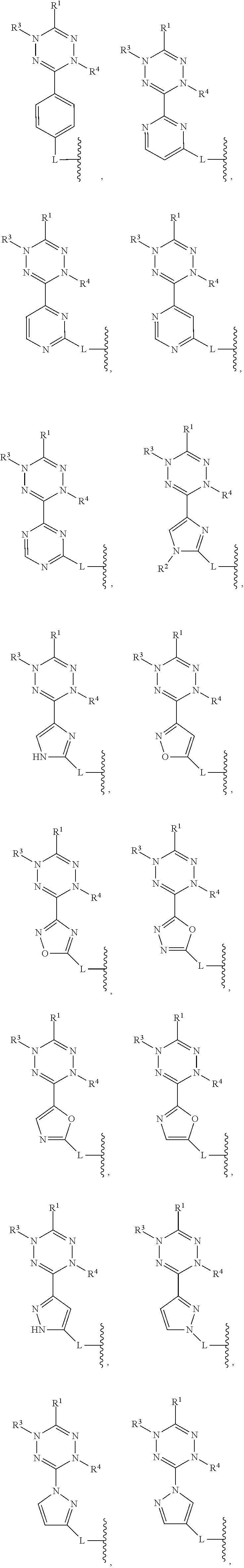

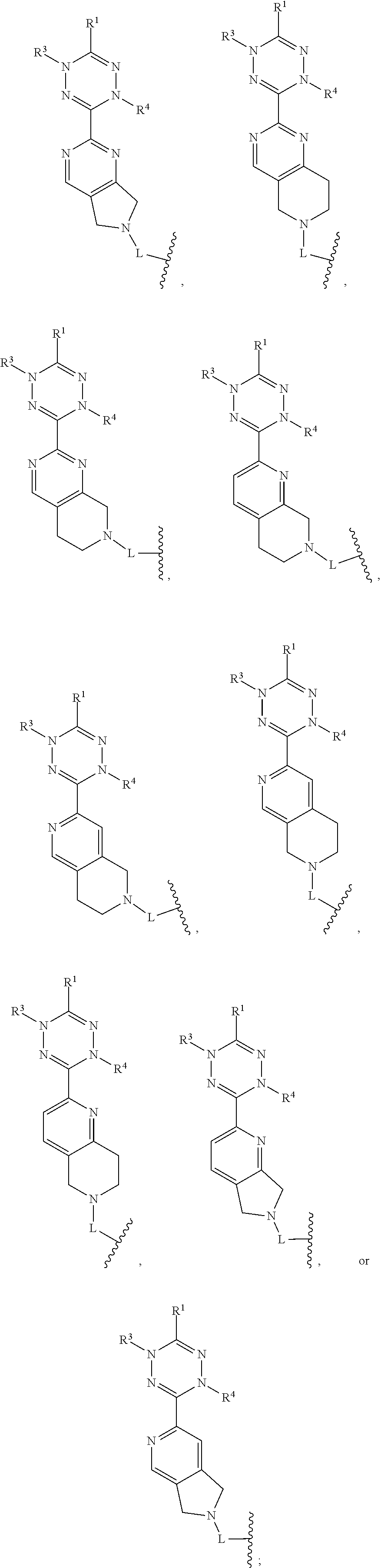

wherein each of R¹, R², R³, and R⁴ are independently as defined herein, and optionally the ring A portion may be substituted with one or more R² moieties.

In some embodiments, at least one of the moiety:

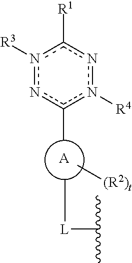

is represented by a formula selected from:

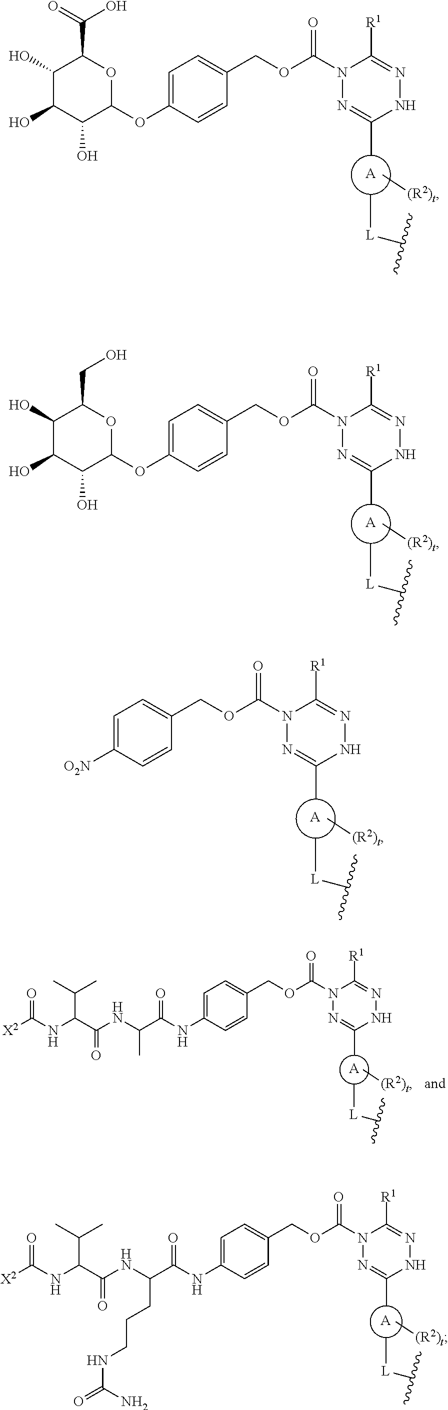

wherein X² is alkyl (e.g., methyl) optionally substituted with a PEG, an amino acid, ester, amide, amine, —C(O)OH, —SO₂, —SO₃, —PO₃, —PO₄, or other solubility enhancing substituent; and each of L, ring A, R¹, R², t, p, and X are independently as defined herein.

In some embodiments, ring A is cycloalkyl. In some embodiments, ring A is heterocyclyl. In some embodiments, ring A is heteroaryl. In some embodiments, ring A is aryl.

In some embodiments, ring A is pyrimidinyl, triazinyl, oxazolyl, isoxazole, imidazolyl, oxadiazolyl, 6,7-dihydro-5H-pyrrolo[3,4-d]pyrimidinyl, 5,6,7,8-tetrahydropyrido[4,3-d]pyrimidinyl, or 5,6,7,8-tetrahydropyrido[3,4-d]pyrimidinyl.

In some embodiments, ring A is phenyl.

In some embodiments, at least one of the moiety:

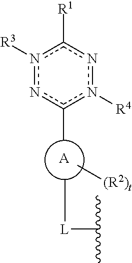

is represented by a formula selected from:

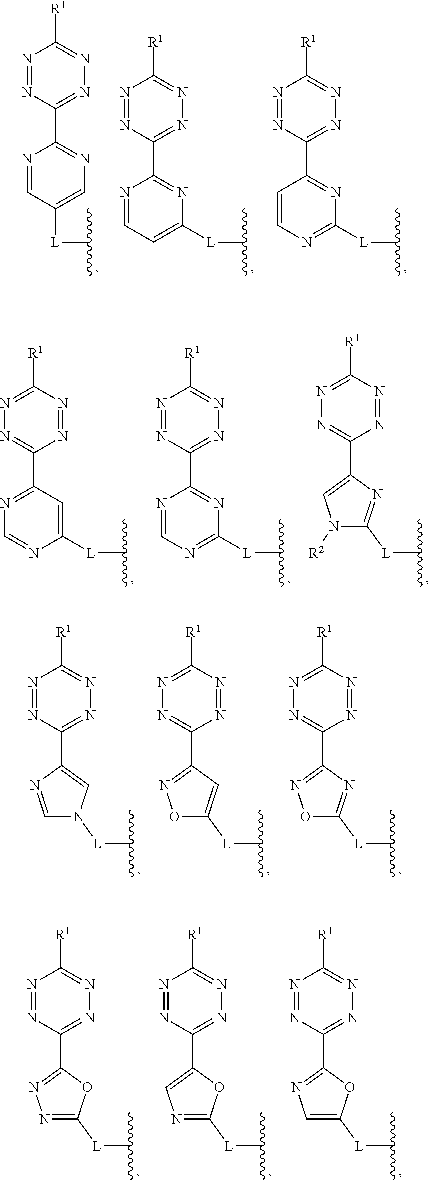

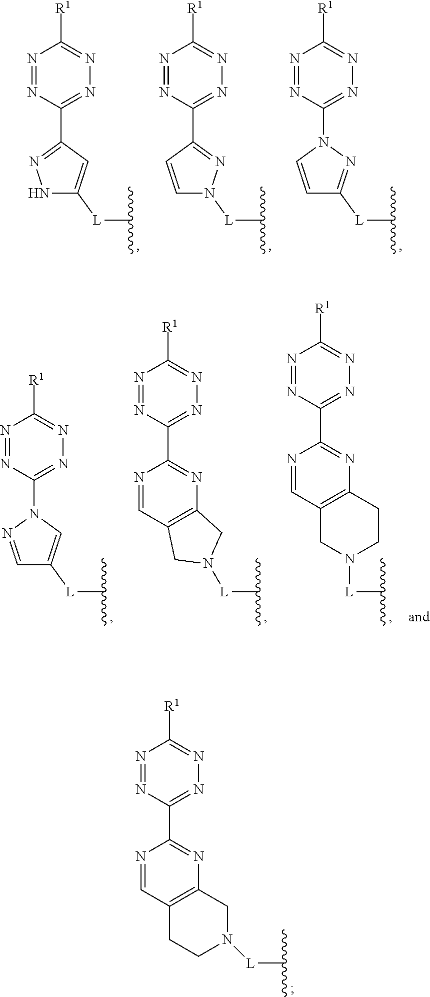

wherein each of R¹ and R² are independently as defined herein.

In some embodiments, R¹, at each occurrence, is independently hydrogen, alkyl, alkenyl, alkynyl, haloalkyl, heteroalkyl, aryl, heteroaryl, heterocyclyl, or cycloalkyl; wherein each alkyl, alkenyl, alkynyl, haloalkyl, heteroalkyl, aryl, heteroaryl, heterocyclyl, or cycloalkyl is optionally substituted with one to three Z¹.

In some embodiments, R¹, at each occurrence, is independently hydrogen or alkyl optionally substituted with one to three Z¹.

In some embodiments, Z¹, at each occurrence, is independently selected from halo, hydroxy, alkoxy, and OC(═O)OR′.

In some embodiments, R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, or cycloalkyl. In some embodiments, R², at each occurrence, is independently halo, alkyl, or haloalkyl. In some embodiments, R², at each occurrence, is independently halo or alkyl.

In some embodiments, t at each occurrence, is 0.

Also provided is a targeting moiety of Formula VA:

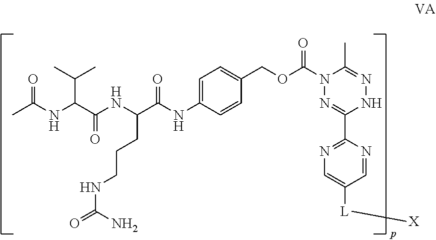

wherein p and X are each independently as defined herein. Also provided is a targeting moiety of Formula VB:

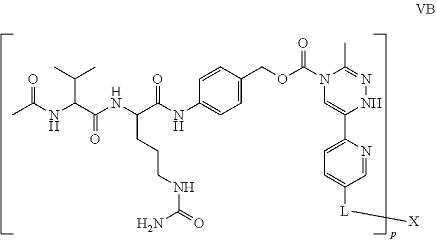

wherein p and X are each independently as defined herein.

In some embodiments, X is a biocompatible support.

In some embodiments, ring A is other than pyridyl. In some embodiments, ring A is other than aryl. In some embodiments, ring A is other than phenyl.

In some embodiments, X is a biocompatible support which comprises a particle, polymer, viscous or non-viscous liquid material, gel, hydrogel, a cross-linked polymer matrix, a metal, a ceramic, a plastic, a bone graft material, or a protein.

In some embodiments, X is a biocompatible support which comprises a polysaccharide hydrogel, alginate, cellulose, hyaluronic acid, chitosan, chitosin, chitin, hyaluronic acid, chondroitin sulfate, heparin, a suitable sugar-based biomaterial, a polyphosphazene, polyanhydride, polyacetal, poly(ortho ester), polyphosphoester, polycaprolactone, polyurethane, polylactide, polycarbonate, polyamide, polyether, a blend/composites/or co-polymer thereof, collagen, gelatin, elastin, an elastin-like polypeptide, albumin, fibrin, poly(gamma-glutamic acid), poly(L-lysine), poly(L-glutamic acid), or poly(aspartic acid). In some embodiments, X is a biocompatible support comprising hyaluronic acid with a molecular weight of about 5-25 kD, or 26-75 kD, or 76-200 kD, or >201 kD.

In some embodiments, X is an antibody or antibody fragment moiety.

### Antibody and Antibody Fragment Moieties

In certain embodiments, the targeting agent, or X, is an antibody, or antibody fragment moiety, that targets one or more of CD25 (NCBI Gene ID 3559), CEA (NCBI Gene ID 634), CEACAM5 (NCBI Gene ID 1048), ASPH (NCBI Gene ID 444), EGFR (NCBI Gene ID 1956), EPCAM (NCBI Gene ID 4072), VEGFR (NCBI Gene ID 3791), PDGFR (NCBI Gene ID 5159), TROP2 (NCBI Gene ID 4070), Nectin4 (NCBI Gene ID 81607), PSMA (NCBI Gene ID 2346), BCMA (NCBI Gene ID 608), CD22 (NCBI Gene ID 933), CD20 (NCBI Gene ID 920), CD19 (NCBI Gene ID 930), CD79b (NCBI Gene ID 974), CD38 (NCBI Gene ID 952), CD45 (NCBI Gene ID 5788), Endoglin (NCBI Gene ID 2022), FGFR2 (NCBI Gene ID 14183), C4.4A (NCBI Gene ID 27076), Claudin-18.2 (NCBI Gene ID 51208), MMP9 (NCBI Gene ID 4318), Folate receptor (NCBI Gene ID 2348), DLL3 (NCBI Gene ID 10683), CD138 (NCBI Gene ID 6382), CD56 (NCBI Gene ID 4684), CD37 (NCBI Gene ID 951), CD74 (NCBI Gene ID 972), mesothelin (NCBI Gene ID 10232), IL-6R (NCBI Gene ID 3570), SLAMF7 (NCBI Gene ID 57823), BAFF (NCBI Gene ID 10673), MUC1 (NCBI Gene ID 4582), GPC3 (NCBI Gene ID 2719), HER2 (NCBI Gene ID 2064), HER3 (NCBI Gene ID 2065), CD30 (NCBI Gene ID 943), CD33 (NCBI Gene ID 945), CD123 (NCBI Gene ID 3563), GPNMB (NCBI Gene ID 10457), cMET (NCBI Gene ID 4233), CD142 (NCBI Gene ID 2152), NaPi2B (NCBI Gene ID 10568), GCC(NCBI Gene ID 2984), STEAP1 (NCBI Gene ID 26872), MUC16 (NCBI Gene ID 94025), CD70 (NCBI Gene ID 970), CD44 (NCBI Gene ID 960), (NCBI Gene ID), Antibody fragments (NCBI Gene ID), vWF (NCBI Gene ID 7450), TNF (NCBI Gene ID 7124), IL-6R (NCBI Gene ID 3570), BCMA (NCBI Gene ID 608), ADAMTS5 (NCBI Gene ID 11096), CX3CR1 (NCBI Gene ID 1524), CXCR4 (NCBI Gene ID 7852), TfR1 (NCBI Gene ID 7037), VEGFR (NCBI Gene ID 3791), or PSMA (NCBI Gene ID 2346).

In some embodiments, X is an antibody or antibody fragment moiety which targets CEA, CEACAM5, ASPH, EGFR, EPCAM, VEGFR, PDGFR, TROP2, Nectin4, PSMA, BCMA, HER2, CD25, CLDN4 (NCBI Gene ID 1364), TNC (NCBI Gene ID 3371), FN1 (NCBI Gene ID 2335), ITGAV (NCBI Gene ID 3685), TACSTD2 (NCBI Gene ID 4070), CD174 (NCBI Gene ID 2525), GPNMB (NCBI Gene ID 10457), GPC1 (NCBI Gene ID 2817), ITGB6 (NCBI Gene ID 3694), SEZ6 (NCBI Gene ID 124925), SLITRK6 (NCBI Gene ID 84189), NaPi-2b (NCBI Gene ID 20531), ZIP6 (NCBI Gene ID 25800), ROR1 (NCBI Gene ID 4919), or ROR2 (NCBI Gene ID 4920).

In certain embodiments, X is is an antibody, or antibody fragment moiety, that targets one or more of CD25 (NCBI Gene ID 3559), CEA (NCBI Gene ID 634), CEACAM5 (NCBI Gene ID 1048), ASPH (NCBI Gene ID 444), EGFR (NCBI Gene ID 1956), EPCAM (NCBI Gene ID 4072), VEGFR (NCBI Gene ID 3791), PDGFR (NCBI Gene ID 5159), TROP2 (NCBI Gene ID 4070), Nectin4 (NCBI Gene ID 81607), PSMA (NCBI Gene ID 2346), BCMA (NCBI Gene ID 608), CD22 (NCBI Gene ID 933), CD20 (NCBI Gene ID 920), CD19 (NCBI Gene ID 930), CD79b (NCBI Gene ID 974), CD38 (NCBI Gene ID 952), CD45 (NCBI Gene ID 5788), Endoglin (NCBI Gene ID 2022), FGFR2 (NCBI Gene ID 14183), C4.4A (NCBI Gene ID 27076), Claudin-18.2 (NCBI Gene ID 51208), MMP9 (NCBI Gene ID 4318), Folate receptor (NCBI Gene ID 2348), DLL3 (NCBI Gene ID 10683), CD138 (NCBI Gene ID 6382), CD56 (NCBI Gene ID 4684), CD37 (NCBI Gene ID 951), CD74 (NCBI Gene ID 972), mesothelin (NCBI Gene ID 10232), IL-6R (NCBI Gene ID 3570), SLAMF7 (NCBI Gene ID 57823), BAFF (NCBI Gene ID 10673), MUC1 (NCBI Gene ID 4582), GPC3 (NCBI Gene ID 2719), HER2 (NCBI Gene ID 2064), HER3 (NCBI Gene ID 2065), CD30 (NCBI Gene ID 943), CD33 (NCBI Gene ID 945), CD123 (NCBI Gene ID 3563), GPNMB (NCBI Gene ID 10457), cMET (NCBI Gene ID 4233), CD142 (NCBI Gene ID 2152), NaPi2B (NCBI Gene ID 10568), GCC(NCBI Gene ID 2984), STEAP1 (NCBI Gene ID 26872), MUC16 (NCBI Gene ID 94025), CD70 (NCBI Gene ID 970), CD44 (NCBI Gene ID 960), (NCBI Gene ID), Antibody fragments (NCBI Gene ID), vWF (NCBI Gene ID 7450), TNF (NCBI Gene ID 7124), IL-6R (NCBI Gene ID 3570), BCMA (NCBI Gene ID 608), ADAMTS5 (NCBI Gene ID 11096), CX3CR1 (NCBI Gene ID 1524), CXCR4 (NCBI Gene ID 7852), TfR1 (NCBI Gene ID 7037), VEGFR (NCBI Gene ID 3791), PSMA (NCBI Gene ID 2346), ANTXR1 (NCBI Gene ID 84168), or FAP (NCBI Gene ID 2191).

In some embodiments, X is an antibody or antibody fragment moiety which targets CEA, CEACAM5, ASPH, EGFR, EPCAM, VEGFR, PDGFR, TROP2, Nectin4, PSMA, BCMA, HER2, CD25, ANTXR1, or FAP.

In some embodiments, X is an antibody or antibody fragment moiety that targets HER2, TROP2, Nectin-4, Claudin-18.2, MMP9, mesothelin, FN1, FAP, TNC, or ECM, EPCAM, CEA, or CEACAM5.

In some embodiments, X is an antibody or antibody fragment moiety which targets CEA, CEACAM5, ASPH, EGFR, EPCAM, VEGFR, PDGFR, TROP2, Nectin4, PSMA, BCMA, HER2, or CD25.

In certain embodiments, X is an antibody, or antibody fragment moiety, that targets CD25, such as daclizumab, RG6292, basiliximab, or HuMax-TAC, or an antibody fragment moiety derived therefrom.

In certain embodiments, X is an antibody, or antibody fragment moiety, that targets CEA, such as labetuzumab, 15-1-32, PR1A3, or cT84.66, or an antibody fragment moiety derived therefrom.

In certain embodiments, X is an antibody, or antibody fragment moiety, that targets CEACAM5, such as Tusamitiamab or CC4, or an antibody fragment moiety derived therefrom.

In certain embodiments, X is an antibody, or antibody fragment moiety, that targets ASPH, such as PAN-622, or an antibody fragment moiety derived therefrom.

In certain embodiments, X is an antibody, or antibody fragment moiety, that targets EGFR, such as cetuximab, necitumumab, nimotuzumab, matuzumab, AMG595, depatuxizumab, dapatuxizumab, duligotuzumab, futuximab, GC1118, imgatuzumab, panitumumab, alutumumab, tomuzotuximab, or laprituximab, or an antibody fragment moiety derived therefrom.

In certain embodiments, X is an antibody, or antibody fragment moiety, that targets EPCAM, such as oportuzumab, citatuzumab, tucotuzumab, catumaxomab, edrecolomab, or adecatumumab, or an antibody fragment moiety derived therefrom.

In certain embodiments, X is an antibody, or antibody fragment moiety, that targets VEGFR, such as ramucizumab, ramucirumab, or vulinacimab, or an antibody fragment moiety derived therefrom.

In certain embodiments, X is an antibody, or antibody fragment moiety, that targets PDGFR, such as olaratumab or ramucirumab, or an antibody fragment moiety derived therefrom.

In certain embodiments, X is an antibody, or antibody fragment moiety, that targets TROP2, such as Sacituzumab or Pr1E11, or an antibody fragment moiety derived therefrom.

In certain embodiments, X is an antibody, or antibody fragment moiety, that targets Nectin4, such as enfortumab, or an antibody fragment moiety derived therefrom.

In certain embodiments, X is an antibody, or antibody fragment moiety, that targets PSMA, such as J591 or MLN591, or an antibody fragment moiety derived therefrom.

In certain embodiments, X is an antibody, or antibody fragment moiety, that targets BCMA, such as Belantamab, or an antibody fragment moiety derived therefrom.

In certain embodiments, X is an antibody, or antibody fragment moiety, that targets CD22, such as moxetumomab, inotuzumab, epratuzumab, or pinatuzumab, or an antibody fragment moiety derived therefrom.

In certain embodiments, X is an antibody, or antibody fragment moiety, that targets CD20, such as ublituximab, ofatumumab, rituximab, obinutuzumab, tositumomab, or ibritumomab, or an antibody fragment moiety derived therefrom.

In certain embodiments, X is an antibody, or antibody fragment moiety, that targets CD19, such as loncastuximab, XMAB-5574, MOR208, coltuximab, denintuzumab, taplitumomab, or MDX-1342, or an antibody fragment moiety derived therefrom.

In certain embodiments, X is an antibody, or antibody fragment moiety, that targets CD79b, such as polatuzumab, or an antibody fragment moiety derived therefrom.

In certain embodiments, X is an antibody, or antibody fragment moiety, that targets CD38, such as isatuximab, daratumumab, MOR202, or TAK-079, or an antibody fragment moiety derived therefrom.

In certain embodiments, X is an antibody, or antibody fragment moiety, that targets CD45, such as I-131-BC8, or Iomab-B, or an antibody fragment moiety derived therefrom.

In certain embodiments, X is an antibody, or antibody fragment moiety, that targets endoglin, such as carotuximab, or an antibody fragment moiety derived therefrom.

In certain embodiments, X is an antibody, or antibody fragment moiety, that targets FGFR2, such as bemarituzumab or aprutumab, or an antibody fragment moiety derived therefrom.

In certain embodiments, X is an antibody, or antibody fragment moiety, that targets C4.4A, such as lupartumab, or an antibody fragment moiety derived therefrom.

In certain embodiments, X is an antibody, or antibody fragment moiety, that targets Claudin-18.2, such as zolbetuximab, or claudiximab, or an antibody fragment moiety derived therefrom.

In certain embodiments, X is an antibody, or antibody fragment moiety, that targets MMP9, such as andecaliximab, or an antibody fragment moiety derived therefrom.

In certain embodiments, X is an antibody, or antibody fragment moiety, that targets folate receptor, such as mirvetuximab, farletuzumab, MORAb-202, MORAb-003, or SP8166, or an antibody fragment moiety derived therefrom.

In certain embodiments, X is an antibody, or antibody fragment moiety, that targets DLL3, such as rovalpituzumab, or an antibody fragment moiety derived therefrom.

In certain embodiments, X is an antibody, or antibody fragment moiety, that targets CD138, such as indatuximab, or an antibody fragment moiety derived therefrom.

In certain embodiments, X is an antibody, or antibody fragment moiety, that targets CD56, such as lorvotuzumab, promiximab, or an antibody fragment moiety derived therefrom.

In certain embodiments, X is an antibody, or antibody fragment moiety, that targets CD37, such as BI 836826, otlertuzumab, or naratuximab, or an antibody fragment moiety derived therefrom.

In certain embodiments, X is an antibody, or antibody fragment moiety, that targets CD74, such as milatuzumab, or an antibody fragment moiety derived therefrom.

In certain embodiments, X is an antibody, or antibody fragment moiety, that targets mesothelin, such as anetumab, amatuximab, or MMOT-0530A, or an antibody fragment moiety derived therefrom.

In certain embodiments, X is an antibody, or antibody fragment moiety, that targets IL-6R, such as tocilizumab or sarilumab, or an antibody fragment moiety derived therefrom.

In certain embodiments, X is an antibody, or antibody fragment moiety, that targets SLAMF7, such as elotuzumab, or an antibody fragment moiety derived therefrom.

In certain embodiments, X is an antibody, or antibody fragment moiety, that targets BAFF, such as belimumab, or an antibody fragment moiety derived therefrom.

In certain embodiments, X is an antibody, or antibody fragment moiety, that targets MUC1, such as KL-6, MY.1E12, hMUC1-1H7, TAB004, huC242, clivatuzumab, 8HuDS6, gatipotuzumab, AR20.5, or cantuzumab, or an antibody fragment moiety derived therefrom.

In certain embodiments, X is an antibody, or antibody fragment moiety, that targets GPC3, such as codrituzumab, ECT204, or MDX-1414, or an antibody fragment moiety derived therefrom.

In certain embodiments, X is an antibody, or antibody fragment moiety, that targets HER2, such as pertuzumab, trastuzumab, or margetuximab, or an antibody fragment moiety derived therefrom.

In certain embodiments, X is an antibody, or antibody fragment moiety, that targets HER3, such as patritumab, seribantumab, lumretuzumab, elgemtumab, AV-203, CDX-3379, or GSK284933, or an antibody fragment moiety derived therefrom.

In certain embodiments, X is an antibody, or antibody fragment moiety, that targets CD30, such as brentuximab, or an antibody fragment moiety derived therefrom.

In certain embodiments, X is an antibody, or antibody fragment moiety, that targets CD33, such as gemtuzumab, BI 835858, vadastuximab, or lintuzumab, or an antibody fragment moiety derived therefrom.

In certain embodiments, X is an antibody, or antibody fragment moiety, that targets CD123, such as KHK2823, taclotuzumab, or G4723A, or an antibody fragment moiety derived therefrom.

In certain embodiments, X is an antibody, or antibody fragment moiety, that targets GPNMB, such as glembatumumab, or an antibody fragment moiety derived therefrom.

In certain embodiments, X is an antibody, or antibody fragment moiety, that targets cMET, such as telisotuzumab, onartuzumab, or SAIT301, or an antibody fragment moiety derived therefrom.

In certain embodiments, X is an antibody, or antibody fragment moiety, that targets CD142, such as tisotumab, or an antibody fragment moiety derived therefrom.

In certain embodiments, X is an antibody, or antibody fragment moiety, that targets NaPi2B, such as lifastuzumab, or an antibody fragment moiety derived therefrom.

In certain embodiments, X is an antibody, or antibody fragment moiety, that targets GCC, such as indusatumab, or an antibody fragment moiety derived therefrom.

In certain embodiments, X is an antibody, or antibody fragment moiety, that targets STEAP1, such as vandortuzumab, or an antibody fragment moiety derived therefrom.

In certain embodiments, X is an antibody, or antibody fragment moiety, that targets MUC16, such as sofituzumab, or an antibody fragment moiety derived therefrom.

In certain embodiments, X is an antibody, or antibody fragment moiety, that targets CD70, such as vorsetuzumab, or an antibody fragment moiety derived therefrom.

In certain embodiments, X is an antibody, or antibody fragment moiety, that targets CD44, such as bivatuzumab, or an antibody fragment moiety derived therefrom.

In certain embodiments, X is an antibody, or antibody fragment moiety, that targets vWF, such as caplacizumab, or an antibody fragment moiety derived therefrom.

In certain embodiments, X is an antibody, or antibody fragment moiety, that targets TNF, such as ozoralizumab, V565, or PF-05230905, or an antibody fragment moiety derived therefrom.

In certain embodiments, X is an antibody, or antibody fragment moiety, that targets IL-6R, such as vobarilizumab, or an antibody fragment moiety derived therefrom.

In certain embodiments, X is an antibody, or antibody fragment moiety, that targets BCMA, such as LCAR-B38M, or an antibody fragment moiety derived therefrom.

In certain embodiments, X is an antibody, or antibody fragment moiety, that targets ADAMTS5, such as M6495, or an antibody fragment moiety derived therefrom.

In certain embodiments, X is an antibody, or antibody fragment moiety, that targets CX3CR1, such as BI 655088, or an antibody fragment moiety derived therefrom.

In certain embodiments, X is an antibody, or antibody fragment moiety, that targets CXCR4, such as AD-214 or ALX-0651, or an antibody fragment moiety derived therefrom.

In certain embodiments, X is an antibody, or antibody fragment moiety, that targets TfR1, such as TXB4, or an antibody fragment moiety derived therefrom.

In certain embodiments, X is an antibody, or antibody fragment moiety, that targets VEGFR, such as CDP791, or an antibody fragment moiety derived therefrom.

In certain embodiments, X is an antibody, or antibody fragment moiety, that targets PSMA, such as GY1, or an antibody fragment moiety derived therefrom.

In certain embodiments, X is an antibody, or antibody fragment moiety, that targets FN1, such as L19 or NJB2, or an antibody fragment moiety derived therefrom.

In certain embodiments, X is an antibody, or antibody fragment moiety, that targets FAP, such as F19, OMTX005 or sibrotuzumab, or an antibody fragment moiety derived therefrom.

In certain embodiments, X is an antibody, or antibody fragment moiety, that targets TNC, such as F16 or R6N or an antibody fragment moiety derived therefrom.

In some embodiments, X is an antibody.

In some embodiments, the antibody is daclizumab, RG6292, basiliximab, HuMax-TAC, labetuzumab, 15-1-32, PR1A3, cT84.66, tusamitiamab, CC4, PAN-622, cetuximab, necitumumab, nimotuzumab, matuzumab, AMG595, depatuxizumab, dapatuxizumab, duligotuzumab, Futuximab, GC1118, imgatuzumab, panitumumab, alutumumab, tomuzotuximab, laprituximab, oportuzumab, citatuzumab, tucotuzumab, catumaxomab, edrecolomab, adecatumumab, ramucizumab, ramucirumab, vulinacimab, olaratumab, ramucirumab, sacituzumab, Pr1E11, enfortumab, J591, MLN591, belantamab, moxetumomab, inotuzumab, epratuzumab, pinatuzumab, ublituximab, ofatumumab, rituximab, obinutuzumab, tositumomab, ibritumomab, loncastuximab, XMAB-5574, MOR208, coltuximab, denintuzumab, taplitumomab, MDX-1342, polatuzumab, Isatuximab, daratumumab, MOR202, TAK-079, I-131-BC8, Iomab-B, carotuximab, bemarituzumab, aprutumab, lupartumab, zolbetuximab, claudiximab, andecaliximab, mirvetuximab, farletuzumab, MORAb-202, MORAb-003, SP8166, rovalpituzumab, indatuximab, lorvotuzumab, promiximab, BI 836826, otlertuzumab, naratuximab, milatuzumab, anetumab, amatuximab, MMOT-0530A, sarilumab, elotuzumab, belimumab, KL-6, MY.1E12, hMUC1-1H7, TAB004, huC242, clivatuzumab, 8HuDS6, gatipotuzumab, AR20.5, cantuzumab, codrituzumab, ECT204, MDX-1414, pertuzumab, trastuzumab, margetuximab, patritumab, seribantumab, lumretuzumab, elgemtumab, AV-203, CDX-3379, GSK284933, brentuximab, gemtuzumab, BI 835858, vadastuximab, lintuzumab, KHK2823, taclotuzumab, G4723A, glembatumumab, telisotuzumab, onartuzumab, SAIT301, tisotumab, lifastuzumab, indusatumab, vandortuzumab, sofituzumab, vorsetuzumab, bivatuzumab, caplacizumab, ozoralizumab, V565, PF-05230905, vobarilizumab, LCAR-B38M, BI 655088, AD-214, ALX-0651, TXB4, CDP791, GY1, L19, NJB2, F19, OMTX005, sibrotuzumab, F16, or R6N.

In some embodiments, X is an antibody selected from atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, and trastuzumab.

In some embodiments, X is an antibody fragment moiety.

In some embodiments, X or the antibody fragment moiety is selected from the group consisting of a single-chain variable fragment (scFv), a divalent (or bivalent) single-chain variable fragment (di-scFvs, bi-scFvs), an antigen-binding fragment (Fab), a single-domain antibody (sdAb), a single-domain antibody (sdAb), an antigen-binding protein, a DotBody, an affibody, a DARPin, a DART, a TandAb, a diabody, a ribobody, a centyrin, a knottin, an affilin, an affimer, an alphabody, an anticalin, an atrimer, an avimer, a fynomer, a kunitz domain, an obody, a pronectin, a repebody, and a bicyclic peptide or a Humabody.

In some embodiments, X is an antibody fragment moiety selected from the group consisting of a single-chain variable fragment (scFv), a divalent (or bivalent) single-chain variable fragment (di-scFvs, bi-scFvs), an antigen-binding fragment (Fab), a single-domain antibody (sdAb), and a single-domain antibody (sdAb).

In some embodiments, the antibody fragment moiety is an antigen-binding protein a DotBody, affibody, DARPin, DART, TandAb, diabody, ribobody, centyrin, knottin, affilin, affimer, alphabody, anticalin, atrimer, avimer, fynomer, kunitz domain, obody, pronectin, repebody, bicyclic peptide or Humabody.

In some embodiments, X or the antibody fragment moiety is an antigen-binding fragment (Fab). The Fab is a region on an antibody that binds to antigens, and is comprised of one constant and one variable domain of each of the heavy and the light chain. In some embodiments, the Fab comprises four domains: VH, CH1, VL and CL1. In some embodiments, the Fab comprises 400-500 amino acids, or 440-480 amino acids. In some embodiments, the Fab has a molecular weight of about 50 kDa, or 40-55 kDa, or 45-50 kDa, or 45-55 kDa.

In some embodiments, the Fab of trastuzumab, enfortumab, brentuximab, sacituzumab, L19-binding to FN-1 (Gene ID 2335); F16-binding to TNC (Gene ID 3371), or NJB2 (ECM-targeting sequences).

In some embodiments, the antibody fragment moiety comprises one or more PEG units, which may enhance circulation life.

In some embodiments, the antibody fragment moiety is an antigen-binding protein. Antigen-binding proteins are proteins which are designed to be antibody-mimetics, exhibiting a high affinity and specificity for a given target. In some embodiments, the antigen-binding protein is a single-chain antigen-binding proteins are novel recombinant polypeptides, composed of an antibody variable light-chain amino acid sequence (VL) tethered to a variable heavy-chain sequence (VH) by a designed peptide that links the carboxyl terminus of the VL sequence to the amino terminus of the VH sequence.

In some embodiments, the antigen-binding protein is about 5-10 kDa, or about 7 kDa. In some embodiments, the antigen-binding protein is about are about 50-80, or 60-70, or 66 amino acids in length. In some embodiments, the antigen-binding protein comprises a cysteine only at the N- or C-terminus. In some embodiments, the antigen-binding protein comprises a cysteine only at the N-terminus. In some embodiments, the antigen-binding protein comprises a cysteine only at the C-terminus.

In some embodiments, the antibody fragment moiety is an antigen-binding protein that targets TNC, FN1, CLDN4, MMP9, EpCAM, ITGAV, CEA, CEACAM5, ASPH, EGFR, EPCAM, VEGFR, PDGFR, TROP2, Nectin4, PSMA, BCMA, HER2, or CD25. In some embodiments, the antibody fragment moiety is an antigen-binding protein that targets HER2. Antigen-binding proteins can be prepared and tested according to standard methods or purchased from commercial sources (e.g., Affilogic).

In some embodiments, the antibody fragment moiety is derived from daclizumab, RG6292, basiliximab, HuMax-TAC, labetuzumab, 15-1-32, PR1A3, cT84.66, tusamitiamab, CC4, PAN-622, cetuximab, necitumumab, nimotuzumab, matuzumab, AMG595, depatuxizumab, dapatuxizumab, duligotuzumab, futuximab, gc1118, imgatuzumab, panitumumab, alutumumab, tomuzotuximab, laprituximab, oportuzumab, citatuzumab, tucotuzumab, catumaxomab, edrecolomab, adecatumumab, ramucizumab, ramucirumab, vulinacimab, olaratumab, ramucirumab, sacituzumab, Pr1E11, enfortumab, J591, MLN591, belantamab, moxetumomab, inotuzumab, epratuzumab, pinatuzumab, ublituximab, ofatumumab, rituximab, obinutuzumab, tositumomab, ibritumomab, loncastuximab, XMAB-5574, MOR208, coltuximab, denintuzumab, taplitumomab, MDX-1342, polatuzumab, isatuximab, daratumumab, MOR202, TAK-079, I-131-BC8, Iomab-B, carotuximab, bemarituzumab, aprutumab, lupartumab, zolbetuximab, claudiximab, andecaliximab, mirvetuximab, farletuzumab, MORAb-202, MORAb-003, SP8166, rovalpituzumab, indatuximab, lorvotuzumab, promiximab, BI 836826, otlertuzumab, naratuximab, milatuzumab, anetumab, amatuximab, MMOT-0530A, sarilumab, elotuzumab, belimumab, KL-6, MY.1E12, hMUC1-1H7, TAB004, huC242, clivatuzumab, 8HuDS6, gatipotuzumab, AR20.5, cantuzumab, codrituzumab, ECT204, MDX-1414, pertuzumab, trastuzumab, margetuximab, patritumab, seribantumab, lumretuzumab, elgemtumab, AV-203, CDX-3379, GSK284933, brentuximab, gemtuzumab, BI 835858, vadastuximab, lintuzumab, KHK2823, taclotuzumab, G4723A, glembatumumab, telisotuzumab, onartuzumab, sait301, tisotumab, lifastuzumab, indusatumab, vandortuzumab, sofituzumab, vorsetuzumab, bivatuzumab, caplacizumab, ozoralizumab, V565, PF-05230905, vobarilizumab, LCAR-B38M, BI 655088, AD-214, ALX-0651, TXB4, CDP791, GY1, L19, NJB2, F19, OMTX005, sibrotuzumab, F16 or R6N.

In some embodiments, X is an antibody fragment moiety derived from atezolizumab, avelumab, bevacizumab, cemiplimab, cetuximab, daratumumab, dinutuximab, durvalumab, elotuzumab, ipilimumab, isatuximab, mogamulizumab, necitumumab, nivolumab, obinutuzumab, ofatumumab, olaratumab, panitumumab, pembrolizumab, pertuzumab, ramucirumab, rituximab, or trastuzumab.

In certain embodiments, X is an antibody, or antibody fragment moiety, that targets vWF, such as Caplacizumab.

In certain embodiments, X is an antibody, or antibody fragment moiety, that targets TNF, such as Ozoralizumab, V565, or PF-05230905.

In certain embodiments, X is an antibody, or antibody fragment moiety, that targets IL-6R, such as Vobarilizumab.

In certain embodiments, X is an antibody, or antibody fragment moiety, that targets BCMA, such as LCAR-B38M.

In certain embodiments, X is an antibody, or antibody fragment moiety, that targets ADAMTS5, such as M6495.

In certain embodiments, X is an antibody, or antibody fragment moiety, that targets CX3CR1, such as BI 655088.

In certain embodiments, X is an antibody, or antibody fragment moiety, that targets CXCR4, such as AD-214 or ALX-0651.

In certain embodiments, X is an antibody, or antibody fragment moiety, that targets TfR1, such as TXB4.

In certain embodiments, X is an antibody, or antibody fragment moiety, that targets VEGFR, such as CDP791.

In certain embodiments, X is an antibody, or antibody fragment moiety, that targets PSMA, such as GY1.

In some embodiments, the antibody fragment moiety is caplacizumab, ozoralizumab, V565, PF-05230905, vobarilizumab, LCAR-B38M, M6495, BI 655088, AD-214, ALX-0651, TXB4, CDP791, or GY1.

In some embodiments, X further comprises an imaging contrast agent. In some embodiments, the imaging contrast agent is a protein.

### Linker Moieties

In some embodiments, L is bonded to X via a cystine or lysine residue on X.

In some embodiments, L is a non-cleavable linker.

In some embodiments, L is a cleavable linker.

In some embodiments, L comprises one or more amino acids.

In some embodiments, L comprises a polypeptide.

In some embodiments, L comprises one or more of a hydrazone, a hydrazide, a disulfide, a N-succinimidyl-4-(2-pyridyldithio) pentanoate (SPP), a N-succinimidyl-4-(2-pyridyldithio) butyrate (SPDB), a 4-(4′-acetylphenoxy) butanoic acid (AcBut), one or more linear or branched, natural or unnatural amino acid, a valine-citrulline (Val-Cit) moiety, or a phenylalanine-lysine (Phe-Lys) moiety.

In some embodiments, L comprises 1 to 100 linking atoms, from 1 to 50 linking atoms, or from 5 to 50 linking atoms, or from 10 to 50 linking atoms, or from 1 to 40 linking atoms, or from 1 to 30 linking atoms, or from 1 to 20 linking atoms, or from 1 to 10 linking atoms, or from 1 to 5 linking atoms, or from 5 to 30 linking atoms, or from 10 to 30 linking atoms, or from 5 to 40 linking atoms, or from 5 to 50 linking atoms, or from 10 to 50 linking atoms.

In some embodiments, L comprises one or more chain heteroatoms and one or more alkylene, alkenylene, alkynylene, arylene, heteroarylene, cycloalkylene or heterocycloalkylene moieties; wherein each alkylene, alkenylene, alkynylene, arylene, heteroarylene, cycloalkylene or heterocycloalkylene moiety, may be independently optionally substituted with one to five substituents independently selected from oxo, halo, C₁₋₄ alkyl, C₁₋₄ alkoxy, and C₁₋₄ haloalkyl.

In some embodiments, L is an alkylene linker optionally comprising one or more —O—, —S—, amine, ester, amide, carbamate, carbonate, thio-succinimide, or ketone functional groups.

In some embodiments, linker L is of the formula:

—Y¹⁰—(CHR¹³⁰)_{n′}—Y²⁰—(CHR¹⁴⁰)_{n″}—Y³⁰—(CHR¹⁵⁰)_{m″}—Y⁴⁰—
wherein:
each of Y¹⁰, Y²⁰, Y³⁰, and Y⁴⁰ are independently a bond, —NR¹¹⁰—, —O—, —S(O)₀₋₂—, —NR¹¹⁰C(O)—, —C(O)NR¹¹⁰—, —NR¹¹⁰S(O)₂—, —S(O)₂NR¹¹⁰—, —CR¹²⁰═N—NR¹¹⁰—, —NR¹¹⁰—N═CR¹²⁰—, —C(O)—, —OC(O)—, —OC(O)O—, —(CH₂CH₂O)₁₋₅—, —C(O)O—, alkylene, alkenylene, alkynylene, arylene, or heteroarylene; wherein each alkylene, alkenylene, alkynylene, arylene, or heteroarylene is independently optionally substituted with one to five substituents independently selected from oxo, halo, C₁₋₄ alkyl, C₁₋₄ alkoxy, and C₁₋₄ haloalkyl;
each R¹¹⁰ is independently hydrogen, C₁₋₄ alkyl, C₁₋₄ haloalkyl, aryl, heteroaryl, cycloalkyl, or heterocyclyl;
each R¹²⁰ is independently hydrogen, C₁₋₄ alkyl, C₁₋₄ haloalkyl, aryl, heteroaryl, cycloalkyl, or heterocyclyl;
each R¹³⁰ is independently hydrogen, C₁₋₄ alkyl, C₁₋₄ haloalkyl, aryl, heteroaryl, cycloalkyl, heterocyclyl or an amino acid side chain;
each R¹⁴⁰ is independently hydrogen, C₁₋₄ alkyl, C₁₋₄ haloalkyl, aryl, heteroaryl, cycloalkyl, heterocyclyl, or an amino acid side chain;
each R¹⁵⁰ is independently hydrogen, C₁₋₄ alkyl, C₁₋₄ haloalkyl, aryl, heteroaryl, cycloalkyl, heterocyclyl or an amino acid side chain; and
n′, n″, and m″ are each independently 0, 1, 2, 3, 4, 5, 6, 7, or 8.

In some embodiments, L is of the formula:

—Y¹⁰—(CH₂)_{n′}—Y²⁰—(CH₂)_{m″}—Y³⁰—
wherein:
each of Y¹⁰, Y²⁰, and Y³⁰ are independently a bond, —NR¹¹⁰—, —O—, —S(O)₀₋₂—, —NR¹¹⁰C(O)—, —C(O)NR¹¹⁰—, —NR¹¹⁰S(O)₂—, —S(O)₂NR¹¹⁰—, —CR¹²⁰═N—NR¹¹⁰—, —NR¹¹⁰—N═CR¹²⁰—, —C(O)—, —OC(O)—, —OC(O)O—, alkylene, alkenylene, alkynylene, arylene, heteroarylene, cycloalkylene or heterocycloalkylene; wherein each alkylene, alkenylene, alkynylene, arylene, heteroarylene, cycloalkylene or heterocycloalkylene is independently optionally substituted with one to five substituents independently selected from oxo, halo, C₁₋₄ alkyl, C₁₋₄ alkoxy, and C₁₋₄ haloalkyl;
each R¹¹⁰ is independently hydrogen, C₁₋₄ alkyl, C₁₋₄ haloalkyl, aryl, heteroaryl, cycloalkyl or heterocyclyl;
each R¹²⁰ is independently hydrogen, C₁₋₄ alkyl, C₁₋₄ haloalkyl, aryl, heteroaryl, cycloalkyl or heterocyclyl; and
n′ and m″ are each independently 0, 1, 2, 3, 4, 5, 6, 7, or 8.

In certain embodiments, each R¹¹⁰ is independently hydrogen, C₁₋₄ alkyl, C₁₋₄ haloalkyl, aryl, heteroaryl, cycloalkyl or heterocyclyl; and each R¹²⁰ is independently hydrogen, C₁₋₄ alkyl, C₁₋₄ haloalkyl, aryl, heteroaryl, cycloalkyl or heterocyclyl. In certain embodiments, the linker is not a bond.

The linker L may comprise one or more of polyethylene glycol (e.g., PEG having an average molecular weight of from 300 g/mol to 10,000 g/mol), ethylene-1,2-diylbis(methylcarbamate, an arylene (e.e., phenylene), ethylene-oxy, amine, ester, amide, carbamate, ketone (i.e., formyl), or carbonate.

In some embodiments, the linker comprises one or more of:

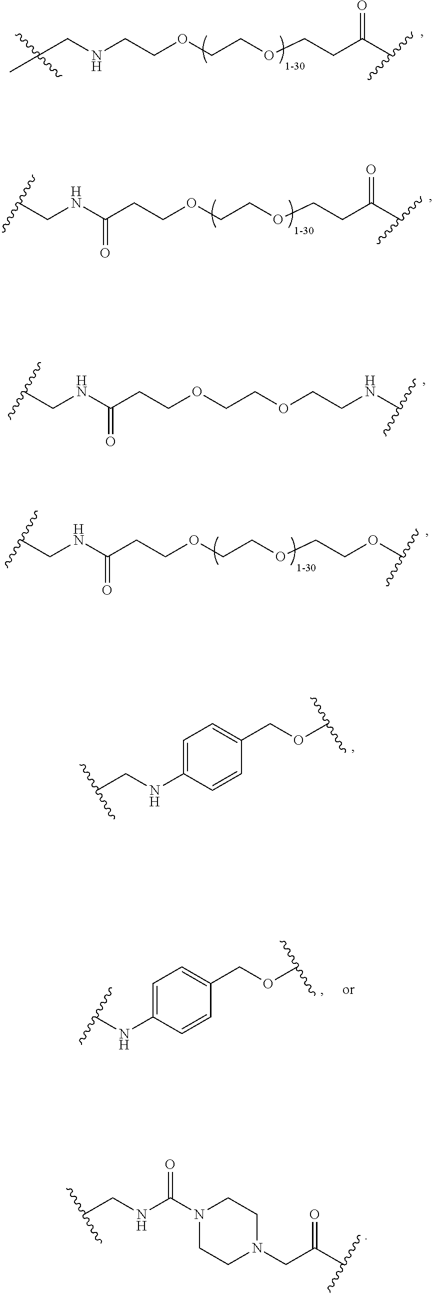

In some embodiments, the linker comprises one or more of:

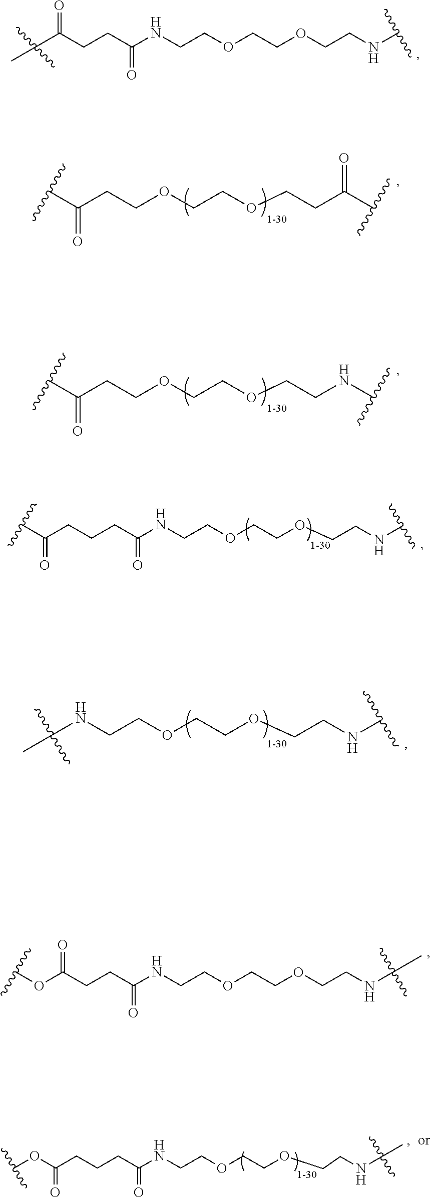

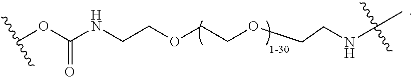

In some embodiments, the linker comprises one or more of:

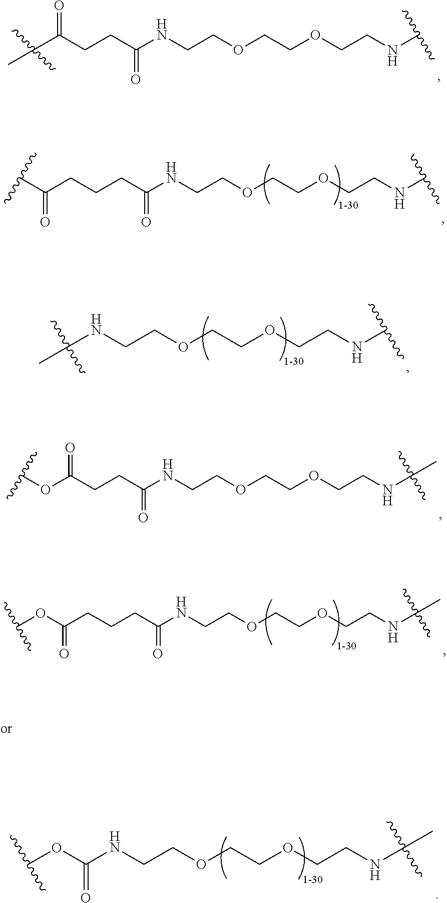

In some embodiments, the linker comprises one or more

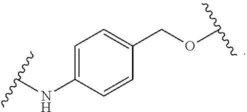 [SMILES: *Nc1ccc(CO*)cc1]

In some embodiments, the linker comprises one or more

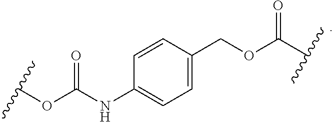 [SMILES: *OC(=O)Nc1ccc(COC(*)=O)cc1]

In some embodiments, the linker is, or comprises one or more:

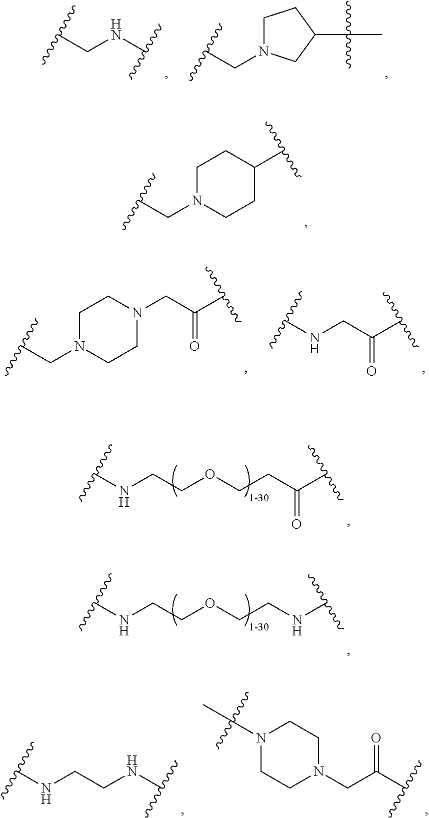

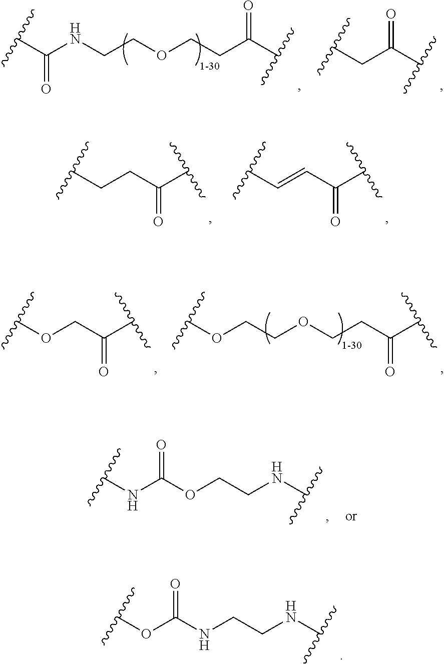

In some embodiments, the linker is, or comprises one or more:

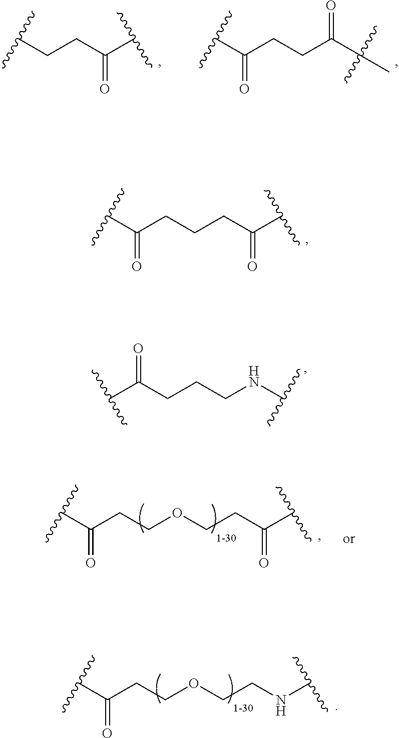

In some embodiments, the linker comprises one or more natural or unnatural amino acids, which may be referred to as a peptide linker. The linker may be a peptide linker made up of a carboxylic acyl unit, and one or more amino acids making up a protein or peptide sequence. The linker may also contain a self-immolating spacer which spaces the drug and the protein peptide sequence.

In some embodiments, the linker may be a peptide containing linker represented by “A-Y—Z—X²—W” in which “A” is the carboxylic acyl unit, “Y” and “Z” are each one or more natural or unnatural amino acids and together form a peptide sequence, and “X²” and “W” are optional additional linkers having from 1 to 50 linking atoms, or from 5 to 10 linking atoms, or from 1 to 10 linking atoms which spaces the peptide and the payload, D, or the bioorthogonal moiety. In certain embodiments, one or more of the amino acids in the peptide linker is N-methylated.

In some embodiments, Y may be at least one amino acid selected from the group consisting of alanine, valine, leucine, isoleucine, methionine, phenylalanine, tryptophan, and proline. In some embodiments Y may be at least one amino acid selected from the group consisting of phenylalanine, alanine, and valine.

In some embodiments, Z may be at least one amino acid selected from the group consisting of alanine, lysine, lysine protected with acetyl or formyl, arginine, arginine protected with tosyl or nitro groups, histidine, ornithine, ornithine protected with acetyl or formyl, and citrulline. In some embodiments Z may be at least one amino acid selected from the group consisting of alanine, lysine, and citrulline.

Exemplary Y—Z combinations include Valine-Citrulline; Valine-Alanine; and Alanine-Alanine.

In some embodiments, A is —OC(O)—.

In some embodiments, X² is —OC(O)—.

In some embodiments, W is —OC(O)—. In some embodiments, X² is absent and W is —OC(O)—.

In some embodiments, the moiety —X²—W comprises

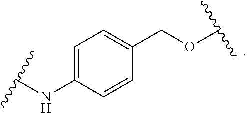 [SMILES: *Nc1ccc(CO*)cc1]

In some embodiments, the moiety —X² is

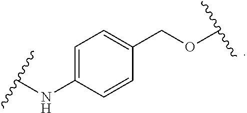 [SMILES: *Nc1ccc(CO*)cc1]

In some embodiments, —X—W is

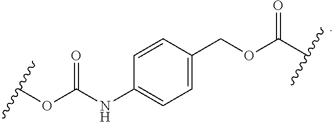 [SMILES: *OC(=O)Nc1ccc(COC(*)=O)cc1]

In some embodiments, —X—W is

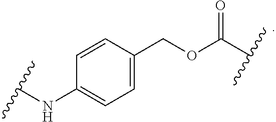 [SMILES: *Nc1ccc(COC(*)=O)cc1]

In some embodiments, the peptide linker is specifically tailored so that it will be selectively cleaved (e.g., enzymatically cleaved) releasing the drug, such as by one or more of the tumor-associated proteases.

In some embodiments, the peptide linker has a chain length of two to four amino acid residues (i.e., a di-, tri-, or tetra-peptide). It will be understood, however, that peptide linkers up to five, six, seven, or eight amino acid residues may also suitably be employed.

In some embodiments, the peptide linker is Phe-Lys, Val-Lys, Val-Ala, Ala-Ala, Phe-Phe-Lys, D-Phe-Phe-Lys, Gly-Phe-Lys, Ala-Lys, Val-Cit, Phe-Cit, Leu-Cit, Ile-Cit, Trp-Cit, Phe-Ala, Gly-Phe-Leu-Gly [SEQ ID NO: 1], Ala-Leu-Ala-Leu [SEQ ID NO: 2], Phe-N⁹-tosyl-Arg, or Phe-N⁹-Nitro-Arg. In certain embodiments, the peptide linker is Phe-Lys, Val-Lys, Val-Ala, Ala-Ala, Val-Val, Val-Cit, or D-Phe-L-Phe-Lys. In certain embodiments, the peptide linker is Val-Cit, Val-Ala, or Ala-Ala.

In some embodiments, the linker L is, or comprises one or more of:

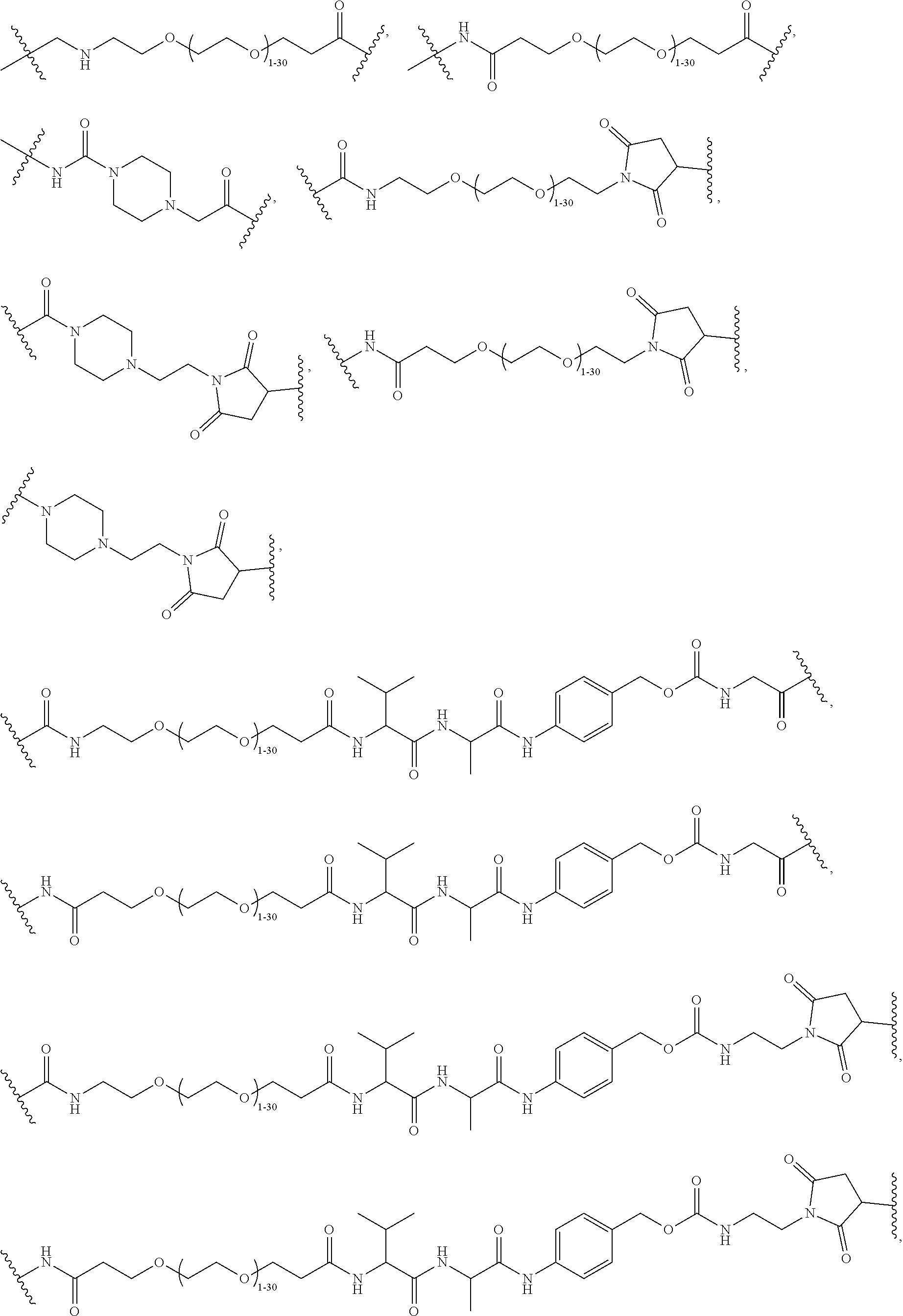

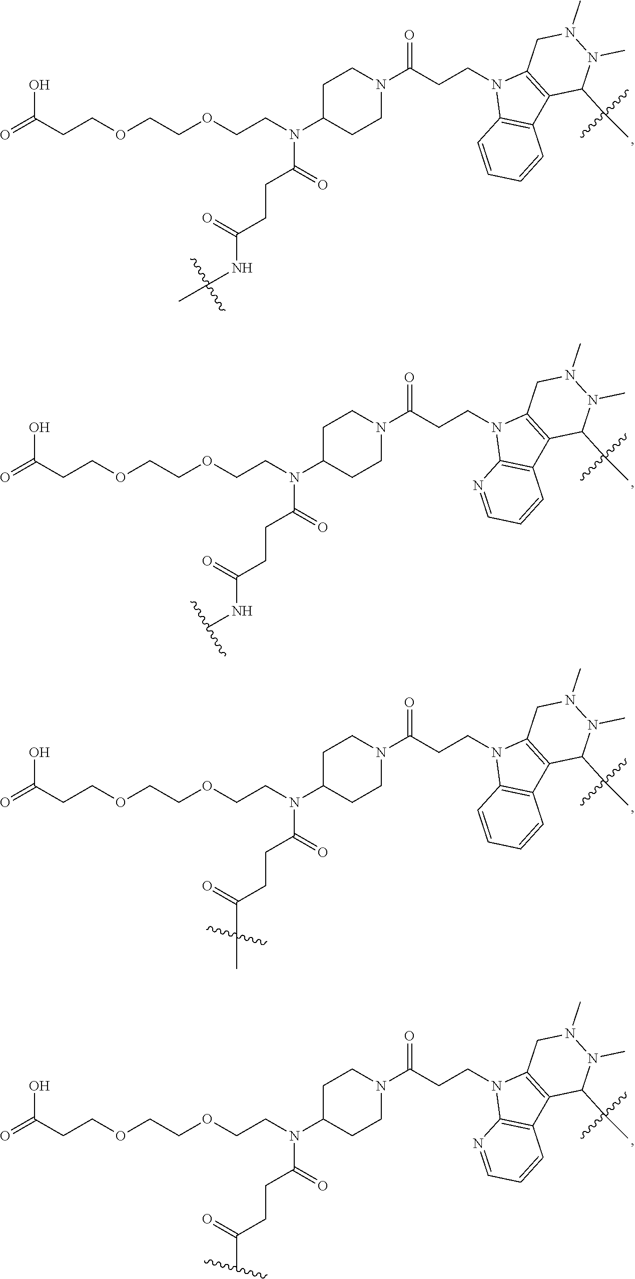 [SMILES: *NC(=O)CCC(=O)N(CCOCCOCCC(=O)O)C1CCN(C(=O)CCn2c3c(c4ccccc42)C(*)N(C)N(C)C3)CC1 ; *NC(=O)CCC(=O)N(CCOCCOCCC(=O)O)C1CCN(C(=O)CCn2c3c(c4cccnc42)C(*)N(C)N(C)C3)CC1 ; *C(=O)CCC(=O)N(CCOCCOCCC(=O)O)C1CCN(C(=O)CCn2c3c(c4ccccc42)C(*)N(C)N(C)C3)CC1 ; *C(=O)CCC(=O)N(CCOCCOCCC(=O)O)C1CCN(C(=O)CCn2c3c(c4cccnc42)C(*)N(C)N(C)C3)CC1]

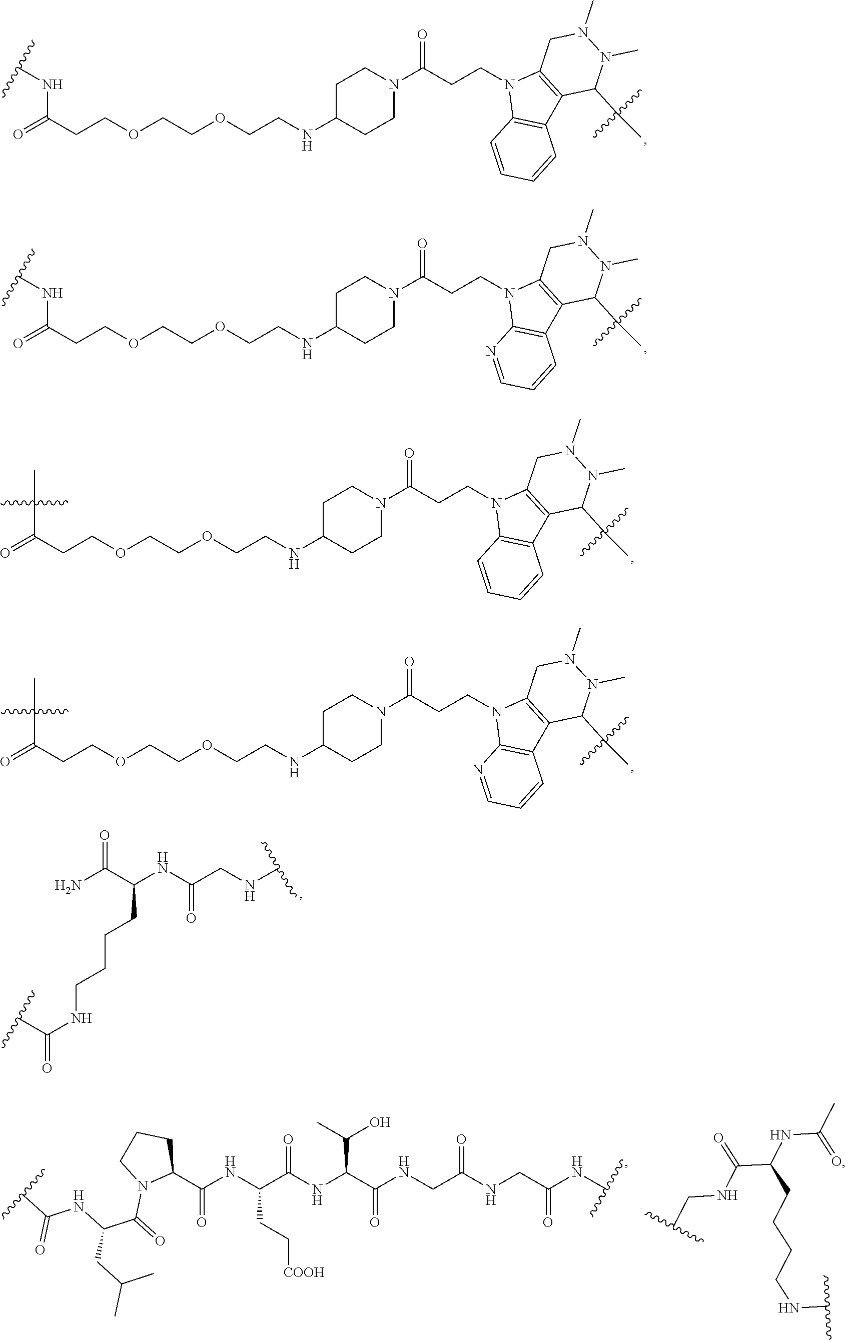

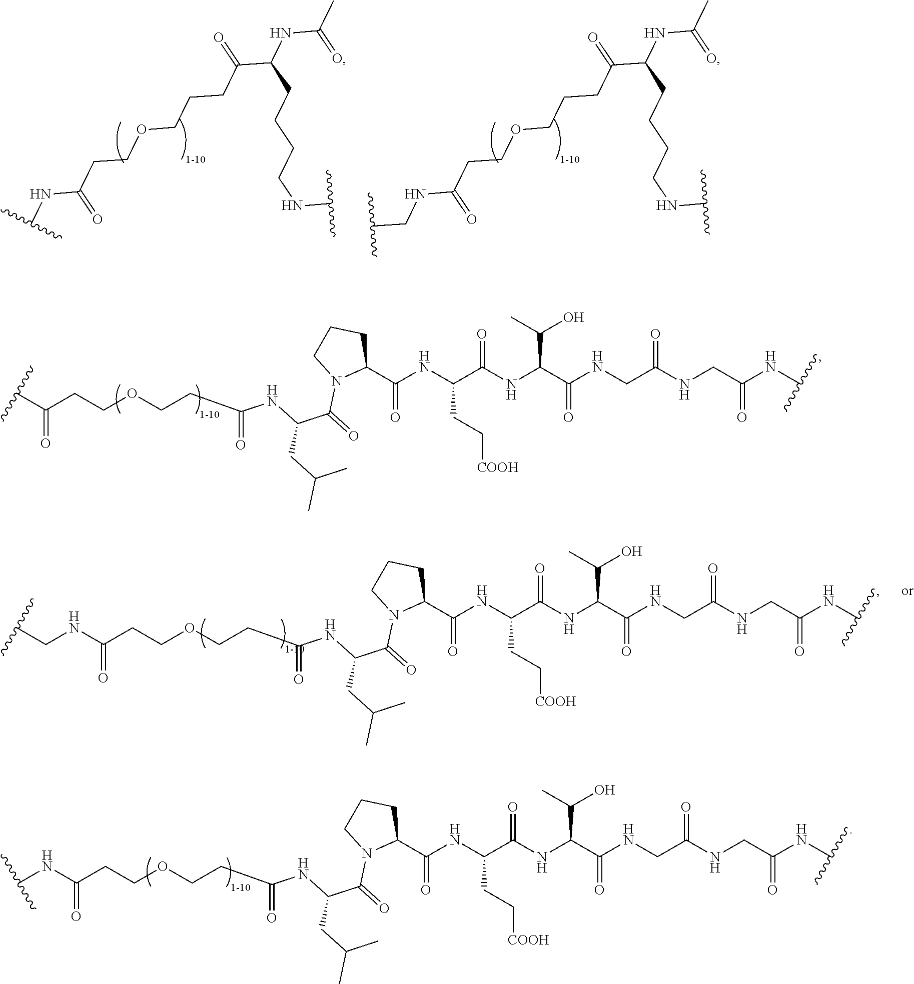

In some embodiments, the linker L comprises one or more of:

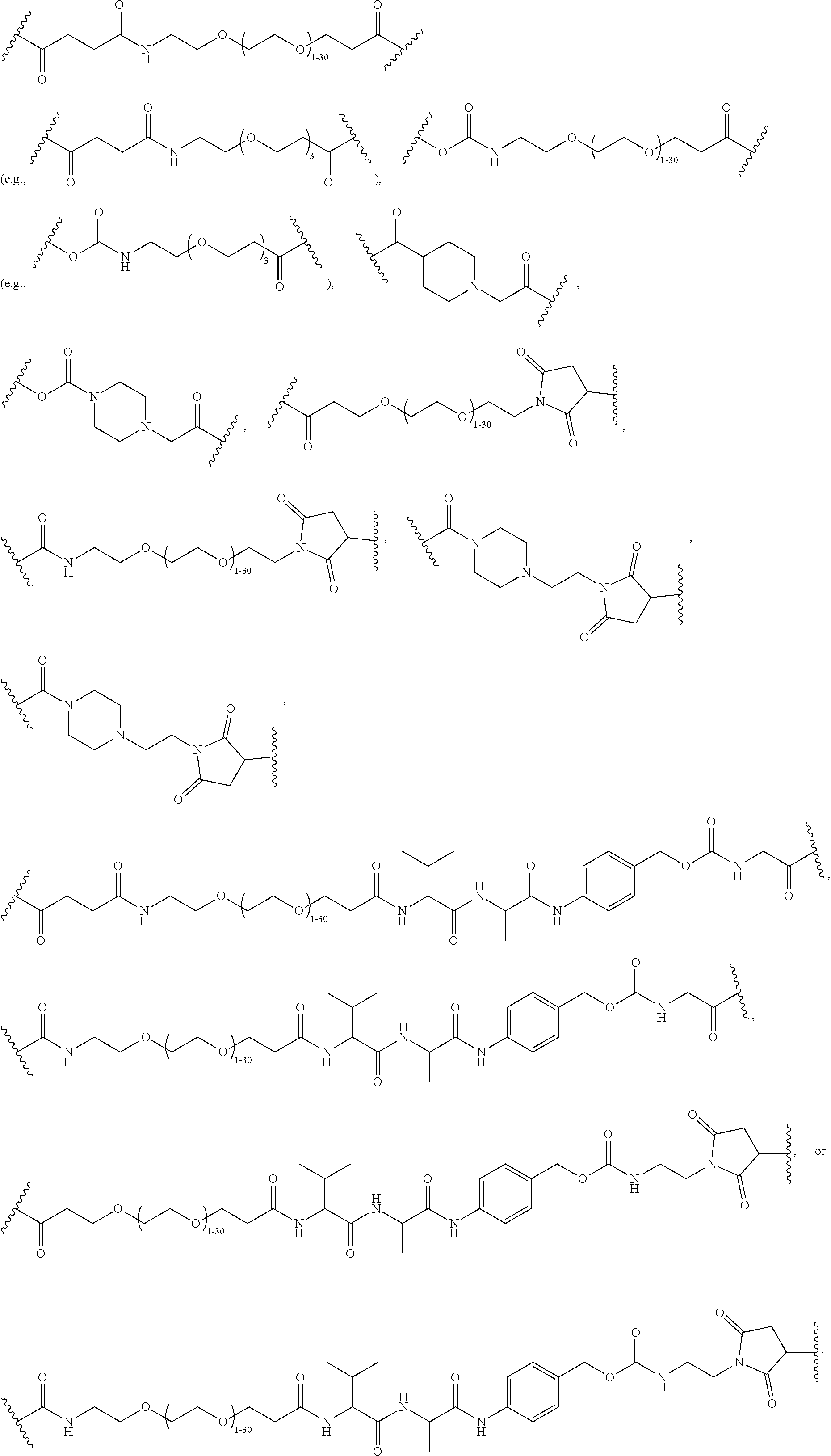

In some embodiments, the linker L comprises one or more of:

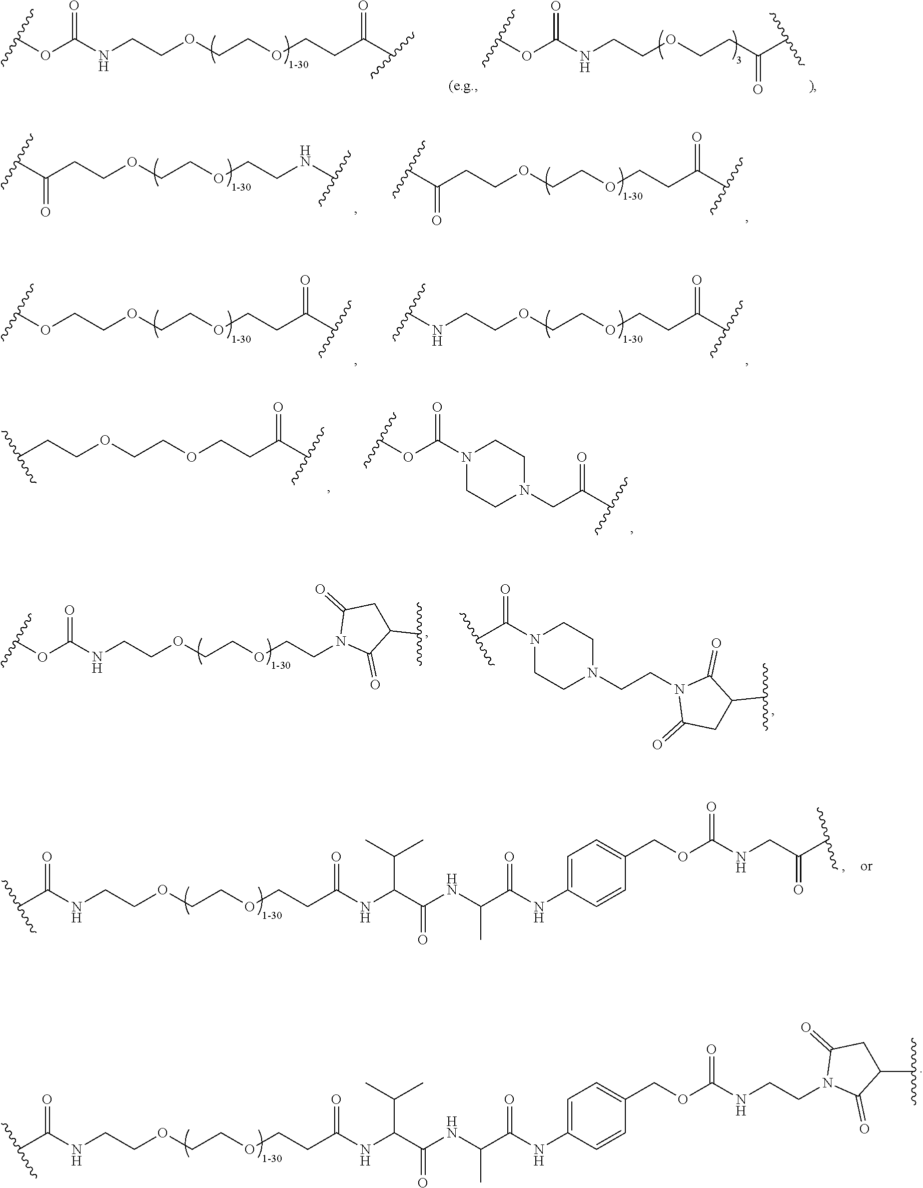

The foregoing linkers may bond to an amino acid side chain present on X, such as a lysine or cysteine

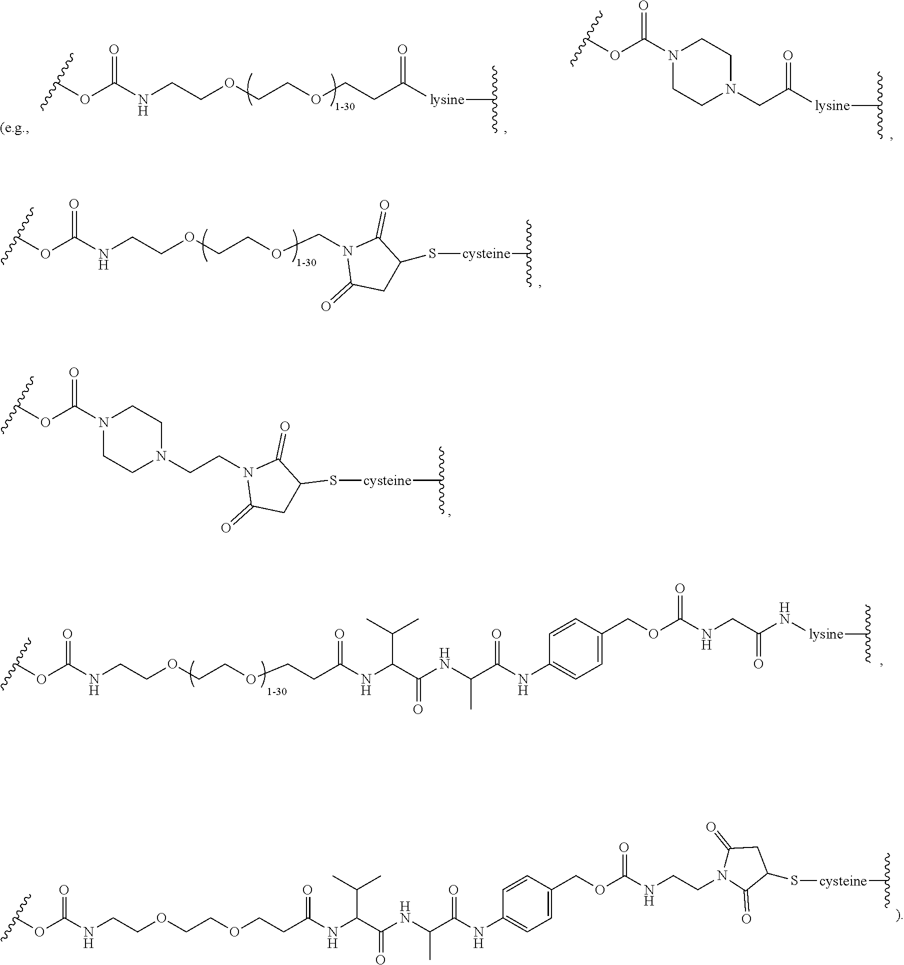

In some embodiments, the linker L is —C(O)L⁴- or —C(O)C₁₋₆alkyleneC(O)L⁴-;
L⁴ is a bond, —N(R¹²)—C₂₋₃alkylene-N(R¹³)C(O)—, —CH(NHC(O)R¹⁴)C₁₋₄alkylene-S—S—C₁₋₄alkylene-OC(O)—, —NHNHC(O)CH(NHC(O)R¹⁵)CH₂C(O)—, —C₁₋₆alkylene-CH(G^{x})OC(O)—,

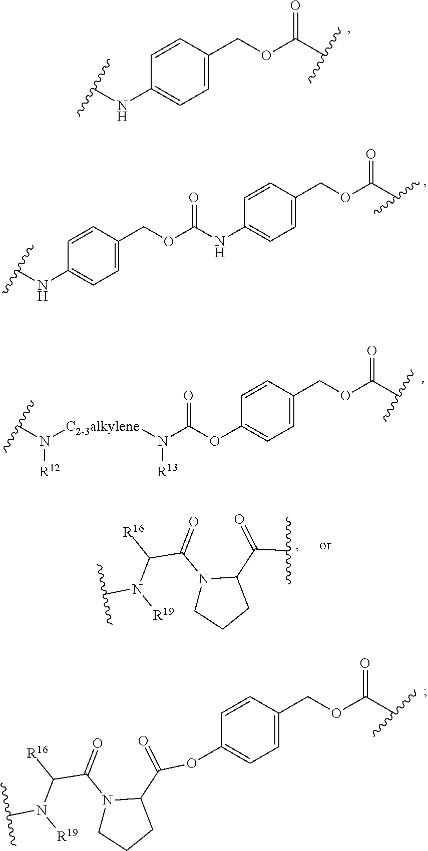

R¹², R¹³, R¹⁴, R¹⁵, and R¹⁹ are each independently hydrogen or C₁₋₄alkyl;
R¹⁶ is hydrogen, C₁₋₄alkyl, —C₁₋₄alkylene-OH, —C₁₋₄alkylene-OC₁₋₄alkyl, —C₁₋₄alkylene-CO₂H, or —C₁₋₄alkylene-CONH₂; and
G^{x} is phenyl optionally substituted with 1-5 substituents independently selected from the group consisting of halogen, C₁₋₄alkyl, C₁₋₄haloalkyl, C₁₋₄alkoxy, cyano, and nitro.

In some embodiments, the linker L comprises a carbonyl moiety for conjugating the tetrazine moiety to the linker or X. For example, the linker may comprise a polypeptide moiety (PPM) having the lysine residue and lysine side chain and the PPM may also have additional lysines, or other amino acid side chains conjugated to the carbonyl moiety. In some embodiments, the linker L may comprise

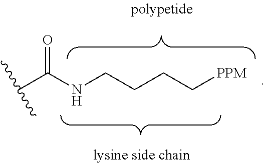

In some embodiments, the linker L is, or comprises one or more of:

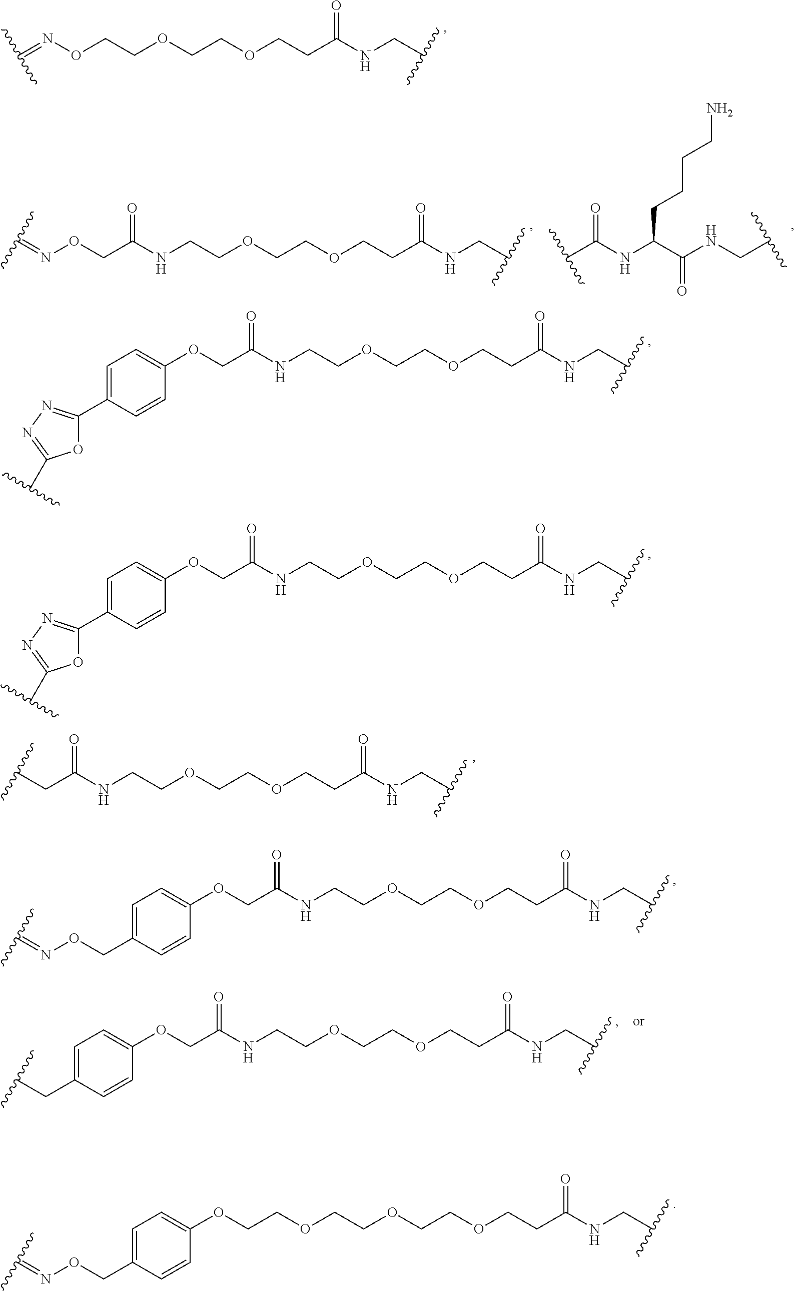

In some embodiments, the linker L is, or comprises one or more of:

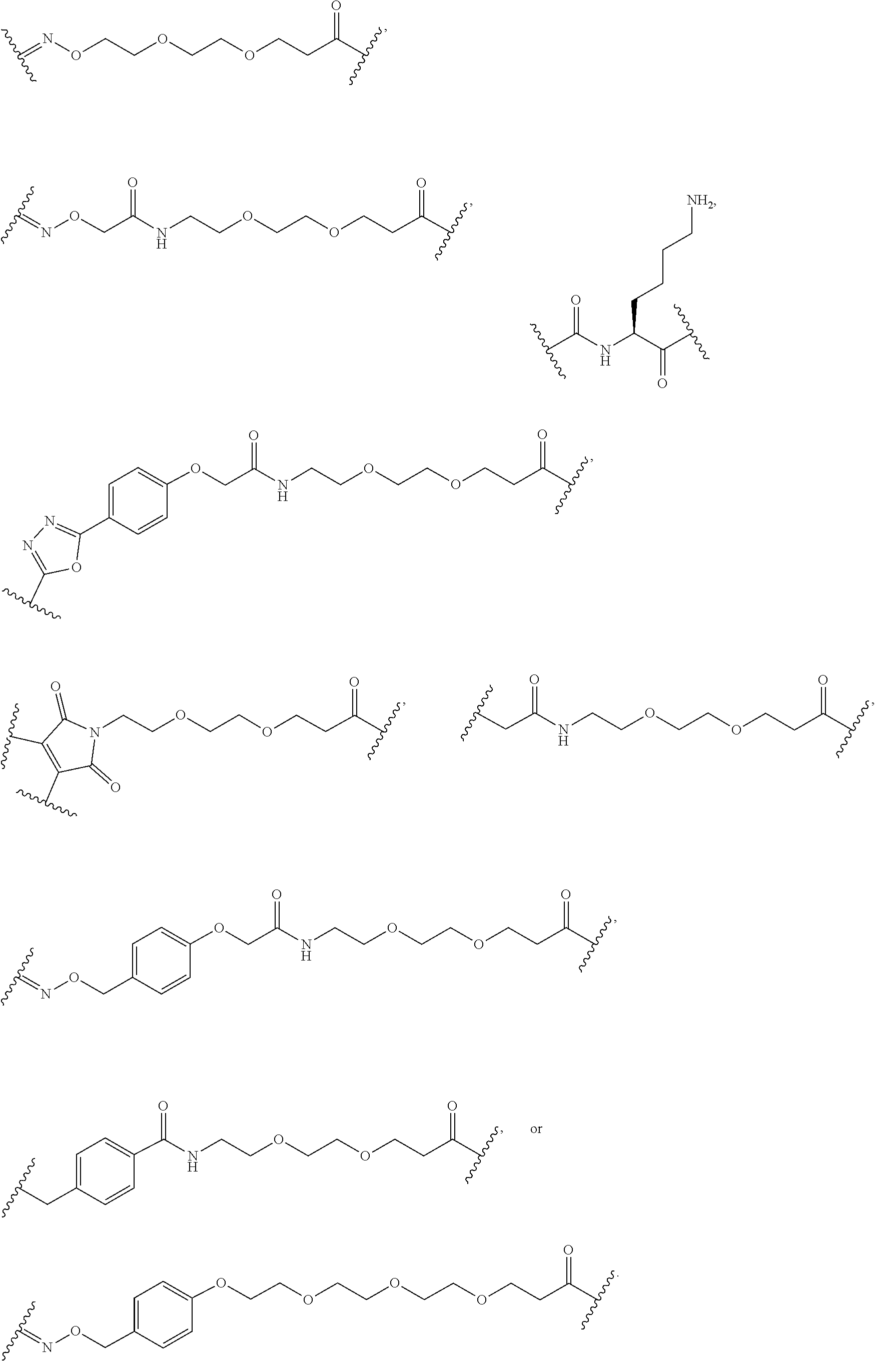

In some embodiments, the linker L is, or comprises one or more of:

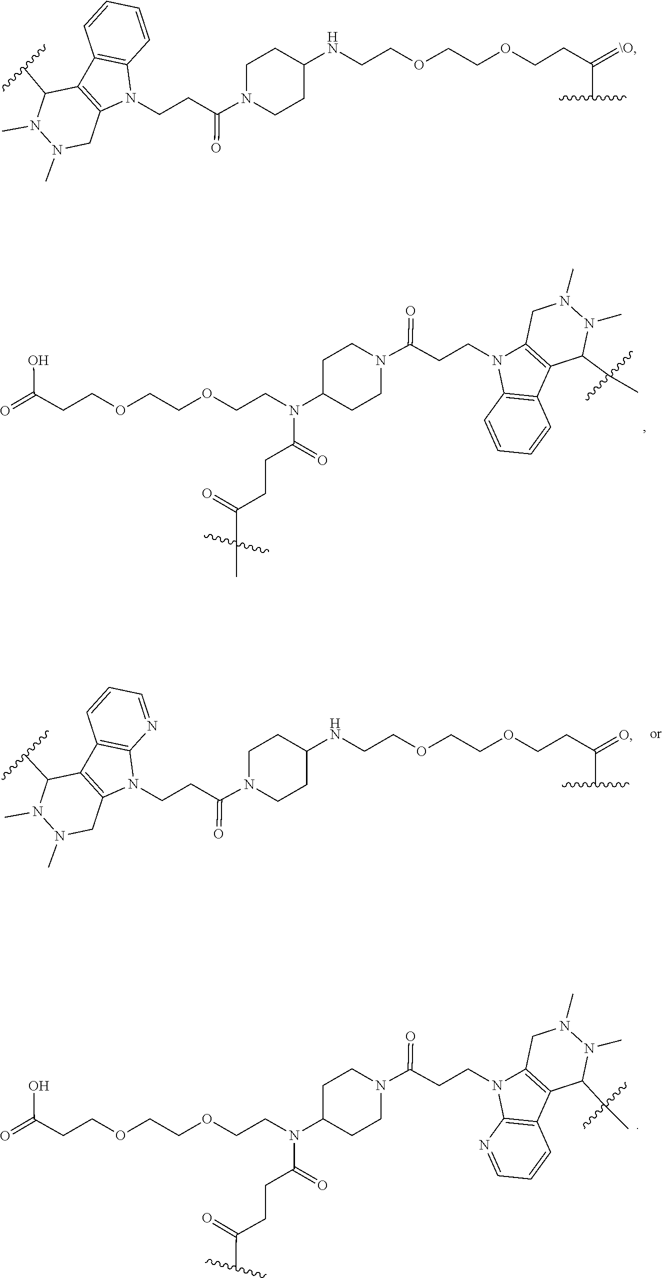

In some embodiments, the linker L is:

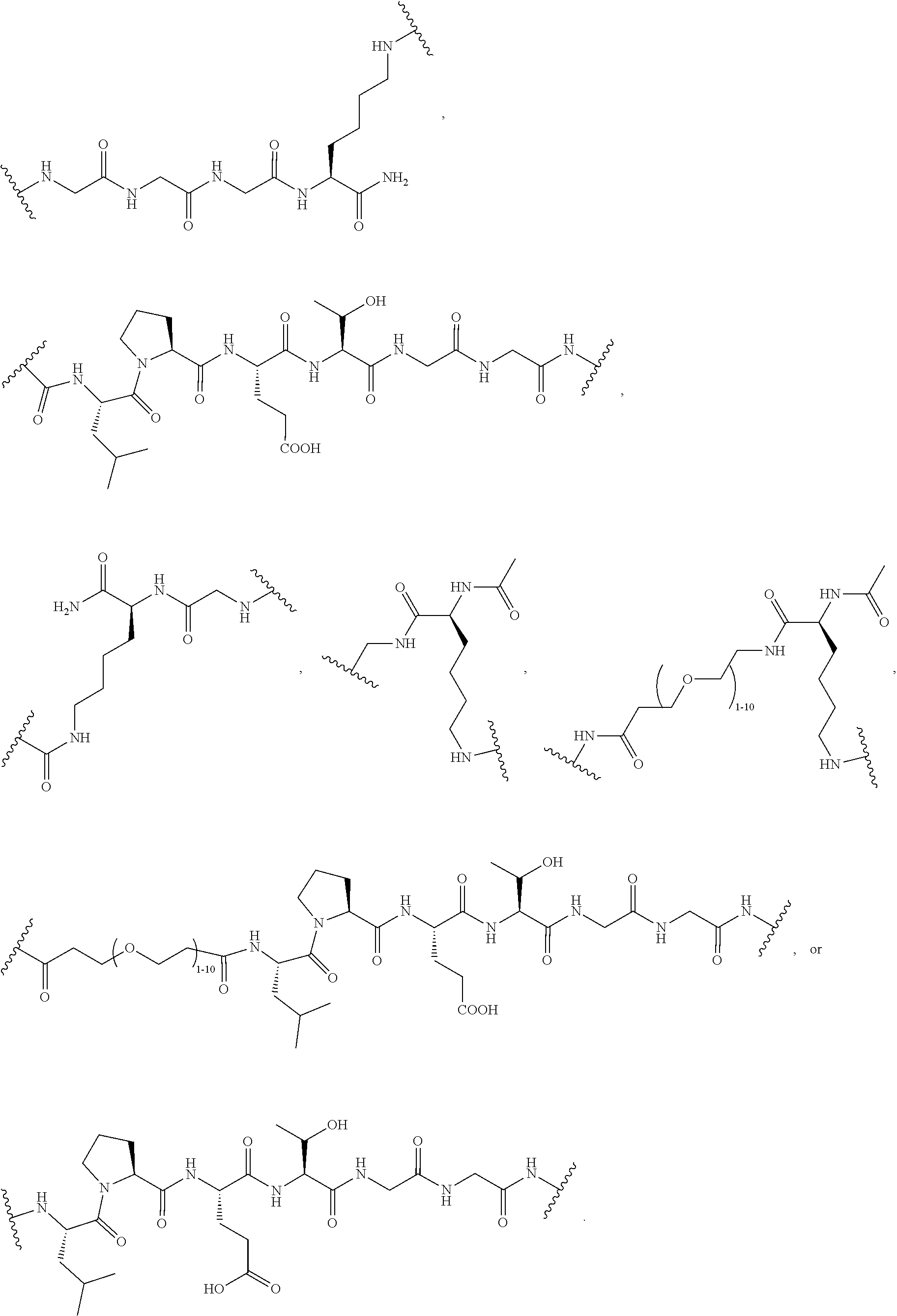

In some embodiments, the linker L is:

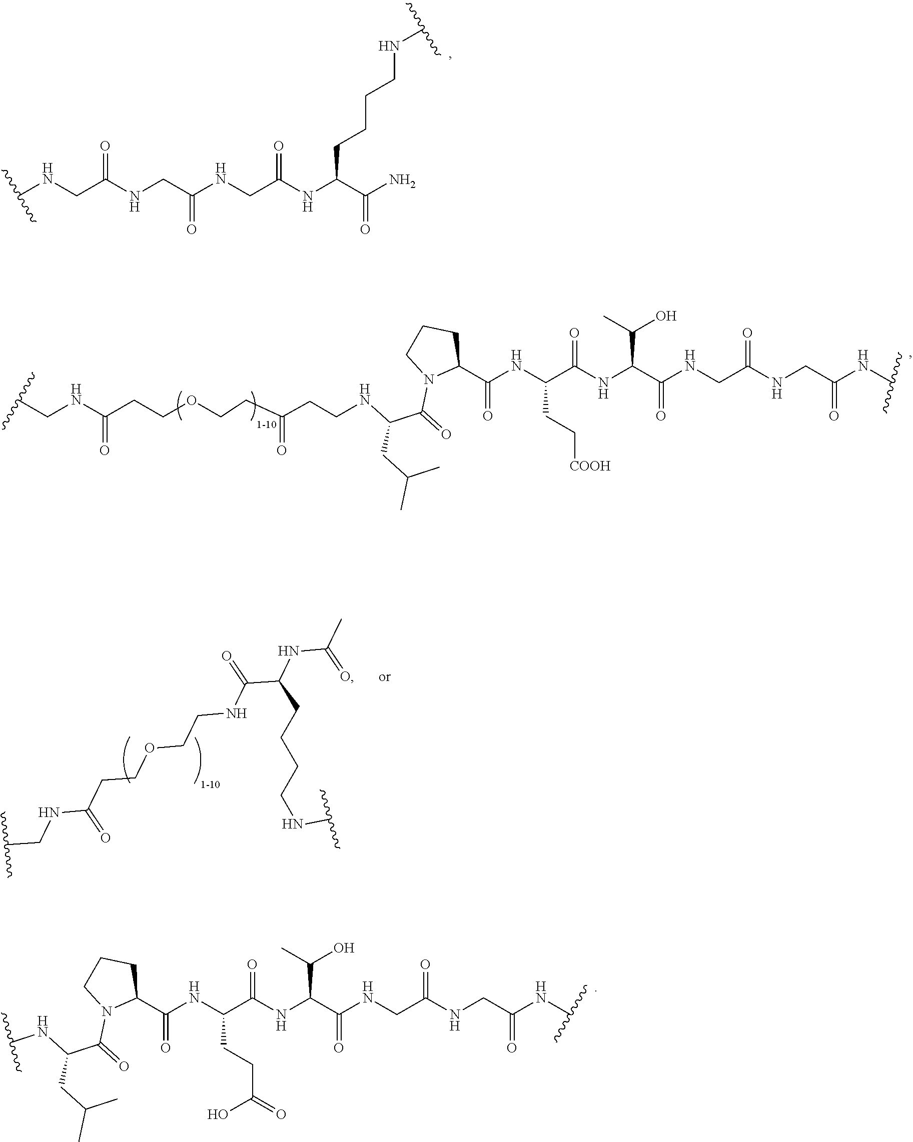

In some embodiments, the linker L is, or comprises one or more of

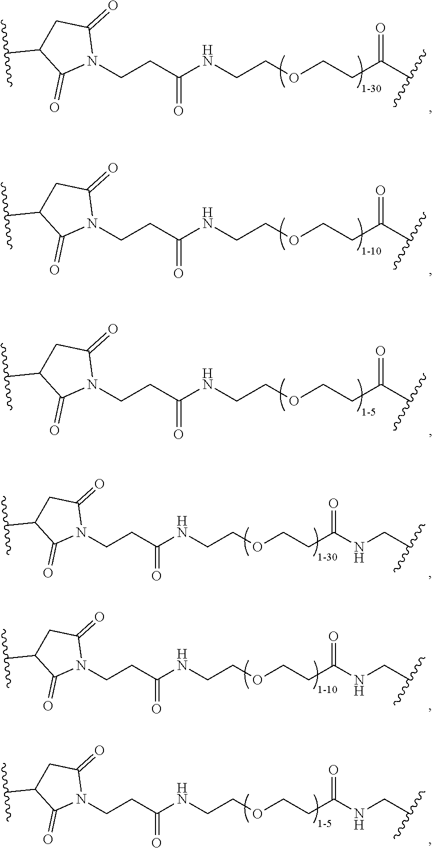

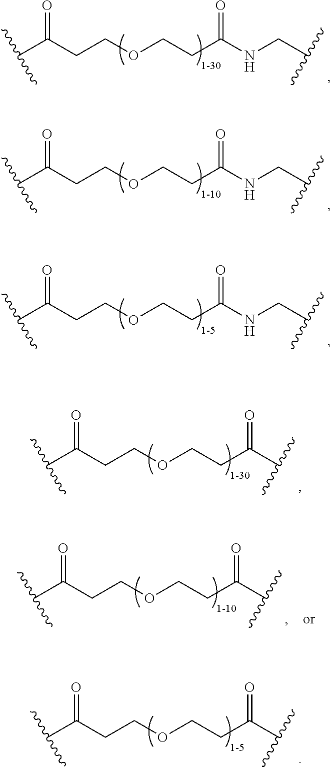

In some embodiments, the linker L is, or comprises one or more of:

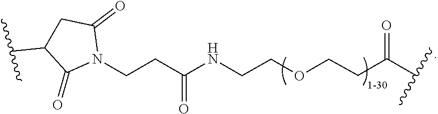

In some embodiments, the linker L is, or comprises one or more of:

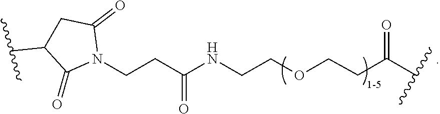

In some embodiments, the linker L is, or comprises one or more of:

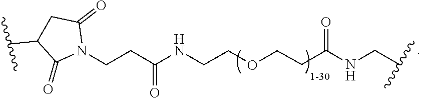

In some embodiments, the linker L is, or comprises one or more of:

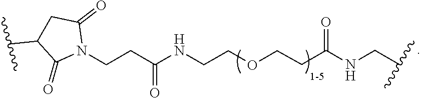

In some embodiments, the linker L is, or comprises one or more of:

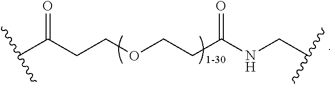

In some embodiments, the linker L is, or comprises one or more of:

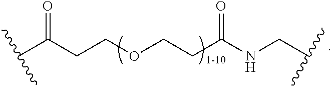

In some embodiments, the linker L is, or comprises one or more of:

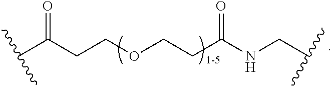

In some embodiments, the linker L is, or comprises one or more of:

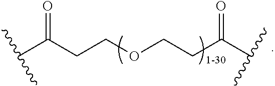

In some embodiments, the linker L is, or comprises one or more of:

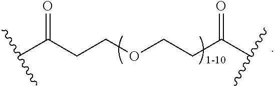

In some embodiments, the linker L is, or comprises one or more of:

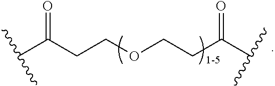

In one embodiment, provided is a targeting moiety of Formula I:

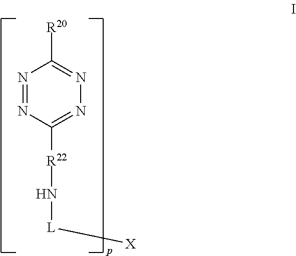

wherein:
p is 1-16;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R″, S(═O)₂NR′R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R²², at each occurrence, is independently a linker of 1 to 100 linking atoms optionally comprising one or more ethylene-oxy, amine, ester, amide, carbamate, carbonate, or ketone functional group;
R′ and R″, at each occurrence, are independently selected from hydrogen, aryl, and alkyl;
R′″ at each occurrence is independently selected from aryl and alkyl;
X is an antibody or antibody fragment moiety that targets HER2, TROP2, Nectin-4, Claudin-18.2, MMP9, mesothelin, FN1, FAP, TNC, or ECM, EPCAM, CEA, or CEACAM5; and
L, at each occurrence, is independently a linker selected from the group consisting of:

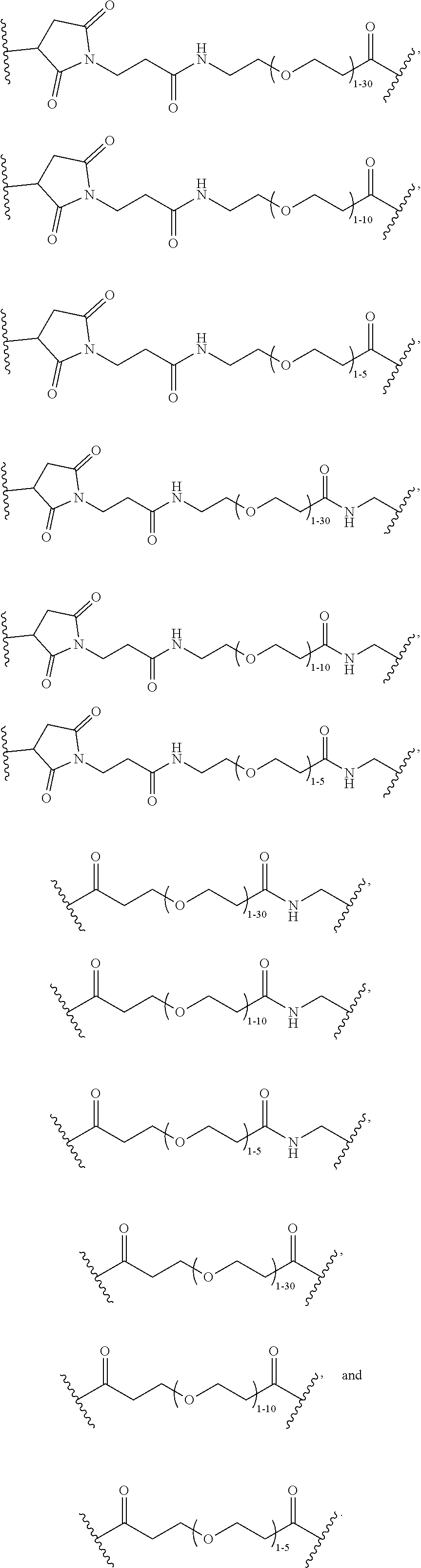

In one embodiment, provided is a targeting moiety of Formula II:

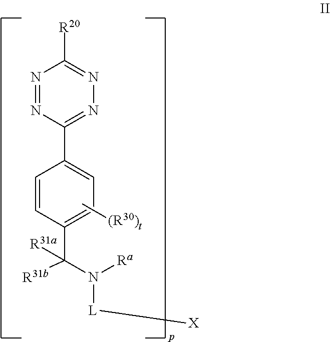

wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′ R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl;
R^{a}, R^{31a} and R^{31b} are each independently hydrogen, C₁-C₆-alkyl, or C₁-C₆-haloalkyl;
R′ and R″, at each occurrence, are independently selected from hydrogen, aryl, and alkyl;
R′″, at each occurrence, is independently selected from aryl and alkyl;
t is independently is 0, 1, 2, 3, or 4;
X is an antibody or antibody fragment moiety that targets HER2, TROP2, Nectin-4, Claudin-18.2, MMP9, mesothelin, FN1, FAP, TNC, or ECM, EPCAM, CEA, or CEACAM5; and
L, at each occurrence, is independently a linker selected from the group consisting of:

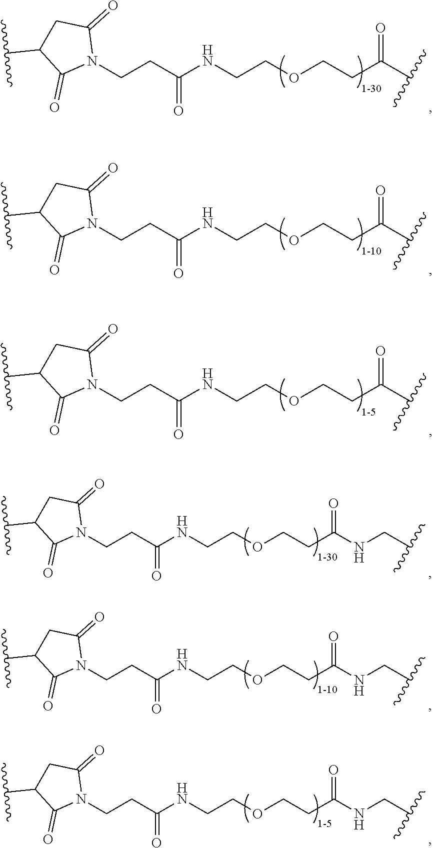

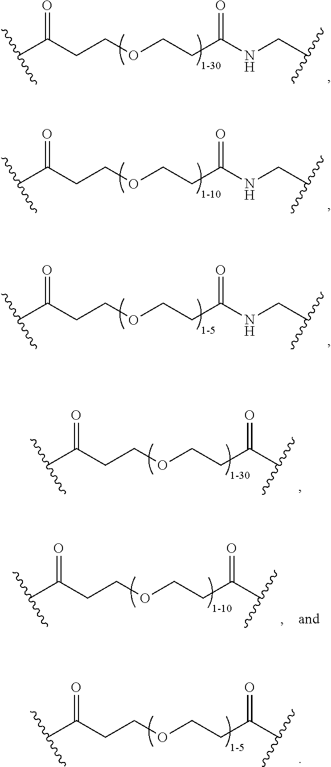

In one embodiment, provided is a targeting moiety of Formula V:

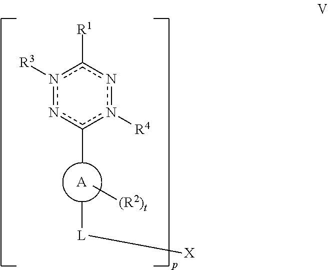

wherein:
ring A is aryl, cycloalkyl, heterocyclyl, or heteroaryl;
the dotted lines represent additional bonds to form a tetrazine when R³ and R⁴ are both absent, or a dihydrotetrazine when R³ and R⁴ are both present; provided that when ring A is aryl, then R³ and R⁴ are both present;
X is a biocompatible support, antibody, or antibody fragment moiety;
p is 1-15;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R″, SC(═O)R″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or R³ and R⁴ are each independently hydrogen or a group capable of being removed after a triggering event;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl;
t, at each occurrence, is independently is 0, 1, 2, 3, or 4;
X is an antibody or antibody fragment moiety that targets HER2, TROP2, Nectin-4, Claudin-18.2, MMP9, mesothelin, FN1, FAP, TNC, or ECM, EPCAM, CEA, or CEACAM5; and
L, at each occurrence, is independently a linker selected from the group consisting of:

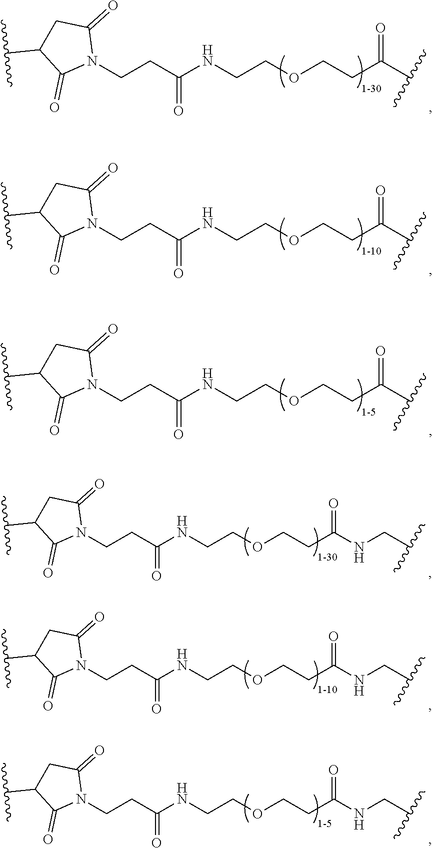

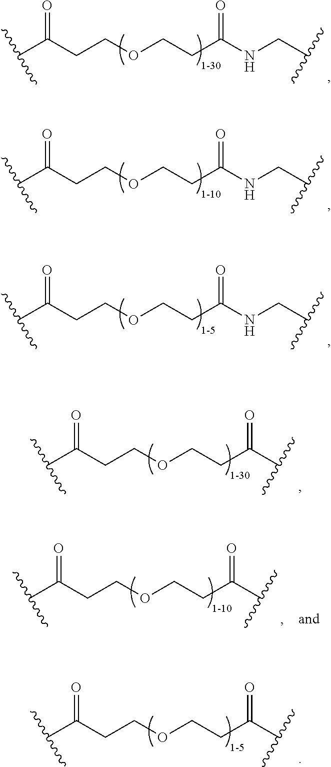

In some embodiments, ring A is pyrimidinyl, triazinyl, oxazolyl, isoxazole, imidazolyl, oxadiazolyl, 6,7-dihydro-5H-pyrrolo[3,4-d]pyrimidinyl, 5,6,7,8-tetrahydropyrido[4,3-d]pyrimidinyl, or 5,6,7,8-tetrahydropyrido[3,4-d]pyrimidinyl; wherein each may be optionally substituted.

In some embodiments, X is an antibody, or antibody fragment moiety, that targets HER2, TROP2, Nectin-4, FN1, FAP, TNC, or ECM.

In some embodiments, X is zolbetuximab, claudiximab, andecaliximab, anetumab, amatuximab, MMOT-0530A, L19, NJB2, F19, OMTX005, sibrotuzumab, F16, or R6N, trastuzumab, enfortumab, or sacituzumab, or an antibody fragment moiety derived therefrom.

In some embodiments, X is L19, NJB2, F19, OMTX005, sibrotuzumab, F16, or R6N, trastuzumab, enfortumab, or sacituzumab, or an antibody fragment moiety derived therefrom.

In some embodiments, p is 1-100. In some embodiments, p is 1-75. In some embodiments, p is 1-50. In some embodiments, p is 1-30. In some embodiments, p is 1-20. In some embodiments, p is 1-10. In some embodiments, p is 5-10. In some embodiments, p is 1-15. In some embodiments, p is 8-12.

In some embodiments, p is 1-12. In some embodiments, X is an antibody. In some embodiments, p is 1-6, or 5-6. In some embodiments, X is an antibody fragment moiety (e.g., Fab).

In some embodiments, p is 1-10, or 1-9, or 1-8, or 1-7, or 1-6, or 1-5, or 1-4, or 1-3, or 1-2, or 2-10, or 2-9, or 2-8, or 2-7, or 2-6, or 2-5, or 2-4, or 2-3, or 3-10, or 3-9, or 3-8, or 3-7, or 3-6, or 3-5, or 3-4, or 4-10, or 4-9, or 4-8, or 4-7, or 4-6, or 4-5, or 5-10, or 5-9, or 5-8, or 5-7, or 5-6, 6-10, or 6-9, or 6-8, or 6-7, 7-10, or 7-9, or 7-8, 8-10, or 8-9, or 9-10. In some embodiments, X is an antibody or an antibody fragment moiety.

In some embodiments, p is 1-16, or 1-8, or 1-7, or 1-6, or 1-5, or 1-4, or 1-3, or 1-2. In some embodiments, X is an antibody fragment moiety (e.g., Fab).

In certain embodiments, p is 1-10, or 1-9, or 1-8, or 1-7, or 1-6, or 1-5, or 1-4, or 1-3, or 1-2, or 2-10, or 2-9, or 2-8, or 2-7, or 2-6, or 2-5, or 2-4, or 2-3, or 3-10, or 3-9, or 3-8, or 3-7, or 3-6, or 3-5, or 3-4, or 4-10, or 4-9, or 4-8, or 4-7, or 4-6, or 4-5, or 5-10, or 5-9, or 5-8, or 5-7, or 5-6, 6-10, or 6-9, or 6-8, or 6-7, 7-10, or 7-9, or 7-8, 8-10, or 8-9, or 9-10, and X is an antibody or an antibody fragment moiety of from 15 KDa to 75 KDa, or 25-75 KDa, or 45-55 KDa, or less than 25 KDa, or less than 35 KDa, or less than 45 KDa, or about 50 KDa, or less than 55 KDa, or less than 65 KDa, or less than 75 KDa, or greater than 75 KDa.

In certain embodiments, p is dependent on the size and/or number of available binding sites on X for forming a covalent bond to L. In certain embodiments, when X is an antibody or an antibody fragment moiety greater than 75 KDa, p is 2-6. In certain embodiments, when X is an antibody or an antibody fragment moiety between 25-75 KDa, p is 1-4. In certain embodiments, when X is an antibody or an antibody fragment moiety between 45-55 KDa, p is 1-4. In certain embodiments, when X is an antibody or an antibody fragment moiety of less than 25 KDa, p is 1-3, or 2-3, or 1-2, or about 1, about 2, or about 3.

### C. Support Compositions

The support composition comprises a support. In certain embodiments, the The support composition is a therapeutic support composition. Supports may be biocompatible supports compositions, i.e., compatible with the subject's body. In some instances, a support is non-toxic to the subject and does not substantially react with tissue or biological compounds in the subject. For example, the support can be a hydrogel, among others. A support is capable of implantation into a subject's body and supporting binding agents (e.g., tetrazine-containing group), as well as payloads after the binding agents conjugate. Representative supports include, but are not limited to polymers, viscous or non-viscous liquid materials, gels, hydrogels, polysaccharide hydrogels, a cross-linked polymer matrix, a metal, a ceramic, a plastic, a bone graft material, alginate, cellulose, chitosan, hyaluronic acid, chondroitin sulfate, heparin, and the like. Supports also include particles, such as nanoparticles, microparticles, and the like.

Hydrogels may be polysaccharide hydrogels, alginate, cellulose, hyaluronic acid, chitosan, chitosin, chitin, hyaluronic acid, chondroitin sulfate, heparin, and the like. Other suitable sugar-based biomaterials include those described in Polymer Advanced Technology, 2014, 25, 448-460. Polymers that may be used as the support can include, but are not limited to, polyphosphazenes, polyanhydrides, polyacetals, poly(ortho esters), polyphosphoesters, polycaprolactones, polyurethanes, polylactides, polycarbonates, polyamides, and polyethers, and blends/composites/co-polymers thereof. Representative polyethers include, but are not limited to, poly(ethylene glycol) (PEG), polypropylene glycol) (PPG), triblock Pluronic ([PEG]ₙ-[PPG]ₘ-[PEG]n), PEG diacrylate (PEGDA), and PEG dimethacrylate (PEGDMA). The support can also include proteins and other poly(amino acids), such as collagen, gelatin, elastin and elastin-like polypeptides, albumin, fibrin, poly(gamma-glutamic acid), poly(L-lysine), poly(L-glutamic acid), poly(aspartic acid), and the like.

In some embodiments, the support is a hydrogel. In some embodiments, the support is an alginate. In some embodiments, the support is chitin. In some embodiments, the support is a hyaluronic acid (e.g., a non-hydrogel hyaluronic acid substantially without crosslinks). In some embodiments, the support is chitosin.

In certain embodiments, the support is a particle. Particles of the present disclosure can have a diameter that is 2 cm or less, such as 1.5 cm or less, or 1 cm or less, or 0.5 cm or less. For example, the particles can be nanoparticles or microparticles. Nanoparticles include particles having average dimensions in the nanometer scale (e.g., 1000 nm or less). Microparticles are particles having average dimensions in the micrometer scale (e.g., 1000 μm or less). By “average” is meant the arithmetic mean. In some embodiments, the nanoparticles have a diameter ranging from 1 nm to 1 μm, such as from 10 nm to 1 μm, or 25 nm to 1 μm, or 50 nm to 1 μm, or 75 nm to 1 μm, or 100 nm to 1 μm, or 150 nm to 1 μm, or 200 nm to 1 μm, or 250 nm to 1 μm, or 300 nm to 1 μm, or 350 nm to 1 μm, or 400 nm to 1 μm, or 450 nm to 1 μm, or 500 nm to 1 μm. In other embodiments, the microparticles have a diameter ranging from 1 μm to 1 mm, such as from 10 μm to 1 mm, or 25 μm to 1 mm, or 50 μm to 1 mm, or 75 μm to 1 mm, or 100 μm to 1 mm, or 150 μm to 1 mm, or 200 μm to 1 mm, or 250 μm to 1 mm, or 300 μm to 1 mm, or 350 μm to 1 mm, or 400 μm to 1 mm, or 450 μm to 1 mm, or 500 μm to 1 mm. In further embodiments, small particles on the order of 10-100 nm in diameter may be assembled to form larger complexes, such as clusters or assemblies on the order of 1-10 μm. Particles of the present disclosure may be substantially spherical, such that the particles have a substantially circular cross-section. Other particle shapes may also be used, such as, but not limited to, ellipsoid, cubic, cylindrical, conical, needle, or other irregular shapes.

A “particle” may take the form of any fabricated material, a molecule, cryptophan, a virus, a phage, etc. The particle may be composed of a material, such as, but not limited to, a metal, a ceramic, a plastic, a glass, a composite, a polymer, a hydrogel, and the like. For example, the particles may be made of an inert material, such as alginate or iron oxide. In some examples, the particles may be magnetic and can be formed from a paramagnetic, super-paramagnetic or ferromagnetic material, or other material that responds to a magnetic field. Further, a particle may be of any shape, for example, spheres, rods, non-symmetrical shapes, etc. The particles, or a group of several particles in a complex, may be functionalized with a receptor that has a specific affinity to bind to or interact with a clinically relevant substrate. The receptor may be inherent to the particle itself. For example, the particle itself may be a virus or a phage with an inherent affinity for certain substrates. Additionally or alternatively, the particles can be functionalized by covalently or otherwise attaching or associating a receptor that specifically binds or otherwise recognizes a particular clinically relevant substrate. The functionalized receptor can be an antibody, peptide, nucleic acid, phage, bacteria, virus, or any other molecule with a defined affinity for a target substrate. Examples of material that may be used for the “particles” and/or “carrier” include polylactic acid, polyglycolic acid, PLGA polymers, alginates and alginate derivatives, gelatin, collagen, fibrin, hyaluronic acid, laminin rich gels, agarose, natural and synthetic polysaccharides, polyamino acids, polypeptides, polyesters, poly anhydrides, polyphosphazines, poly(vinyl alcohols), poly(alkylene oxides), poly(allylamines) (PAM), poly(acrylates), modified styrene polymers, pluronic polyols, polyoxamers, poly(uronic acids), poly(vinylpyrrolidone) and copolymers or graft copolymers of any of the above. These examples do not limit their concentration, their cross-linking with different agents, their method of administration, their tailored degradation profiles and other characteristics known to those skilled in the art.

The particles, or a group of several particles in a complex, may be functionalized with a targeting agent (e.g., a ligand or antibody) that specifically binds (or substantially specifically binds) to a target (e.g., a target receptor or a cell surface target, such as a clinically relevant receptor or cell surface target (e.g., antigen)). The targeting agent may be attached directly to the particle itself. The targeting agent can be an antibody, peptide, nucleic acid, phage, bacteria, virus, or any other molecule with a specific affinity for a target receptor or cell surface target. In some instances, the receptor or cell surface target is PD-1, CTLA-4, HER2/neu, HER1/EGFR, VEGFR, 4-1BB, GITR, or other cellular receptors or cell surface targets. Other compounds or molecules, such as fluorophores or autofluorescent or luminescent markers, which may assist in detecting the particles (e.g., in vivo detection), may also be attached to the particles. The ligands and/or detectable labels may be attached directly to the particle or attached to the particle through bioorthogonal functional groups as described herein.

In certain embodiments, the support is a bone graft material, such as a bone graft substitute material. A bone graft substitute material is a material structurally similar to bone. In some instances, a bone graft substitute material is bioresorbable such that the bone graft substitute material can dissolve or be absorbed in the body over time. A bone graft substitute material can be osteoconductive, such that it facilitates blood vessel and new bone formation into the bone graft substitute material. In some instances, the bone graft substitute material is osteoinductive, such that facilitates the formation of new bone through active recruitment of mesenchymal stem cells from the surrounding tissue. For example, growth factors, such as bone morphogenetic proteins, may be included in the bone graft substitute material. Bone graft substitute materials include, but are not limited to, hydroxyapatite, tricalcium phosphate, demineralized bone matrix, bovine collagen, calcium sulfate, calcium phosphate, cancellous bone chips, and the like, and combinations thereof.

In certain embodiments, the support compositions comprise substituted alginate having units of formula:

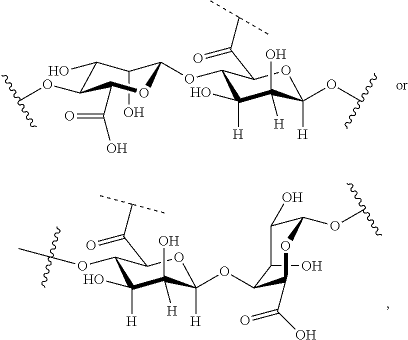

or a salt thereof, wherein the dashed line represents a bond to L.

In some embodiments, the support compositions comprise substituted hyaluronic acid having units of formula:

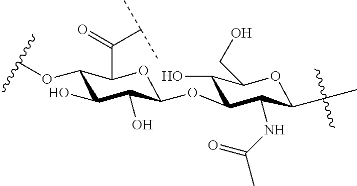

or a salt thereof, wherein the dashed line represents a bond to L.

The hyaluronic acid derivative includes a hyaluronic acid having a plurality of glucuronic acid units and a tetrazine-containing group linked or directly bonded to a glucuronic acid unit of the hyaluronic acid. The hyaluronic acid may also have a plurality of N-acetylglucosamine units. In certain embodiments, the N-acetylglucosamine units of the hyaluronic acid are not linked or conjugated to the tetrazine-containing group.

The tetrazine-containing group can be linked or directly bonded through a carboxylic acid of a glucuronic acid unit. The tetrazine-containing group can be incorporated into the hyaluronic acid from about 0.1% to about 80% as measured by the % of carboxylic acids being linked or conjugated to the tetrazine-containing group, such as about 1% to about 75%, about 5% to about 75%, about 10% to about 50%, or about 40% to about 75% as measured by the % of carboxylic acids being linked or conjugated to L of the tetrazine-containing group.

Additional support compositions are exemplified in WO2017/044983, WO2015/139025, and WO2014/205126, the entire contents of each of which is incorporated herein by reference in their entirety.

### D. Trans-Cyclooctene Functionalized Prodrugs

Trans-cyclooctene functionalized prodrugs are known in the art, including prodrugs of anticancer agents, as described in WO2018/187740, WO2014/205126, WO2015/139025, and WO2017/044983, which are incorporated herein by reference. Further embodiments using trans-cyclooctene functionalized prodrugs follow.

In some embodiments, the trans-cyclooctene functionalized prodrugs is a conjugate comprised of a payload linked to one or more trans-cyclooctene moieties.

In some embodiments, the conjugate (or trans-cyclooctene functionalized prodrug) comprises an immunomodulatory agent payload, such as for example, an immunomodulatory agent payload selected from the group consisting of a cytokine, chemokine, chemokine antagonist, therapeutic monoclonal antibody, and immune checkpoint inhibitor payload; or a pharmaceutically acceptable salt thereof.

In some embodiments, the immunomodulatory agent payload is an inhibitor of a cytokine payload, or a pharmaceutically acceptable salt thereof.

In some embodiments, the inhibitor of a cytokine payload is an inhibitor of TNF-α, infliximab, certolizumab, TGF-β, galunisertib, fresolimumab, M7824, CSF-1, pexidartinib, or cabiralizumab.

In some embodiments, the conjugate comprises a monoclonal antibody, or a pharmaceutically acceptable salt thereof.

In some embodiments, the conjugate comprises a therapeutic protein payload, or a pharmaceutically acceptable salt thereof.

In some embodiments, the therapeutic protein payload is an antibody-based drug, Fc fusion protein, anticoagulant, blood factor, bone morphogenetic protein, engineered protein scaffold, enzyme, growth factor, hormone, interferon, interleukin, or thrombolytic.

In some embodiments, the therapeutic protein payload is a cytokine, chemokine, growth factor, hormone, antibody, or antigen.

In some embodiments, the therapeutic protein payload is a payload of erythropoietin (EPO, e.g., native EPO or synthetic EPO (see, e.g., US 2003/0191291), such as, but not limited to, e.g., PROCRIT®, EPREX®, or EPOGEN® (epoetin-α), ARANESP® (darbepoietin-α), NEORECORMON®, EPOGIN® (epoetin-β), and the like); a growth hormone (e.g., a somatotropin, e.g., GENOTROPIN®, NUTROPIN®, NORDITROPIN®, SAIZEN®, SEROSTIM®, HUMATROPE®, etc.); theraputic monoclonal antibody (e.g Atezolizumab, Avelumab, Bevacizumab, Cemiplimab, Cetuximab, Daratumumab, Dinutuximab, Durvalumab, Elotuzumab, Ipilimumab, Isatuximab, Mogamulizumab, Necitumumab, Nivolumab, Obinutuzumab, Ofatumumab, Olaratumab, Panitumumab, Pembrolizumab, Pertuzumab, Ramucirumab, Rituximab, Trastuzumab etc); human growth hormone (hGH); bovine growth hormone (bGH); follicle stimulating hormone (FSH); interferon (e.g., IFN-γ, IFN-α, IFN-β, IFN-ω; IFN-τ, consensus interferon, and the like); insulin (e.g., Novolin, Humulin, Humalog, Lantus, Ultralente, etc.), insulin-like growth factor (e.g., IGF-I, IGF-II); blood factors (e.g., Factor X, tissue plasminogen activator (TPA), and the like, such as, but not limited to, e.g., ACTIVASE® (alteplase) tissue plasminogen activator, NOVOSEVEN® (recombinant human factor VIIa), Factor VIIa, Factor VIII (e.g., KOGENATE®), Factor IX, β-globin, hemoglobin, and the like); colony stimulating factors (e.g., granulocyte-CSF (G-CSF, e.g., NEUPOGEN® (filgrastim)), macrophage-CSF (M-CSF), granulocyte-macrophage-CSF (GM-CSF), Neulasta (pegfilgrastim), granulocyte-monocyte colony stimulating factor, megakaryocyte colony stimulating factor, and the like), transforming growth factors (e.g., TGF-beta, TGF-alpha); interleukins (e.g., IL-1, IL-2 (e.g., Proleukin®), IL-3, IL-4, IL-5, IL-6, IL-7, IL-8, IL-9, IE-12, and the like); a growth factor (e.g., epidermal growth factor (EGF), platelet-derived growth factor (PDGF, e.g., REGRANEX® (beclapermin)), fibroblast growth factors (FGFs, e.g., aFGF, bFGF, such as FIB LAST® (trafermin)), glial cell line-derived growth factor (GDNF), nerve growth factor (NGF), stem cell factor (e.g., STEMGEN® (ancestim)), keratinocyte growth factor, a hepatocyte growth factor, and the like); a soluble receptor (e.g., a TNF-α-binding soluble receptor such as ENBREL® (etanercept), a soluble VEGF receptor, a soluble interleukin receptor, a soluble γ/δ T cell receptor, and the like); an enzyme (e.g., α-glucosidase, CERAZYME® (imiglucarase, β-glucocerebrosidase, CEREDASE® (alglucerase); an enzyme activator (e.g., tissue plasminogen activator); a chemokine (e.g., IP-10, Mig, Groα/IL-8, regulated and normal T cell expressed and secreted (RANTES), MIP-1α, MIP-1ρ, MCP-1, PF-4, and the like); an angiogenic agent (e.g., vascular endothelial growth factor (VEGF); an anti-angiogenic agent (e.g., a soluble VEGF receptor); a protein vaccine; a neuroactive peptide such as bradykinin, cholecystokinin, gastin, secretin, oxytocin, gonadotropin-releasing hormone, beta-endorphin, enkephalin, substance P, somatostatin, galanin, growth hormone-releasing hormone, bombesin, warfarin, dynorphin, neurotensin, motilin, thyrotropin, neuropeptide Y, luteinizing hormone, calcitonin, insulin, glucagon, vasopressin, angiotensin II, thyrotropin-releasing hormone, vasoactive intestinal peptide, a sleep peptide, etc.; other proteins such as a thrombolytic agent, an atrial natriuretic peptide, bone morphogenic protein, thrombopoietin, relaxin, glial fibrillary acidic protein, follicle stimulating hormone, a human alpha-1 antitrypsin, a leukemia inhibitory factor, a transforming growth factor, a tissue factor, an insulin-like growth factor, a luteinizing hormone, a follicle stimulating hormone, a macrophage activating factor, tumor necrosis factor, a neutrophil chemotactic factor, a nerve growth factor, a tissue inhibitor of metalloproteinases; a vasoactive intestinal peptide, angiogenin, angiotropin, fibrin; hirudin; a leukemia inhibitory factor; or an IL-1 receptor antagonist (e.g., Kineret® (anakinra)); and the like

In some embodiments, the conjugate is of Formula X, or a pharmaceutically acceptable salt thereof:

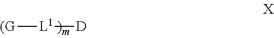

wherein
m is an integer from 1-150;
G, at each occurrence, is independently an optionally substituted trans-cyclooctene moiety;
D is a payload;
L¹, at each occurrence, is independently a linker.

In some embodiments of the conjugates described herein, each trans-cyclooctene moiety is independently:

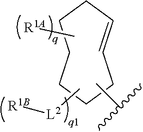

wherein:
R^{1A}, at each occurrence, is independently selected from the group consisting of C₁₋₄alkyl, C₁₋₄haloalkyl, and C₁₋₄alkoxy;
q is 0, 1, or 2;
q1 is 0 or 1;
R^{1B}, at each occurrence, is independently selected from the group consisting of G¹, —OH, —NR^{1c}—C₁₋₄alkylene-G¹, —NR^{1c}—C₁₋₄alkylene-N(R^{1d})₂, —NR^{1c}—C₁₋₆alkylene-N(C₁₋₄alkyl)₃⁺, —N(R^{1c})CHR^{1e}CO₂H, —N(R^{1c})—C₁₋₆alkylene-CO₂H, —N(R^{1f})—C₂₋₄alkylene-(N(C₁₋₄alkylene-CO₂H)—C₂₋₄alkylene)ₙ-N(C₁₋₄alkylene-CO₂H)₂, —N(R^{1c})CHR^{1e}C(O)OC₁₋₆alkyl, —N(R^{1c})—C₁₋₆alkylene-C(O)OC₁₋₆alkyl, —N(R^{1f})—C₂₋₄alkylene-(N(C₁₋₄alkylene-C(O)OC₁₋₆alkyl)-C₂₋₄alkylene)ₙ-N(C₁₋₄alkylene-C(O)OC₁₋₆alkyl)₂, —N(R^{1c})—C₁₋₆alkylene-SO₃H, —N(R^{1c})—(CH₂CH₂O)₁₋₃—CH₂CH₂N ((CH₂CH₂O)₁₋₃—C₁₋₆alkylene-CO₂H)₂, and —N(R^{1c})—CH(CH₂O—(CH₂CH₂O)₀₋₂—C₁₋₆alkylene-CO₂H)₂;
R^{1c} and R^{1d}, at each occurrence, are independently hydrogen or C₁₋₄alkyl;
R^{1e}, at each occurrence, is independently-C₁₋₄alkylene-CO₂H, —C₁₋₄alkylene-CONH₂, or —C₁₋₄alkylene-OH;
R^{1f}, at each occurrence, is independently hydrogen, C₁₋₆alkyl, or C₁₋₄alkylene-CO₂H;
n, at each occurrence, is independently 0, 1, 2, or 3;
L², at each occurrence, is independently selected from the group consisting of —C(O)— and C₁₋₃alkylene; and
G¹, at each occurrence, is independently an optionally substituted heterocyclyl.

In some embodiments, the conjugate is of Formula X, or a pharmaceutically acceptable salt thereof,

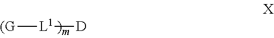

wherein
G is the trans-cyclooctene moiety, and G, at each occurrence, is independently

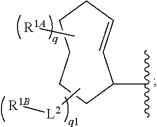

L¹, at each occurrence, is independently a linker;
m is an integer from 1-150;
D is a payload;
R^{1A}, at each occurrence, is independently selected from the group consisting of C₁₋₄alkyl, C₁₋₄haloalkyl, and C₁₋₄alkoxy;
q is 0, 1 or 2;
q1 is 0 or 1;
R^{1B}, at each occurrence, is independently selected from the group consisting of G¹, OH, —NR^{1c}—C₁₋₄alkylene-G¹, —NR^{1c}—C₁₋₄alkylene-N(R^{1d})₂, —NR^{1c}—C₁₋₆alkylene-N(C₁₋₄alkyl)₃⁺, —N(R^{1c})CHR^{1e}CO₂H, —N(R^{1c})—C₁₋₆alkylene-CO₂H, —N(R^{1f})—C₂₋₄alkylene-(N(C₁₋₄alkylene-CO₂H)—C₂₋₄alkylene), —N(C₁₋₄alkylene-CO₂H)₂, —N(R^{1c})CHR¹C(O)OC₁₋₆alkyl, —N(R^{1c})—C₁₋₆alkylene-C(O)OC₁₋₆alkyl, —N(R^{1f})—C₂₋₄alkylene-(N(C₁₋₄alkylene-C(O)OC₁₋₆alkyl)-C₂₋₄alkylene)ₙ-N(C₁₋₄alkylene-C(O)OC₁₋₆alkyl)₂, —N(R^{1c})—C₁₋₆alkylene-SO₃H, —N(R^{1c})—(CH₂CH₂O)₁₋₃—CH₂CH₂N ((CH₂CH₂O)₁₋₃—C₁₋₆alkylene-CO₂H)₂, and —N(R^{1c})—CH(CH₂O—(CH₂CH₂O)₀₋₂—C₁₋₆alkylene-CO₂H)₂;
R^{1c} and R^{1d}, at each occurrence, are independently hydrogen or C₁₋₄alkyl;
R^{1e}, at each occurrence, is independently-C₁₋₄alkylene-CO₂H, —C₁₋₄alkylene-CONH₂, or —C₁₋₄alkylene-OH;
R^{1f}, at each occurrence, is independently hydrogen, C₁₋₆alkyl, or C₁₋₄alkylene-CO₂H;
n, at each occurrence, is independently 0, 1, 2, or 3;
L², at each occurrence, is independently selected from the group consisting of —C(O)— and C₁₋₃alkylene; and
G¹, at each occurrence, is independently an optionally substituted heterocyclyl.

In some embodiments, q1 is 1.

In some embodiments, the payload is an immunomodulatory agent payload.

In some embodiments, the payload is a therapeutic monoclonal antibody, cytokine, chemokine, chemokine antagonist, and immune checkpoint inhibitor payload; or a pharmaceutically acceptable salt thereof.

In some embodiments, the payload is selected from a therapeutic agent for treating cancer (e.g., doxorubicin, daunorubicin, PNU-159682, etoposide, irinotecan, SN-38, docetaxel, paclitaxel, baccatin III, gemcitabine, podophyllotoxin, Carmustine, Ixabepilone, Patupilone (epothelone class), platinum drugs, exatecan, auristatin (dolastatin 10, MMAE, MMAD, MMAF), duocarmycin, pyrrolobenzodiazapene dimer, mitomycin C, bleomycin, calicheamicin, staurosporine, hemiasterlin), an immunosuppressant (e.g., cyclosporin A, rapamycin, and the like), an anti-fungal agent (e.g., Amphotericin, and the like), an antibiotic (e.g., vancomycin, daptomycin, doxycycline, ceftriaxone, trimethoprim, sulfamethoxazole, acyclovir, nystatin, amphotericin B, flucytosine, emtricitabine, gentamicin, colistin, and the like), a matrix metalloproteinase (MMP) inhibitor, L-dopa, oseltamivir, cefalexin, 5-aminolevulinic acid, cysteine, celecoxib, nimodipine, vancomycin, daptomycin, and cyclic-adenosine monophosphatidyl (c-AMP).

In some embodiments, the payload is selected from a therapeutic agent for treating cancer (e.g., paclitaxel, doxorubicin, daunorubicin, etoposide, irinotecan, SN-38, docetaxel, paclitaxel, gemcitabine, podophyllotoxin, Carmustine, Ixabepilone, Patupilone (epothelone class), platinum drugs, exatecan, auristatin (dolastatin 10, MMAE, MMAD, MMAF) mitomycin C, bleomycin, calicheamicin, staurosporine, hemiasterlin, and the like), an immunosuppressant (e.g., cyclosporin A, rapamycin, and the like), an anti-fungal agent (e.g., Amphotericin, and the like), an antibiotic (e.g., vancomycin, daptomycin, doxycycline, ceftriaxone, trimethoprim, sulfamethoxazole, acyclovir, nystatin, amphotericin B, flucytosine, emtricitabine, gentamicin, colistin, and the like), lurbinectedin, gardiquimod, a matrix metalloproteinase (MMP) inhibitor, L-dopa, oseltamivir, cefalexin, 5-aminolevulinic acid, cysteine, celecoxib, nimodipine, vancomycin, daptomycin, and cyclic-adenosine monophosphatidyl (c-AMP).

Reference to a payload, means that one or more atoms, including hydrogen or non-hydrogen atoms, of the original, unmodified payload is replaced by a covalent bond to one or more linker. The payloads are derived from the known nuclear payload and are modified to be covalently bonded to at least one optionally substituted trans-cyclooctene via a linker. The payloads, even after modification to arrive at the compounds described herein, maintain biological activity which is comparable to that observed in the original, unmodified payload. In certain embodiments, the payloads exhibit a binding activity or inhibition which is at least about 98%, about 95%, about 90%, about 85%, about 80%, about 75%, about 70%, about 65%, about 60%, about 55%, or about 50% of that observed in the original, unmodified payload.

In certain embodiments, a hydrogen atom bound to a heteroatom (e.g., N, O, or S) of the original, unmodified payload is replaced by a covalent bond to a linker. In certain embodiments, a halogen atom on a payload is replaced for attachment to the remainder of the compound. In certain embodiments, a hydrogen atom on a payload is replaced for attachment to the remainder of the compound. In certain embodiments, the hydrogen atom is on a heteroatom. In certain embodiments, the hydrogen atom is on a nitrogen. In certain embodiments, the hydrogen atom is on an oxygen. In certain embodiments, the hydrogen atom is on a carbon.

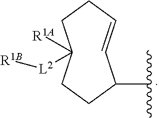

In some embodiments, G, at each occurrence, is independently

In some embodiments, G, at each occurrence, is independently

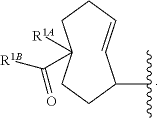

In some embodiments, the payload is a monoclonal antibody payload. A monoclonal antibody for use herein as a payload can be an entire monoclonal antibody, or a fragment thereof (e.g., antigen-binding fragment (Fab)). In some embodiments, the antibody is an immune cell engager, and as such would induce or elicit an immune response. In some embodiments, the monoclonal antibody, or fragment thereof, targets one or more of CD3 (NCBI Gene ID 916), CD28 (NCBI Gene ID 940), CD137 (4-1BB) (NCBI Gene ID 3604), CD16 (NCBI Gene ID 2214), NKG2D (NCBI Gene ID 22914), CD64 (NCBI Gene ID 2209), GITR/TNFRSF18 (NCBI Gene ID 8487), CD25 (NCBI Gene ID 3559), CD40 (NCBI Gene ID 958), CD4 (NCBI Gene ID 920), CXCR4 (NCBI Gene ID 7852), G-CSFR (NCBI Gene ID 1441), GM-CSFR (NCBI Gene ID 1438), CD122 (NCBI Gene ID 3560), PD1 (NCBI Gene ID 5133), CTLA4 (NCBI Gene ID 1493), LAG3 (NCBI Gene ID 3902), TIGIT (NCBI Gene ID 201633), NCR1 (NCBI Gene ID 9437), TIM3 (NCBI Gene ID 84868), VISTA (NCBI Gene ID 64115), CD134 (NCBI Gene ID 7293), CD27 (NCBI Gene ID 939), CD40L (NCBI Gene ID 959), ICOS (NCBI Gene ID 29851), BAFFR (NCBI Gene ID 115650), LFA-1 (NCBI Gene ID 3689), or BTLA (NCBI Gene ID 151888).

In certain embodiments, the payload is an antibody or antibody fragment which targets CD3, such as OKT3, SP34, UCHT1, Teplizumab, Otelixizumab, Visilizumab, or Foralumab, or an antibody fragment derived therefrom.

In certain embodiments, the payload is an antibody or antibody fragment which targets CD28, such as Theralizumab, TGN1412, or FR104, or an antibody fragment derived therefrom.

In certain embodiments, the payload is an antibody or antibody fragment which targets CD137 (4-1BB), such as Utomilumab, Urelumab, LVGN6051, or AGEN2373, or an antibody fragment derived therefrom.

In certain embodiments, the payload is an antibody or antibody fragment which targets CD16, such as AFM13, or an antibody fragment derived therefrom.

In certain embodiments, the payload is an antibody or antibody fragment which targets NKG2D, such as NNC0152-0002 or JNJ-64304500, or an antibody fragment derived therefrom.

In certain embodiments, the payload is an antibody or antibody fragment which targets CD64, such as H22, or an antibody fragment derived therefrom.

In certain embodiments, the payload is an antibody or antibody fragment which targets GITR/TNFRSF18, such as MK-4166, TRX518, MS-986156, AMG-228, or INCAGN01876, or an antibody fragment derived therefrom.

In certain embodiments, the payload is an antibody or antibody fragment which targets CD25, such as Daclizumab, RG6292, basiliximab, or HuMax-TAC, or an antibody fragment derived therefrom.

In certain embodiments, the payload is an antibody or antibody fragment which targets CD40, such as Iscalimab, ABBV-323, bleselumab (ASKP-1240), BI-655064, FFP-104, BMS986090, Dacetuzumab, or Lucatumumab, or an antibody fragment derived therefrom.

In certain embodiments, the payload is an antibody or antibody fragment which targets CD4, such as MAX.16H5, IT1208, Zanolimumab (HuMax-CD4), UB-421, or MTRX1011A, or an antibody fragment derived therefrom.

In certain embodiments, the payload is an antibody or antibody fragment which targets CXCR4, such as F50067, or an antibody fragment derived therefrom.

In certain embodiments, the payload is an antibody or antibody fragment which targets G-CSFR, such as CSL324, or an antibody fragment derived therefrom.

In certain embodiments, the payload is an antibody or antibody fragment which targets GM-CSFR, such as Mavrilimumab, or an antibody fragment derived therefrom.

In certain embodiments, the payload is an antibody or antibody fragment which targets CD122, such as Hu-Mik(beta)1, or an antibody fragment derived therefrom.

In certain embodiments, the payload is an antibody or antibody fragment which targets PD-1, such as CC-90006, Cemiplimab, Camrelizumab, or TSR-042, or an antibody fragment derived therefrom.

In certain embodiments, the payload is an antibody or antibody fragment which targets CTLA4, such as Tremelimumab or ipilimumab, or an antibody fragment derived therefrom.

In certain embodiments, the payload is an antibody or antibody fragment which targets LAG3, such as Relatlimab (BMS-986016), GSK2831781, Cemiplimab (REGN3767), Favezelimab, Ieramilimab, or Mavezelimab, or an antibody fragment derived therefrom.

In certain embodiments, the payload is an antibody or antibody fragment which targets TIGIT, such as BMS-986207, Tiragolumab, Vibostolimab, Etigilimab, Domvanalimab, ASP-8374, IBI939, BGB-A1217, COM902, or M6223, or an antibody fragment derived therefrom.

In certain embodiments, the payload is an antibody or antibody fragment which targets NCR1, such as hNKp46.02, or an antibody fragment derived therefrom.

In certain embodiments, the payload is an antibody or antibody fragment which targets TIM3, such as Cobolimab, Sym023, LY3321367, BMS-986258, SHR-1702, Sabatolimab, or INCAGN02390, or an antibody fragment derived therefrom.

In certain embodiments, the payload is an antibody or antibody fragment which targets VISTA, such as SG7, K01401-020, CI-8993, or JNJ-61610588, or an antibody fragment derived therefrom.

In certain embodiments, the payload is an antibody or antibody fragment which targets CD134, such as KHK4083 or ISB830, or an antibody fragment derived therefrom.

In certain embodiments, the payload is an antibody or antibody fragment which targets CD27, such as Varlilumab, MK-5890, or CDX-527, or an antibody fragment derived therefrom.

In certain embodiments, the payload is an antibody or antibody fragment which targets CD40L, such as Dapirolizumab, or an antibody fragment derived therefrom.

In certain embodiments, the payload is an antibody or antibody fragment which targets ICOS, such as MEDI-570, KY1044, JTX-2011, or GSK3359609, or an antibody fragment derived therefrom.

In certain embodiments, the payload is an antibody or antibody fragment which targets BAFFR, such as Ianalumab, or an antibody fragment derived therefrom.

In certain embodiments, the payload is an antibody or antibody fragment which targets LFA-1, such as Efalizumab, or an antibody fragment therefrom.

In certain embodiments, the payload is an antibody or antibody fragment which targets BTLA, such as Icatolimab, or an antibody fragment derived therefrom.

In some embodiments, the payload is an anti-CD3 (αCD3) monoclonal antibody, or a derivative, or analog thereof. In some embodiments, the anti-CD3 (αCD3) monoclonal antibody is SP34, UCHT1, or OKT3, or a derivative, or analog thereof.

In some embodiments, at least one payload is selected from an inhibitor of poly(ADP-ribose) polymerase (PARP), a duocarmycin, a pyrrolobenzodiazepine (PBD), hemiasterlin, HTI-286, an anti-CD3 (αCD3) monoclonal antibody, lurbinectedin, MSA-2, gardiquimod, ciprofloxacin, Paclitaxel, Gemcitabine, Mitomycin C, Etoposide, exatecan, and MMAE, or a derivative, or analog thereof.

In some embodiments, D is a payload selected from an inhibitor of poly(ADP-ribose) polymerase (PARP), a duocarmycin, a pyrrolobenzodiazepine (PBD), hemiasterlin, HTI-286, and an anti-CD3 (αCD3) monoclonal antibody, or a derivative, or analog thereof.

In some embodiments, at least one payload is selected from lurbinectedin, MSA-2, gardiquimod, ciprofloxacin, Paclitaxel, Gemcitabine, Mitomycin C, Etoposide, exatecan, Seco-Duocarmycin SA, and MMAE, or a derivative, or analog thereof.

In some embodiments, a payload is an inhibitor of poly(ADP-ribose) polymerase (PARP), or a derivative, or analog thereof. In some embodiments, the inhibitor of poly(ADP-ribose) polymerase (PARP inhibitor) is niraparib, talazoparib, olaparib, pamiparib, rucaparib, veliparib, iniparib, 3-aminobenzamide, CEP-9722, E7016, or a derivative, or analog thereof.

In some embodiments, a payload is:

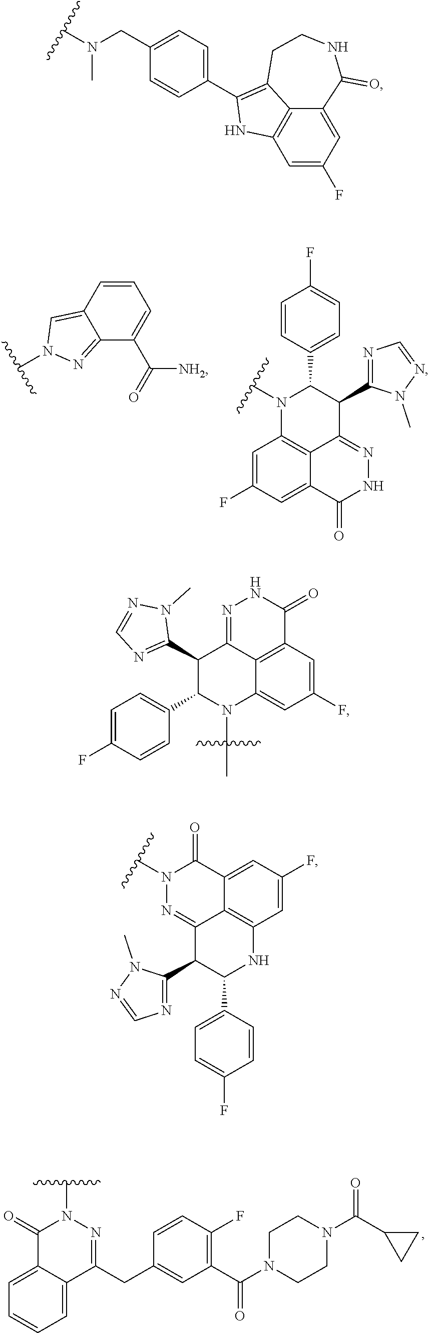 [SMILES: *N(C)Cc1ccc(-c2[nH]c3cc(F)cc4c3c2CCNC4=O)cc1 ; *n1cc2cccc(C(N)=O)c2n1 ; *N1c2cc(F)cc3c(=O)[nH]nc(c23)[C@H](c2ncnn2C)[C@H]1c1ccc(F)cc1 ; *N1c2cc(F)cc3c(=O)[nH]nc(c23)[C@H](c2ncnn2C)[C@H]1c1ccc(F)cc1 ; *n1nc2c3c(cc(F)cc3c1=O)N[C@H](c1ccc(F)cc1)[C@H]2c1ncnn1C ; *n1nc(Cc2ccc(F)c(C(=O)N3CCN(C(=O)C4CC4)CC3)c2)c2ccccc2c1=O]

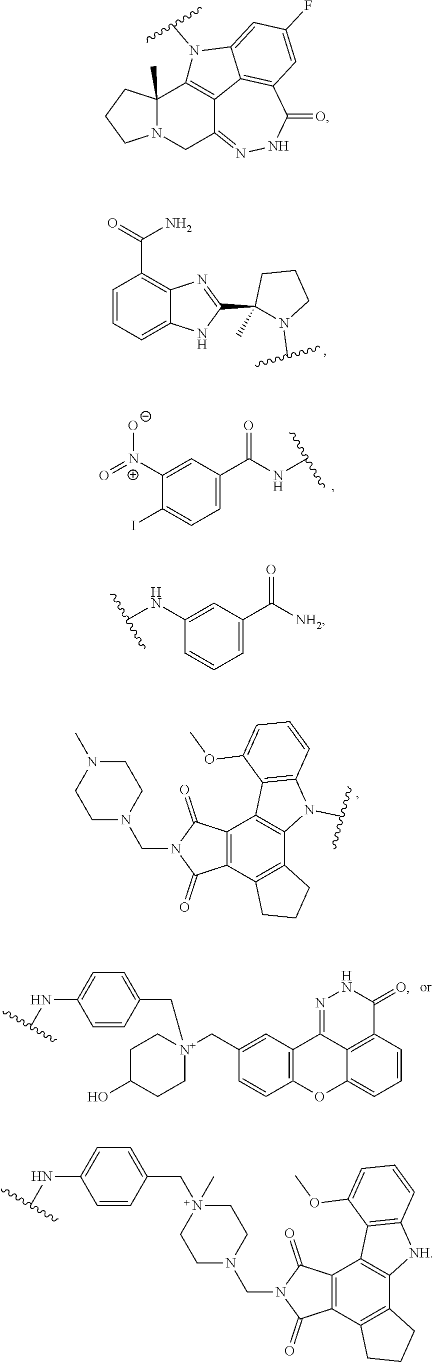

In some embodiments, a payload is a duocarmycin, or a derivative, or analog thereof. In some embodiments, the duocarmycin is Duocarmycin A, Duocarmycin B1, Duocarmycin B2, Duocarmycin C1, Duocarmycin C2, Duocarmycin D, Duocarmycin SA, CC-1065, adozelesin, carzelesin, bizelesin, or a derivative, or analog thereof.

In some embodiments, a payload is:

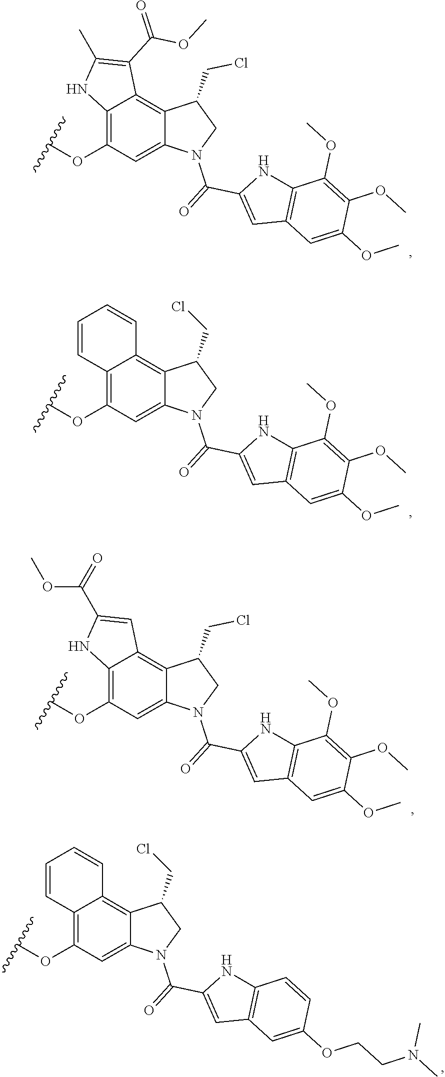

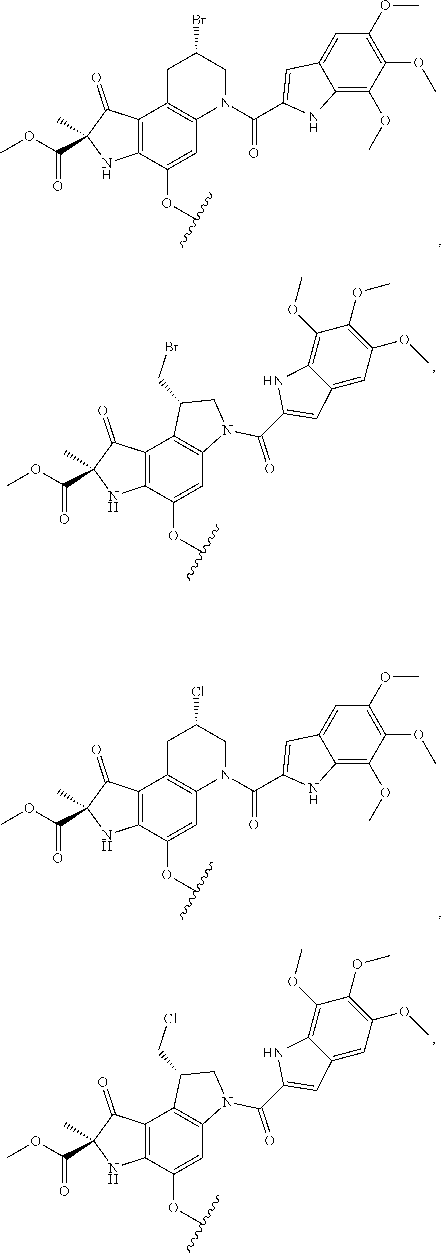

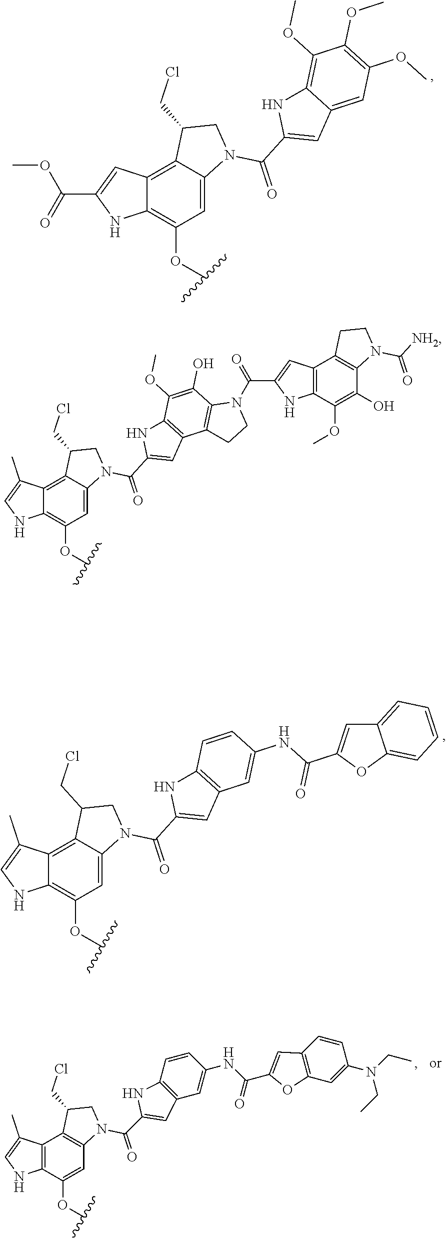

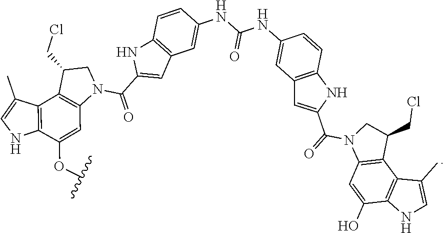 [SMILES: *Oc1cc2c(c3c(C)c[nH]c13)[C@H](CCl)CN2C(=O)c1cc2cc(NC(=O)Nc3ccc4[nH]c(C(=O)N5C[C@@H](CCl)c6c5cc(O)c5[nH]cc(C)c65)cc4c3)ccc2[nH]1]

In some embodiments, a payload is a pyrrolobenzodiazepine (PBD), or a derivative, or analog thereof. In some embodiments, the pyrrolobenzodiazepine (PBD) is [1,2]diazepino [3,4-e]indole, or a derivative, or analog thereof.

In some embodiments, a payload is:

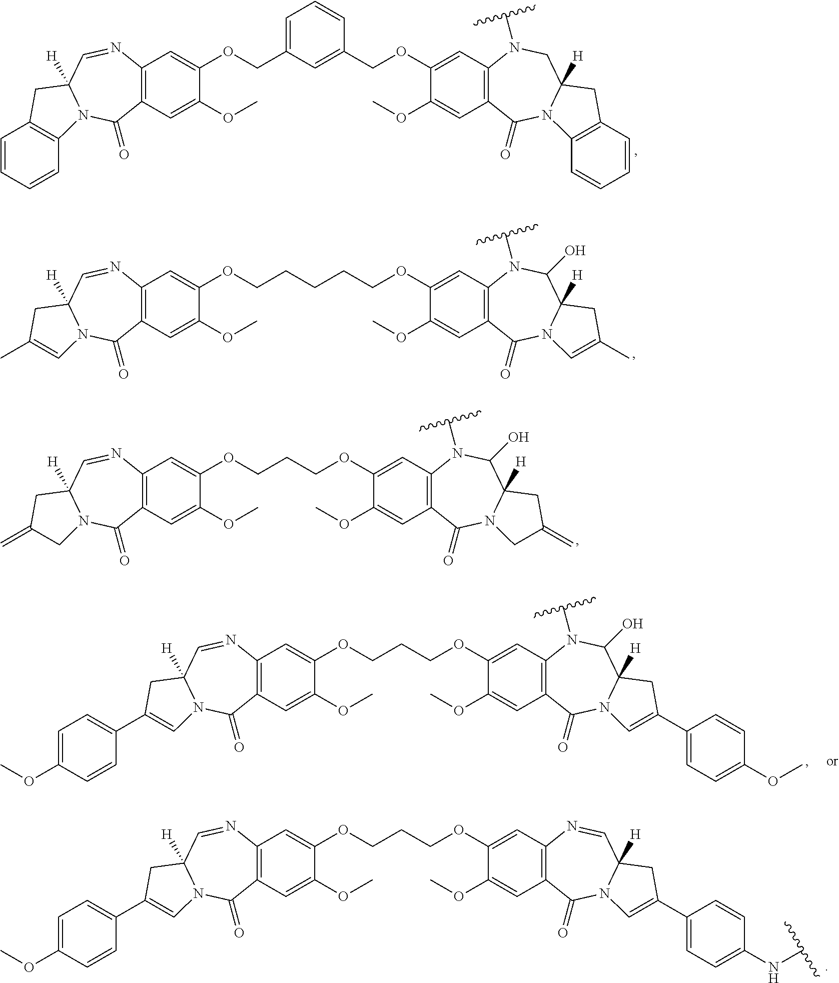

In some embodiments, a payload is an inhibitor of tubulin polymerization. In some embodiments, a payload is hemiasterlin, HTI-286, or a derivative, or analog thereof.

In some embodiments, a payload is derived from:

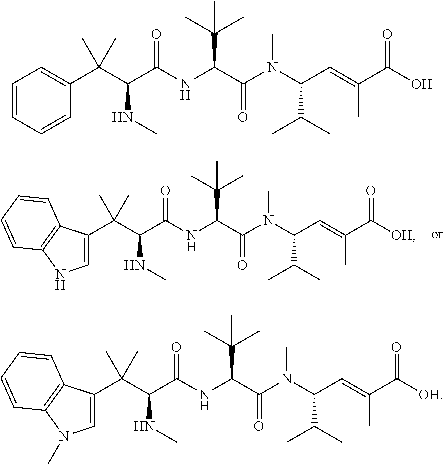 [SMILES: CN[C@H](C(=O)N[C@H](C(=O)N(C)[C@H](/C=C(\C)C(=O)O)C(C)C)C(C)(C)C)C(C)(C)c1ccccc1 ; CN[C@H](C(=O)N[C@H](C(=O)N(C)[C@H](/C=C(\C)C(=O)O)C(C)C)C(C)(C)C)C(C)(C)c1c[nH]c2ccccc12 ; "or" ; CN[C@H](C(=O)N[C@H](C(=O)N(C)[C@H](/C=C(\C)C(=O)O)C(C)C)C(C)(C)C)C(C)(C)c1cn(C)c2ccccc12]

In some embodiments, a payload is:

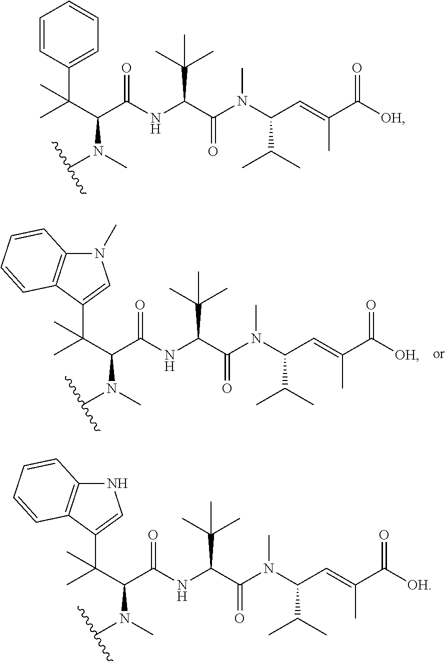 [SMILES: *N(C)[C@H](C(=O)N[C@H](C(=O)N(C)[C@H](/C=C(\C)C(=O)O)C(C)C)C(C)(C)C)C(C)(C)c1ccccc1 ; *N(C)[C@H](C(=O)N[C@H](C(=O)N(C)[C@H](/C=C(\C)C(=O)O)C(C)C)C(C)(C)C)C(C)(C)c1cn(C)c2ccccc12 ; "or" ; *N(C)[C@H](C(=O)N[C@H](C(=O)N(C)[C@H](/C=C(\C)C(=O)O)C(C)C)C(C)(C)C)C(C)(C)c1c[nH]c2ccccc12]

In some embodiments, the payload comprises a topoisomerase inhibitor. In some embodiments, the payload comprises camptothecin, or a derivative, or analog thereof. In some embodiments, the payload comprises topotecan, irinotecan, silatecan, cositecan, exatecan, lurtotecan, gimatecan, belotecan, or rubitecan.

In some embodiments, the payload comprises

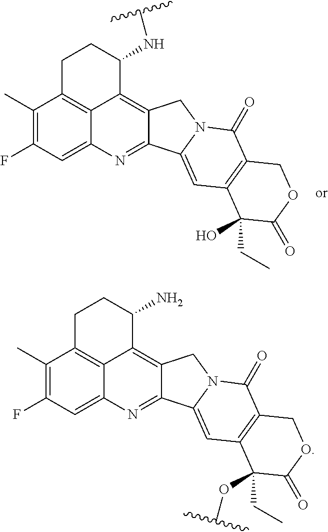 [SMILES: *N[C@H]1CCc2c(C)c(F)cc3nc4c(c1c23)Cn1c-4cc2c(c1=O)COC(=O)[C@]2(O)CC ; *O[C@]1(CC)C(=O)OCc2c1cc1n(c2=O)Cc2c-1nc1cc(F)c(C)c3c1c2[C@@H](N)CC3]

In some embodiments, the payload comprises

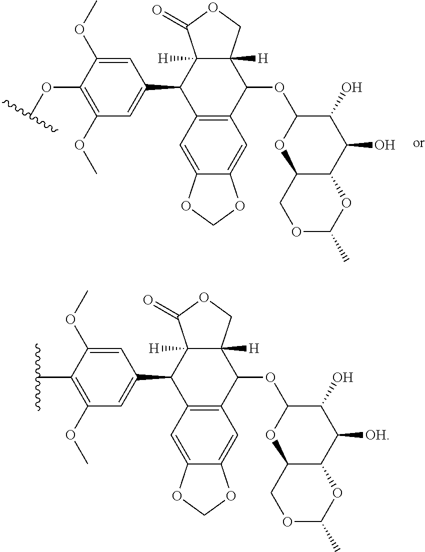

In some embodiments, the payload comprises

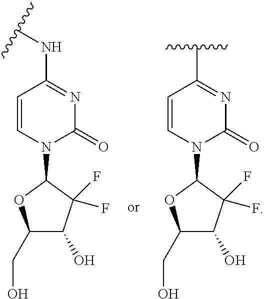 [SMILES: *Nc1ccn([C@@H]2O[C@H](CO)[C@@H](O)C2(F)F)c(=O)n1 ; "or" ; *c1ccn([C@@H]2O[C@H](CO)[C@@H](O)C2(F)F)c(=O)n1]

In some embodiments, the payload comprises

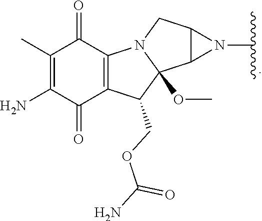 [SMILES: *N1C2CN3C4=C(C(=O)C(N)=C(C)C4=O)[C@@H](COC(N)=O)[C@@]3(OC)C21]

In some embodiments, the payload comprises

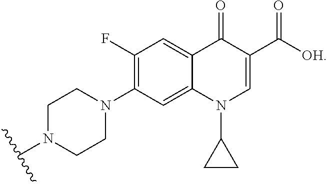 [SMILES: *N1CCN(c2cc3c(cc2F)c(=O)c(C(=O)O)cn3C2CC2)CC1]

In some embodiments, the payload comprises

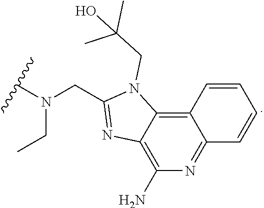 [SMILES: *N(CC)Cc1nc2c(N)nc3ccccc3c2n1CC(C)(C)O]

In some embodiments, the payload comprises

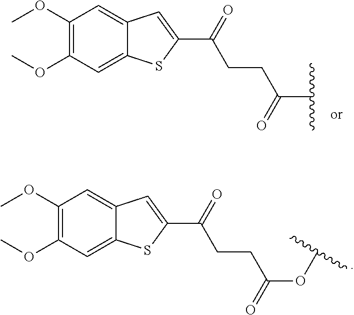 [SMILES: *C(=O)CCC(=O)c1cc2cc(OC)c(OC)cc2s1 ; *OC(=O)CCC(=O)c1cc2cc(OC)c(OC)cc2s1]

In some embodiments, the payload comprises a polypeptide. In some embodiments, the polypeptide comprises one or more lysine, serine, threonine, or tyrosine residues. In some embodiments, the linker L¹ is covalently bonded to a lysine, serine, threonine, or tyrosine residue present on the payload. In some embodiments, the polypeptide comprises one or more lysine residues. In some embodiments, the linker L¹ is covalently bonded to a lysine residue present on the payload.

In some embodiments, the payload comprises an N-terminal amino acid, wherein the linker L¹ is covalently bonded to a N-terminal amino acid.

In some embodiments, m is 1-20.

In some embodiments, the payload is an immunomodulatory agent payload.

In some embodiments, the immunomodulatory agent payload is an antibody payload.

In some embodiments, the immunomodulatory agent payload is the immune checkpoint inhibitor payload. In some embodiments, the immune checkpoint inhibitor payload is a payload of pidilizumab, sintilimab, AMP-224, atezolizumab, durvalumab, BMS-936559, tremelimumab, indoximod, epacadostat, a TIGIT inhibitor (e.g., LAG-3, such as an anti-LAG-3 antibody; TIM-3, such as an anti-TIM-3 antibody), a B7 molecule, or a BTLA pathway antagonist.

In some embodiments, the immune checkpoint inhibitor payload is an immune checkpoint inhibitor antibody payload. In some embodiments, the immune checkpoint inhibitor antibody payload is a PD-1 inhibitor payload. In some embodiments, the PD-1 inhibitor payload is a nivolumab, pembrolizumab, pidilizumab, sintilimab, or AMP-224 payload.

In some embodiments, the immune checkpoint inhibitor antibody payload is a PD-L1 inhibitor payload. In some embodiments, the PD-L1 inhibitor payload is an atezolizumab, avelumab, durvalumab, or BMS-936559 payload.

In some embodiments, the immune checkpoint inhibitor antibody payload is a CTLA4 inhibitor payload. In some embodiments, the CTLA4 inhibitor payload is an ipilimumab or tremelimumab payload.

In some embodiments, the immune checkpoint inhibitor payload is an indoleamine 2,3-dioxygenase (IDO) inhibitor payload. In some embodiments, the IDO inhibitor payload is an indoximod or epacadostat payload.

In some embodiments, the immunomodulatory agent payload is a cytokine payload.

In some embodiments, the cytokine payload is an interferon, interleukin, tumor necrosis factor, erythropoietin, MIP3a, ICAM, macrophage colony stimulating factor, Erythropoietin (EPO), granulocyte colony stimulating factor (GCSF), or granulocyte-macrophage colony stimulating factor payload.

In some embodiments, the interleukin payload is chosen from IL-1 to IL-40. In some embodiments, the interleukin payload is IL-2, IL-7, IL-12, IL-15, IL-18, or IL-21.

In some embodiments, the immunomodulatory agent payload is a type 1 cytokine (IL-2, IL-12, TNF-B, IFN-g).

In some embodiments, the cytokine payload is selected from the group consisting of IFN-alpha, IFN-beta, IFN-gamma, pegylated IFN-α, and apolipoprotein A-I fusion protein with IFN-α, interleukin, IL-2, IL-2 covalently bound to immunoglobulins (e.g., cergutuzumab amunaleukin, RO6874281), IL-2 covalently bound to PEG molecules (e.g., NKTR-214), IL-10, PEGylated IL-10 (e.g., pegilodecakin), IL-7, IL-12, IL-15, recombinant aglycosylated IL-15, fusion protein of IL-15 with the binding domain of IL-15Rα (e.g., RLI), triple fusion protein comprising human IL-15, the binding domain of IL-15Rα and apolipoprotein A-I, ALT-803 (IL-15 fused to IgG1 Fc domain), IL-18, IL-21, tumor necrosis factor, TNF-alpha, TNF-beta), erythropoietin (EPO), MIP3a, ICAM, macrophage colony stimulating factor (M-CSF), granulocyte colony stimulating factor (GCSF), granulocyte-macrophage colony stimulating factor (GM-CSF), GM-CSF, and talimogene laherparepvec.

In some embodiments, the immunomodulatory agent payload is the chemokine payload.

In some embodiments, the chemokine payload is a CCL27, CCL28, CCL2, CCL3, CCL5, CXCL1, CXCL2, CXCL3, CXCL5, CXCL6, CXCL8, CXCL9, CXCL10, CXCL11, CXCL12, or CXCL14 payload.

In some embodiments, the immunomodulatory agent payload is the chemokine antagonist payload. In some embodiments, the chemokine antagonist payload is a plerixafor payload.

In some embodiments, the immunomodulatory agent is a monoclonal antibody specific to a cytokine or a cytokine receptor.

In some embodiments, the immunomodulatory agent payload comprises a polypeptide.

In some embodiments, the polypeptide comprises one or more lysine residues.

In some embodiments, the polypeptide comprises one or more lysine, serine, threonine, or tyrosine residues.

In some embodiments, the trans-cyclooctene is linked to one of the one or more lysine residues.

In some embodiments, the trans-cyclooctene is independently linked to one or more lysine, serine, threonine, or tyrosine residues.

In some embodiments, the polypeptide comprises an N-terminal amino acid, wherein an occurrence of the bioorthogonal moiety is linked to the N-terminal amino acid.

In some embodiments, m is 1-20. In some embodiments, m is 1-10. In some embodiments, m is 1-5. In some embodiments, m is 20, 19, 18, 17, 16, 15, 14, 13, 12, 11, 10, 9, 8, 7, 6, 5, 4, 3, 2, or 1. In some embodiments, m is 1.

In some embodiments, the trans-cyclooctene functionalized prodrug of Formula XI:

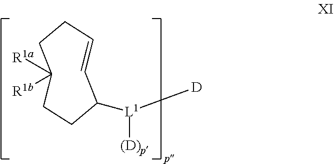

or a pharmaceutically acceptable salt thereof, wherein:
R^{1a}, at each occurrence, is independently selected from the group consisting of hydrogen, C₁₋₄alkyl, and C₁₋₄haloalkyl;
R^{1b}, at each occurrence, is independently selected from the group consisting of hydrogen, C₁₋₄alkyl, C₁₋₄haloalkyl, C(O)OH, C(O)OC₁₋₄alkyl, C(O)N(R^{1c})CHR^{1e}CO₂H, C(O)N(R^{1c})CHR^{1e}C(O)OC₁₋₄alkyl, C(O)N(R^{1c})—C₁₋₆alkylene-CO₂H, and C(O)N(R^{1c})—C₁₋₆alkylene-C(O)OC₁₋₄alkyl;
R^{1c}, at each occurrence, is independently hydrogen or C₁₋₄alkyl;
R^{1e}, at each occurrence, is independently-C₁₋₄alkylene-CO₂H, —C₁₋₄alkylene-CONH₂, or —C₁₋₄alkylene-OH;
D, at each occurrence, is independently a payload;
L¹, at each occurrence, is independently a linker;
p′, at each occurrence, is independently 0, 1, or 2; and
p″, at each occurrence, is independently 1, 2, or 3.

In some embodiments, D, at each occurrence, is independently selected from the group consisting of an anticancer agent payload, a toll-like receptor (TLR) agonist payload and a stimulator of interferon genes (STING) agonist payload.

In some embodiments, R^{1a} is hydrogen.

In some embodiments, R^{1a} is C₁₋₄alkyl.

In some embodiments, R^{1a} is CH₃.

In some embodiments, R^{1b} is selected from the group consisting of C(O)OH, C(O)OC₁₋₄alkyl, C(O)N(R^{1c})CHR^{1e}CO₂H, C(O)N(R^{1c})CHR^{1e}C(O)OC₁₋₄alkyl, C(O)N(R^{1c})—C₁₋₆alkylene-CO₂H, and C(O)N(R^{1c})—C₁₋₆alkylene-C(O)OC₁₋₄alkyl.

In some embodiments, R^{1b} is selected from the group consisting of C(O)OH, C(O)N(R^{1c})CHR^{1e}CO₂H, and C(O)N(R^{1c})CH₂CO₂H.

In some embodiments, R^{1b} is selected from the group consisting of —NR^{1c}—CH₂CH₂—N(CH₃)₃⁺, —N(R^{1c})—CH₂CH₂—SO₃H, —N(R^{1c})—(CH₂CH₂O)₃—CH₂CH₂N ((CH₂CH₂O)₃—CH₂CH₂—CO₂H)₂, and —N(R^{1c})—CH(CH₂O—CH₂CH₂—CO₂H)₂.

In some embodiments, the trans-cyclooctene moiety (G) is:

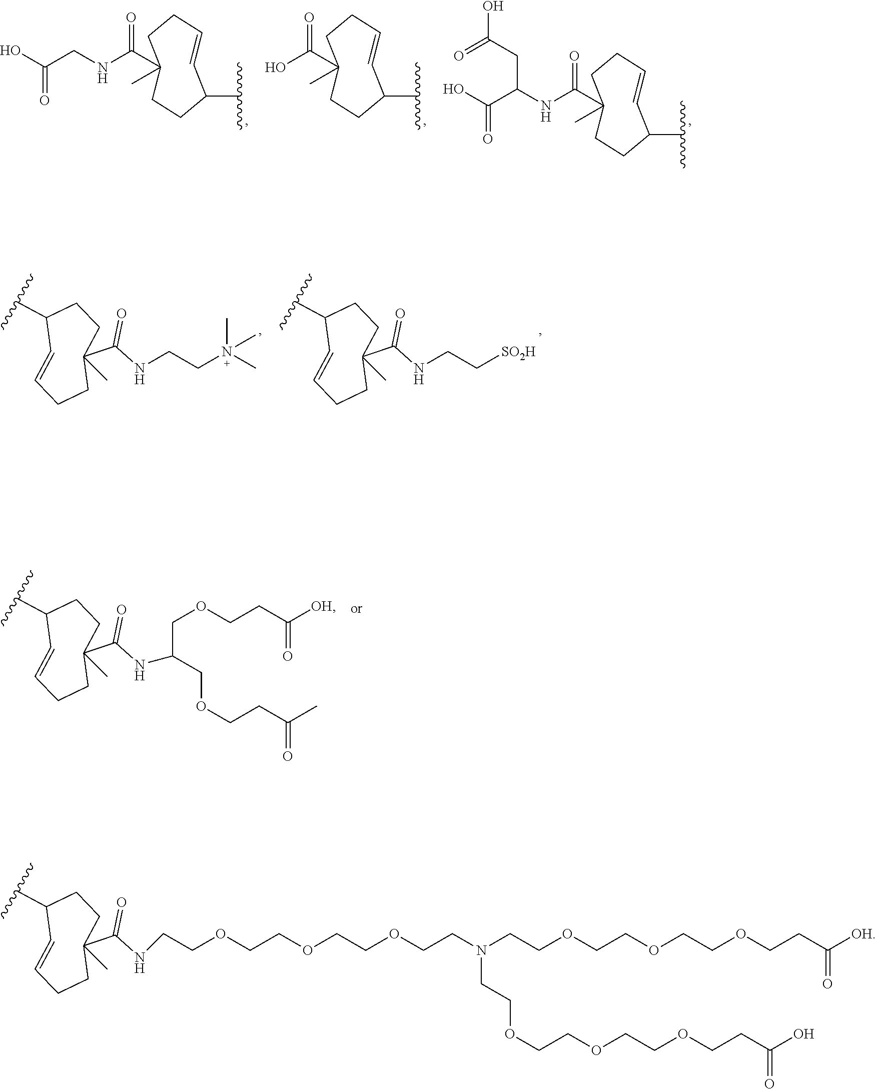

In some embodiments, the trans-cyclooctene moiety is:

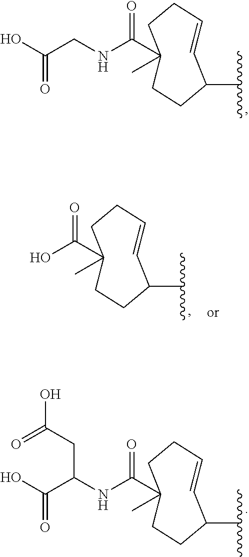 [SMILES: *C1/C=C/CCC(C)(C(=O)NCC(=O)O)CC1 ; *C1/C=C/CCC(C)(C(=O)O)CC1 ; *C1/C=C/CCC(C)(C(=O)NC(CC(=O)O)C(=O)O)CC1]

In some embodiments, the trans-cyclooctene moiety is

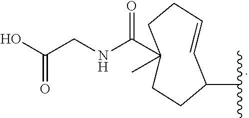 [SMILES: *C1/C=C/CCC(C)(C(=O)NCC(=O)O)CC1]

In some embodiments, the trans-cyclooctene moiety is

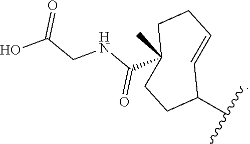

In some embodiments, the trans-cyclooctene moiety is

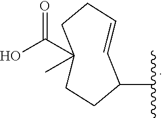 [SMILES: *C1/C=C/CCC(C)(C(=O)O)CC1]

In some embodiments, the trans-cyclooctene moiety is

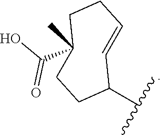

In some embodiments, the trans-cyclooctene moiety is

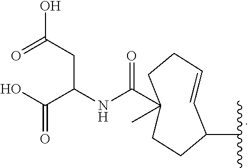 [SMILES: *C1/C=C/CCC(C)(C(=O)NC(CC(=O)O)C(=O)O)CC1]

In some embodiments, the trans-cyclooctene moiety is

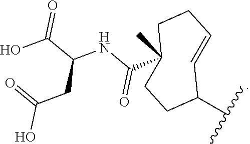

In some embodiments, the trans-cyclooctene moiety is

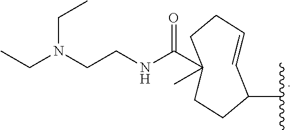 [SMILES: *C1/C=C/CCC(C)(C(=O)NCCN(CC)CC)CC1]

In some embodiments, the trans-cyclooctene moiety is

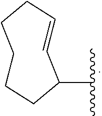 [SMILES: *C1/C=C/CCCCC1]

In some embodiments, the trans-cyclooctene moiety is

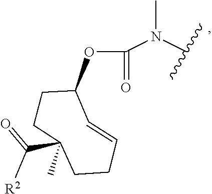

and R² is —OH, 2-aminoethanesulfonic acid, an N-linked natural or unnatural amino acid, or an optionally substituted ethylenediamine; wherein R² may be optionally further substituted with a polyether.

In some embodiments, the trans-cyclooctene moiety comprises

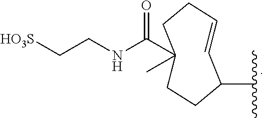 [SMILES: *C1/C=C/CCC(C)(C(=O)NCCS(=O)(=O)O)CC1]

In some embodiments, the trans-cyclooctene moiety comprises

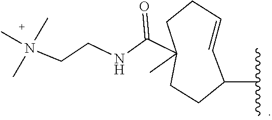

In some embodiments, the trans-cyclooctene moiety of comprises

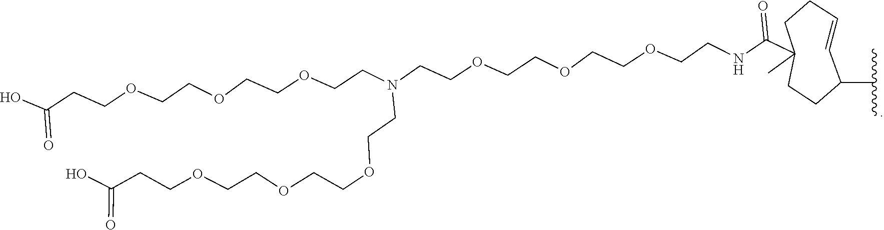 [SMILES: *C1/C=C/CCC(C)(C(=O)NCCOCCOCCOCCN(CCOCCOCCOCCC(=O)O)CCOCCOCCOCCC(=O)O)CC1]

In some embodiments, the trans-cyclooctene moiety of comprises

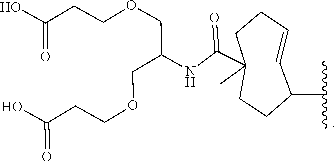 [SMILES: *C1/C=C/CCC(C)(C(=O)NC(COCCC(=O)O)COCCC(=O)O)CC1]

In some embodiments, R^{1e} is —CH₂CO₂H, —CH₂CH₂CO₂H, —CH₂CONH₂, —CH₂CH₂CONH₂, —CH₂OH, or —CH(CH₃)OH.

In some embodiments, R^{1e} is —C₁₋₄alkylene-CO₂H.

In some embodiments, R^{1e} is —CH₂CO₂H.

In some embodiments, R^{1b} is —C(O)N(R^{1c})—C₁₋₆alkylene-CO₂H.

In some embodiments, R^{1b} is —C(O)N(R^{1c})CH₂CO₂H.

In some embodiments, R^{1c} is hydrogen.

In some embodiments, R^{1b} is hydrogen.

In some embodiments, R^{1b} is C(O)OH.

In some embodiments, linker L¹ may have 1 to 100 linking atoms, and may include ethylene-oxy groups, amines, esters, amides, carbamates, carbonates, and ketone functional groups. For example, linkers may have from 1 to 50 linking atoms, or from 5 to 50 linking atoms, or from 10 to 50 linking atoms, or from 1 to 40 linking atoms, or from 1 to 30 linking atoms, or from 1 to 20 linking atoms, or from 1 to 10 linking atoms, or from 1 to 5 linking atoms, or from 5 to 30 linking atoms, or from 10 to 30 linking atoms, or from 5 to 40 linking atoms, or from 5 to 50 linking atoms, or from 10 to 50 linking atoms.

In some embodiments, linker L¹ may comprise one or more (e.g., 1-10 or 1-5) chain heteroatoms (e.g., O, N, S) and one or more (e.g., 1-10 or 1-5) alkylene, alkenylene, alkynylene, arylene, heteroarylene, cycloalkylene or heterocycloalkylene moieties; wherein each alkylene, alkenylene, alkynylene, arylene, heteroarylene, cycloalkylene or heterocycloalkylene moiety, may be independently optionally substituted with one to five substituents independently selected from oxo, halo, C₁₋₄ alkyl, C₁₋₄ alkoxy, and C₁₋₄ haloalkyl.

In some embodiments, linker L¹ may be of the formula:

—Y¹⁰—(CH₂)_{n′}—Y²⁰—(CH₂)_{m″}—Y³⁰—
wherein:
each of Y¹⁰, Y²⁰, and Y³⁰ are independently a bond, —NR¹¹⁰—, —O—, —S(O)₀₋₂—, —NR¹¹⁰° C. (O)—, —C(O)NR¹¹⁰—, —NR¹¹⁰S(O)₂—, —S(O)₂NR¹¹⁰—, —CR¹²⁰═N—NR¹¹⁰—, —NR¹¹⁰—N═CR¹²⁰—, —C(O)—, —OC(O)—, —OC(O)O—, alkylene, alkenylene, alkynylene, arylene, heteroarylene, cycloalkylene or heterocycloalkylene; wherein each alkylene, alkenylene, alkynylene, arylene, heteroarylene, cycloalkylene or heterocycloalkylene is independently optionally substituted with one to five substituents independently selected from oxo, halo, C₁₋₄ alkyl, C₁₋₄ alkoxy, and C₁₋₄ haloalkyl;
each R¹¹⁰ is independently hydrogen, C₁₋₄ alkyl, C₁₋₄ haloalkyl, aryl, heteroaryl, cycloalkyl or heterocyclyl;
each R¹²⁰ is independently hydrogen, C₁₋₄ alkyl, C₁₋₄ haloalkyl, aryl, heteroaryl, cycloalkyl or heterocyclyl; and
n′ and m″ are each independently 0, 1, 2, 3, 4, 5, 6, 7, or 8.

In certain embodiments, the linker is a bond.

In certain embodiments, the linker is not a bond. In certain embodiments, each R¹¹⁰ is independently hydrogen, C₁₋₄ alkyl, C₁₋₄ haloalkyl, aryl, heteroaryl, cycloalkyl or heterocyclyl; and each R¹²⁰ is independently hydrogen, C₁₋₄ alkyl, C₁₋₄ haloalkyl, aryl, heteroaryl, cycloalkyl or heterocyclyl.

Representative linkers include, but are not limited to, those shown below:

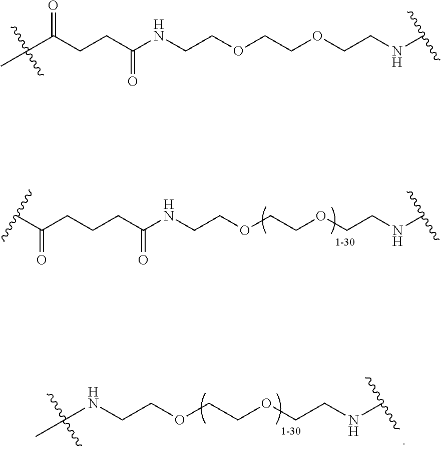

Representative linkers include, but are not limited to, those shown below:

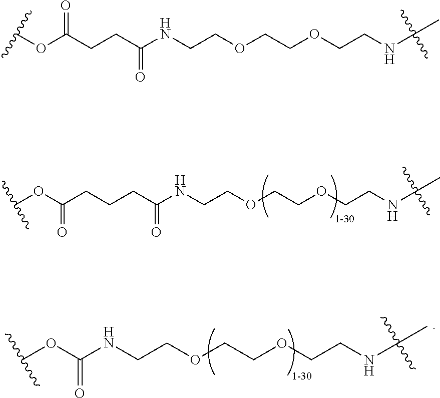

In some embodiments, linker L¹ may comprise one or more of polyethylene glycol (e.g., PEG having an average molecular weight of from 300 g/mol to 10,000 g/mol), ethylene-1,2-diylbis(methylcarbamate, an arylene (e.e., phenylene), ethylene-oxy, amine, ester, amide, carbamate, ketone (i.e., formyl), or carbonate. In some embodiments, linker L¹ may comprise

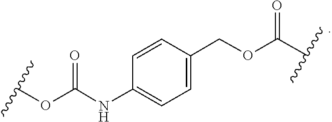 [SMILES: *OC(=O)Nc1ccc(COC(*)=O)cc1]

In some embodiments, linker L¹ may comprise one or more natural or unnatural amino acids, which may be referred to as a peptide linker. Where the drug (D) comprises an amino moiety, the linker may be bound thereto using a peptide linker made up of a carboxylic acyl unit, and one or more amino acids making up a protein or peptide sequence. In some embodiments, linker L¹ may also contain a self-immolating spacer which spaces the drug and the protein peptide sequence.

In some embodiments, linker L¹ may be a peptide linker represented by “A-Y—Z—X—W” in which “A” is the carboxylic acyl unit, “Y” and “Z” are each one or more natural or unnatural amino acids and together form a peptide sequence, and “X” and “W” are optional additional linkers having from 1 to 50 linking atoms, or from 5 to 10 linking atoms, or from 1 to 10 linking atoms which spaces the peptide and the drug, D, or the bioorthogonal moiety. In certain embodiments, one or more of the amino acids in the peptide linker is N-methylated.

In some embodiments, Y may be at least one amino acid selected from the group consisting of alanine, valine, leucine, isoleucine, methionine, phenylalanine, tryptophan and proline. In some embodiments, Y may be at least one amino acid selected from the group consisting of phenylalanine, alanine, and valine.

In some embodiments, Z may be at least one amino acid selected from the group consisting of alanine, lysine, lysine protected with acetyl or formyl, arginine, arginine protected with tosyl or nitro groups, histidine, ornithine, ornithine protected with acetyl or formyl, and citrulline. In some embodiments, Z may be at least one amino acid selected from the group consisting of alanine, lysine and citrulline.

In some embodiments, exemplary Y—Z combinations include Valine-Citrulline; Valine-Alanine; and Alanine-Alanine.

In certain embodiments, A is —OC(O)—.

In certain embodiments, X is —OC(O)—.

In certain embodiments, W is —OC(O)—. In certain embodiments, X is absent and W is —OC(O)—.

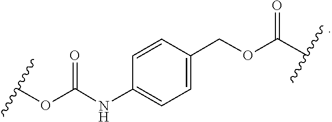 [SMILES: *OC(=O)Nc1ccc(COC(*)=O)cc1]

In certain embodiments, —X—W is

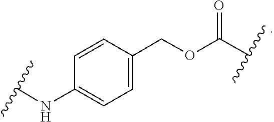 [SMILES: *Nc1ccc(COC(*)=O)cc1]

In certain embodiments, —X—W is

In certain embodiments, the peptide linker is specifically tailored so that it will be selectively cleaved (e.g., enzymatically cleaved) releasing the drug, such as by one or more of the tumor-associated proteases.

In certain embodiments, the peptide linker has a chain length of two to four amino acid residues (i.e., a di-, tri-, or tetra-peptide). It will be understood, however, that peptide linkers up to five, six, seven, or eight amino acid residues may also suitably be employed.

In certain embodiments, the peptide linker is Phe-Lys, Val-Lys, Val-Ala, Ala-Ala, Phe-Phe-Lys, D-Phe-Phe-Lys, Gly-Phe-Lys, Ala-Lys, Val-Cit, Phe-Cit, Leu-Cit, Ile-Cit, Trp-Cit, Phe-Ala, Gly-Phe-Leu-Gly [SEQ ID NO:], Ala-Leu-Ala-Leu [SEQ ID NO:], Phe-N⁹-tosyl-Arg, or Phe-N⁹-Nitro-Arg. In certain embodiments, the peptide linker is Phe-Lys, Val-Lys, Val-Ala, Ala-Ala, Val-Val, Val-Cit, or D-Phe-L-Phe-Lys. In certain embodiments, the peptide linker is Val-Cit, Val-Ala, or Ala-Ala.

In certain embodiments, linker L¹ is:

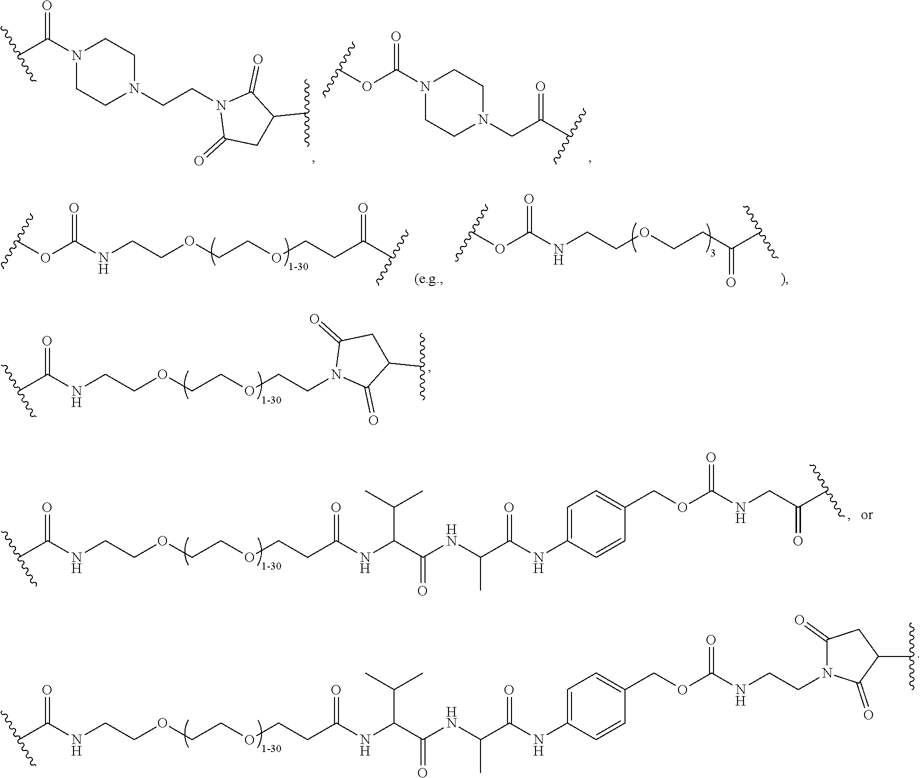

The foregoing linkers may attach on the right-hand side to amino acid side chains of D such lysine or cysteine

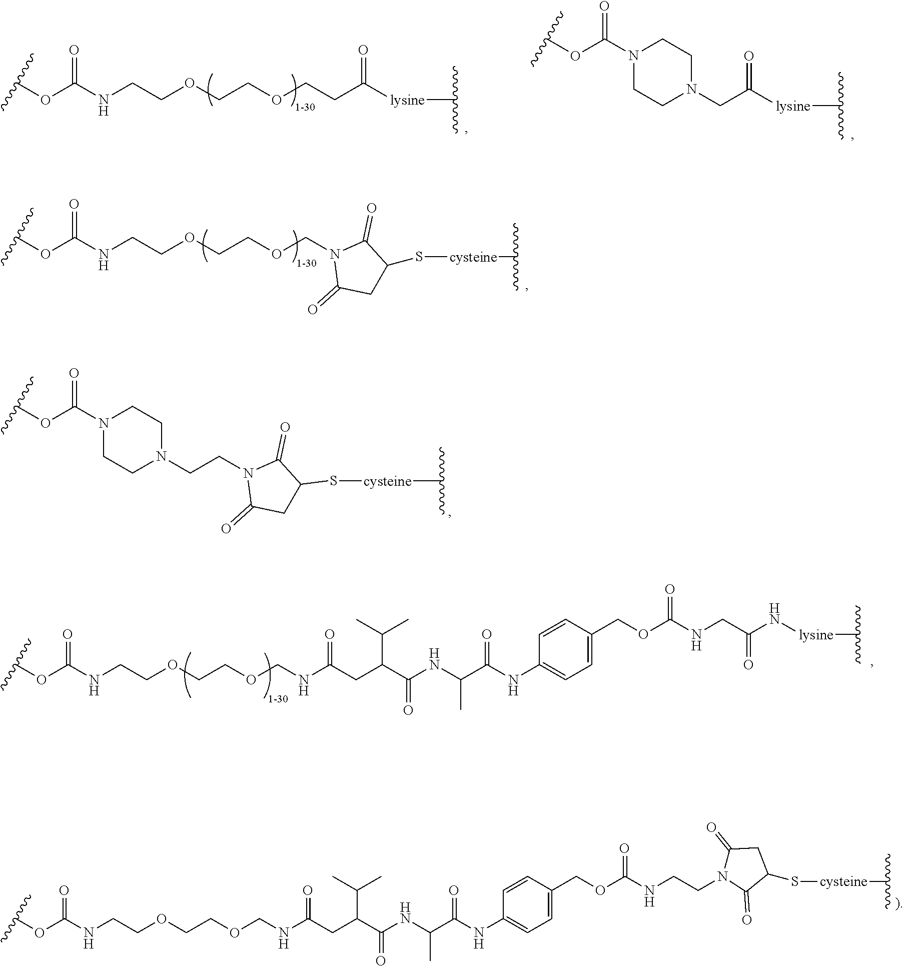

In some embodiments, the payload is covalently bonded to the linker through an amide bond; e.g., the payload may be an amine-containing payload for attachment of the payload to a carbonyl group of the linker, or, in other cases, the payload may be a carboxyl-containing payload for attachment of the payload to an amine group of the linker. In some instances, the payload and linker, together form a carbamate group; e.g., the payload may be an amine-containing payload for attachment of the payload to an acyloxy group of the linker. In some instances, the payload and linker, together form a carbonate group; e.g., the payload may be a hydroxy-containing payload for attachment of the payload to an acyloxy group of the linker.

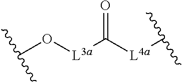

In some embodiments, L¹ is or —O—;
L^{3a} is a bond or C₁₋₆alkylene;
L^{4a} is a bond, —NHN:, —N(R¹⁰)—C₂₋₆alkylene-N(R¹¹)—, —N(R¹²)—C₂₋₃alkylene-N(R¹³)C(O)—, —N(R¹⁰)—C₁₋₆alkylene-C(O)NHN:, —NHNHC(O)C₁₋₆alkylene-C(O)NHN:, —CH(NHC(O)R¹⁴)C₁₋₄alkylene-S—S—C₁₋₄alkylene-OC(O)—, —NHNHC(O)CH(NHC(O)R¹⁵)CH₂C(O)—, —C₁₋₆alkylene-CH(G^{x})OC(O)—,

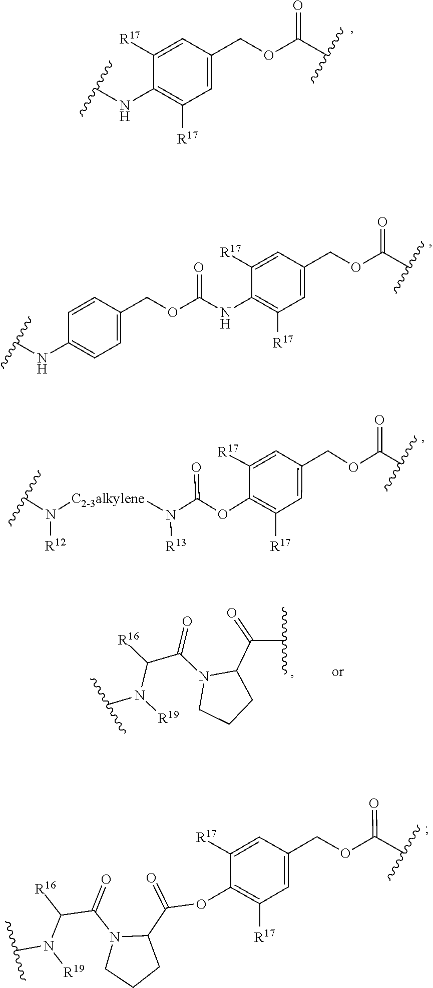

R¹⁰, R¹¹, R¹², R¹³, R¹⁴, R¹⁵, and R¹⁹ are each independently hydrogen or C₁₋₄alkyl;
R¹⁶ is hydrogen, C₁₋₄alkyl, —C₁₋₄alkylene-OH, —C₁₋₄alkylene-OC₁₋₄alkyl, —C₁₋₄alkylene-CO₂H, or C₁₋₄alkylene-CONH₂;
R¹⁷, at each occurrence, is independently hydrogen or —CH₂OC(O)—; and
G^{x} is phenyl optionally substituted with 1-5 substituents independently selected from the group consisting of halogen, C₁₋₄alkyl, C₁₋₄haloalkyl, C₁₋₄alkoxy, cyano, and nitro.

In certain embodiments, linker L¹ is —OC(O)—.

In some embodiments, L¹ is

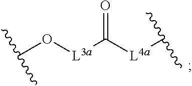

L^{3a} is a bond;
L^{4a} is

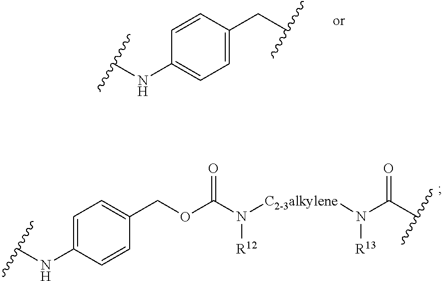

and
R¹² and R¹³ are each independently hydrogen or C₁₋₄alkyl.

In some embodiments, p″ is 1. In some embodiments, p′ is 1.

In some embodiments,

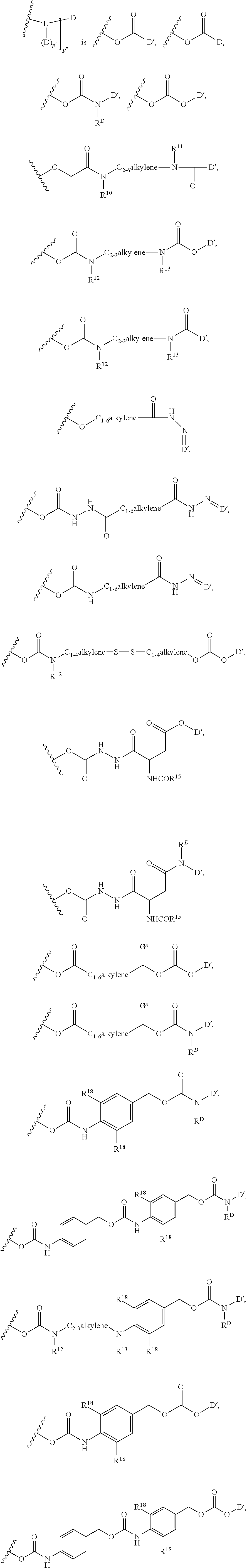

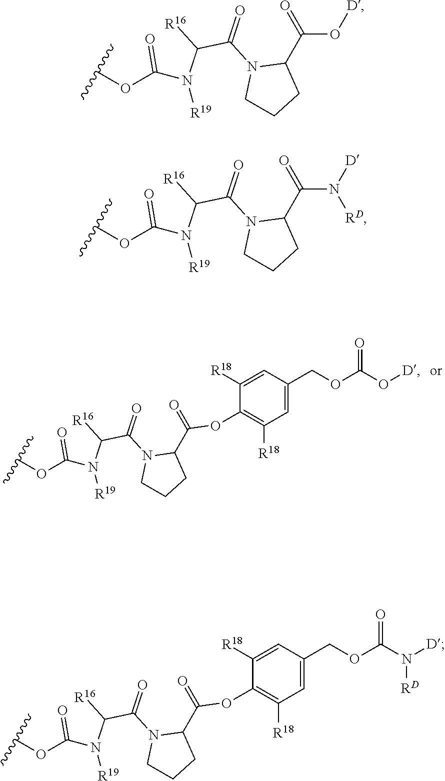

R¹⁸, at each occurrence, is independently hydrogen or —CH₂OC(O)NHD′;
R^{D} is hydrogen or C₁₋₄alkyl on a nitrogen atom of a payload; and
D and D′ are independently a payload moiety.

In some embodiments, D or D′ is a cyclic dinucleotide payload moiety, imidazo[4,5-c]quinolin-4-amine payload moiety, TLR agonist payload moiety, STING agonist payload moiety, or anticancer agent payload moiety.

In some embodiments,

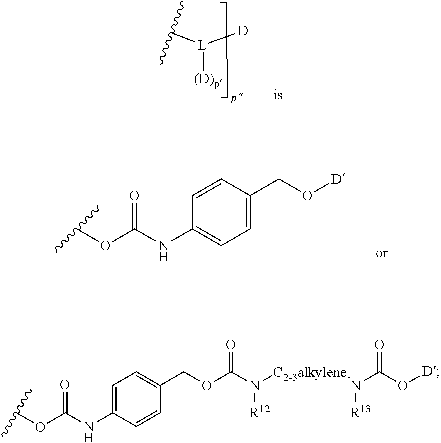

R¹² and R¹³ are each independently hydrogen or C₁₋₄alkyl; and
D and D′ are independently a payload moiety (e.g., anticancer agent payload moiety).

In some embodiments, p′ is 0.

In some embodiments, p″ is 2 or 3.

In some embodiments, p is 2 and

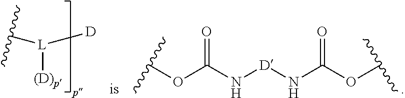

The person skilled in the art will recognize that a payload (D or D′) bonded to a linker does not refer to a payload molecule per se, but refers to the portion of the payload molecule bonded to the linker. Release of the payload (D or D′) from a prodrug, releases the payload per se.

A payload (D or D′) may be an anticancer agent payload of any of the anticancer agents described herein.

In some embodiments, the payload comprises a TLR7/8 agonist, and X is a biocompatible support. In some embodiments, the payload comprises gardiquimod, and X is a biocompatible support.

In some embodiments, the payload comprises a TLR7/8 agonist, and X is an antibody or antibody fragment moiety which targets HER2, TROP2, Nectin4, or extracellular matrix (ECM). In some embodiments, the payload comprises gardiquimod, and X is an antibody or antibody fragment moiety which targets HER2, TROP2, Nectin4, or extracellular matrix (ECM).

In some embodiments, the payload comprises camptothecin, or derivative thereof, and X is a biocompatible support. In some embodiments, the payload comprises exatecan, and X is a biocompatible support.

In some embodiments, the payload comprises camptothecin, or derivative thereof, and X is an antibody or antibody fragment moiety which targets HER2, TROP2, Nectin4, or extracellular matrix (ECM). In some embodiments, the payload comprises exatecan, and X is an antibody or antibody fragment moiety which targets HER2, TROP2, Nectin4, or extracellular matrix (ECM).

In some embodiments, the payload comprises MMAE, and X is a biocompatible support.

In some embodiments, the payload comprises MMAE, or derivative thereof, and X is an antibody or antibody fragment moiety which targets HER2, TROP2, Nectin4, or extracellular matrix (ECM).

In some embodiments, the payload comprises paclitaxel, or derivative thereof, and X is a biocompatible support.

In some embodiments, the payload comprises paclitaxel, or derivative thereof, and X is an antibody or antibody fragment moiety which targets HER2, TROP2, Nectin4, or extracellular matrix (ECM).

In some embodiments, the payload comprises docetaxel, or derivative thereof, and X is a biocompatible support.

In some embodiments, the payload comprises docetaxel, or derivative thereof, and X is an antibody or antibody fragment moiety which targets HER2, TROP2, Nectin4, or extracellular matrix (ECM).

In some embodiments, the payload has the structure:

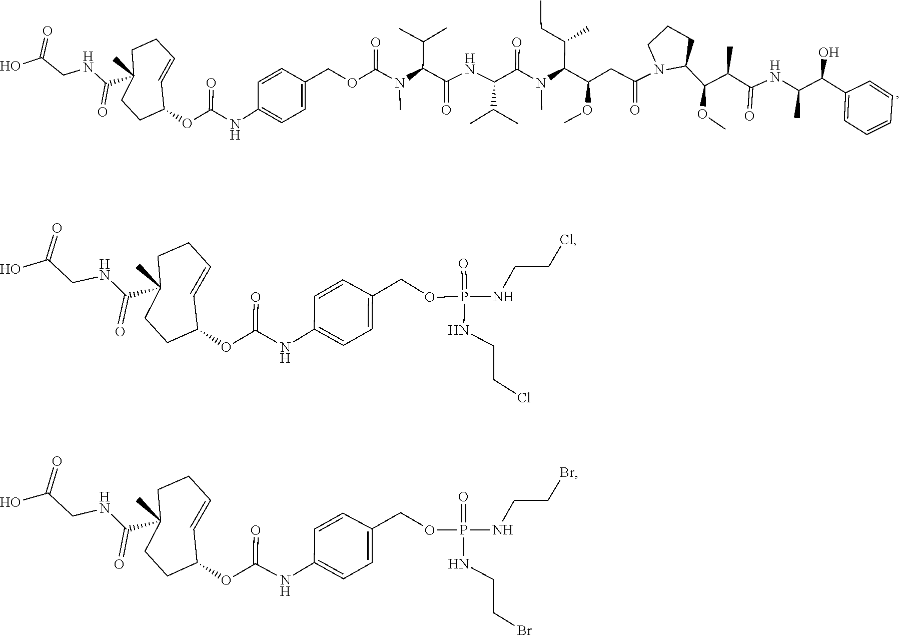

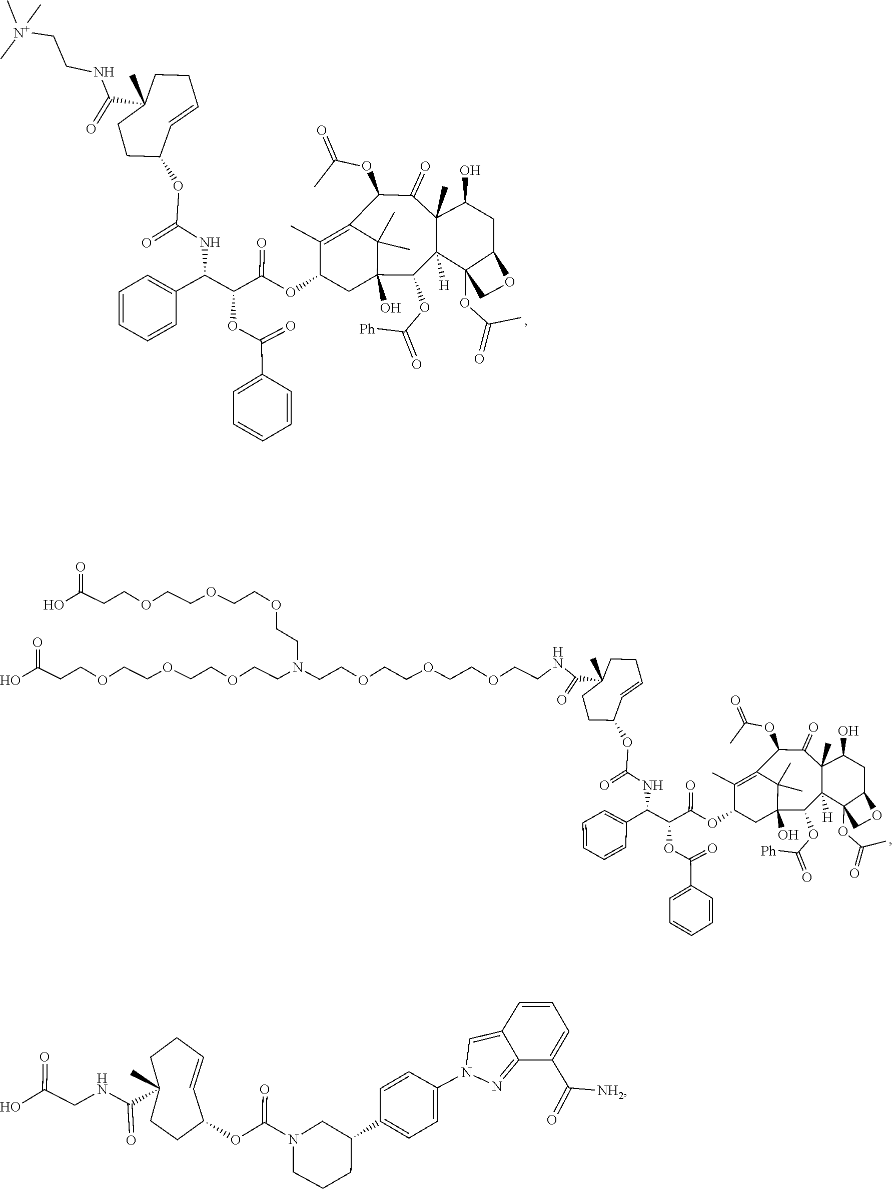

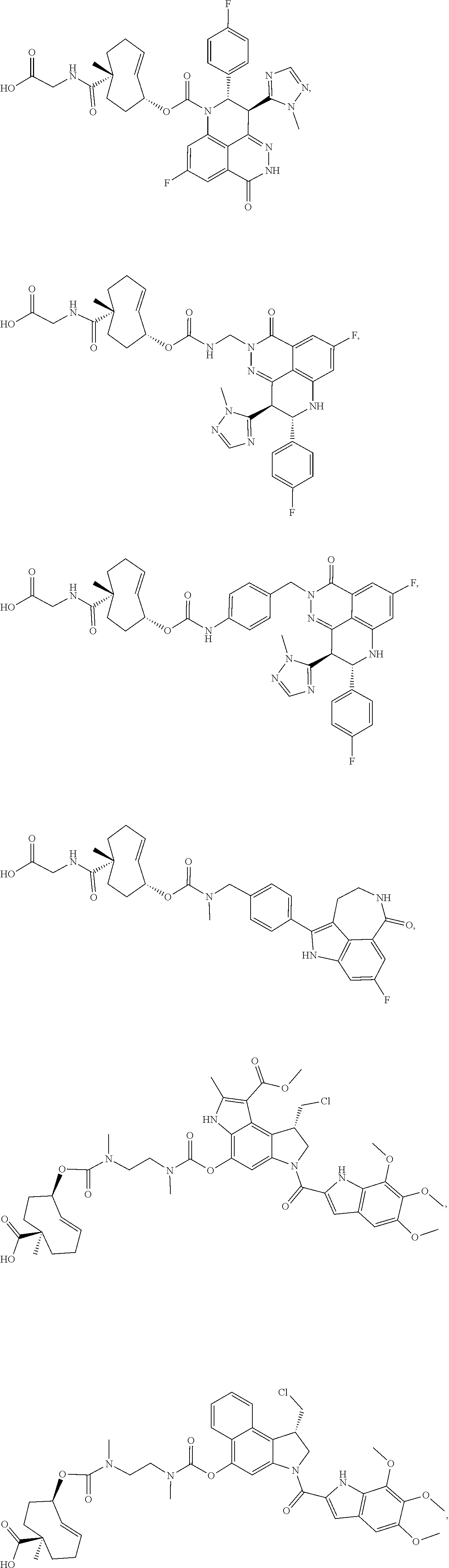

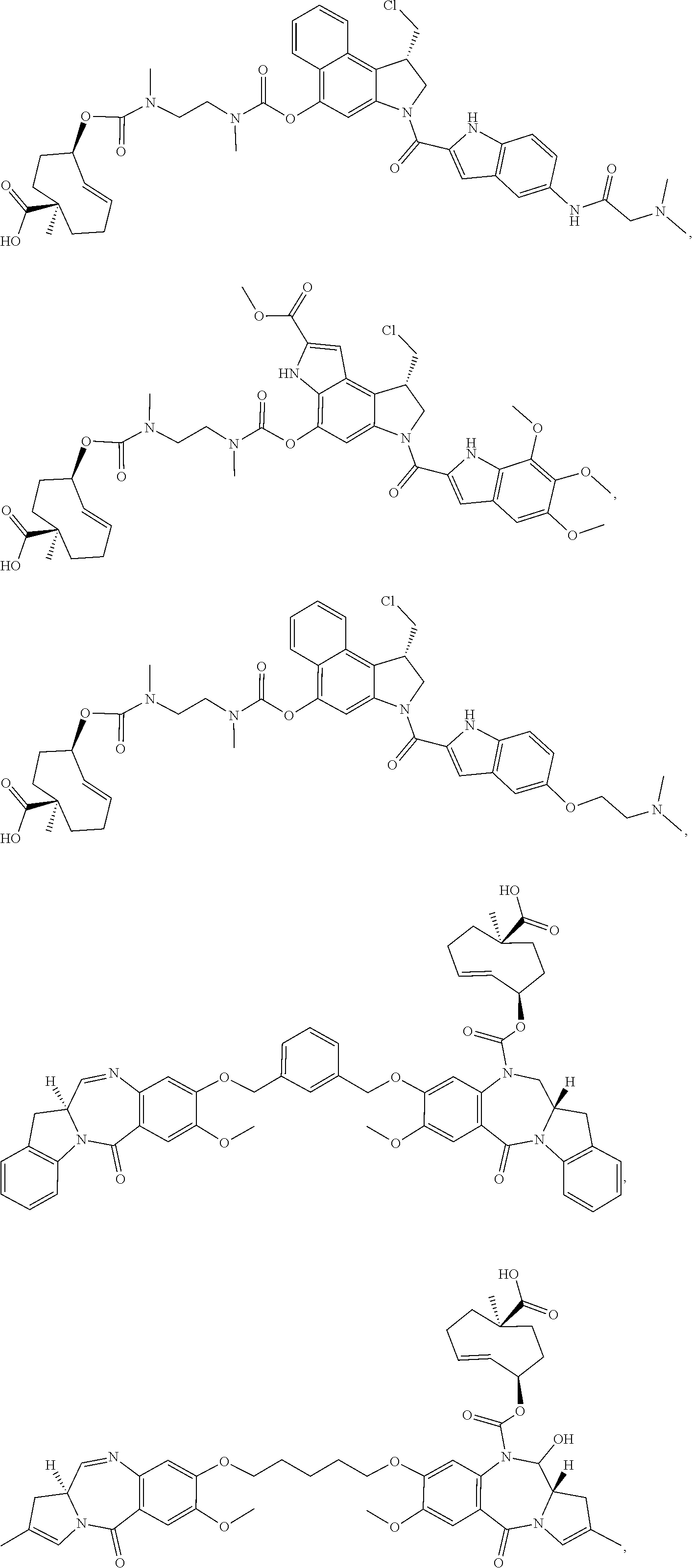

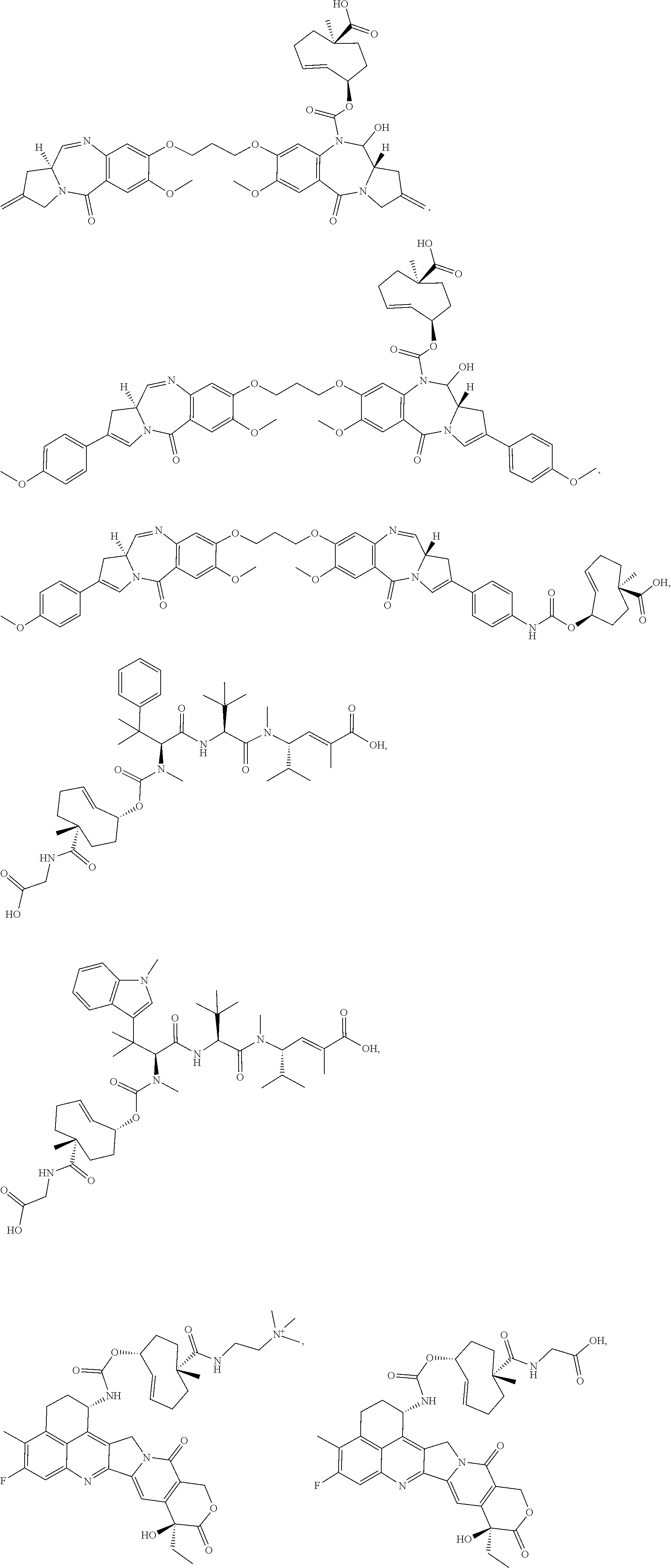

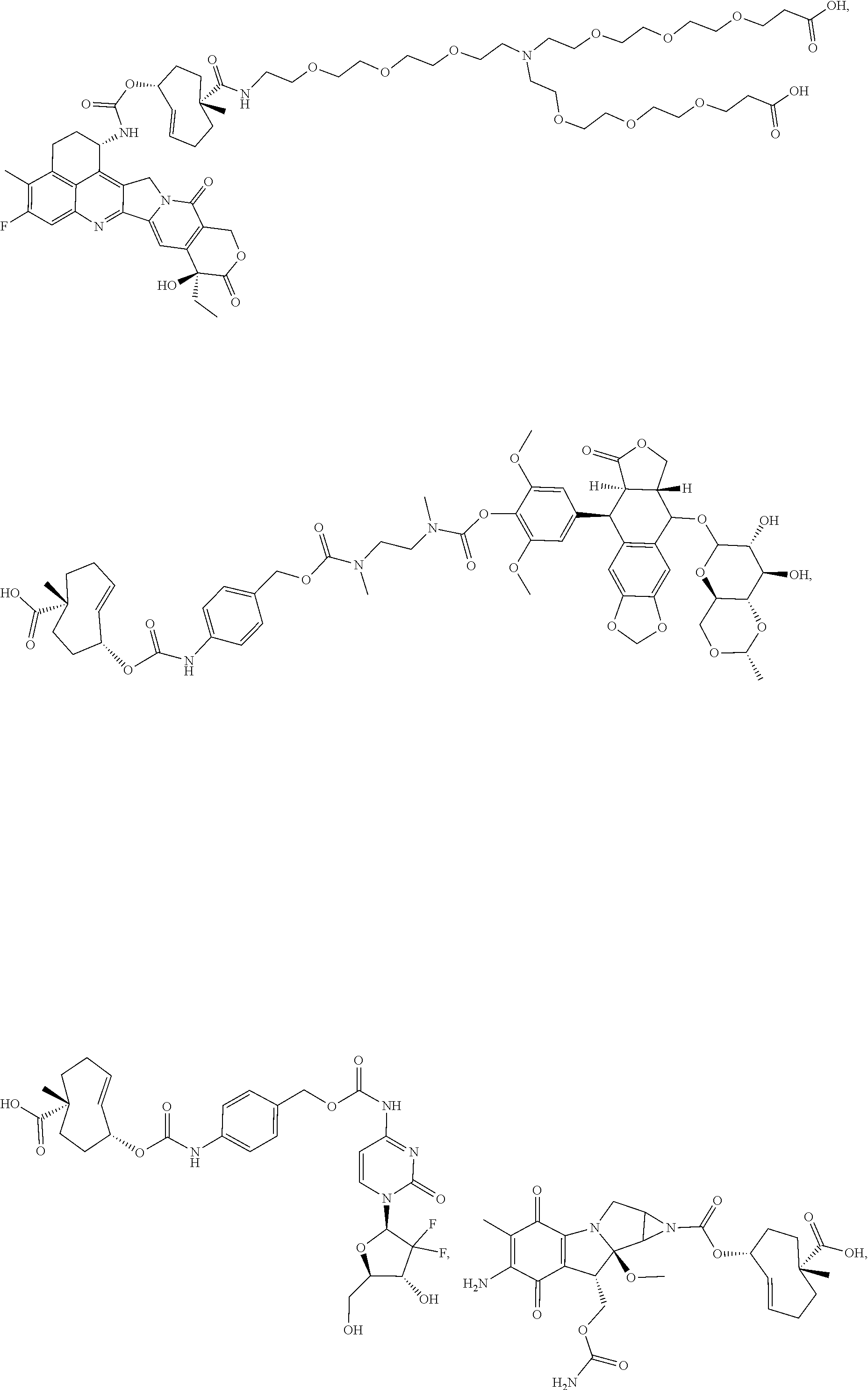

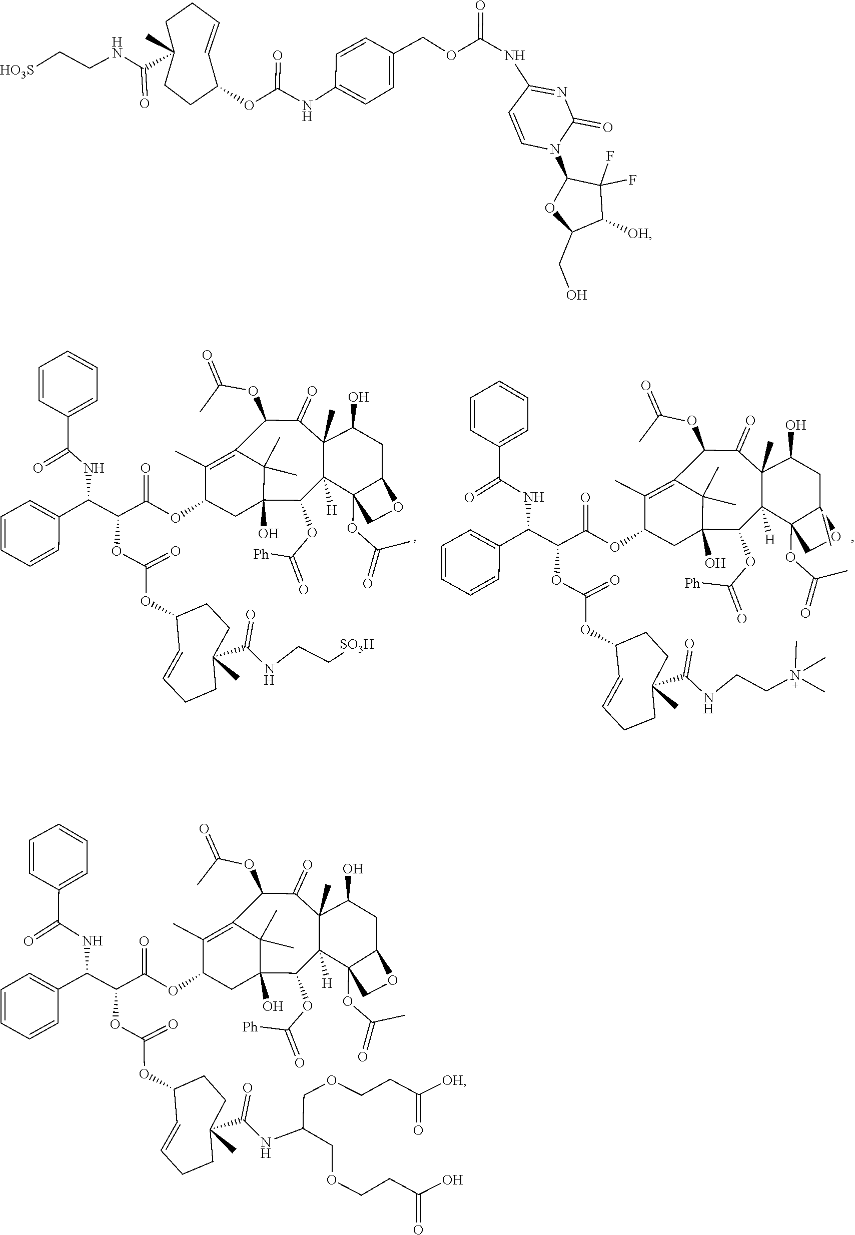

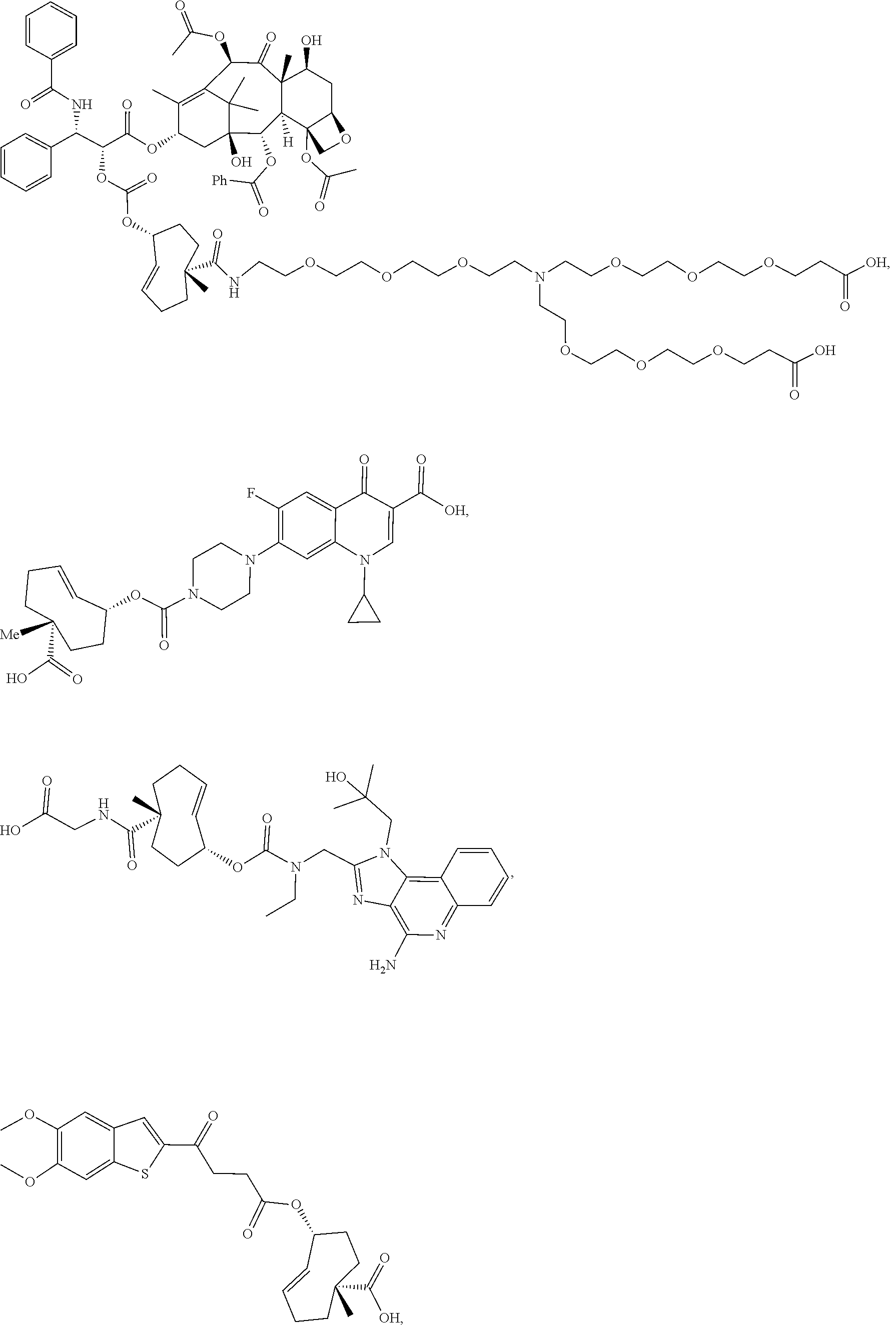

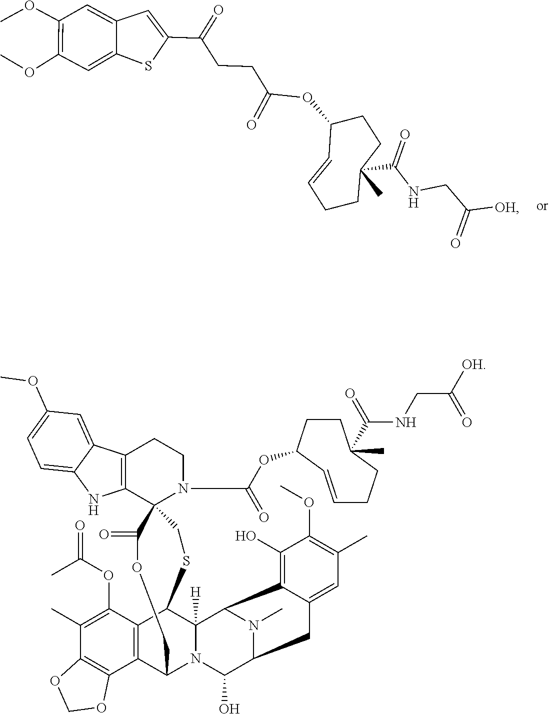

In certain embodiments, the trans-cyclooctene functionalized prodrug is selected from:

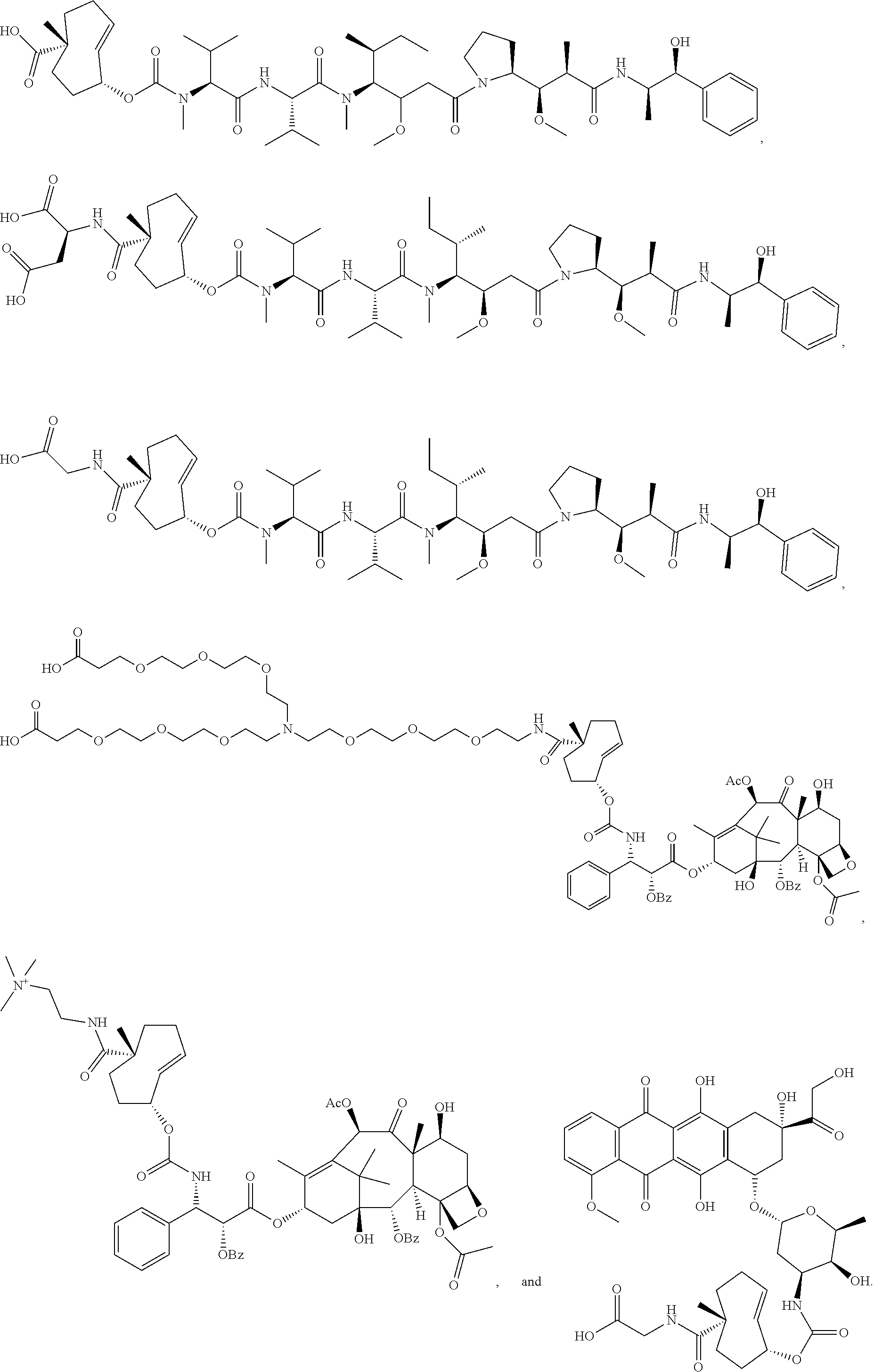

### E. Methods of Treatment

Aspects of the present disclosure include methods for delivering a payload to a target location in a subject. In certain embodiments, the method includes selectively delivering a payload to the target location in a subject. Selective delivery of the payload includes delivering the payload to the target location (e.g., an organ or tissue, or portion thereof), without targeting other locations in the subject (e.g., other organs or tissues, or portions thereof) that do not need administration of the payload. Selective delivery of the payload may be achieved through use of the targeting moiety and the functionalized payloads described herein.

In some instances, a targeting moiety of the present disclosure may be localized to a desired target location in a subject. For example, methods of the present disclosure may include administering to a subject a targeting moiety as described herein. The targeting moiety may be administered to the subject at a desired target location in the subject. In some instances, the targeting moiety may be injected locally into the subject at the desired target location in the subject. In some embodiments, the targeting moiety is administered systemically. In these embodiments, the targeting moiety may localize at a desired target location in the subject through specific binding of the targeting agent to its target (e.g., antibody-antigen interaction, and the like), or may localize on the surface of a desired target (e.g., a cell surface) through specific binding of the targeting agent to its target (e.g., antibody-antigen interaction, and the like).

As described herein, selective binding between bioorthogonal binding partners (e.g., between a tetrazine of the targeting moiety and its complementary trans-cyclooctene of a prodrug may occur. Due to the localized administration of the targeting moiety to a desired location in the subject as described above, the selective binding between the trans-cyclooctene and its complementary binding agent of the prodrug will localize the payload to the desired target location.

Provided herein is a method of treating cancer comprising administering to a subject in need thereof, a therapeutically effective amount of a targeting moiety as described herein, or a pharmaceutically acceptable salt thereof, and a trans-cyclooctene prodrug.

In some embodiments, the cancer is metastatic. In some embodiments the cancer is melanoma, renal cancer, prostate cancer, ovarian cancer, endometrial carcinoma, breast cancer, glioblastoma, lung cancer, soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, non-Hodgkin's lymphoma, Hodgkin's lymphoma Wilm's tumor/neuroblastoma, bladder cancer, thyroid adenocarcinoma, pancreatic neuroendocrine tumors, prostatic adenocarcinoma, nasopharyngeal carcinoma, or cutaneous T-cell lymphoma.

In some embodiments, the cancer is a melanoma, renal cancer, prostate cancer, ovarian cancer, breast cancer, glioma, lung cancer, soft tissue carcinoma, soft tissue sarcoma, osteosarcoma, or pancreatic cancer.

In some embodiments, the cancer is a solid tumor.

In some embodiments, the cancer is a soft tissue sarcoma.

In some embodiments, the soft tissue sarcoma is a fibrosarcoma, rhabdomyosarcoma, or Ewing's sarcoma.

In some embodiments, the method also comprises enhancing or eliciting an immune response. In some embodiments the immune response is an increase in one or more of leukocytes, lymphocytes, monocytes, and eosinophils.

In some embodiments, the method further comprising administering a therapeutically effective amount of an additional therapeutic agent selected from the group consisting of an anticancer agent, an immunomodulatory agent, or a trans-cyclooctene prodrug thereof. Anticancer agents, immunomodulatory agents, and their trans-cyclooctene prodrugs are known in the art.

Indications for this approach include cancer, both hematological and solid cancers. In certain embodiments, the approach can be used for the treatment and/or diagnosis of soft tissue sarcomas: rhabdomyosarcoma, fibrosarcoma, Ewing's sarcoma, and all the different subtypes of soft tissue sarcoma as well as osteosarcoma. The compositions can be for the treatment and/or diagnosis of pigmented vilonodular synovitis.

In certain embodiments, the approach can be used for the treatment and/or diagnosis of hematological malignancies such as myelodysplastic syndromes, acute myeloid leukemia, myelodisplastic syndromes, chronic myelogenous leukemia, chronic myelomonocytic leukemia, primary myelofibrosis, diffuse large B-cell lymphoma, chronic lymphocytic leukemia, monoclonal gammopathy, plasma cell myeloma, follicular lymphoma, marginal zone lymphoma, classical Hodgkin's lymphoma, monoclonal B-cell lymphocytosis, lymphoproliferative disorder NOS, T-cell lymphoma, precursor B-lymphoblastic leukemia, mantle cell lymphoma, plasmacytoma, Burkitt lymphoma, T-cell leukemia, hairy-cell leukemia, precursor T-lymphoblastic leukemia, nodular lymphocyte predominant Hodgkin's lymphoma, as well as others.

The compositions of the present disclosure find use in treatment and/or diagnosis of a condition or disease in a subject that is amenable to treatment or diagnosis by administration of the payload (e.g., the parent drug (i.e., the drug prior to conjugation to the composition)). By “treatment” is meant that at least an amelioration of the symptoms associated with the condition afflicting the subject is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, are completely inhibited, e.g., prevented from happening, or stopped, e.g., terminated, such that the subject no longer suffers from the condition, or at least the symptoms that characterize the condition. Treatment may include inhibition, that is, arresting the development or further development of clinical symptoms, e.g., mitigating or completely inhibiting an active disease. Treatment may include relief, that is, causing the regression of clinical symptoms. For example, in the context of cancer, the term “treating” includes any or all of: reducing growth of a solid tumor, inhibiting replication of cancer cells, reducing overall tumor burden, prolonged survival and ameliorating one or more symptoms associated with a cancer.

The subject to be treated can be one that is in need of therapy, where the subject to be treated is one amenable to treatment using the parent drug. Accordingly, a variety of subjects may be amenable to treatment using the compositions disclosed herein. Generally, such subjects are “mammals,” with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

In certain embodiments, additional therapeutic agents, and methods can be used for the treatment, prevention, and/or diagnosis of solid tumors, including but not limited to, melanoma (e.g., unresectable, metastatic melanoma), renal cancer (e.g., renal cell carcinoma), prostate cancer (e.g., metastatic castration resistant prostate cancer), ovarian cancer (e.g., epithelial ovarian cancer, such as metastatic epithelial ovarian cancer), endometrial carcinoma, breast cancer (e.g., triple negative breast cancer), glioblastoma (e.g., glioblastoma multiforme), and lung cancer (e.g., non-small cell lung cancer), soft tissue sarcoma, fibrosarcoma, osteosarcoma, pancreatic cancer, gastric carcinoma, squamous cell carcinoma of head/neck, anal/vulvar carcinoma, esophageal carcinoma, pancreatic adenocarcinoma, cervical carcinoma, hepatocellular carcinoma, Kaposi's sarcoma, Non-Hodgkin's lymphoma, Hodgkin's lymphoma Wilm's tumor/neuroblastoma, bladder cancer, thyroid adenocarcinoma, pancreatic neuroendocrine tumors, prostatic adenocarcinoma, nasopharyngeal carcinoma, cutaneous T-cell lymphoma, among others. The disclosed approach lends itself well as an adjuvant/neoadjuvant system. For example, particles as disclosed herein could be placed during the biopsy, once the results from the study come back, the practitioner could deliver the appropriate cocktail to the desired site in the body. This would minimize the size of the tumor particularly in the context of a surgically resectable tumor. Then at the end of the surgery, the surgeon could administer additional targeting moiety to the subject to target the surgical cavity and treat the patient with further doses of treatment (e.g. chemotherapy through the disclosed approach) to minimize the risk of any cancer cells that may have been missed in the surgical margins.

In certain embodiments, a targeting moiety as disclosed herein could be administered and the practitioner could deliver the appropriate cocktail to the desired site in the body. This would minimize the size of the tumor particularly in the context of a surgically resectable tumor. Then at the end of the surgery, the surgeon could administer additional targeting moiety to the subject to target the surgical cavity and treat the patient with further doses of treatment (e.g. chemotherapy through the disclosed approach) to minimize the risk of any cancer cells that may have been missed in the surgical margins.

In certain embodiments, the disclosed methods provide the ability to place particles as disclosed herein at the time of the biopsy. When the results return, the practitioner can deliver through to the biopsy site immunomodulatory agents.

In certain embodiments, the disclosed methods provide the ability for a practitioner to deliver immunomodulatory agents, such as TLR agonists, STING agonists, chemokines (agents that attract cancerous cells and/or immune cells) and adjuvants to enhance the immune system with fewer side effects as well as the chemotherapeutics agents combined with immunotherapy agents. This combination approach would be beneficial to patients. The chemotherapy agent would treat the solid tumor or specific location, while the enhanced response of the immunotherapy would help with distant metastatic sites. For example, in certain embodiments, the disclosed compositions and methods could employ or be used with anthracyclines, taxanes, gemcitabine and other agents to enhance the efficacy of one or more immunomodulatory agents such as ipilimumab, nivolumab, pembrolizumab, avelumab (also known as MSB0010718C; Pfizer).

### Cancer

The disclosed methods may be used to treat or prevent cancer, including metastatic cancer. Cancer is a group of related diseases that may include sustained proliferative signaling, evasion of growth suppressors, resistance to cell death, enablement of replicative immortality, induction of angiogenesis, and the activation of invasion and metastasis. The disclosed methods may enhance or elicits an immune response against a cancer in the subject. The immune response may lead to an increase in one or more of leukocytes, lymphocytes, monocytes, and eosinophils.

Cancer that may be treated by the disclosed methods, includes, but is not limited to, astrocytoma, adrenocortical carcinoma, appendix cancer, basal cell carcinoma, bile duct cancer, bladder cancer, bone cancer, brain cancer, brain stem cancer, brain stem glioma, breast cancer, cervical cancer, colon cancer, colorectal cancer, cutaneous T-cell lymphoma, diffuse intrinsic pontine glioma, ductal cancer, endometrial cancer, ependymoma, Ewing's sarcoma, esophageal cancer, eye cancer, fibrosarcoma, gallbladder cancer, gastric cancer, gastrointestinal cancer, germ cell tumor, glioma, hepatocellular cancer, histiocytosis, Hodgkin's lymphoma, hypopharyngeal cancer, intraocular melanoma, Kaposi sarcoma, kidney cancer, laryngeal cancer, leukemia, liver cancer, lung cancer, lymphoma, macroglobulinemia, melanoma, mesothelioma, mouth cancer, multiple myeloma, nasopharyngeal cancer, neuroblastoma, non-Hodgkin's lymphoma, osteosarcoma, ovarian cancer, pancreatic cancer, parathyroid cancer, penile cancer, pharyngeal cancer, pituitary cancer, prostate cancer, rectal cancer, renal cell cancer, retinoblastoma, rhabdomyosarcoma, sarcoma, skin cancer, small cell lung cancer, small intestine cancer, soft tissue carcinoma, soft tissue sarcoma, solid tumor, squamous cell carcinoma, stomach cancer, T-cell lymphoma, testicular cancer, throat cancer, thymoma, thyroid cancer, trophoblastic tumor, urethral cancer, uterine cancer, uterine sarcoma, vaginal cancer, vulvar cancer, Wilms tumor, non-small cell lung cancer (NSCLC), diffuse large B-cell lymphoma (DLBCL), or oral tongue squamous cell carcinoma (OTSCC).

In some embodiments, the cancer that may be treated by the disclosed methods is melanoma, renal cancer, prostate cancer, ovarian cancer, breast cancer, glioma, lung cancer, soft tissue carcinoma, soft tissue sarcoma, osteosarcoma, or pancreatic cancer. In some embodiments, the cancer is a solid tumor. In some embodiments, the cancer is a soft tissue carcinoma. In some embodiments, the cancer is afibrosarcoma. In some embodiments, the cancer is diffuse intrinsic pontine glioma. In some embodiments, the cancer is a metastatic cancer.

In some embodiments, the cancer that may be treated by the disclosed methods is a hematological malignancy, such as myelodysplastic syndromes, acute myeloid leukemia, myelodisplastic syndromes, chronic myelogenous leukemia, chronic myelomonocytic leukemia, primary myelofibrosis, diffuse large B-cell lymphoma, chronic lymphocytic leukemia, monoclonal gammopathy, plasma cell myeloma, follicular lymphoma, marginal zone lymphoma, classical Hodgkin's lymphoma, monoclonal B-cell lymphocytosis, lymphoproliferative disorder NOS, T-cell lymphoma, precursor B-lymphoblastic leukemia, mantle cell lymphoma, plasmacytoma, Burkitt lymphoma, T-cell leukemia, hairy-cell leukemia, precursor T-lymphoblastic leukemia, nodular lymphocyte predominant Hodgkin's lymphoma, as well as others.

Without being bound by a particular theory, local release of certain anti-cancer agents using the compounds and methods of the disclosure may produce or contribute to immunogenic cell death (ICD). For example, certain anti-cancer agents (e.g., anthracyclines, cyclophosphamide, oxaliplatin) have been reported to induce ICD. Kroemer et al. Annu. Rev. Immunol. 2013 (31), 51-72. Immunogenic apoptosis of cancer cells can induce an effective antitumor immune response through activation of dendritic cells (DCs) and consequent activation of specific T cell response. ICD is characterized by secretion of damage-associated molecular patterns (DAMPs). Three important DAMPs which are exposed to the cell surface during ICD. Calreticulin (CRT), one of the DAMP molecules, which is normally in the lumen of endoplasmic reticulum (ER), is translocated after the induction of immunogenic apoptosis to the surface of dying cell where it functions as an “eat me” signal for professional phagocytes. Other important surface exposed DAMPs are heat-shock proteins (HSPs), namely HSP70 and HSP90, which are under stress condition also translocated to the plasma membrane. On the cell surface they have an immunostimulatory effect, based on their interaction with number of antigen-presenting cell (APC) surface receptors like CD91 and CD40 and also facilitate cross presentation of antigens derived from tumor cells on MHC class I molecule, which than leads to the CD8+ T cell response. Other important DAMPs, characteristic for ICD are secreted amphoterin (HMGB1) and ATP. HMGB1 is considered to be late apoptotic marker and its release to the extracellular space seems to be required for the optimal release and presentation of tumor antigens to dendritic cells. It binds to several pattern recognition receptors (PRRs) such as Toll-like receptor (TLR) 2 and 4, which are expressed on APCs. The most recently found DAMP released during immunogenic cell death is ATP, which functions as a “find-me” signal for monocytes when secreted and induces their attraction to the site of apoptosis. Kroemer et. al. Curr. Op. Immunol. 2008 (20), 504-511.

Thus, local release of ICD inducers using the compounds and methods of the disclosure may be beneficially combined with one or more immunomodulatory agents.

In certain embodiments, the targeting moiety can be used for the treatment, prevention, and/or diagnosis of solid tumors, including but not limited to, melanoma (e.g., unresectable, metastatic melanoma), renal cancer (e.g., renal cell carcinoma), prostate cancer (e.g., metastatic castration resistant prostate cancer), ovarian cancer (e.g., epithelial ovarian cancer, such as metastatic epithelial ovarian cancer), breast cancer (e.g., triple negative breast cancer), glioblastoma (e.g., glioblastoma multiforme), and lung cancer (e.g., non-small cell lung cancer), soft tissue sarcoma, fibrosarcoma, osteosarcoma, pancreatic cancer, among others.

The disclosed approach lends itself well as an adjuvant/neoadjuvant system. For example, targeting moieties as disclosed herein could be placed during the biopsy, once the results from the study come back, the practitioner could administer the appropriate cocktail to deliver treatment to the desired site in the body (compounds as disclosed herein and optional additional therapeutic agent(s)). The results of the biopsy may indicate the amount and type of treatment to deliver to the site of a tumor. For example, chemokines (agents that attract cancerous cells and/or immune cells) and adjuvants to enhance the immune system with fewer side effects as well as the chemotherapeutics agents could be delivered and combined with immunotherapy agents.

The disclosed methods may include one or multiple systemic doses of targeting moieties that focus at one location or more locations. The disclosed methods may be used to deliver a functionalized payload to these location through systemic or local administration. In some embodiments, the targeting moiety is delivered systemically. In some embodiments, the targeting moiety and the payload (i.e., a TCO-labeled payload) are both delivered systemically. In some embodiments, the targeting moiety is delivered locally.

The disclosed compounds and compositions may be administered prior to surgical resection. The disclosed methods may minimize the size of the tumor prior to surgical resection. This would minimize the size of the tumor particularly in the context of a surgically resectable tumor. The disclosed conjugates, compounds and compositions may be administered during surgical resection. The disclosed conjugates, compounds and compositions may be administered after surgical resection. The targeting moiety may be placed around the surgical cavity at the end of surgical resection and the subject may then be treated with further doses of a treatment to minimize the risk of any cancer cells that may have been missed in the surgical margins.

The disclosed methods may include multiple systemic doses of functionalized payload that focus at one location. The disclosed methods may be used to deliver a second payload. The disclosed methods may be used to administer a second functionalized payload if the tumor is resistant to the first payload. A second payload may be a TCO-labeled payload of gemcitabine or docetaxel. The TCO-labeled payload of gemcitabine, paclitaxel, or docetaxel may be administered in combination with doxorubicin. The second functionalized payload may be activated by the targeting moiety used for the first prodrug.

The functionalized payloads disclosed herein may function as adjuvants. This combination approach would be beneficial to patients. The chemotherapy agent would treat the solid tumor or specific location and may enhance or elicit an immune response, while the enhanced response of the immunotherapy of the functionalized payload and/or separate agent may help with distant metastatic sites. For example, in certain embodiments, the disclosed compositions and methods could employ or be used with anthracyclines, auristatins, vinca alkaloids, taxanes, gemcitabine, camptothecin analogues and other agents to enhance the efficacy of ipilimumab, nivolumab, pembrolizumab, avelumab (also known as MSB0010718C; Pfizer).

The disclosed methods may be used to treat diffuse intrinsic pontine gliomas. Diffuse intrinsic pontine gliomas (DIPG) are pediatric brainstem tumors that may be highly malignant and may be difficult to treat. There is no known curative treatment for DIPG, and survival odds have remained dismal over the past four decades. DIPG patients have a median overall survival of just 11 months, with a two-year survival rate below 10%. DIPG account for 75-80% of brainstem tumors in children, affecting an estimated 200-300 children in the U.S. each year. The rarity of this devastating disease and previous lack of experimental model systems has impeded research, and over the past four decades survival odds have remained the same. Diagnosis of DIPG may begin with clinical symptoms and may be confirmed by MRI. The disease may begin with several months of generalized symptoms, including behavioral changes and difficulties in school, double vision, abnormal or limited eye movements, an asymmetric smile, loss of balance, and weakness. Alternately, severe neurologic deterioration may happen more quickly, with symptoms present for less than a month prior to diagnosis. Clinical examination may reveal the triad of multiple cranial neuropathies, long tract signs such as hyperreflexia and clonus, as well as ataxia. Expansion of the pons section of the brainstem may cause obstructive hydrocephalus and increased intracranial pressure.

Nuclei critical for life-sustaining function such as breathing and heartbeat in are located in the pons and without treatment, breathing and heartbeat may be damaged by DIPG.

The disclosed methods may include multiple systemic doses of functionalized payload that focus at one location. The disclosed methods may be used to deliver a second payload. The disclosed methods may be used to administer a second functionalized payload if the tumor is resistant to the first payload. A second payload may be a TCO-labeled payload of gemcitabine or docetaxel. The TCO-labeled payload of gemcitabine or docetaxel may be administered in combination with doxorubicin. The second functionalized payload may be activated by the targeting moiety used for the first prodrug.

### Modes of Administration

Methods of treatment may include any number of modes of administering a disclosed conjugate, compound or composition. Modes of administration may include tablets, pills, dragees, hard and soft gel capsules, granules, pellets, skin patches, skin creams, skin gels, aqueous, lipid, oily or other solutions, emulsions such as oil-in-water emulsions, liposomes, aqueous or oily suspensions, syrups, elixirs, solid emulsions, solid dispersions or dispersible powders. In the pharmaceutical composition, the conjugate, compound or compositions disclosed herein may also be dispersed in a microparticle, e.g. a nanoparticulate composition.

For parenteral administration, the conjugates, compounds or compositions disclosed herein may be dissolved or suspended in a physiologically acceptable diluent, such as water, buffer, oils with or without solubilizers, surface-active agents, dispersants or emulsifiers. Suitable oils may include, for example, olive oil, peanut oil, cottonseed oil, soybean oil, castor oil and sesame oil. For parenteral administration, the conjugates, compounds or compositions disclosed herein may be administered in the form of an aqueous, lipid, oily or other kind of solution or suspension, or even administered in the form of liposomes or nano-suspensions.

The term “parenterally,” as used herein, refers to modes of administration which include intravenous, intramuscular, intraperitoneal, intrasternal, subcutaneous and intraarticular injection and infusion.

The amount of composition administered to a subject can be initially determined based on guidance of a dose and/or dosage regimen of the parent drug. In general, the compositions can provide for targeted delivery and/or enhanced serum half-life of the bound drug, thus providing for at least one of reduced dose or reduced administrations in a dosage regimen. Thus, the compositions can provide for reduced dose and/or reduced administration in a dosage regimen relative to the parent drug prior to being conjugated in a composition of the present disclosure.

The pharmaceutical formulation may be provided in unit dosage form. In such form the pharmaceutical formulation may be subdivided into unit doses containing appropriate quantities of the compositions of the present disclosure. The unit dosage form can be a packaged preparation, the package containing discrete quantities of the preparation, such as packeted tablets, capsules, and powders in pouches, vials or ampoules.

In some embodiments, provided is a kit comprising a targeting moiety, or a pharmaceutically acceptable salt thereof, as described herein, or the pharmaceutical composition comprising the same, and instructions for use thereof.

In some embodiments, the kit further comprising a prodrug.

Compositions of the present disclosure can be present in any suitable amount, and can depend on various factors including, but not limited to, weight and age of the subject, state of the disease, etc. Suitable dosage ranges for the composition of the present disclosure include from 0.1 mg to 10,000 mg, or 1 mg to 1000 mg, or 10 mg to 750 mg, or 25 mg to 500 mg, or 50 mg to 250 mg. For instance, suitable dosages for the composition of the present disclosure include 1 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg, or 1000 mg.

In some embodiments, multiple doses of a composition are administered. The frequency of administration of a composition can vary depending on any of a variety of factors, e.g., severity of the symptoms, condition of the subject, etc. For example, in some embodiments, a composition is administered once per month, twice per month, three times per month, every other week (qow), once per week (qw), twice per week (biw), three times per week (tiw), four times per week, five times per week, six times per week, every other day (qod), daily (qd), twice a day (qid), or three times a day (tid).

The compositions of the present disclosure can be administered at any suitable frequency, interval and duration. For example, the composition of the present disclosure can be administered once an hour, or two, three or more times an hour, once a day, or two, three, or more times per day, or once every 2 days, 3 days, 4 days, 5 days, 6 days, or 7 days, so as to provide the desired dosage level to the subject. When the composition of the present disclosure is administered more than once a day, representative intervals include 5 min, 10 min, 15 min, 20 min, 30 min, 45 min and 60 minutes, as well as 1 hr, 2 hr, 4 hr, 6 hr, 8 hr, 10 hr, 12 hr, 16 hr, 20 hr, and 24 hours. The composition of the present disclosure can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

The compositions of the present disclosure can be co-administered with another active agent. Co-administration includes administering the composition of the present disclosure and active agent within 0.5 hr, 1 hr, 2 hr, 4 hr, 6 hr, 8 hr, 10 hr, 12 hr, 16 hr, 20 hr, or 24 hours of each other. Co-administration also includes administering the composition of the present disclosure and active agent simultaneously or approximately simultaneously (e.g., within about 1 min, 5 min, 10 min, 15 min, 20 min, or 30 minutes of each other), or sequentially in any order. In addition, the composition of the present disclosure and the active agent can each be administered once a day, or two, three, or more times per day so as to provide the desired dosage level per day.

Co-administration can be accomplished by coimplantation or coinjection.

In some embodiments, co-administration can be accomplished by co-formulation, e.g., preparing a single pharmaceutical formulation including both the composition of the present disclosure and the active agent. In other embodiments, the composition of the present disclosure and the active agent can be formulated separately and co-administered to the subject.

The composition of the present disclosure and the active agent can be present in a formulation in any suitable weight ratio, such as from 1:100 to 100:1 (w/w), or 1:50 to 50:1, or 1:25 to 25:1, or 1:10 to 10:1, or 1:5 to 5:1 (w/w). The composition of the present disclosure and the other active agent can be present in any suitable weight ratio, such as 1:100 (w/w), 1:75, 1:50, 1:25, 1:10, 1:5, 1:4, 1:3, 1:2, 1:1, 2:1, 3:1, 4:1, 5:1, 10:1, 25:1, 50:1, 75:1, or 100:1 (w/w). Other dosages and dosage ratios of the composition of the present disclosure and the active agent are suitable in the formulations and methods described herein.

### Combination Therapies

In one aspect, provided is a method of treating cancer or enhancing or eliciting an immune response comprising administering to a subject in need thereof: a therapeutically effective amount of a targeting moiety of the disclosure, or a pharmaceutically acceptable salt or composition thereof; and a prodrug, such as those as described herein; and optionally a therapeutically effective amount of an additional therapeutic agent selected from the group consisting of an anticancer agent, an immunomodulatory agent, or a trans-cyclooctene prodrug thereof.

The disclosure also provides a pharmaceutical combination comprising a targeting moiety described herein, or a pharmaceutically acceptable salt, or composition thereof; a prodrug as described herein; and optionally an additional therapeutic agent selected from the group consisting of an anticancer agent, an immunomodulatory agent, or a trans-cyclooctene prodrug thereof, for use in the treatment or prevention of a cancer or for use in enhancing or eliciting an immune response.

The disclosure also provides the use of a pharmaceutical combination comprising a targeting moiety as described herein, or a pharmaceutically acceptable salt, or composition thereof; a prodrug, such as those described herein; and optionally a therapeutically effective amount of an additional therapeutic agent selected from the group consisting of an anticancer agent, an immunomodulatory agent, or a trans-cyclooctene prodrug thereof for the treatment or prevention of a cancer or for use in enhancing or eliciting an immune response.

In the methods and uses described herein, the components of the pharmaceutical combinations may be administered/used simultaneously, separately, or sequentially, and in any order, and the components may be administered separately or as a fixed combination. For example, the delay of progression or treatment of diseases according to the disclosure may comprise administration of the first active ingredient in free or pharmaceutically acceptable salt form and administration of the second active ingredient in free or pharmaceutically acceptable salt form, simultaneously or sequentially in any order, in jointly therapeutically effective amounts or effective amounts, e.g. in daily dosages corresponding to the amounts described herein. The individual active ingredients of the combination can be administered separately at different times during the course of therapy or concurrently in divided or single dosage forms. The instant disclosure is therefore to be understood as embracing all such regimes of simultaneous or alternating treatment and the term “administering” is to be interpreted accordingly. Thus, a pharmaceutical combination, as used herein, defines either a fixed combination in one dosage unit form or separate dosages forms for the combined administration where the combined administration may be independently at the same time or at different times. As a further example, the targeting moiety (or therapeutic targeting moiety) and prodrug may be administered/used simultaneously (e.g., through coinjection or coimplantation), separately, or sequentially, followed by administration of the additional therapeutic agent selected from the group consisting of an anticancer agent, an immunomodulatory agent, or a trans-cyclooctene prodrug thereof.

The methods and uses in treating cancer include administering/localizing the targeting moiety at a tumor. In the methods and uses disclosed herein, the administration of the prodrug, or a pharmaceutically acceptable salt, or composition thereof; the targeting moiety; and optionally an additional therapeutic agent may inhibit the growth of the tumor.

Additional therapeutic agent(s) may be administered simultaneously or sequentially with the disclosed conjugates and compositions. Sequential administration includes administration before or after the disclosed conjugates and compositions. An additional therapeutic agent may be administered before the disclosed conjugates and compositions. An additional therapeutic agent may be administered after the disclosed conjugates and compositions. An additional therapeutic agent may be administered at the same time as the disclosed conjugates and compositions. In some embodiments, the additional therapeutic agent or agents may be administered in the same composition as the disclosed conjugates. In other embodiments, there may be an interval of time between administration of the additional therapeutic agent and the disclosed conjugates or compositions. In some embodiments, administration of an additional therapeutic agent with a disclosed conjugate or composition may allow lower doses of the other therapeutic agents and/or administration at less frequent intervals. When used in combination with one or more other active ingredients, the conjugates or compositions of the present disclosure and the other active ingredients may be used in lower doses than when each is used singly. Accordingly, the pharmaceutical compositions of the present disclosure include those that contain one or more other active ingredients, in addition to a conjugates of the present disclosure.

### Anticancer Agents

Exemplary anti-cancer agents include, but are not limited to, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Adrucil (Fluorouracil), Afatinib Dimaleate, Afinitor (Everolimus), Aldara (Imiquimod), Aldesleukin, Alemtuzumab, Alimta (Pemetrexed Disodium), Aloxi (Palonosetron Hydrochloride), Ambochlorin (Chlorambucil), Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Avastin (Bevacizumab), Axitinib, Azacitidine, BEACOPP, Bendamustine Hydrochloride, BEP, Bevacizumab, Bexarotene, Bexxar (Tositumomab and I 131 Iodine Tositumomab), Bicalutamide, Bleomycin, Bortezomib, Bosulif (Bosutinib), Bosutinib, Brentuximab Vedotin, Busulfan, Busulfex (Busulfan), Cabazitaxel, Cabozantinib-S-Malate, CAF, Campath (Alemtuzumab), Camptosar (Irinotecan Hydrochloride), Capecitabine, CAPOX, Carboplatin, Carboplatin-Taxol, Carfilzomib, Casodex (Bicalutamide), CeeNU (Lomustine), Cerubidine (Daunorubicin Hydrochloride), Cervarix (Recombinant HPV Bivalent Vaccine), Cetuximab, Chlorambucil, Chlorambucil-Prednisone, CHOP, Cisplatin, Clafen (Cyclophosphamide), Clofarabine, Clofarex (Clofarabine), Clolar (Clofarabine), CMF, Cometriq (Cabozantinib-S-Malate), COPP, COPP-ABV, Cosmegen (Dactinomycin), Crizotinib, CVP, Cyclophosphamide, Cyfos (Ifosfamide), Cytarabine, Cytarabine liposomal, Cytosar-U (Cytarabine), Cytoxan (Cyclophosphamide), Dabrafenib, Dacarbazine, Dacogen (Decitabine), Dactinomycin, Dasatinib, Daunorubicin Hydrochloride, Decitabine, Degarelix, Denileukin Diftitox, Denosumab, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Efudex (Fluorouracil), Elitek (Rasburicase), Ellence (Epirubicin Hydrochloride), Eloxatin (Oxaliplatin), Eltrombopag Olamine, Emend (Aprepitant), Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Etopophos (Etoposide Phosphate), Etoposide, Etoposide Phosphate, Evacet (Doxorubicin Hydrochloride Liposome), Everolimus, Evista (Raloxifene Hydrochloride), Exemestane, Fareston (Toremifene), Faslodex (Fulvestrant), FEC, Femara (Letrozole), Filgrastim, Fludara (Fludarabine Phosphate), Fludarabine Phosphate, Fluoroplex (Fluorouracil), Fluorouracil, Folex (Methotrexate), Folex PFS (Methotrexate), Folfiri, Folfiri-Bevacizumab, Folfiri-Cetuximab, Folfirinox, Folfox (Leucovorin, Fluorouracil, Oxaliplatin), Folotyn (Pralatrexate), FU-LV, Fulvestrant, Gardasil (Recombinant HPV Quadrivalent Vaccine), Gazyva (Obinutuzumab), Gefitinib, Gemcitabine Hydrochloride, Gemcitabine-Cisplatin, Gemcitabine-Oxaliplatin, Gemtuzumab Ozogamicin, Gemzar (Gemcitabine Hydrochloride), Gilotrif (Afatinib Dimaleate), Gleevec (Imatinib Mesylate), Glucarpidase, Goserelin Acetate, Halaven (Eribulin Mesylate), Herceptin (Trastuzumab), HPV Bivalent Vaccine, Recombinant, HPV Quadrivalent Vaccine, Recombinant, Hycamtin (Topotecan Hydrochloride), Hyper-CVAD, Ibritumomab Tiuxetan, Ibrutinib, ICE, Iclusig (Ponatinib Hydrochloride), Ifex (Ifosfamide), Ifosf amide, Ifosfamidum (Ifosfamide), Imatinib Mesylate, Imbruvica (Ibrutinib), Imiquimod, Inlyta (Axitinib), Intron A (Recombinant Interferon Alfa-2b), Iodine 131 Tositumomab and Tositumomab, Ipilimumab, Iressa (Gefitinib), Irinotecan Hydrochloride, Istodax (Romidepsin), Ixabepilone, Ixempra (Ixabepilone), Jakafi (Ruxolitinib Phosphate), Jevtana (Cabazitaxel), Kadcyla (Ado-Trastuzumab Emtansine), Keoxifene (Raloxifene Hydrochloride), Kepivance (Palifermin), Kyprolis (Carfilzomib), Lapatinib Ditosylate, Lenalidomide, Letrozole, Leucovorin Calcium, Leukeran (Chlorambucil), Leuprolide Acetate, Levulan (Aminolevulinic Acid), Linfolizin (Chlorambucil), LipoDox (Doxorubicin Hydrochloride Liposome), Liposomal Cytarabine, Lomustine, Lupron (Leuprolide Acetate), Lupron Depot (Leuprolide Acetate), Lupron Depot-Ped (Leuprolide Acetate), Lupron Depot-3 Month (Leuprolide Acetate), Lupron Depot-4 Month (Leuprolide Acetate), Marqibo (Vincristine Sulfate Liposome), Matulane (Procarbazine Hydrochloride), Mechlorethamine Hydrochloride, Megace (Megestrol Acetate), Megestrol Acetate, Mekinist (Trametinib), Mercaptopurine, Mesna, Mesnex (Mesna), Methazolastone (Temozolomide), Methotrexate, Methotrexate LPF (Methotrexate), Mexate (Methotrexate), Mexate-AQ (Methotrexate), Mitomycin C, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Nelarabine, Neosar (Cyclophosphamide), Neupogen (Filgrastim), Nexavar (Sorafenib Tosylate), Nilotinib, Nolvadex (Tamoxifen Citrate), Nplate (Romiplostim), Obinutuzumab, Ofatumumab, Omacetaxine Mepesuccinate, Oncaspar (Pegaspargase), Ontak (Denileukin Diftitox), OEPA, OPPA, Oxaliplatin, Paclitaxel, Paclitaxel Albumin-stabilized Nanoparticle Formulation, Palifermin, Palonosetron Hydrochloride, Pamidronate Disodium, Panitumumab, Paraplat (Carboplatin), Paraplatin (Carboplatin), Pazopanib Hydrochloride, Pegaspargase, Peginterferon Alfa-2b, PEG-Intron (Peginterferon Alfa-2b), Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Pralatrexate, Prednisone, Procarbazine Hydrochloride, Proleukin (Aldesleukin), Prolia (Denosumab), Promacta (Eltrombopag Olamine), Provenge (Sipuleucel-T), Purinethol (Mercaptopurine), Radium 223 Dichloride, Raloxifene Hydrochloride, Rasburicase, R-CHOP, R-CVP, Recombinant HPV Bivalent Vaccine, Recombinant HPV Quadrivalent Vaccine, Recombinant Interferon Alfa-2b, Regorafenib, Revlimid (Lenalidomide), Rheumatrex (Methotrexate), Rituxan (Rituximab), Rituximab, Romidepsin, Romiplostim, Rubidomycin (Daunorubicin Hydrochloride), Ruxolitinib Phosphate, Sclerosol Intrapleural Aerosol (Talc), Sipuleucel-T, Sorafenib Tosylate, Sprycel (Dasatinib), Stanford V, Sterile Talc Powder (Talc), Steritalc (Talc), Stivarga (Regorafenib), Sunitinib Malate, Sutent (Sunitinib Malate), Sylatron (Peginterferon Alfa-2b), Synovir (Thalidomide), Synribo (Omacetaxine Mepesuccinate), Tafinlar (Dabrafenib), Talc, Tamoxifen Citrate, Tarabine PFS(Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalidomide, Thalomid (Thalidomide), Toposar (Etoposide), Topotecan Hydrochloride, Toremifene, Torisel (Temsirolimus), Tositumomab and 1 131 Iodine Tositumomab, Totect (Dexrazoxane Hydrochloride), Trametinib, Trastuzumab, Treanda (Bendamustine Hydrochloride), Trisenox (Arsenic Trioxide), Tykerb (Lapatinib Ditosylate), Vandetanib, VAMP, Vectibix (Panitumumab), VelP, Velban (Vinblastine Sulfate), Velcade (Bortezomib), Velsar (Vinblastine Sulfate), Vemurafenib, VePesid (Etoposide), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, Vismodegib, Voraxaze (Glucarpidase), Vorinostat, Votrient (Pazopanib Hydrochloride), Wellcovorin (Leucovorin Calcium), Xalkori (Crizotinib), Xeloda (Capecitabine), Xelox, Xgeva (Denosumab), Xofigo (Radium 223 Dichloride), Xtandi (Enzalutamide), Yervoy (Ipilimumab), Zaltrap (Ziv-Aflibercept), Zelboraf (Vemurafenib), Zevalin (Ibritumomab Tiuxetan), Zinecard (Dexrazoxane Hydrochloride), Ziv-Aflibercept, Zoladex (Goserelin Acetate), Zoledronic Acid, Zolinza (Vorinostat), Zometa (Zoledronic Acid), and Zytiga (Abiraterone Acetate).

The anticancer agent may be a PBD dimer, calicheamicin, speromycin, tubulysin B, rhizoxin, dolastatin, didemnin B, camptothecin, CBI, temsirolimus, actinomycin D, epothilone B, taxol, cryptophycin, SN38, velcade, bruceantin, DAVLBH, DM1, Phyllanthoside, Alimta, T2 Toxin, MMC, vantalanib, vinorelbine, brefeldin, sunitinib, daunomycin, semaxanib, tarceva, iressa, irinotecan, LY-541503, geldanomycin, gemcitabine, methotrexate, gleevec, topotecan, bleomycin, doxorubicin, cisplatin, N-mustards, etoposide, or 5-FU.

In certain embodiments, an anticancer agent is an anthracycline. In certain embodiments, anticancer agent is a taxane. In certain embodiments, anticancer agent is gemcitabine. In certain embodiments, anticancer agent is doxorubicin. In certain embodiments, anticancer agent is docetaxel. In certain embodiments, anticancer agent is SN38. In certain embodiments, anticancer agent is monomethyl auristatin E.

### Synthesis of the Compounds

The targeting moieties may be prepared using the methods disclosed herein and routine modifications thereof, which will be apparent given the disclosure herein and methods well known in the art. Conventional and well-known synthetic methods may be used in addition to the teachings herein. The synthesis of typical targeting moieties described herein may be accomplished as described in the following examples. If available, reagents and starting materials may be purchased commercially, e.g., from Sigma Aldrich or other chemical suppliers.

It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in Wuts, P. G. M., Greene, T. W., & Greene, T. W. (2006). Greene's protective groups in organic synthesis. Hoboken, N.J., Wiley-Interscience, and references cited therein.

As shown in Scheme I, compounds of Formula V (wherein each of the dotted lines, R¹, R², R³, R⁴, ring A, t, L, p, and X are independently defined herein, and R⁵⁰ is a synthetic handle for bonding to L, such as a leaving group, e.g., halo, or a portion of L capable of linking to X or a further portion of L, e.g., hydroxy, amino, methylamino, etc.) can be prepared by coupling a compound of Formula I-2 with a suitably functionalized biocompatible support, antibody, or antibody fragment moiety X. Suitable methods can be adapted from the literature (see, e.g., WO2020/077140, WO2018/187740, WO2017/044983, WO2015/139025, and WO2014/205126). Compounds of Formula I-2 can be prepared by reacting compound I-1 with a precursor to L under suitable coupling reaction conditions. In some embodiments, X is an antibody or antibody fragment. Suitable coupling methods include, but are not limited to, use of an succinimide functional group which is capable of forming an amide bond with a primary amine on the antibody or antibody fragment, or L can be functionalized with a group capable of forming a covalent bond to a cysteine residue on the antibody or antibody fragment, such as a pyrrole-2,5-dione.

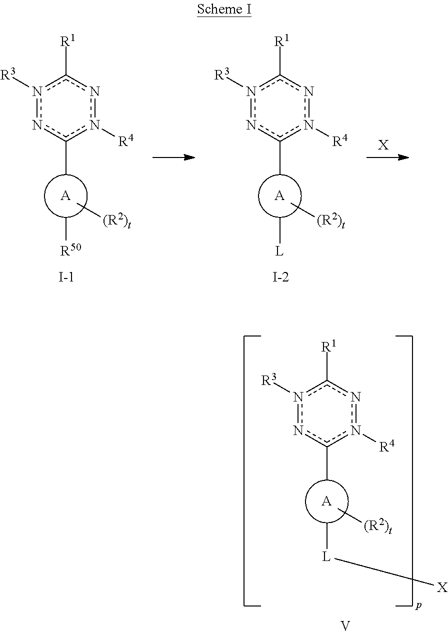

Compounds of Formula I-1 can be prepared according to Scheme II, wherein each of the dotted lines, R¹, R², R³, R⁴, ring A, t, and L are independently defined herein, and R⁵⁰ is a synthetic handle for bonding to L, such as a leaving group, e.g., halo or a thioether, or a portion of L capable of linking to X or a further portion of L, e.g., hydroxy, amino, methylamino, etc., and each LG is independently a leaving group, e.g., halo.

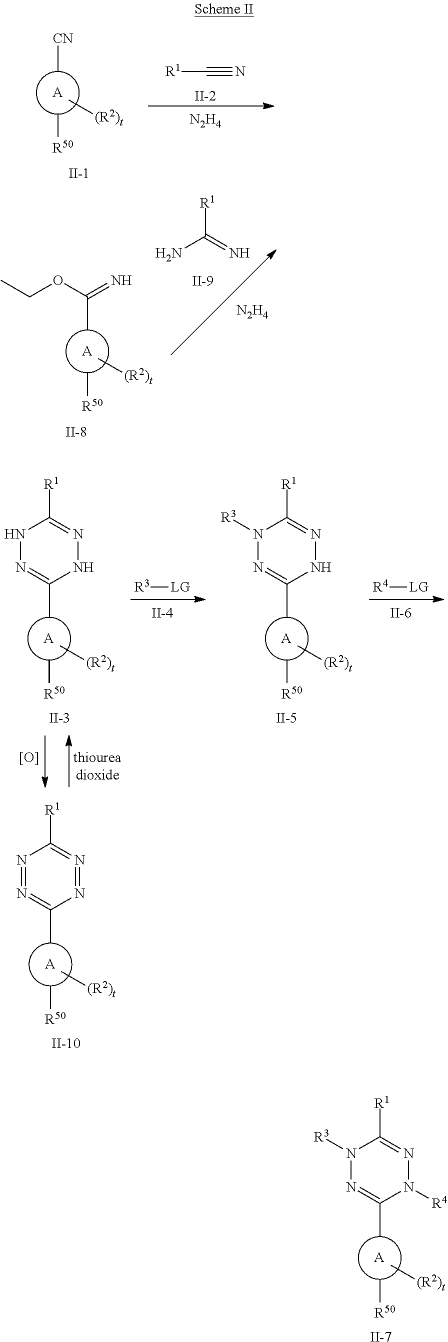

As shown in Scheme II, coupling compound II-1 with compound II-2 in the presence of N₂H₄ provides compound II-3. Further modification of compound II-3 with compound II-4 and/or compound II-6 under standard coupling conditions provides compound II-5 and/or compound II-7. Alternatively, compound II-3 can be provided by coupling compound II-8 with compound II-9 in the presence of N₂H₄. Compound II-10 can be provided by contacting compound II-3 with a suitable oxidizing agent (e.g., NaNO₂). Alternatively, compound II-3 can be provided by contacting compound II-10 with thiourea dioxide.

Upon each reaction completion, each of the intermediate or final compounds can be recovered, and optionally purified, by conventional techniques such as neutralization, extraction, precipitation, chromatography, filtration and the like.

It should be understood that any of the compounds or intermediates shown in Scheme I or II may be prepared using traditional methods or purchased from commercial sources. In addition, any of the intermediates or any product obtained by the process outlined in Scheme I or II can be derivatized at any step to provide various compounds of Formula V.

Exemplary payloads can be prepared can be prepared according to methods adapted from the literature (see, e.g., WO2022/032191, WO2021/007160, WO2020/077140, WO2018/187740, WO2017/044983, WO2015/139025, and WO2014/205126, which methods are incorporated herein in their entirety). Exemplary procedures for MMAE payloads are shown in Examples A, B, and C, which procedures can be adapted to prepare other payloads such as those disclosed herein.

### EXAMPLES

The following examples are included to demonstrate specific embodiments of the disclosure. It should be appreciated by those of skill in the art that the techniques disclosed in the examples which follow represent techniques to function well in the practice of the disclosure, and thus can be considered to constitute specific modes for its practice. However, those of skill in the art should, in light of the present disclosure, appreciate that many changes can be made in the specific embodiments which are disclosed and still obtain a like or similar result without departing from the spirit and scope of the disclosure.

LCMS Analysis Method: Test articles were subjected to PNGaseF (IgG) and DTT or DTT alone (Fab) in RapiGest according to the manufacturer's protocol. Samples were diluted to 100 μg/mL with water and centrifuged at 16.1k RCF for 10 min at 4° C. The samples were then analyzed by LCMS (LC-Q-TOF) and the mass spectra reconstructed from the charge ladder.

### Example 1: Synthesis of Tetrazine-Trastuzumab Targeting Moiety

Trastuzumab (22.1 mg/mL, 1.1 mL) in 0.01 M PBS was mixed with 20 equivalents of methyltetrazine-PEG4-NHS(Clickchemtools #1069-10).

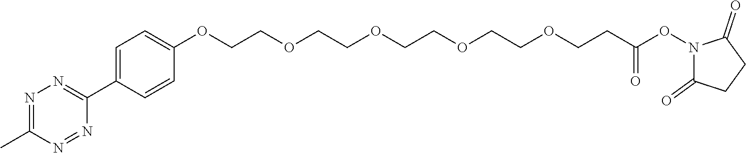 [SMILES: Cc1nnc(-c2ccc(OCCOCCOCCOCCOCCC(=O)ON3C(=O)CCC3=O)cc2)nn1]

methyltetrazine-PEG4-NHS

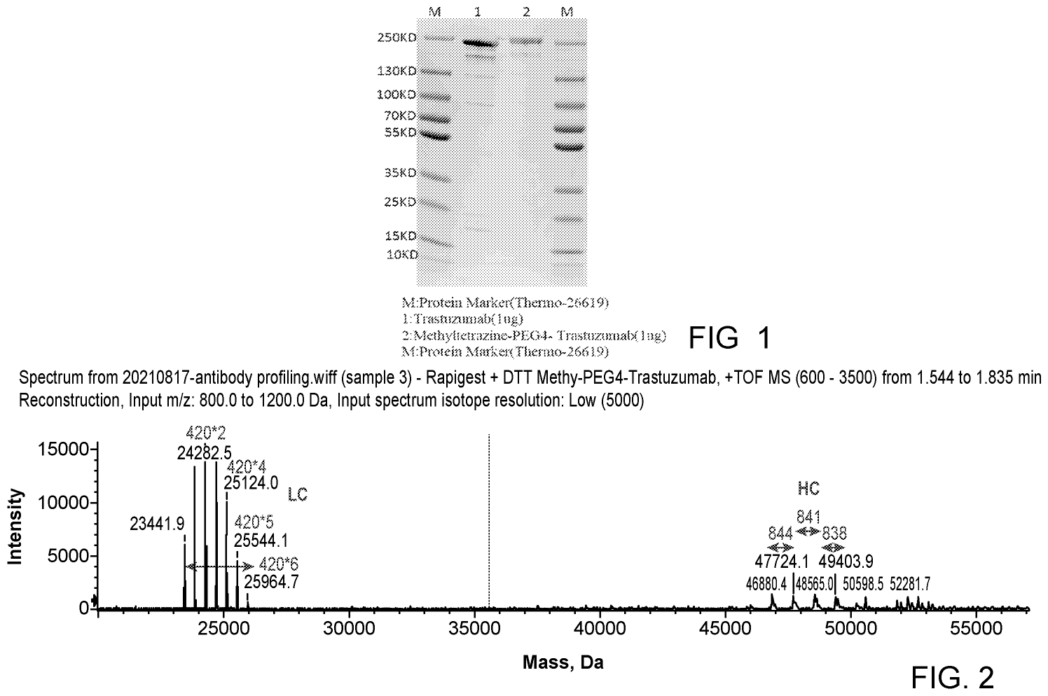

The reaction was mixed thoroughly and aged at room temperature for 1 hour, at which time the reaction was quenched by the addition of 1 volume of 0.1 M Tris buffer. The resulting solution was buffer exchanged to 0.01 M PBS to remove excess reagent and buffer salts. The resulting solution of targeting moiety (6.3 mg/mL, 1.6 mL) was analyzed by SDS-Page (FIG. 1) and LCMS (FIG. 2) confirming the formation of the targeting moiety, and thus was used for subsequent studies. Based on the analysis, it is contemplated that up to 12 methyltetrazine-PEG4 units are covalently bonded to the antibody.

### Example 2: Tetrazine-Fab Targeting Moiety

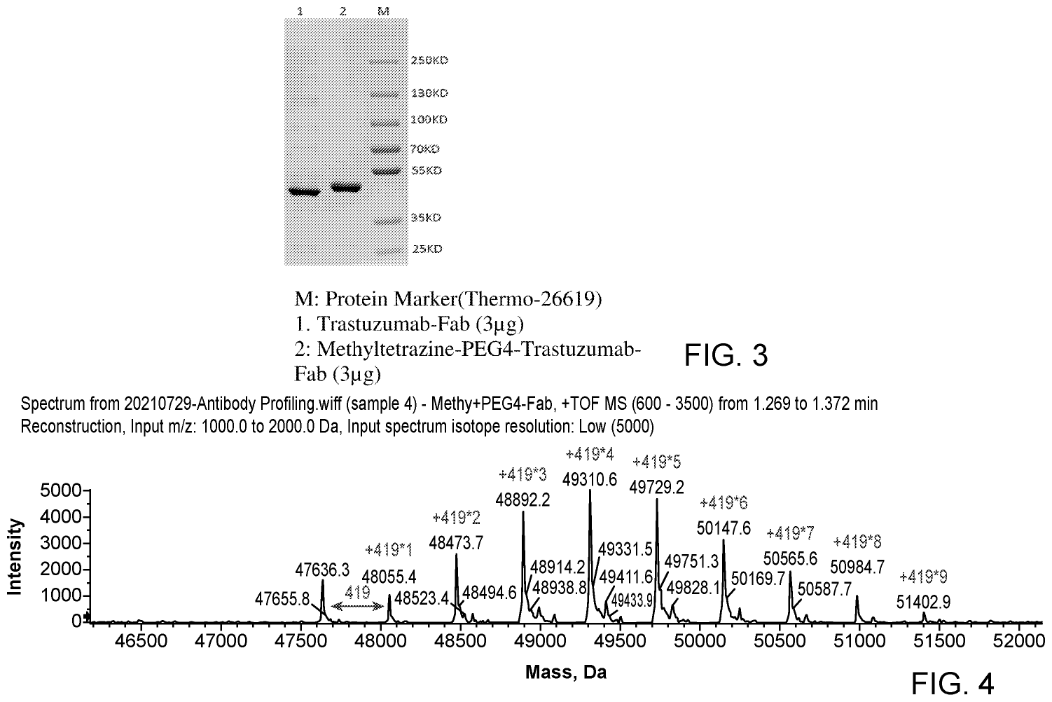

Fab was prepared from Trastuzumab using a commercial kit (Pierce™ Fab Preparation Kit #44985) according to the manufacturers protocol and purified by protein G resin (BioVision #6511-25). The purified Fab in 0.01 M PBS was mixed with 20 equivalents of methyltetrazine-PEG4-NHS (Clickchemtools #1069-10). The reaction was mixed thoroughly and aged at room temperature for 1 hour, at which time the reaction was quenched by the addition of 1 volume of 0.1 M Tris buffer. The resulting solution was buffer exchanged to 0.01 M PBS to remove excess reagent and buffer salts. The resulting solution of targeting moiety (1.0 mg/mL, 10.3 mL) was analyzed by SDS-Page (FIG. 3) and LCMS (FIG. 4) confirming the formation of the targeting moiety, and thus was used for subsequent studies. Based on the analysis, it is contemplated that up to about 6 methyltetrazine-PEG4 units are covalently bonded to the Fab.

### Example 3: Tetrazine-Fab Targeting Moiety

Fab is prepared from Enfortumab using the following method. 0.1 mg papain was pretreated with 1 mM DTT at 0.5 mg/ml concentration and 2 mM EDTA by incubating at 37° C. for 30 min. The antibody was prepared in PBS buffer, pH 7.4 (10 mg, 0.5 mg/mL). The pretreated papain was mixed with the antibody at (1:100) molar ratio and incubated at 37° C. for 2 hours. The digestion mixture was loaded onto an anti-CH1 affinity column, washed with 25 mM Tris, 150 mM NaCl, pH 8.0, and eluted with 50 mM sodium citrate, 150 mM NaCl, pH 3.0. The filtrate containing the product Fab was dialyzed into PBS. The purified Fab was in PBS was concentrated to 0.2 mg/mL. The Fab was mixed with Me-Tet-PEG9-NHS (prepared in DMSO at 10 mM) at 3:1 molar ratio and incubated at 37° C. for 2 hours. The resulting conjugate was analyzed by LCMS and the DAR calculated to be 2.66.

### Example 4: Tetrazine-Fab Targeting Moiety

Fab is prepared from Brentuximab using the following method. 0.1 mg papain was pretreated with 1 mM DTT at 0.5 mg/ml concentration and 2 mM EDTA by incubating at 37° C. for 30 min. The antibody was prepared in PBS buffer, pH 7.4 (10 mg, 0.5 mg/mL). The pretreated papain was mixed with the antibody at (1:100) molar ratio and incubated at 37° C. for 2 hours. The digestion mixture was loaded onto an anti-CH1 affinity column, washed with 25 mM Tris, 150 mM NaCl, pH 8.0, and eluted with 50 mM sodium citrate, 150 mM NaCl, pH 3.0. The filtrate containing the product Fab was dialyzed into PBS. The purified Fab was in PBS was concentrated to 0.2 mg/mL. The Fab was mixed with Me-Tet-PEG9-NHS (prepared in DMSO at 10 mM) at 3:1 molar ratio and incubated at 37° C. for 2 hours. The resulting conjugate was analyzed by LCMS and the DAR calculated to be 4.57.

### Example 5: Tetrazine-Fab Targeting Moiety

Fab is prepared from Sacituzumab using a commercial kit (Pierce™ Fab Preparation Kit #44985) according to the manufacturers protocol and purified by protein G resin (BioVision #6511-25). The purified Fab 10 mM Me-Tet-PEG9-NHS is prepared in DMSO. The two components are reacted at 3:1 drug to protein molar ratio at 25° C. for 2 hours before it is dialyzed against PBS, pH 7.4 to remove excess Me-Tet-PEG9-NHS compound from the protein component. The resulting solution of targeting moiety is analyzed by SDS-Page and LCMS to confirm the formation of the targeting moiety. It is contemplated that approximately two methyltetrazines will be covalently bonded to each Fab, on average, as confirmed by LCMS.

### Example 6: Tetrazine-Antigen-Binding Protein Targeting Moiety

Antigen-binding proteins are engineered proteins that can bind an antigen. The proteins are approximately 66 amino acids in length with a molecular weight of 7 kDa. The protein can be expressed in E. coli and a cysteine residue can be included at the N- or C-terminus of the sequence that can be conjugated with a cysteine-reactive group and can be purchased from commercial sources (e.g., Nanofitins® from Affilogic).

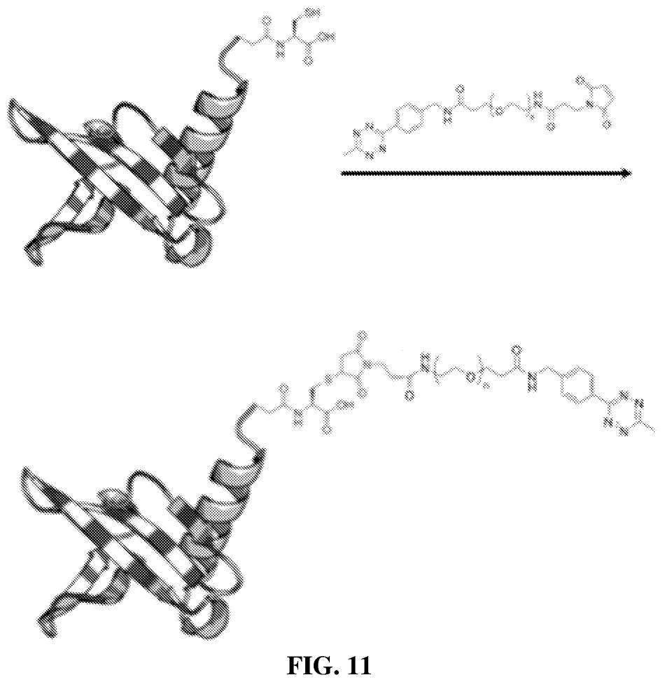

An antigen-binding protein targeting HER2 with a C-terminal cysteine is expressed in E. coli and purified to homogeneity. The protein is treated with a reducing agent, such as TCEP, at ambient temperature or on ice, followed by buffer exchange into fresh buffer. The protein is then treated with maleimide-PEG-tetrazine (n=3). After the reaction is complete, the protein is exchanged into fresh buffer to remove the excess reagent to yield conjugate (Ab-Tz, FIG. 11). The conjugate is analyzed by SDS-PAGE, analytical HPLC, mass spectrometry to confirm the expected properties. The conjugate can also be treated with a trans-cyclooctene-functionalized fluorophore to confirm the reactivity of the tetrazine.

An non-binding control protein with a C-terminal cysteine is expressed in E. coli and purified to homogeneity. The protein is treated with a reducing agent, such as TCEP, at ambient temperature or on ice, followed by buffer exchange into fresh buffer. The protein is then treated with maleimide-PEG-tetrazine (n=3). After the reaction is complete, the protein is exchanged into fresh buffer to remove the excess reagent to yield conjugate (Ab-Tz). The conjugate is analyzed by SDS-PAGE, analytical HPLC, mass spectrometry to confirm the expected properties. The conjugate can also be treated with a trans-cyclooctene-functionalized fluorophore to confirm the reactivity of the tetrazine.

### Example 7: HCC1954 Xenograft Model

Animal studies were conducted in accordance with IACUC protocols following the guidance of the AAALAC. Female Balb/c nude mice were implanted with HCC1954 grown in exponential phase in right flank (5e6 cells+Matrigel) in 0.2 mL PBS. The animals were randomized when the tumor volume reached ˜200 mm³. Animals were dosed IV with saline (days 1-4), the methyltetrazine-Fab targeting moiety from Example 2 (day 0, 5 mg/kg)+saline (days 1-4), or the methyltetrazine-Fab targeting moiety from Example 2 (day 0, 5 mg/kg)+doxorubicin-TCO prodrug (days 1, 2, 3 & 4, 120 mg/kg).

The doxorubicin-TCO prodrug has the structure shown below, and was prepared according to the method described in WO2020/077140.

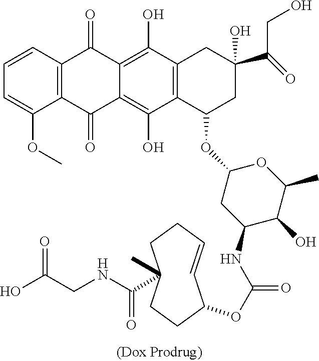

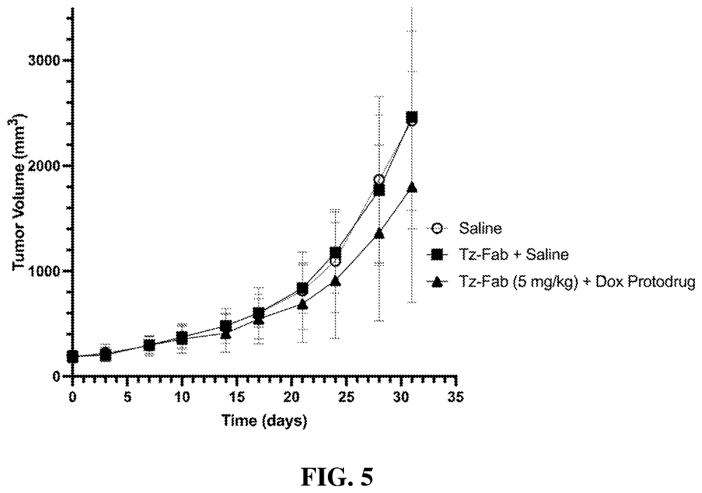

Tumor volume was measured twice weekly in two dimensions using a caliper, and the volume was expressed in mm³ using the formula: V=0.5 a×b² where a and b are the long and short diameters of the tumor, respectively. FIG. 5 shows the tumor growth over 33 days. A decrease in tumor growth with administration of the prodrug shows efficacy of the system described therein.

Suitable prodrugs for use in the methods disclosed herein can be prepared and administered as described in WO2020/077140, WO2018/187740, WO2017/044983, WO2015/139025, and WO2014/205126.

### Example 8: General Procedure for Preparation of Compound A

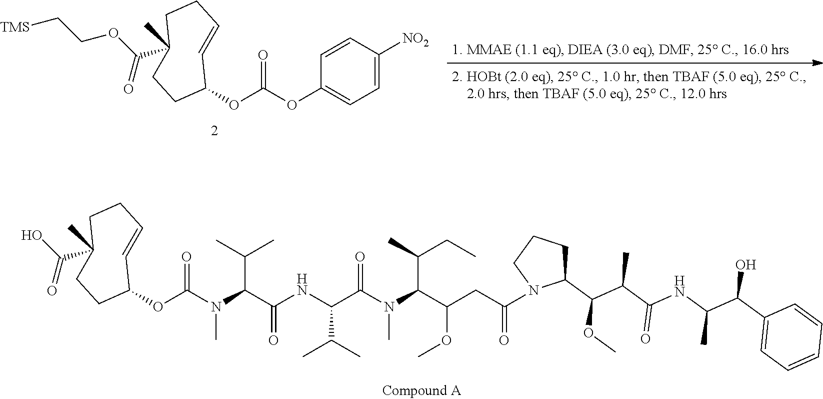

To a solution of MMAE (1.40 g, 1.95 mmol) and DIEA (690 mg, 5.34 mmol) in DMF (4.00 mL) was added compound 2 (800 mg, 1.79 mmol) in DMF (4.00 mL) at 0° C., the mixture was stirred at 25° C. for 16 hrs. Then HOBt (480 mg, 3.56 mmol) in DMF (0.50 mL) was added to the above reaction mixture at 0° C., after the reaction mixture was stirred at 25° C. for 1.0 hr, the reaction mixture was cooled to 0° C., and TBAF (1 M in THF, 4.45 mL) was added. After the mixture was stirred for 2.0 hrs at 25° C., another batch of TBAF (1 M in THF, 4.45 mL) was added at 0° C., the reaction mixture was continued to stirring for 12.0 hrs at 25° C. LC-MS showed one main peak was desired mass. The resulting reaction mixture was purified by Prep-HPLC (column: Welch XB-C18 7 μm 110 A 250*50 mm; mobile phase: [water (0.1% TFA)-ACN]; B %: 50-70%-40 min. number of injections: 2, Retention time: 37 min, flow rate: 60 mL/min) to give Compound A (450 mg, 99.0% purity; 64.6 mg, 99.2%, 31.2% yield).

LCMS (m/z): 928.6 [M+H]⁺

¹HNMR: (400 MHZ, DMSO-d₆): δ 8.46-8.28 (m, 1H), 8.03-7.84 (m, 1H), 7.64 (d, J=8.8 Hz, 1H), 7.35-7.23 (m, 4H), 7.21-7.13 (m, 1H), 6.05-5.57 (m, 2H), 5.10 (s, 1H), 4.81-4.39 (m, 3H), 4.35-4.19 (m, 1H), 4.05-3.92 (m, 2H), 3.40-3.09 (m, 11H), 3.08-2.83 (m, 5H), 2.48-2.37 (m, 2H), 2.31-2.09 (m, 5H), 2.07-1.89 (m, 3H), 1.88-1.64 (m, 6H), 1.63-1.33 (m, 4H), 1.32-1.16 (m, 1H), 1.08-0.97 (m, 9H), 0.90-0.68 (m, 18H).

### Example 9: General Procedure for Preparation of Compound B

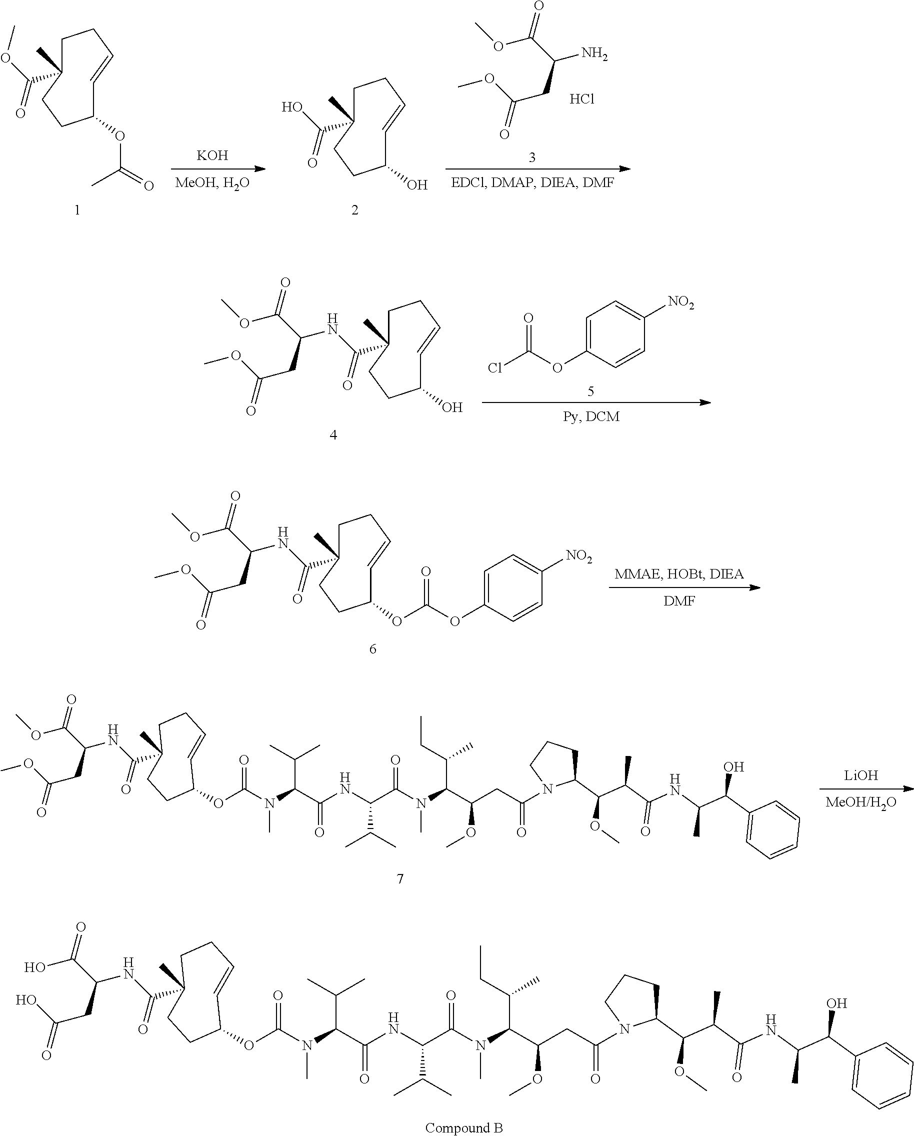

### General Procedure for Preparation of Compound 4

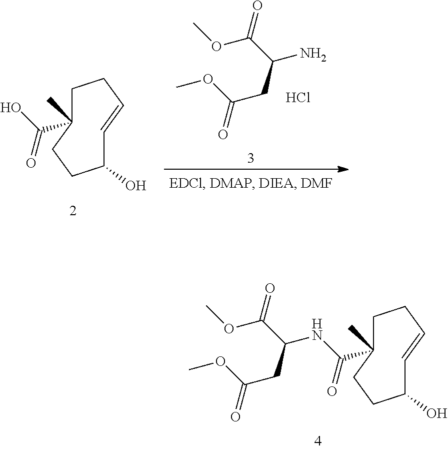

To a solution of compound 2 (1.00 g, 5.43 mmol) in DCM (10 mL) was added DIEA (2.10 g, 16.3 mmol), EDCI (2.08 g, 10.9 mmol) and DMAP (1.33 g, 10.9 mmol) and compound 3 (1.61 g, 8.14 mmol). The mixture was stirred at 25° C. for 16 hrs. TLC indicated compound 2 was consumed completely and one new spot formed. The reaction mixture was partitioned between DCM (20 mL) and H₂O (10 mL). The organic phase was separated, washed with sat. citric acid aq. (3 mL) and brine (20 mL), then dried over Na₂SO₄, filtered and concentrated under reduced pressure to give a residue. The residue was purified by column chromatography (Petroleum ether/Ethyl acetate=3/1 to 1/1) to give compound 4 (700 mg, 39.4% yield).

¹HNMR (400 MHZ, CDCl₃): δ ppm 1.12 (s, 3H), 1.60 (dd, J=15.45, 6.19 Hz, 1H), 1.79-1.87 (m, 2H), 1.92 (br d, J=5.88 Hz, 1H), 1.95 (s, 1H), 1.98-2.00 (m, 1H), 2.02 (br d, J=4.13 Hz, 1H), 2.26 (dd, J=11.63, 3.88 Hz, 1H), 2.30-2.36 (m, 1H), 2.77-2.89 (m, 1H), 2.88-2.88 (m, 1H), 3.00 (dd, J=16.95, 4.57 Hz, 1H), 3.70 (s, 4H), 3.75 (s, 3H), 4.80 (dt, J=8.00, 4.50 Hz, 1H), 5.66 (dd, J=16.63, 2.38 Hz, 1H), 6.02-6.12 (m, 1H), 6.54 (br d, J=7.88 Hz, 2H).

### General Procedure for Preparation of Compound 6

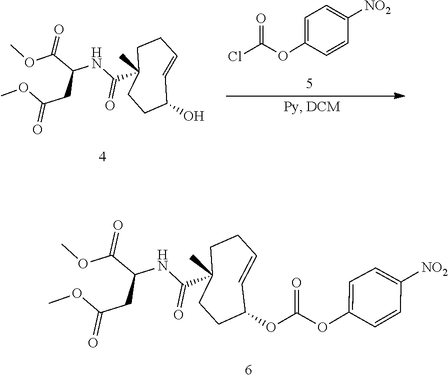

To a solution of compound 4 (700 mg, 2.14 mmol) in DCM (5 mL) was added Py (846 mg, 10.7 mmol) and compound 5 (1.72 g, 8.55 mmol) in DCM (5 mL). The mixture was stirred at 25° C. for 1 hrs. TLC indicated compound 4 was consumed completely and one new spot formed. The reaction mixture was partitioned between DCM (20 mL) and H₂O (10 mL). The organic phase was separated, washed with sat. citric acid aq. (3 mL) and brine (20 mL), dried over Na₂SO₄, filtered and concentrated under reduced pressure to give a residue. The residue was purified by column chromatography (Petroleum ether/Ethyl acetate=3/1 to 1/1) to give compound 6 (490 mg, 46.5% yield).

¹HNMR (400 MHZ, CDCl₃): δ ppm 1.17 (s, 3H), 1.55-1.61 (m, 1H), 1.58 (br s, 1H), 1.76 (dd, J=14.76, 6.25 Hz, 1H), 1.87-2.03 (m, 3H), 2.06-2.15 (m, 1H), 2.19-2.41 (m, 3H), 2.82 (dd, J=17.13, 4.50 Hz, 1H), 3.03 (dd, J=17.07, 4.44 Hz, 1H), 3.72 (s, 3H), 3.77 (s, 3H), 4.78-4.86 (m, 1H), 5.67 (dd, J=16.70, 2.44 Hz, 1H), 6.03-6.14 (m, 1H), 6.58 (br d, J=7.88 Hz, 1H), 7.40-7.45 (m, 2H), 8.27-8.33 (m, 2H).

### General Procedure for Preparation of Compound 7

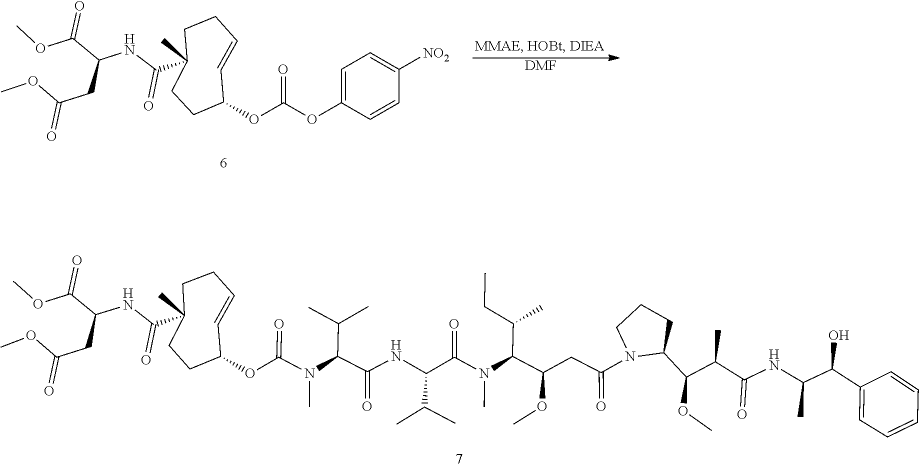

To a solution of compound 6 (490 mg, 995 μmol) and MMAE (714 mg, 995 μmol) in DMF (4 mL) was added DIEA (64.3 mg, 497 μmol) and HOBt (202 mg, 1.49 mmol). The mixture was stirred at 25° C. for 16 hrs. LC-MS showed compound 6 was consumed completely and one main peak with desired mass was detected. The residue was purified by prep-HPLC (0.1% TFA conditions) to give compound 7 (500 mg, 46.9% yield).

¹HNMR (400 MHZ, CDCl₃): δ ppm 0.84 (br d, J=6.75 Hz, 4H), 0.89 (br d, J=4.50 Hz, 5H), 0.92 (br d, J=6.63 Hz, 4H), 0.98 (br d, J=6.25 Hz, 3H), 1.04 (br d, J=6.88 Hz, 3H), 1.16 (s, 3H), 1.25-1.27 (m, 3H), 1.59-1.74 (m, 3H), 1.88 (br d, J=9.38 Hz, 4H), 2.07 (br d, J=8.38 Hz, 5H), 2.27 (br s, 4H), 2.36-2.43 (m, 2H), 2.45-2.53 (m, 1H), 2.89 (br s, 6H), 2.95-3.01 (m, 4H), 3.04 (br s, 2H), 3.29-3.34 (m, 3H), 3.36-3.47 (m, 5H), 3.67-3.73 (m, 4H), 3.76 (s, 3H), 3.82-3.89 (m, 1 H), 4.05-4.19 (m, 3H), 4.28 (br s, 1H), 4.63-4.86 (m, 3H), 4.96 (d, J=2.50 Hz, 1H), 5.24 (br s, 1H), 5.63 (br d, J=18.14 Hz, 1H), 5.82 (br s, 1H), 6.53-6.74 (m, 3H), 7.30-7.41 (m, 5H).

### General Procedure for Preparation of Compound B

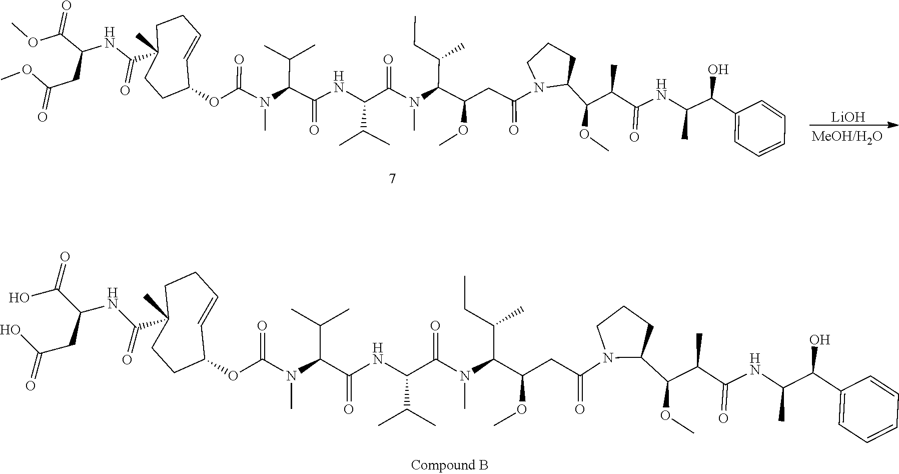

To a solution of compound 7 (500 mg, 467 μmol) in MeOH (5 mL) was added LiOH. H₂O (196 mg, 4.67 mmol) in H₂O (2 mL). The mixture was stirred at 25° C. for 16 hrs. LC-MS showed compound 7 was consumed completely and one main peak with desired mass was detected. The residue was adjusted pH ˜2 with sat. citric acid aq., then purified by prep-HPLC (0.1% TFA condition) to give Compound B (265 mg 53.4% yield).

¹HNMR (400 MHz, CDCl₃): δ ppm 0.80-1.04 (m, 25H), 1.09 (s, 3H), 1.24 (d, J=6.88 Hz, 3 H), 1.69-1.82 (m, 2H), 1.88-1.94 (m, 3H), 2.02-2.11 (m, 4H), 2.16 (br d, J=18.64 Hz, 1H), 2.11-2.24 (m, 2H), 2.32 (br d, J=5.25 Hz, 2H), 2.40-2.45 (m, 1H), 2.52 (br d, J=5.50 Hz, 2H), 2.81 (br dd, J=14.01, 4.88 Hz, 1H), 2.94-3.04 (m, 2H), 3.08 (s, 2H), 3.14-3.27 (m, 6H), 3.33 (s, 1H), 3.39 (s, 3H), 3.48-3.57 (m, 2H), 3.94 (br d, J=1.25 Hz, 1H), 4.05-4.18 (m, 4H), 4.30 (br dd, J=6.19, 4.82 Hz, 2H), 4.54-4.67 (m, 4H), 4.91 (br d, J=2.00 Hz, 2H), 5.30 (br s, 1H), 5.64-5.73 (m, 1H), 5.79-5.89 (m, 1H), 6.61 (br d, J=7.38 Hz, 1H), 7.30-7.42 (m, 5H), 7.55-7.64 (m, 1H).

### Example 10: Alternative Route to Compound B and Synthesis of Compound C

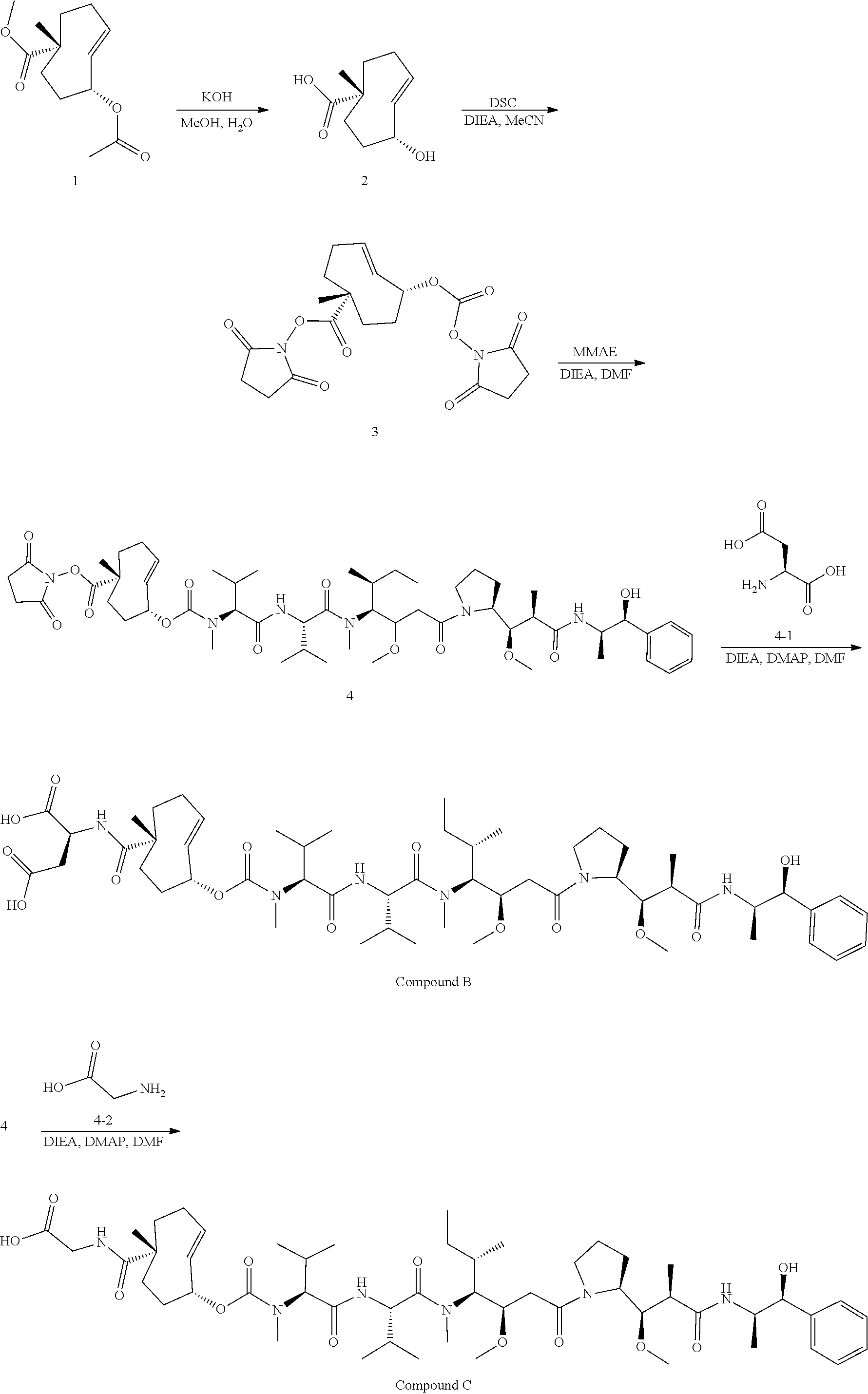

### General Procedure for Preparation of Compound 2

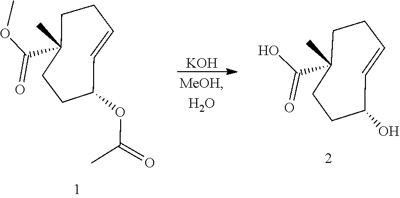

To a solution of compound 1 (20.0 g, 83.2 mmol) in MeOH (80 mL) was added KOH (8.19 g, 124 mmol) in H₂O (80 mL). The mixture was stirred at 25° C. for 24 hrs. The reaction was monitored by TLC (compound 1, PE/EtOAc=5/1, R_{f}=0.5). The reaction mixture was extracted with MTBE (3×400 mL). The combined organic layers were washed with water (100 mL), dried with Na₂SO₄, filtered and concentrated in vacuo to provide the undesired ester. The aqueous layer was acidified with 1 M HCl until pH=4 while cooling in an ice-water bath (T<7° C.). The aqueous layer was extracted with MTBE (3×400 mL). The combined MTBE layers were was dried with Na₂SO₄, filtered and concentrated in vacuo to provide the compound 2 (5.50 g, 35.9% yield). The crude product was used into the next step without further purification.

¹H NMR: (400 MHZ, DMSO-d6): δ ppm 11.9 (br s, 1H), 5.81-5.94 (m, 1H), 5.58 (dd, J=16.45, 2.31 Hz, 1H), 4.65 (br s, 1H), 4.24 (br s, 1H), 2.04-2.24 (m, 2H), 1.87-2.03 (m, 1H), 1.61-1.86 (m, 4H), 1.36-1.46 (m, 1H), 0.97 (s, 3H).

### General Procedure for Preparation of Compound 3

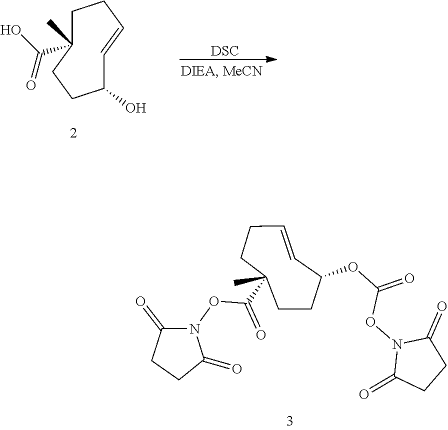

To a solution of compound 2 (8.00 g, 43.4 mmol) in MeCN (160 mL) was added DIEA (39.3 g, 304 mmol) and DSC (47.8 g, 186.4 mmol). The mixture was stirred at 40° C. for 14 hrs. The completion of the reaction was confirmed by TLC (compound 2, DCM/MeOH=10/1, R_{f}=0.5). The reaction mixture was poured in water (400 mL), then the temperature rose from 20° C. to 27° C. After 15 mins, the mixture was cooled to 17° C. in an ice-water bath and stirred for 15 mins. The solid was filtered, washed with water (3×20 mL) and was dried under vacuum at 35° C. for 4 hrs to give crude compound 3 (9.60 g). Acetonitrile (20 mL) was added to the crude and the mixture was heated at 40° C. for 1 hrs using mechanical stirring. The heating was stopped and the mixture was cooled to 8° C. in an ice-water bath for 15 mins. The solid was filtered, washed with acetonitrile (2×10 mL) and dried under vacuum at 35° C. for 3 hrs to give compound 3 (6.65 g, 36.3% yield).

¹H NMR: (400 MHZ, CDCl₃): δ 6.03-6.14 (m, 1H), 5.60-5.67 (m, 1H), 5.29 (br s, 1H), 2.80-2.88 (m, 8H), 2.25-2.47 (m, 4H), 1.94-2.18 (m, 4H), 1.29 (s, 3H).

### General Procedure for Preparation of Compound 4

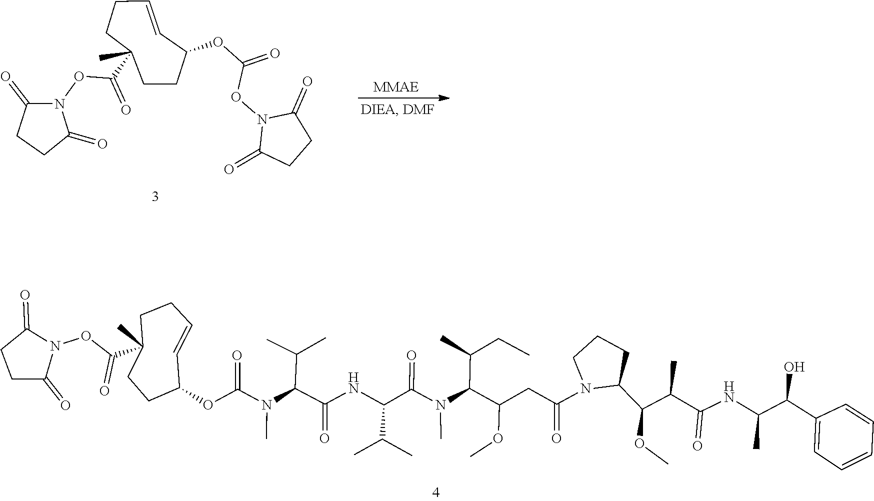

To a solution of compound 3 (100 mg, 0.24 mmol) in DMF (20 mL) was added MMAE (136 mg, 0.19 mmol) and DIEA (61.2 mg, 0.47 mmol). The mixture was stirred at 25° C. for 16 hrs. LC-MS showed one main peak with desired mass was detected. The residue was purified by prep-HPLC (Water (0.1% FA)-ACN) to give compound 4 (41.0 mg, 16.9% yield).

LCMS (m/z): 1025.6 (M+H)⁺.

### General Procedure for Preparation of Compound B

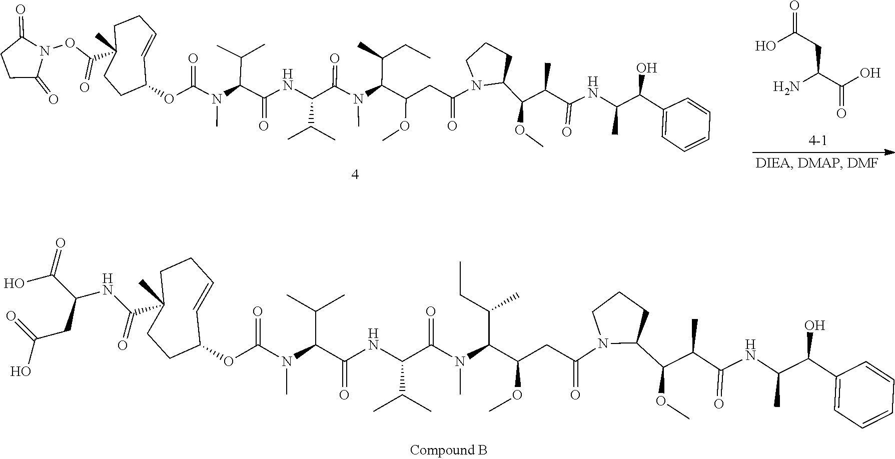

To a solution of compound 4 (500 mg, 0.49 mmol) and compound 4-1 (519 mg, 3.90 mmol) in DMF (10 mL) was added DIEA (378 mg, 2.93 mmol) and DMAP (119 mg, 0.97 mmol). The mixture was stirred at 25° C. for 12 hrs. LC-MS showed one main peak with desired mass was detected. The residue was purified by prep-HPLC (Water (0.1% TFA)-ACN) to give Compound B (161 mg, 31.6% yield).

¹H NMR: (400 MHZ, MeOD): δ 7.73-8.00 (m, 1H), 7.20-7.39 (m, 4H), 5.78-5.96 (m, 1H), 5.73 (br s, 1H), 5.20-5.28 (m, 1H), 5.13-5.20 (m, 1H), 4.49-4.74 (m, 3H), 4.17-4.28 (m, 2H), 4.04-4.10 (m, 1H), 3.85-3.90 (m, 1H), 3.50-3.80 (m, 2H), 3.31-3.50 (m, 9H), 3.28-3.30 (m, 3H), 2.77-3.12 (m, 6H), 2.44-2.58 (m, 2H), 1.78-2.36 (m, 13H), 1.56-1.72 (m, 2H), 1.22-1.48 (m, 3 H), 1.08-1.23 (m, 9H), 0.80-1.07 (m, 18H).

LCMS (m/z): 1043.62 (M+H)⁺; 1065.61 (M+Na)⁺.

### General Procedure for Preparation of Compound C

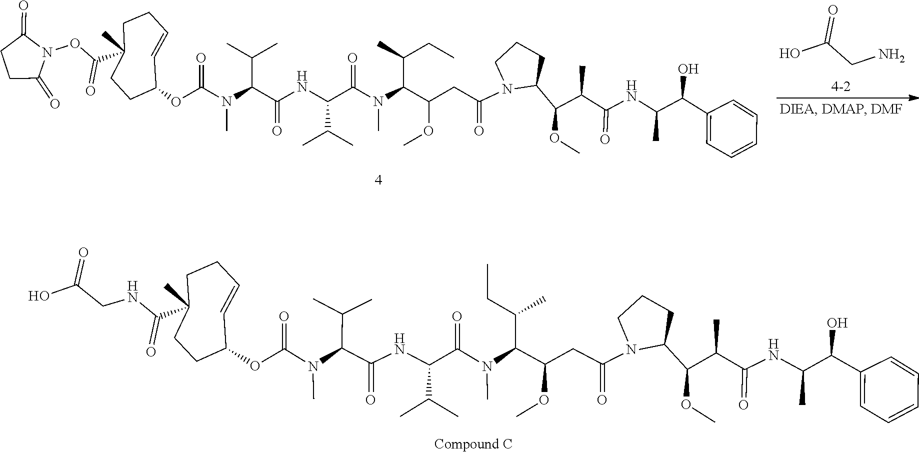

To a solution of compound 4 (320 mg, 0.31 mmol) and compound 4-2 (187 mg, 2.50 mmol) in DMF (3.2 mL) was added DIEA (242 mg, 1.87 mmol) and DMAP (76.3 mg, 0.62 mmol). The mixture was stirred at 25° C. for 12 hrs. LC-MS showed compound 4 was consumed completely and one main peak with desired mass was detected. The residue was purified by prep-HPLC (Water (0.1% TFA)-ACN) to give Compound C (92.0 mg, 29.9% yield).

¹H NMR (400 MHZ, MeOD): δ 7.86-8.00 (m, 1H), 7.15-7.45 (m, 5H), 5.67-5.98 (m, 2H), 5.17 (br s, 1H), 4.50-4.74 (m, 2H), 4.03-4.29 (m, 3H), 3.81-3.89 (m, 2H), 3.51-3.77 (m, 2H), 3.46-3.50 (m, 1H), 3.33-3.45 (m, 5H), 3.30 (br s, 4H), 3.20 (dt, J=11.57, 7.47 Hz, 1H), 3.02-3.15 (m, 3H), 2.90-3.01 (m, 1H), 2.41-2.57 (m, 2H), 1.65-2.39 (m, 15H), 1.52-1.64 (m, 1H), 1.26-1.51 (m, 2H), 1.08-1.23 (m, 9H), 0.82-1.07 (m, 18H).

LCMS (m/z): 985.6 (M+H)⁺.

### Example 11: Synthesis of Compound D

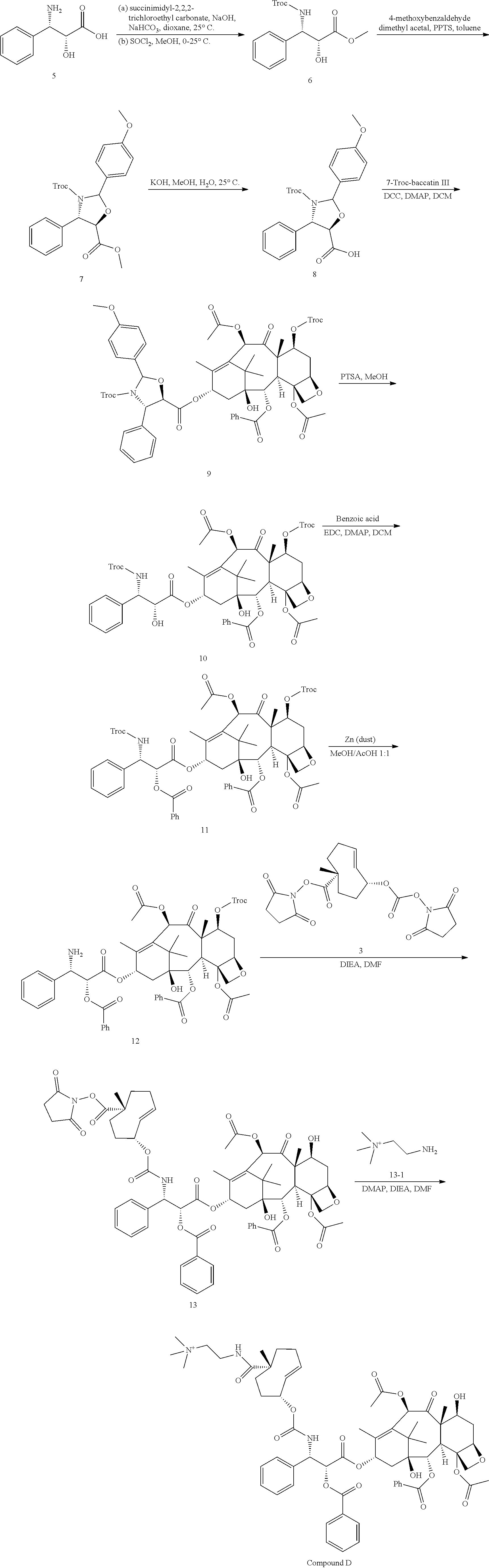

### General Procedure for Preparation of Compound 6

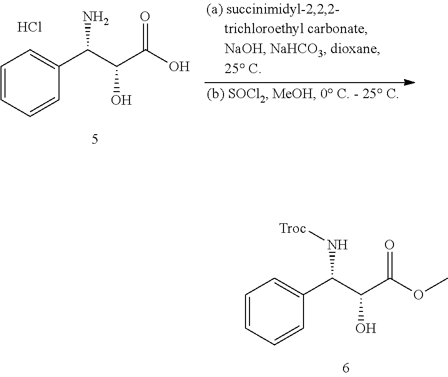

To a solution of compound 5 (150 g, 689 mmol, HCl) in NaOH (1 M, 1.38 L) and NaHCO₃ (1 M, 1.38 L) was added (2,5-dioxopyrrolidin-1-yl) 2,2,2-trichloroethyl carbonate (210 g, 723 mmol) in dioxane (1 L). The mixture was stirred at 25° C. for 2 hrs. The reaction mixture was concentrated under reduced pressure to remove dioxane. The residue was extracted with MTBE (5 L), then the aqueous phase was adjusted pH˜4 with Sat. KHSO₄ aq. and extracted with EtOAc (5 L). The combined organic layers were dried over Na₂SO₄, filtered and concentrated under reduced pressure to give a residue. To a solution of above crude in MeOH (2 L) was added SOCl₂ (90.2 g, 758 mmol) and the mixture was stirred at 25° C. for 2 hrs. LC-MS showed reaction was completed and one main peak with desired mass was detected. The reaction mixture was adjusted pH˜9-10 with Sat. NaHCO₃ aq., then extracted with EtOAc (5 L). The combined organic layers were dried over Na₂SO₄, filtered and concentrated under reduced pressure to give crude. The crude was precipitated by PE (10 Vol) to give compound 6 (190 g, 74.4% yield).

¹H NMR: (400 MHZ, CDCl₃): δ 3.25 (br s, 1H) 3.85 (s, 3H) 4.64-4.83 (m, 2H) 5.30 (dd, J=9.51, 1.13 Hz, 1H) 5.92 (br d, J=9.38 Hz, 1H) 7.30-7.45 (m, 5H).

LCMS (m/z): 391.9/393.9 (M+H)⁺.

### General Procedure for Preparation of Compound 7

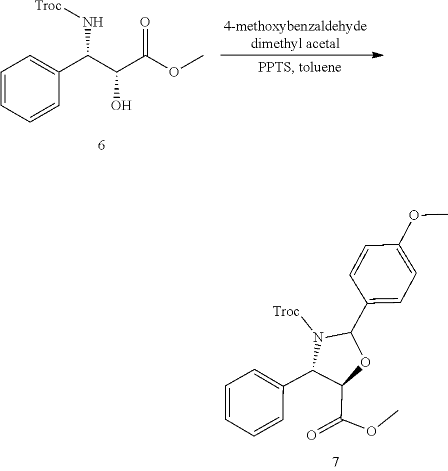

To a solution of compound 6 (185 g, 499 mmol) in toluene (1.9 L) was added 4-methylbenzenesulfonic acid pyridine (3.90 g, 15.4 mmol) and 4-methoxybenzaldehyde dimethyl acetal (121 g, 666 mmol). The mixture was stirred at 110° C. for 4 hrs. LC-MS showed one main peak with desired mass was detected. Then reaction mixture was allowed to cool to 25° C., The reaction mixture was concentrated under reduced pressure to remove toluene. The residue was diluted with H₂O (500 mL), then extracted with EtOAc (500 mL). The combined organic layers were dried over Na₂SO₄, filtered and concentrated under reduced pressure to give compound 7 (285 g, crude) which was carried forward as is.

### General Procedure for Preparation of Compound 8

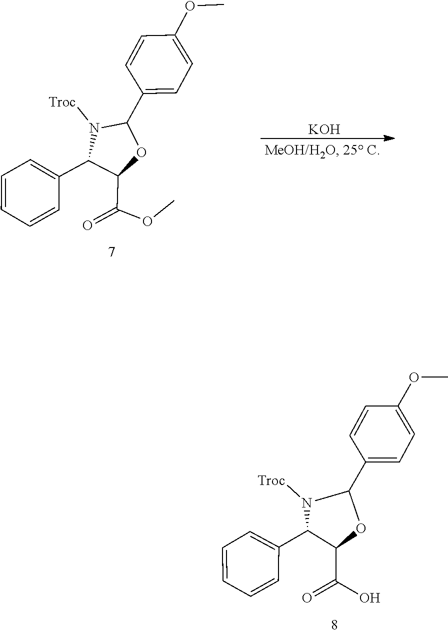

To a solution of compound 7 (285 g, crude) in MeOH (2000 mL) was added KOH (42.5 g, 758 mmol) in H₂O (1000 mL). The mixture was stirred at 25° C. for 1 hrs. LC-MS showed compound 7 was consumed completely and one main peak with desired mass was detected. The reaction mixture was concentrated under reduced pressure to remove MeOH. The residue was extracted with MTBE (5 L). The aqueous phase layers were diluted with sat. KHSO₄ (1L) aq. extracted with EtOAc (5 L), the combined organic layers were dried over Na₂SO₄, filtered and concentrated under reduced pressure to give crude. The crude was precipitated by PE (10 Vol) to give compound 8 (95.0 g, 34.3% yield).

¹H NMR (400 MHZ, MeOD): δ 3.82 (s, 3H) 4.41-4.47 (m, 1H) 4.50-4.56 (m, 1H) 4.60 (d, J=4.88 Hz, 1H) 5.47 (d, J=4.75 Hz, 1H) 6.46 (s, 1H) 6.86-6.94 (m, 2H) 7.34-7.46 (m, 7H).

LCMS (m/z): 495.9 (M+Na)⁺.

### General Procedure for Preparation of 7-Troc-Baccatin II

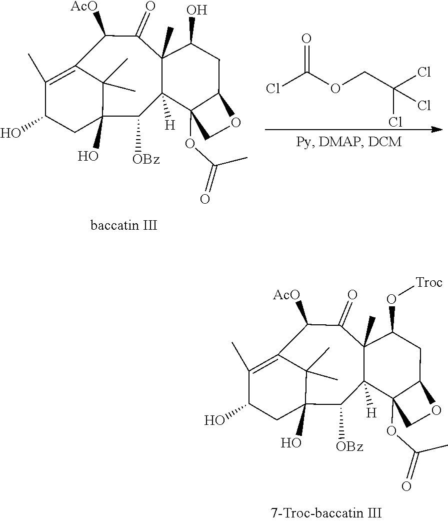

To a solution of baccatin III (30.0 g, 51.1 mmol) in DCM (300 mL) was added DMAP (625 mg, 5.11 mmol) and pyridine (14.2 g, 179 mmol) and 2,2,2-trichloroethyl carbonochloridate (15.2 g, 71.6 mmol). The mixture was stirred at 25° C. for 0.5 hrs. LC-MS showed baccatin III was consumed completely and one main peak with desired mass was detected. The residue was diluted with water (300 mL) and extracted with DCM (300 mL) and washed with water (200 mL) and brine (200 mL), dried over Na₂SO₄, filtered and concentrated under reduced pressure to give 7-Troc-baccatin III (45.0 g, 34.3% yield).

LCMS (m/z): 761.5/763.5 (M+Na)⁺.

### General Procedure for Preparation of Compound 9

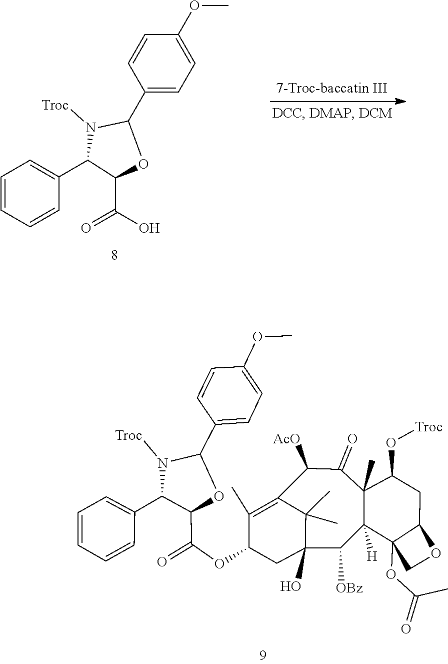

To a solution of 7-Troc-baccatin III (26.0 g, 34.1 mmol) and compound 8 (32.4 g, 68.2 mmol) in DCM (1000 mL) was added DMAP (4.20 g, 34.1 mmol) and DCC (21.1 g, 102 mmol). The mixture was stirred at 0° C. for 1 hrs. LC-MS showed compound 8 was consumed completely and one main peak with desired mass was detected. The reaction mixture filtered. The crude was washed by sat. NH₄Cl aq. (100 mL) and water (1000 mL) dried over Na₂SO₄, filtered and concentrated under reduced pressure to give Compound 9 (35.0 g, crude).

LCMS (m/z): 1240.0/1242.0 (M+Na)⁺.

### General Procedure for Preparation of Compound 10

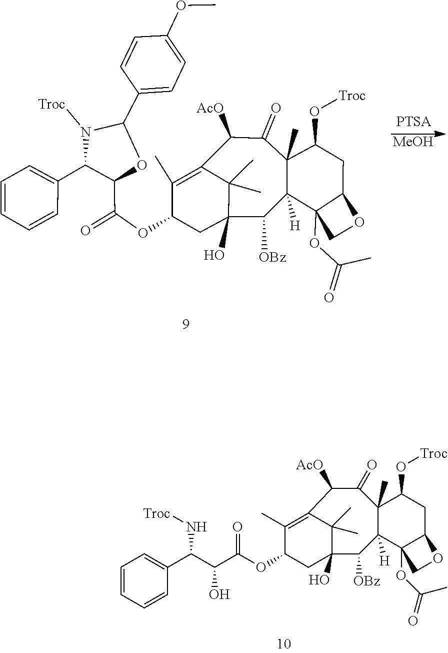

To a solution of compound 9 (80.0 g, 65.6 mmol) in MeOH (350 mL) was added 4-methylbenzenesulfonic acid; hydrate (24.9 g, 131 mmol). The mixture was stirred at 25° C. for 16 hrs. LC-MS showed ˜50% compound 9 was remained and one main peak with desired mass was detected. The reaction mixture filtered, concentrated and the residue was purified by prep-HPLC (Water (0.1% TFA)-ACN). The elution was concentrated under reduced pressure to remove solvent, then, extracted with EtOAc (500 mL). The combined organic layers were dried over Na₂SO₄, filtered and concentrated under reduced pressure to give compound 10 (13.0 g, 17.9% yield).

LCMS (m/z): 1120.2 (M+Na)⁺.

### General Procedure for Preparation of Compound 11

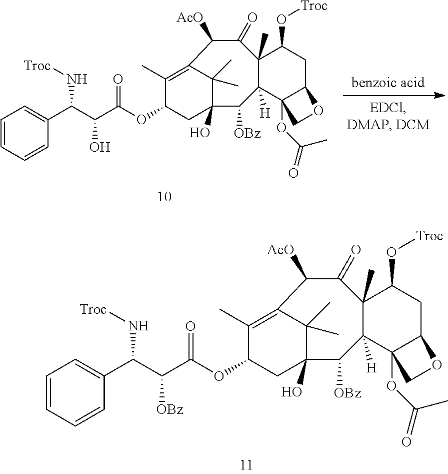

To a solution of compound 10 (13.0 g, 11.8 mmol) and DMAP (722 mg, 5.90 mmol) and EDCI (2.70 g, 14.2 mmol) and benzoic acid (1.70 g, 14.2 mmol) in DCM (260 mL). The mixture was stirred at 25° C. for 1 hrs. LC-MS showed compound 10 was consumed completely and one main peak with desired mass was detected. The reaction mixture was washed with sat. citric acid aq. (100 mL), sat. NaHCO₃ aq. (100 mL) and water (200 mL), dried over NaSO₄, filtered and concentrated under reduced pressure to give compound 11 (11.0 g, 77.3% yield).

LCMS (m/z): 1204.1 (M+H)⁺.

### General Procedure for Preparation of Compound 12

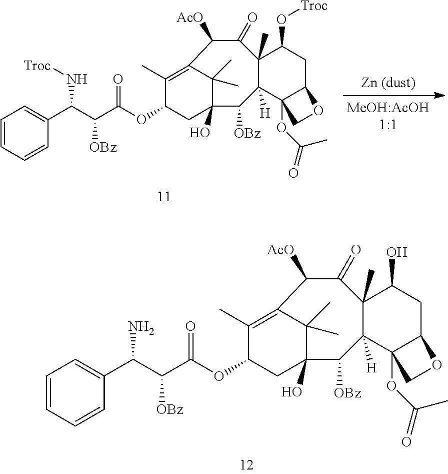

To a solution of compound 11 (20.0 g, 16.6 mmol) in MeOH (200 mL) and AcOH (200 mL) was added Zn dust (21.6 g, 331 mmol). The mixture was stirred at 25° C. for 1 hrs. LC-MS showed compound 11 was consumed completely and one main peak with desired mass was detected. The reaction mixture was filtered and diluted with H₂O (500 mL), then extracted with EtOAc (100 mL*3). The combined organic layers were washed with sat. NaHCO₃ aq. (200 mL) and brine (100 mL), dried over Na₂SO₄, filtered and concentrated under reduced pressure to give a crude product. The residue was purified by prep-HPLC (Water (0.1% TFA)-ACN) to give Compound 12 (5.0 g, 21% yield).

LCMS (m/z): 854.3 (M+H)⁺.

### General Procedure for Preparation of Compound 13

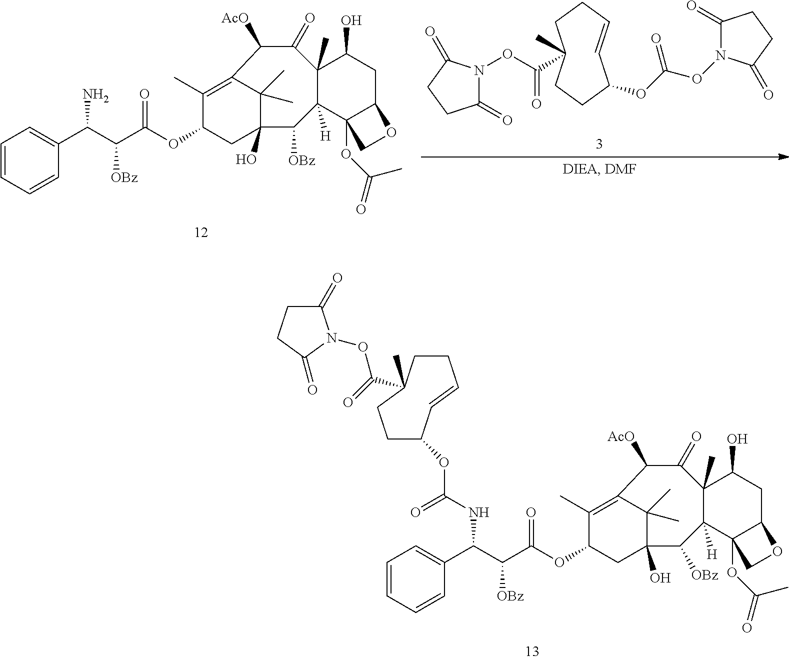

To a solution of compound 12 (5.00 g, 5.90 mmol), DIEA (1.50 g, 11.7 mmol) and compound 3 (3.90 g, 8.80 mmol) in DMF (50 mL). The mixture was stirred at 25° C. for 16 hrs. LC-MS showed ˜50% compound 12 was remained and one main peak with desired mass was detected. The residue was purified by prep-HPLC (Water (0.1% TFA)-ACN) to give compound 13 (505 mg, 7.4% yield).

LCMS (m/z): 1161.4 (M+H)⁺.

### General Procedure for Preparation of Compound D

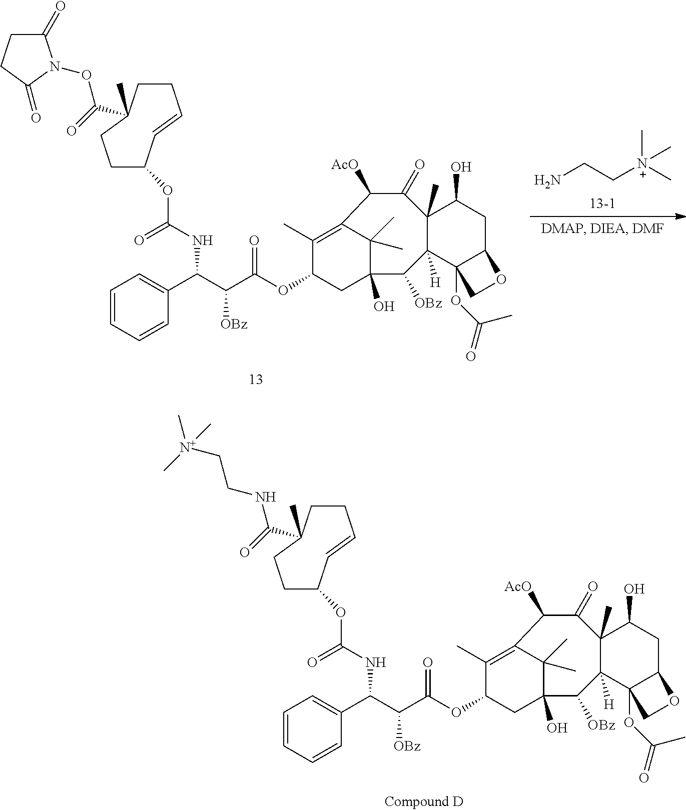

To a solution of compound 13 (150 mg, 0.13 mmol) in DMF (1.50 mL) was added DMAP (94.7 mg, 0.78 mmol) and compound 13-1 (66.7 mg, 0.65 mmol) and DIEA (100 mg, 0.78 mmol). The mixture was stirred at 25° C. for 16 hrs. LC-MS showed compound 13 was consumed completely and one main peak with desired mass was detected. The residue was purified by prep-HPLC (Water (0.1% TFA)-ACN) to give Compound D (75.0 mg, 50.5% yield).

LCMS (m/z): 1148.5 (M)⁺.

### Example 12: General Procedure for Preparation of Compound E

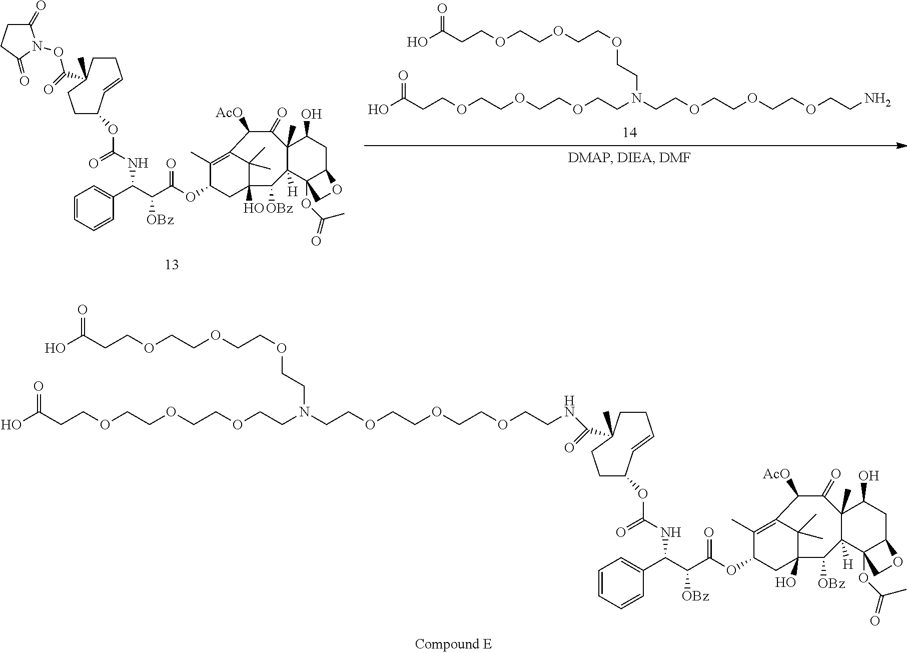

To a solution of compound 13 (350 mg, 0.30 μmol) and DMAP (221 mg, 1.81 mmol) and compound 14 (249 mg, 0.39 mmol, HCl) in DMF (0.3 mL). The mixture was stirred at 25° C. for 16 hrs. LC-MS showed compound 13 was consumed completely and one main peak with desired mass was detected. The residue was purified by prep-HPLC (Water (0.1% TFA)-ACN) to give Compound E (205 mg, 41.3% yield).

LCMS (m/z): 1646.5 (M+H)⁺.

### Example 13:3-(5-aminomethyl-pyrimidine)-6-methyl-1,2,4,5-tetrazine

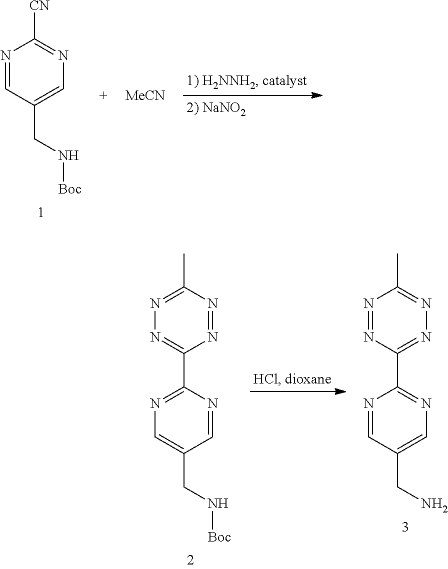 [SMILES: "MeCN" ; CC(C)(C)OC(=O)NCc1cnc(C#N)nc1 ; Cc1nnc(-c2ncc(CNC(=O)OC(C)(C)C)cn2)nn1 ; Cc1nnc(-c2ncc(CN)cn2)nn1]

N-Boc-3-(5-aminomethyl-pyrimidine)-6-methyl-1,2,4,5-tetrazine (2). To a solution of N-Boc-2-cyano-5-aminomethyl-pyrimidine (1) in dry acetonitrile is added hydrazine and nickel (II) triflate. The reaction mixture is then heated overnight; the starting material is consumed by TLC. To the reaction mixture is added sodium nitrite (dissolved in water), followed by 1 M hydrochloric acid. The reaction mixture is then stirred at ambient temperature until the reaction is determined to be complete by HPLC. The reaction mixture is then partitioned between ethyl acetate and water. The organic layer is washed with water (3×), followed by brine (1×), and then is dried over sodium sulfate. The solution is filtered and the filtrate is concentrated under reduced pressure to yield the product, which can be carried forward without further purification.

3-(5-aminomethyl-pyrimidine)-6-methyl-1,2,4,5-tetrazine (3). To a solution 2 in dioxane is added hydrochloric acid (4 M in dioxane). The reaction mixture is then stirred at ambient temperature until the starting material is consumed. The product is isolated by filtration and the precipitate is washed with diethyl ether to yield the product, optionally as the HCl salt.

6-(6-Methyl-1,2,4,5-tetrazin-3-yl)-3-pyridinemethanamine can also be utilized in the compounds and methods described herein, which compound can be prepared according to the art or purchased from a commercial source (e.g., Enamine US Inc., New Jersey, USA).

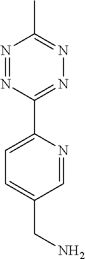

### Example 14: Val-Cit-PABC-dihydrotetrazine

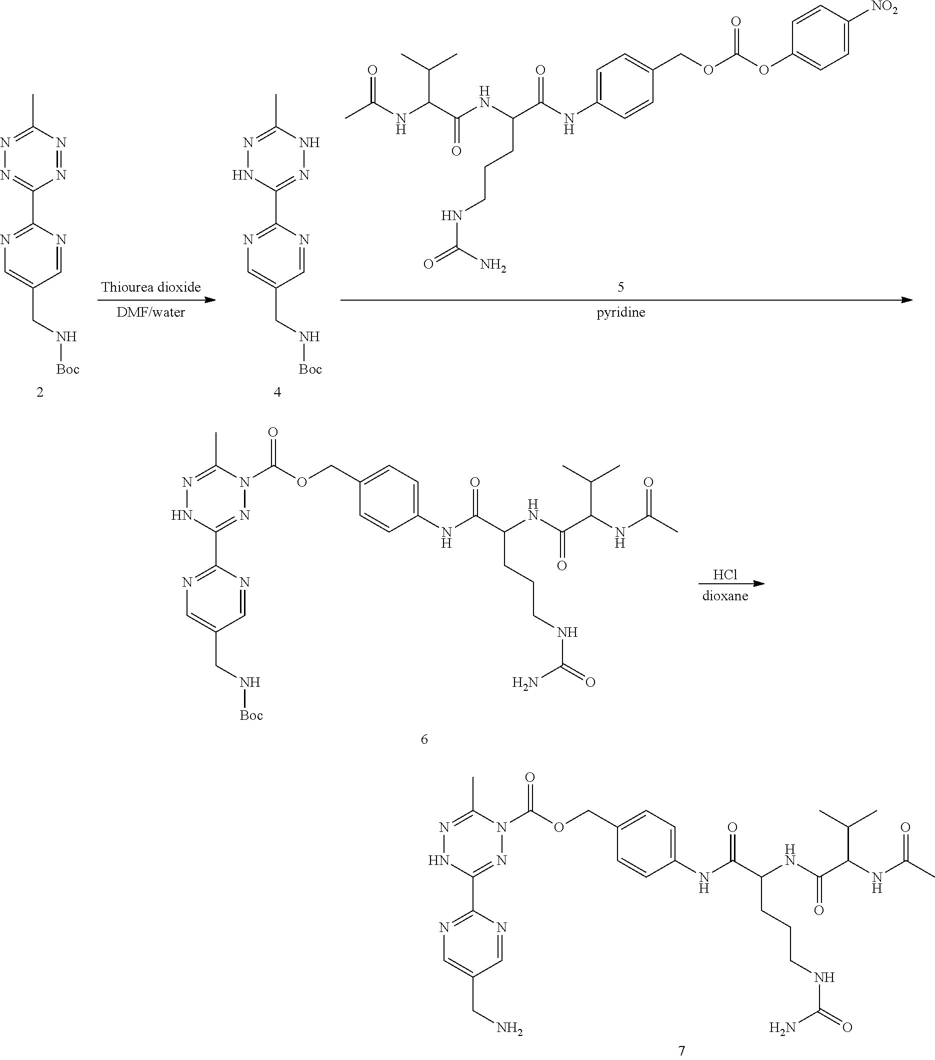 [SMILES: Cc1nnc(-c2ncc(CNC(=O)OC(C)(C)C)cn2)nn1 ; CC1=NNC(c2ncc(CNC(=O)OC(C)(C)C)cn2)=NN1 ; CC(=O)NC(C(=O)NC(CCCNC(N)=O)C(=O)Nc1ccc(COC(=O)Oc2ccc([N+](=O)[O-])cc2)cc1)C(C)C ; CC(=O)NC(C(=O)NC(CCCNC(N)=O)C(=O)Nc1ccc(COC(=O)N2N=C(c3ncc(CNC(=O)OC(C)(C)C)cn3)NN=C2C)cc1)C(C)C ; Cl ; CC(=O)NC(C(=O)NC(CCCNC(N)=O)C(=O)Nc1ccc(COC(=O)N2N=C(c3ncc(CN)cn3)NN=C2C)cc1)C(C)C]

N-Boc-3-(5-aminomethyl-pyrimidine)-6-methyl-1,2,4,5-dihydrotetrazine (4). In a sealed flask, thiourea dioxide is added to a solution of tetrazine 2 in DMF/H₂O (v/v=10/1) at ambient temperature. The reaction mixture is then heated with stirring until the solution color changes from pink to colorless. The reaction mixture is concentrated under reduced pressure and the resulting residue is dried under vacuum, providing dihydrotetrazine 4, which can be used directly without further purification.

To a solution of dihydrotetrazine 4 is added a solution of nitrophenyl carbonate 5 in toluene. The reaction mixture is then stirred at ambient temperature. Upon completion, the reaction mixture is concentrated under reduced pressure and the resulting residue is purified by flash chromatography to yield compound 6.

1-(N-Acyl-(Val-Cit)-PABC)-3-(5-aminomethyl-pyrimidine)-6-methyl-1,2,4,5-dihydrotetrazine (7). To a solution 6 in dioxane is added hydrochloric acid (4 M in dioxane). The reaction mixture is then stirred at ambient temperature until the starting material is consumed. The product is isolated by filtration and the precipitate is washed with diethyl ether to yield the product, optionally as the HCl salt.

### Example 15: Dihydrotetrazine (Target 5)

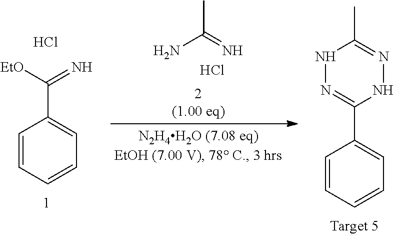

To a solution of N₂H₄·H₂O (9.74 g, 190 mmol, 9.44 mL, 98% purity, 7.08 eq) in EtOH (35.0 mL) was added compound 1 (5.00 g, 26.9 mmol, 1.00 eq, HCl) and compound 2 (2.55 g, 26.9 mmol, 1.00 eq, HCl) at 20° C. The mixture was stirred at 78° C. for 3 hrs. LCMS analysis of the reaction mixture showed compound 1 was consumed completely. To the reaction mixture was added H₂O (100 mL) at 20° C. and then the resulting solution was concentrated under reduced pressure at 40° C. to remove EtOH. The reaction mixture was extracted with ethyl acetate 150 mL (50.0 mL×3) and the combined organic layers were washed with brine 20.0 mL (20.0 mL×1), dried over Na₂SO₄, filtered and concentrated under reduced pressure to give a residue. The residue was purified by column chromatography (SiO₂, n-heptane/Ethyl acetate=100/1 to 1/1) and then further purified by prep-HPLC(HCl condition) to give Target 5 (100 mg, 2.87 mmol) as a white solid.

¹H NMR (400 MHZ, METHANOL-d₄) δ ppm 7.94-8.13 (m, 2H), 7.53-7.75 (m, 3H), 2.64 (s, 3H).

LCMS: M+H⁺=175.1

### Example 16:3-methyl-6-(1-methyl-1H-imidazol-4-yl)-1,2,4,5-tetrazine (Target 4)

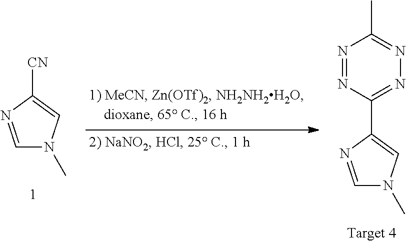

3-methyl-6-(1-methyl-1H-imidazol-4-yl)-1,2,4,5-tetrazine (Target 4): To a solution of 1-methylimidazole-4-carbonitrile (200 mg, 1.87 mmol), MeCN (268 mg, 6.54 mmol) and zinc; trifluoromethanesulfonate (68 mg, 0.19 mmol) in dioxane (1 mL) was added NH₂NH₂·H₂O (2.34 g, 46.68 mmol) at 25° C. and the mixture was stirred at 65° C. for 16 h under N₂. Then the mixture was cooled to 25° C. and added with a solution of NaNO₂ (387 mg, 5.60 mmol) in H₂O (3 mL) dropwise at 25° C. The mixture was stirred at 25° C. for 3 h. The mixture was cooled to room temperature and adjusted to pH=3 with 1 M aqueous hydrochloric acid. The aqueous phase was extracted with DCM (3×5 mL). The combined organics were dried over anhydrous sodium sulfate, filtered and concentrated under reduced pressure. The residue was purified by prep-TLC(SiO₂, DCM: MeOH=10:1) to give 3-methyl-6-(1-methyl-1H-imidazol-4-yl)-1,2,4,5-tetrazine (Target 4) (20.1 mg, 6.1%).

LCMS(ESI+): m/z=177.2 [M+H]⁺

¹H NMR (400 MHZ, CDCl₃) (ET60578-12-P1M): δ=8.00 (s, 1H), 7.68 (s, 1H), 3.85 (s, 3H), 3.06 (s, 3H).

### Example 17: 3-(6-methyl-1,2,4,5-tetrazin-3-yl) isoxazole (Target 8)

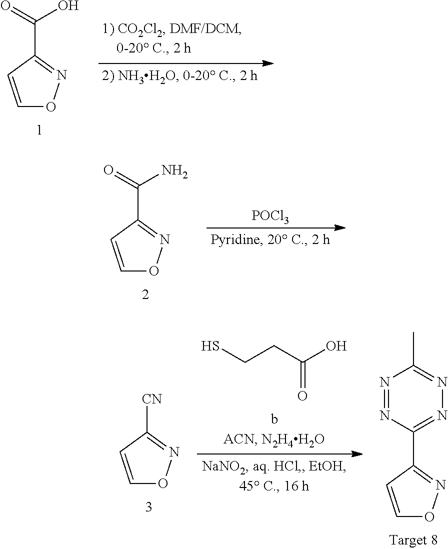

isoxazole-3-carboxamide (2): To a solution of isoxazole-3-carboxylic acid (10 g, 88.44 mmol) in DMF (646 mg, 8.84 mmol) and DCM (100 mL) was added oxalyl dichloride (13.47 g, 106.13 mmol) at 0° C. under N₂. The mixture was stirred at 20° C. for 2 h. The reaction mixture was concentrated under reduced pressure. The residue was dissolved THF (50 ml) and adjusted to pH=9 with NH₃·H₂O at 0° C. The mixture was stirred at 20° C. for 2 h. The reaction mixture was concentrated under reduced pressure to give isoxazole-3-carboxamide (2) (3.3 g, 33.3%).

¹H NMR (400 MHZ, DMSO): δ=9.05 (d, J=1.6 Hz, 1H), 8.12 (s, 1H), 7.82 (s, 1H), 6.85 (d, J=1.6 Hz, 1H)

isoxazole-3-carbonitrile (3): To a solution of isoxazole-3-carboxamide (500 mg, 4.46 mmol) in pyridine (18 mL) was added POCl₃ (1.03 g, 6.69 mmol) at 20° C. under N₂. The mixture was stirred at 20° C. for 2 h. After stirring for 2 h, the mixture was cooled by an ice bath and added with water (10 mL). The aqueous phase was adjusted to pH=4 by addition of aqueous 3 M HCl and extracted with MTBE (3×10 mL). The combined organics were washed with brine (20 mL), dried over Na₂SO₄, filtered and concentrated under reduced pressure to give isoxazole-3-carbonitrile (3) (280 mg, 66.7%).

¹H NMR (400 MHZ, MeOD): δ 9.06 (d, J=1.6 Hz, 1H), 7.02 (d, J=1.2 Hz, 1H)

3-(6-methyl-1,2,4,5-tetrazin-3-yl) isoxazole (Target 8): To a solution of isoxazole-3-carbonitrile (220 mg, 2.34 mmol) in EtOH (2 mL) was added NH₂NH₂·H₂O (1.87 g, 37.42 mmol) at 20° C. for 0.5 h. Then the mixture was added MeCN (384 mg, 9.35 mmol) and 3-sulfanylpropanoic acid (248 mg, 2.34 mmol) at 20° C. under N₂. The mixture was stirred at 45° C. for 12 h. Thereafter, NaNO₂ (500 mg) was added and the reaction mixture was stirred at 20° C. for 0.5 h. 3 M hydrochloric acid was added dropwise until pH=1, and the reaction mixture was stirred at 20° C. for 0.5 h. The aqueous phase was extracted with EtOAc (3×10 mL). The combined organic phase was washed with brine (20 mL), dried over anhydrous Na₂SO₄, filtered and concentrated under reduced pressure. The crude product was purified by prep-HPLC (FA) with the following conditions: column: Phenomenex Luna C18 100*30 mm*5 um; mobile phase: [water (FA)-ACN]; B %: 5%-45%, 8 min to give 3-(6-methyl-1,2,4,5-tetrazin-3-yl) isoxazole (12.45 mg, 3.26%). LCMS(ESI+): m/z=164.1 [M+H]⁺

1H NMR (400 MHZ, MeOD): δ 9.01 (d, J=1.6 Hz, 1H), 7.34 (d, J=1.6 Hz, 1H), 3.11 (s, 3H).

### Example 18: 3-methyl-6-(1H-pyrazol-1-yl)-1,2,4,5-tetrazine (Target 13)

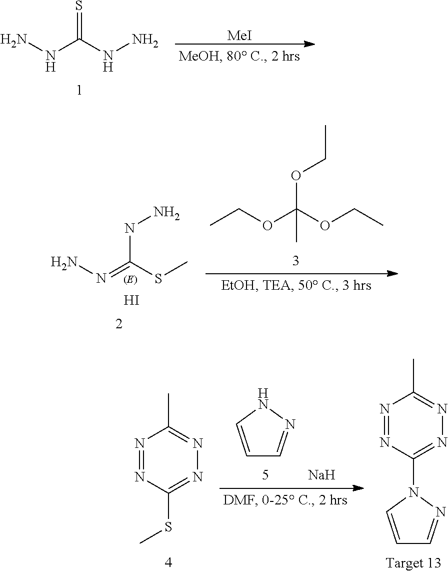

methyl (E)-hydrazinecarbohydrazonothioate (2): To a mixture of 1,3-diaminothiourea (100 g, 942.06 mmol) in MeOH (500 mL) was added Mel (160.46 g, 1.13 mol) at 25° C. and the mixture was stirred at 80° C. for 1.5 h under N₂. H NMR showed the reaction was completed. The resulting pale yellow solution was cooled to room temperature until solid precipitated. And then it was diluted with MTBE (500 mL). The mixture was cooled in ice for 2 h and filtered. The collected solid was washed with MTBE, dried under reduced pressure to give 1,3-diamino-2-methyl-isothiourea; hydroiodide (Compound 2) (132 g, 56.48% yield, HI).

¹H NMR (400 MHZ, DMSO): δ=10.96-9.08 (m, 1H), 5.80-4.81 (m, 2H), 2.37 (s, 3H)

3-methyl-6-(methylthio)-1,2,4,5-tetrazine (4): To a solution of 1,3-diamino-2-methyl-isothiourea (30 g, 249.63 mmol) in DMF (750 mL) was added 1,1,1-triethoxyethane (44.55 g, 274.60 mmol) at 25° C. and the mixture was stirred at 25° C. for 5 min under N₂. Then the mixture was added TEA (25.26 g, 249.63 mmol) at 25° C. and the mixture was stirred at 50° C. for 3 h. TLC showed the reaction was completed. The mixture was poured into water (1000 mL) and extracted with EtOAc (3×500 mL). The combined organic layers were washed with brine (500 mL), dried over Na₂SO₄, filtered and concentrated under reduced pressure. The residue was purified by silica gel column chromatography (Petroleum ether/Ethyl acetate=10/1 to 5/1) to give 3-methyl-6-methylsulfanyl-1,2,4,5-tetrazine (Compound 4) (5.8 g, 16.3%).

¹H NMR (400 MHZ, CDCl₃): δ=2.99 (s, 3H), 2.74 (s, 3H).

3-methyl-6-(1H-pyrazol-1-yl)-1,2,4,5-tetrazine (Target 13): To a mixture of 1H-pyrazole (144 mg, 2.11 mmol) in THF (2 mL) was added NaH (84 mg, 2.11 mmol, 60% purity) at 0° C. and the mixture was stirred at 0° C. for 15 min under N₂. Then the mixture was added 3-methyl-6-methylsulfanyl-1,2,4,5-tetrazine (200 mg, 1.41 mmol) in THF (1 mL) at 0° C. and the mixture was stirred at 25° C. for 2 h under N₂. TLC showed the reaction was completed. The mixture was poured into sat·NH₄Cl (5 mL) and extracted with EtOAc (3×3 mL). The combined organic layers were washed with brine (3 mL), dried over Na₂SO₄, filtered and concentrated under reduced pressure. The residue was purified by prep-TLC (SiO₂, PE: EtOAc=3:1) to give 3-methyl-6-pyrazol-1-yl-1,2,4,5-tetrazine (Target 13) (20 mg, 8.8%). LCMS(ESI+): m/z=163.1 [M+H]⁺

¹H NMR (400 MHZ, CDCl₃): δ=8.74 (d, J=2.8 Hz, 1H), 8.02 (d, J=1.2 Hz, 1H), 6.68 (dd, J=1.6, 2.8 Hz, 1H), 3.14 (s, 3H).

### Example 19: (4-(6-methyl-1,2,4,5-tetrazin-3-yl)-2-(trifluoromethyl)phenyl) methanamine (Target 15)

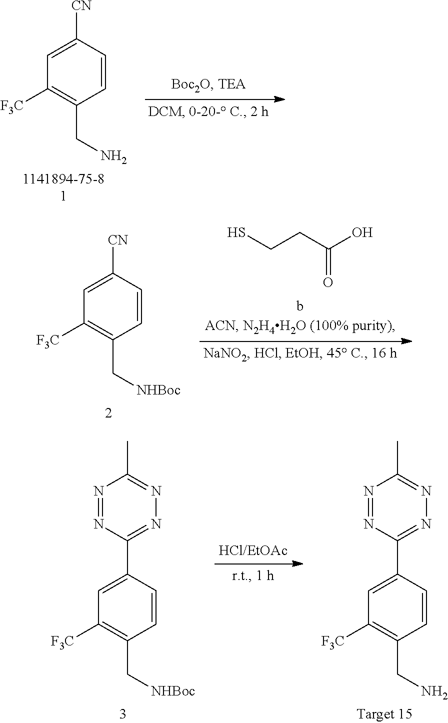 [SMILES: N#Cc1ccc(CN)c(C(F)(F)F)c1 ; CC(C)(C)OC(=O)NCc1ccc(C#N)cc1C(F)(F)F ; O=C(O)CCS ; Cc1nnc(-c2ccc(CNC(=O)OC(C)(C)C)c(C(F)(F)F)c2)nn1 ; Cc1nnc(-c2ccc(CN)c(C(F)(F)F)c2)nn1]

tert-butyl (4-cyano-2-(trifluoromethyl)benzyl) carbamate (2): To a solution of 4-(aminomethyl)-3-(trifluoromethyl)benzonitrile (300 mg, 1.50 mmol) in DCM (10 mL) was added Boc₂O (360 mg, 1.65 mmol) and TEA (227 mg, 2.25 mmol) at 20° C. under N₂. The mixture was stirred at 20° C. for 2 h. The mixture was cooled down and poured into H₂O (10 mL), the aqueous phase was extracted with DCM (3×10 mL). The combined organic phase was washed with brine (10 mL), dried over anhydrous Na₂SO₄, filtered and concentrated under reduced pressure. The crude product was purified by flash silica gel column chromatography (PE: EtOAc=1:0 to 3:1) to give the tert-butyl (4-cyano-2-(trifluoromethyl)benzyl) carbamate (2) (400 mg, 88.9%).

¹H NMR (400 MHZ, CDCl₃): δ 7.94 (s, 1H), 7.87-7.81 (m, 1H), 7.79-7.73 (m, 1H), 5.00 (s, 1H), 4.56 (d, J=6.0 Hz, 2H), 1.47 (s, 9H).

tert-butyl (4-(6-methyl-1,2,4,5-tetrazin-3-yl)-2-(trifluoromethyl)benzyl) carbamate (3): To a solution of tert-butyl N-[[4-cyano-2-(trifluoromethyl)phenyl]methyl]carbamate (150 mg, 0.50 mmol) in EtOH (0.5 mL) was added NH₂NH₂·H₂O (400 mg, 7.99 mmol) at 20° C. for 0.5 h. Then the mixture was added with MeCN (82 mg, 2.00 mmol) and 3-sulfanylpropanoic acid (53 mg, 0.50 mmol) at 20° C. under N₂. Thereafter, NaNO₂ (500 mg) was added and the reaction mixture was stirred at 20° C. for 0.5 h. 3 M hydrochloric acid was added until pH=1, and the reaction mixture was stirred at 20° C. for 0.5 h. The aqueous phase was extracted with EtOAc (3×10 mL). The combined organic phase was washed with brine (10 mL), dried over anhydrous Na₂SO₄, filtered and concentrated under reduced pressure. The crude product was purified by prep-TLC(SiO₂, PE: EtOAc=3:1) to give tert-butyl N-((4-(6-methyl-1,2,4,5-tetrazin-3-yl)-2-(trifluoromethyl)phenyl)methyl) carbamate (3) (50 mg, 14.1%).

¹H NMR (400 MHZ, CDCl₃): δ 8.91 (s, 1H), 8.78 (d, J=8.4 Hz, 1H), 7.84 (d, J=8.4 Hz, 1H), 5.11-4.96 (m, 1H), 4.63 (d, J=6.8 Hz, 2H), 3.14 (s, 3H), 1.49 (s, 9H).

(4-(6-methyl-1,2,4,5-tetrazin-3-yl)-2-(trifluoromethyl)phenyl) methanamine (Target 15): To a solution of tert-butyl N-((4-(6-methyl-1,2,4,5-tetrazin-3-yl)-2-(trifluoromethyl)phenyl)methyl) carbamate (50 mg, 0.14 mmol) in MeOH (2 mL) was added HCl/MeOH (2 mL, 4 M) at 20° C. under N₂. The mixture was stirred at 20° C. for 2 h. The reaction mixture was concentrated under reduced pressure. The crude product was purified by prep-HPLC (FA) with the following conditions: Phenomenex Luna 80*30 mm*3 um phase: [water (FA)-ACN]; B %: 1%-25%, 8 min to give (4-(6-methyl-1,2,4,5-tetrazin-3-yl)-2-(trifluoromethyl)phenyl) methanamine (4.8 mg, 15.9%).

LCMS(ESI+): m/z=270.0 [M+H]⁺

¹H NMR (400 MHZ, MeOD): δ 8.93 (s, 1H), 8.89 (d, J=8.4 Hz, 1H), 8.49 (s, 1H), 7.96 (d, J=8.0 Hz, 1H), 4.37 (s, 2H), 3.09 (s, 3H).

### Example 20: (4-(6-methyl-1,2,4,5-tetrazin-3-yl)-2-(trifluoromethyl)phenyl)methanamine (Target 17)

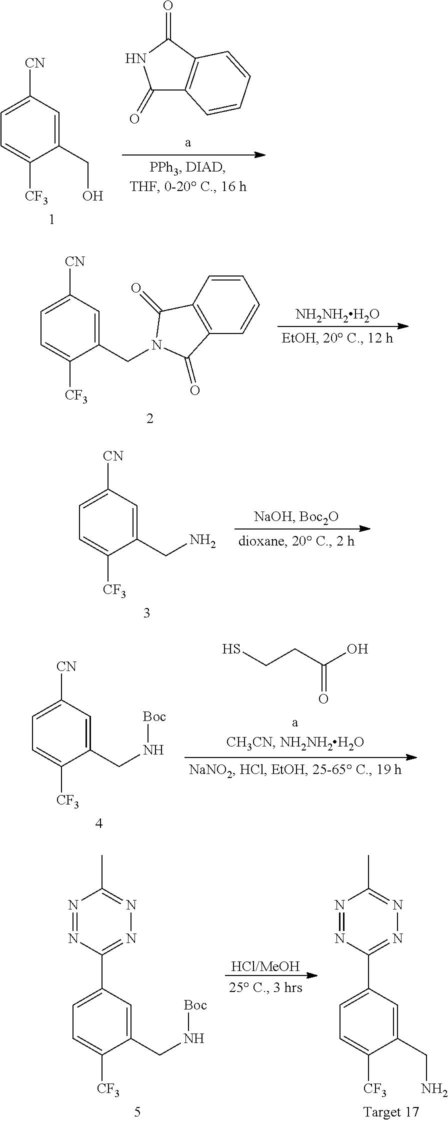

3-((1,3-dioxoisoindolin-2-yl)methyl)-4-(trifluoromethyl)benzonitrile (2): To a solution of 3-(hydroxymethyl)-4-(trifluoromethyl)benzonitrile (4.5 g, 22.38 mmol) in THF (50 mL) was added isoindoline-1,3-dione (3.3 g, 22.38 mmol), PPh₃ (11.7 g, 44.76 mmol) and DIAD (6.79 g, 33.57 mmol) at 0° C. under N₂. Then the resulting mixture was stirred at 20° C. for 16 h. The reaction mixture was quenched by addition of water (15 mL) and extracted with EtOAc (3×10 mL). The combined organic phase was washed with brine (15 mL), dried over sodium sulfate, filtered and the filtrate was concentrated under reduced pressure to give a residue, which was purified by column to give 3-((1,3-dioxoisoindolin-2-yl)methyl)-4-(trifluoromethyl)benzonitrile (2) (5.1 g, 69%).

¹H NMR (400 MHZ, CDCl₃) δ=7.91-8.03 (m, 2H), 7.84 (dd, J=5.6, 2.8 Hz, 3H), 7.70 (d, J=8.0 Hz, 1H), 7.46 (s, 1H), 5.13 (s, 2H).

3-(aminomethyl)-4-(trifluoromethyl)benzonitrile (3): To a solution of 3-((1,3-dioxoisoindolin-2-yl)methyl)-4-(trifluoromethyl)benzonitrile (1 g, 3.03 mmol) in EtOH (20 mL) was added NH₂NH₂·H₂O (3.79 g, 60.56 mmol, 80% purity) at 20° C., then the mixture was stirred at 20° C. for 12 h. 6 M hydrochloric acid was added until pH=1, and the reaction mixture was stirred at 20° C. for 2 h. After the completion of the reaction, the mixture was filtered and the filtrate was concentrated under reduced pressure. The residue was poured into water (50 mL), adjusting pH to 11 with 6 M NaOH aqueous solution (20 mL). Then the mixture was extracted with EtOAc (3×50 mL) and organic layer was washed with brine (200 mL), dried over Na₂SO₄, filtered and concentrated under reduced pressure to give 3-(aminomethyl)-4-(trifluoromethyl)benzonitrile (3) (600 mg, 99.0%).

¹H NMR (400 MHZ, CDCl₃): δ=8.09 (s, 1H), 7.72-7.80 (m, 1H), 7.62-7.71 (m, 1H), 4.12 ppm (s, 2H).

tert-butyl (5-cyano-2-(trifluoromethyl)benzyl) carbamate (4): To a solution of 3-(aminomethyl)-4-(trifluoromethyl)benzonitrile (0.3 g, 1.50 mmol) and NaOH (180 mg, 4.50 mmol) in dioxane (6 mL) and H₂O (3 mL) was added Boc₂O (654 mg, 3.00 mmol) at 20° C., then the mixture was stirred at 20° C. for 2 h. The reaction mixture was quenched with H₂O (10 mL) and extracted with EtOAc (3×10 mL). The combined organic layers were washed with brine (10 mL), dried over anhydrous Na₂SO₄, filtered and concentrated under reduced pressure to give a residue. The residue was purified by silica gel column chromatography (PE: EtOAc=1:0 to 0:1) to give tert-butyl (5-cyano-2-(trifluoromethyl)benzyl) carbamate (4) (300 mg, 66.6%).

¹H NMR (400 MHZ, CDCl₃): δ=7.89 (s, 1H), 7.75 (s, 1H), 7.65-7.71 (m, 1H), 4.88-5.19 (m, 1H), 4.54 (br d, J=6.0 Hz, 2H), 1.48 ppm (s, 9H).

tert-butyl (5-(6-methyl-1,2,4,5-tetrazin-3-yl)-2-(trifluoromethyl)benzyl) carbamate (5): To a solution of tert-butyl (5-cyano-2-(trifluoromethyl)benzyl) carbamate (500 mg, 1.67 mmol), MeCN (273 mg, 6.66 mmol) and 3-sulfanylpropanoic acid (177 mg, 1.67 mmol) in EtOH (3 mL) was added N₂H₄·H₂O (1.33 g, 26.64 mmol) at 25° C. and the mixture was stirred at 65° C. for 16 h under N₂. Then the mixture was cooled to r.t and added with a solution of NaNO₂ (345 mg, 5.00 mmol) in H₂O (2.5 mL) dropwise at 25° C. The mixture was stirred at 25° C. for 3 h. The mixture was cooled to room temperature and adjusted to pH=3 with 1 M aqueous hydrochloric acid. The mixture was extracted with DCM (20 mL). The organic phase was dried over anhydrous sodium sulfate and concentrated under reduced pressure. The residue was purified by silica gel column chromatography (PE: EtOAc=20:1 to 1:1) to give tert-butyl (5-(6-methyl-1,2,4,5-tetrazin-3-yl)-2-(trifluoromethyl)benzyl) carbamate (5) (140 mg, 22.8%).

¹H NMR (400 MHZ, CDCl₃): δ=8.78 (s, 1H), 8.60 (d, J=8.0 Hz, 1H), 7.88 (d, J=8.0 Hz, 1H), 5.08 (s, 1H), 4.67 (s, 1H), 3.15 (s, 3H), 1.49 (s, 9H).

(5-(6-methyl-1,2,4,5-tetrazin-3-yl)-2-(trifluoromethyl)phenyl) methanamine (Target 15): To a solution of tert-butyl N-[[5-(6-methyl-1,2,4,5-tetrazin-3-yl)-2-(trifluoromethyl)phenyl]methyl]carbamate (5) (140 mg, 0.38 mmol) in MeOH (2 mL) was added HCl/MeOH (2 mL, 4 M) at 20° C. under N₂. The mixture was stirred at 20° C. for 2 h. The reaction mixture was concentrated under reduced pressure. The crude product was purified by prep-TLC (SiO₂, DCM: MeOH=10:1) to give [5-(6-methyl-1,2,4,5-tetrazin-3-yl)-2-(trifluoromethyl)phenyl]methanamine (18.0 mg, 17.7%).

LCMS(ESI+): m/z=270.0 [M+H]⁺

¹H NMR (400 MHZ, MeOD): δ 8.88 (s, 1H), 8.60 (d, J=8.4 Hz, 1H), 7.94 (d, J=8.4 Hz, 1H), 4.11 (s, 2H), 3.08 (s, 3H)

### Example 21:1-(2-(6-(4-(aminomethyl)phenyl)-1,2,4,5-tetrazin-3-yl)ethyl)-3-methylurea (Target 6)

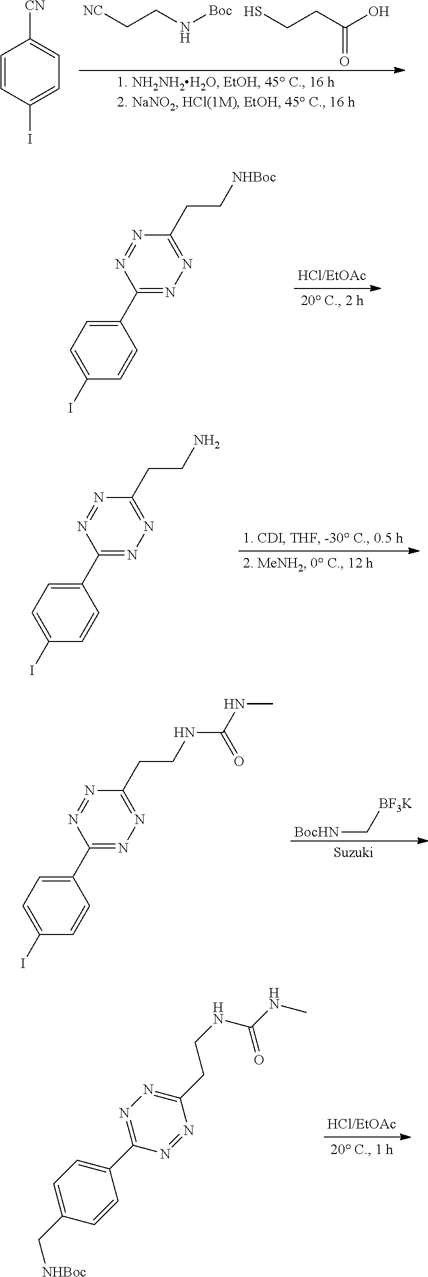

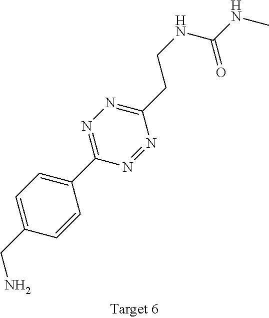 [SMILES: CNC(=O)NCCc1nnc(-c2ccc(CN)cc2)nn1]

tert-butyl (2-(6-(4-iodophenyl)-1,2,4,5-tetrazin-3-yl)ethyl) carbamate: To a mixture of 4-iodobenzonitrile (15 g, 65.50 mmol) in EtOH (140 mL) was added tert-butyl N-(2-cyanoethyl) carbamate (44.59 g, 261.99 mmol), 3-sulfanylpropanoic acid (6.95 g, 65.50 mmol) and NH₂NH₂·H₂O (59.02 g, 1.18 mol) at 0° C. under N₂. The mixture was stirred at 45° C. for 16 h. Then the mixture was cooled to 20° C. and added with a solution of NaNO₂ in H₂O (40 mL) dropwise at 20° C. The mixture was stirred at 20° C. for 1 h. Under ice-cooling, the pH was adjusted to 3 with 1 M aqueous hydrochloride and then extracted with DCM (3×50 mL). The organic phase was dried over anhydrous sodium sulfate, filtered and concentrated under reduced pressure. The residue was purified by silica gel column chromatography (PE: EtOAc=10:1 to 1:1) to give tert-butyl (2-(6-(4-iodophenyl)-1,2,4,5-tetrazin-3-yl)ethyl) carbamate (14 g, 50%).

LCMS: m/z=372.0 [M-BuOH+H]⁺.

2-(6-(4-iodophenyl)-1,2,4,5-tetrazin-3-yl) ethan-1-amine: A mixture of tert-butyl (2-(6-(4-iodophenyl)-1,2,4,5-tetrazin-3-yl)ethyl) carbamate (6.5 g, 5.85 mmol) in HCl/EtOAc (4M, 100 mL) was stirred at 25° C. for 1 h. The reaction was concentrated under reduced pressure to give 2-(6-(4-iodophenyl)-1,2,4,5-tetrazin-3-yl) ethan-1-amine; hydrochloride (5.8 g, 80%).

LCMS: m/z=328.2 [M+H]⁺.

1-(2-(6-(4-iodophenyl)-1,2,4,5-tetrazin-3-yl)ethyl)-3-methylurea: To a mixture of CDI (3.35 g, 20.63 mmol) in DCM (45 mL) was added TEA (4.18 g, 41.27 mmol) and 2-(6-(4-iodophenyl)-1,2,4,5-tetrazin-3-yl) ethan-1-amine (5.8 g, 13.76 mmol) at −40° C. under N₂. The mixture was stirred at −40° C. for 1 h. Then MeNH₂ (2 M, 17 mL) and TEA (4.17 g, 41.24 mmol) were added at 0° C. under N₂. The mixture was stirred at 25° C. for 12 h. The reaction was concentrated under reduced pressure to give a residue. The residue was triturated with DCM. The resulting solid was collected by filtration, washed with DCM (40 mL) and dried under reduced pressure to give 1-[2-[6-(4-iodophenyl)-1,2,4,5-tetrazin-3-yl]ethyl]-3-methyl-urea (3 g, 57%). LCMS: m/z=385.0 [M+H]⁺.

¹H NMR (400 MHZ, DMSO): δ=8.24 (d, J=8.4 Hz, 2H) 8.06 (d, J=8.4 Hz, 2H) 6.09 (br t, J=5.6 Hz, 1H) 5.77 (br d, J=4.8 Hz, 1H) 3.53-3.57 (m, 2H) 3.36-3.40 (m, 2H) 2.45 (d, J=4.4 Hz, 3 H).

tert-butyl (4-(6-(2-(3-methylureido)ethyl)-1,2,4,5-tetrazin-3-yl)benzyl) carbamate: To a mixture of 1-[2-[6-(4-iodophenyl)-1,2,4,5-tetrazin-3-yl]ethyl]-3-methyl-urea (1 g, 2.60 mmol) and (tert-butoxycarbonylamino) methyl-trifluoro-boron; potassium hydride (926 mg, 3.90 mmol) in 2-METHYL-2-BUTANOL (4 mL) and H₂O (1 mL) was added Cs₂CO₃ (1.70 g, 5.21 mmol) and ditert-butyl(cyclopentyl)phosphane; dichloropalladium; iron (170 mg, 0.26 mmol) at 25° C. under N₂. The mixture was stirred at 80° C. for 16 h. The mixture was diluted with H₂O (15 mL) and extracted with EtOAc (3×10 mL). The combined organics were dried over anhydrous sodium sulfate, filtered and concentrated under reduced pressure. The residue was purified by silica gel column chromatography (PE: EtOAc=5:1 to 0:1) to give tert-butyl (4-(6-(2-(3-methylureido)ethyl)-1,2,4,5-tetrazin-3-yl)benzyl) carbamate (200 mg, 20%).

LCMS: m/z=388.3 [M+H]⁺

¹H NMR (400 MHZ, DMSO): δ=8.44 (br d, J=8.0 Hz, 2H), 7.52 (br d, J=7.6 Hz, 3H), 6.08 (br t, J=5.6 Hz, 1H), 5.78 (br s, 1H), 4.26 (br d, J=5.6 Hz, 2H), 3.54-3.58 (m, 2H), 3.38 (br d, J=6.4 Hz, 2H), 2.45 (d, J=4.4 Hz, 3H), 1.41 (s, 9H).

1-(2-(6-(4-(aminomethyl)phenyl)-1,2,4,5-tetrazin-3-yl)ethyl)-3-methylurea (Target 6): To a mixture of tert-butyl (4-(6-(2-(3-methylureido)ethyl)-1,2,4,5-tetrazin-3-yl)benzyl) carbamate (150 mg, 0.39 mmol) was added HCl/EtOAc (4 M, 3 mL) at 25° C. under N₂. The mixture was stirred at 25° C. for 1 h. The reaction was concentrated under reduced pressure. The crude product was purified by prep-HPLC with the following conditions: column: Phenomenex C18 100×30 mm×5 um; mobile phase: A: 10 mM NH₄HCO₃ in water, B: MeCN; B % in A: 20%-50%, 10 min to give 1-(2-(6-(4-(aminomethyl)phenyl)-1,2,4,5-tetrazin-3-yl)ethyl)-3-methylurea (63 mg, 29%). LCMS: m/z=288.2 [M+H]⁺.

¹H NMR (400 MHZ, DMSO): δ=8.52 (d, J=8.0 Hz, 2H), 8.45 (br s, 2H), 7.77 (d, J=8.2 Hz, 2H), 6.14 (br t, J=5.6 Hz, 1H), 5.76-5.83 (m, 1H), 4.18 (br s, 2H), 3.56 (q, J=6.4 Hz, 2H), 3.38-3.41 (m, 2H), 2.44 (d, J=4.4 Hz, 3H).

### Example 22: 4-((S)-2-((S)-2-acetamido-3-methylbutanamido)-5-ureidopentanamido)benzyl-6-methyl-3-phenyl-1,2,4,5-tetrazine-1 (4H)-carboxylate (Target 1b)

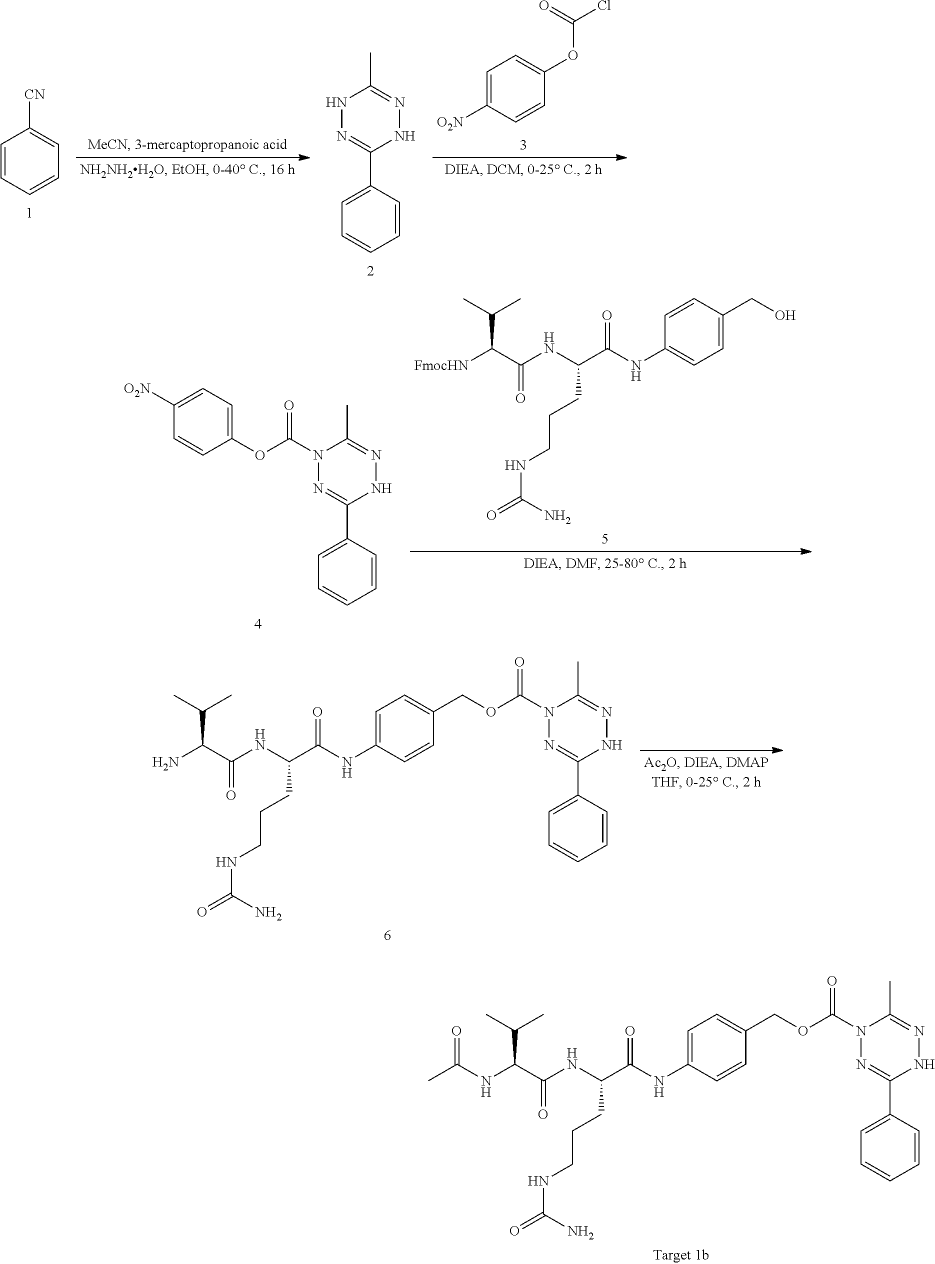

3-methyl-6-phenyl-1,4-dihydro-1,2,4,5-tetrazine (2): To a solution of benzonitrile (10 g, 96.97 mmol), ACN (31.85 g, 775.79 mmol), 3-mercaptopropanoic acid (10.29 g, 96.97 mmol) in EtOH (100 mL) was added dropwise NH₂NH₂·H₂O (79.26 g, 1.55 mol) at 0° C. under N₂. The mixture was stirred for 16 h at 40° C. The mixture was quenched by water (100 mL), adjusted to pH=4 by addition of HCl (1 M), extracted with EtOAc (3×100 mL). The combined organic layers were washed with brine (100 mL), dried over Na₂SO₄, filtered and concentrated under reduced pressure. The mixture was slurried with MTBE: EtOAc (10:1, 100 mL), filtered and the solid was desired. The solid was purified by p-HPLC (FA) under the following condition: column: Phenomenex luna C18 (250×70 mm, 15 um); mobile phase: [water (FA)-ACN]; B %: 8%-35%, 22 min to give 3-methyl-6-phenyl-1,4-dihydro-1,2,4,5-tetrazine (10 g, 29%).

LCMS, m/z=175.1 [M+H]⁺

¹H NMR (400 MHZ, DMSO): δ 8.50 (br s, 1H), 8.29 (br s, 1H), 7.74 (dd, J=1.6, 7.6 Hz, 2H), 7.47-7.35 (m, 3H), 1.78 (s, 3H).

4-nitrophenyl 6-methyl-3-phenyl-1,2,4,5-tetrazine-1 (4/I)-carboxylate (4): To a solution of 3-methyl-6-phenyl-1,4-dihydro-1,2,4,5-tetrazine (3 g, 17.22 mmol) in DCM (30 mL) was added DIEA (6.68 g, 51.66 mmol) and then (4-nitrophenyl) carbonochloridate (3.64 g, 18.08 mmol) at 0° C. under N₂. The mixture was stirred for 2 h at 25° C. The mixture was poured into water (45 mL), extracted with DCM (3×15 mL). The combined organic layers were washed with brine (15 mL), dried over Na₂SO₄, filtered and concentrated under reduced pressure. The crude was purified by silica gel column chromatography (PE: EtOAc=1:0 to 0:1) to give 4-nitrophenyl 6-methyl-3-phenyl-1,2,4,5-tetrazine-1 (4H)-carboxylate (1 g, 17%).

LCMS, m/z=340.0 [M+H]⁺

¹H NMR (400 MHZ, CDCl₃): δ 8.36-8.28 (m, 2H), 7.76 (d, J=7.6 Hz, 2H), 7.56-7.42 (m, 5H), 6.30-6.02 (m, 2H), 2.44 (s, 3H).

4-((S)-2-((S)-2-amino-3-methylbutanamido)-5-ureidopentanamido)benzyl-6-methyl-3-phenyl-1,2,4,5-tetrazine-1 (4H)-carboxylate (6): To a solution of 4-nitrophenyl 6-methyl-3-phenyl-1,2,4,5-tetrazine-1 (4H)-carboxylate (705 mg, 1.66 mmol) and (9H-fluoren-9-yl)methyl ((S)-1-(((S)-1-((4-(hydroxymethyl)phenyl)amino)-1-oxo-5-ureidopentan-2-yl)amino)-3-methyl-1-oxobutan-2-yl) carbamate (1 g, 1.66 mmol) in DMF (10 mL) was added DIEA (644 mg, 4.99 mmol) at 25° C. under N₂. The mixture was heated to 80° C. and stirred for 2 h. The crude was purified by p-HPLC (neutral) under the following condition: column: Phenomenex C18 250×50 mm×10 um; mobile phase: [water (NH₄HCO₃)-ACN]; B %: 14%-44%, 8 min to give 4-((S)-2-((S)-2-amino-3-methylbutanamido)-5-ureidopentanamido)benzyl-6-methyl-3-phenyl-1,2,4,5-tetrazine-1 (4H)-carboxylate (240 mg, 25%).

LCMS, m/z=580.2 [M+H]⁺

¹H NMR (400 MHZ, DMSO): δ 9.97 (s, 1H), 9.84 (s, 1H), 8.42 (d, J=7.6 Hz, 1H), 7.88-7.79 (m, 2H), 7.60-7.47 (m, 5H), 7.23 (d, J=8.4 Hz, 2H), 7.14 (d, J=8.4 Hz, 1H), 5.98 (br t, J=6.0 Hz, 1H), 5.47-5.35 (m, 2H), 5.12-5.04 (m, 1H), 4.43 (d, J=5.6 Hz, 3H), 4.26 (dd, J=5.6, 8.8 Hz, 1H), 3.07-2.94 (m, 2H), 2.18 (s, 3H), 2.14-2.04 (m, 1H), 1.75-1.55 (m, 2H), 1.51-1.30 (m, 2H), 0.92 (d, J=6.8 Hz, 3H), 0.85 (d, J=6.8 Hz, 3H)

4-((S)-2-((S)-2-acetamido-3-methylbutanamido)-5-ureidopentanamido)benzyl-6-methyl-3-phenyl-1,2,4,5-tetrazine-1 (4H)-carboxylate (Target 1b): To a solution of 4-((S)-2-((S)-2-amino-3-methylbutanamido)-5-ureidopentanamido)benzyl 6-methyl-3-phenyl-1,2,4,5-tetrazine-1 (4H)-carboxylate (100 mg, 0.17 mmol) and DIEA (45 mg, 0.35 mmol) in THF (0.5 mL) was added DMAP (4 mg, 0.03 mmol) and then Ac₂O (23 mg, 0.22 mmol) at 0° C. under N₂. The mixture was stirred for 2 h at 25° C. The mixture was concentrated under reduced pressure. The crude was purified by p-HPLC (neutral) under the following condition: column: Waters xbridge 150×25 mm×10 um; mobile phase: [water (NH₄HCO₃)-ACN]; B %: 24%-54%, 8 min to give 4-((S)-2-((S)-2-acetamido-3-methylbutanamido)-5-ureidopentanamido)benzyl 6-methyl-3-phenyl-1,2,4,5-tetrazine-1 (4H)-carboxylate (58.9 mg, 55%).

LCMS, m/z=622.2 [M+H]⁺

¹H NMR (400 MHZ, DMSO): δ 10.09 (s, 1H), 9.85 (s, 1H), 8.44 (d, J=7.2 Hz, 1H), 7.87-7.77 (m, 2H), 7.64-7.47 (m, 5H), 7.30 (d, J=8.4 Hz, 2H), 7.14 (d, J=8.4 Hz, 1H), 5.99 (br t, J=5.6 Hz, 1H), 5.41 (s, 2H), 5.00 (s, 2H), 4.47-4.36 (m, 1H), 4.27 (dd, J=5.6, 8.7 Hz, 1H), 3.10-2.88 (m, 2H), 2.18 (s, 3H), 2.14-2.06 (m, 1H), 2.04 (s, 3H), 1.77-1.57 (m, 2H), 1.52-1.32 (m, 2H), 0.92 (d, J=6.8 Hz, 3H), 0.85 (d, J=6.8 Hz, 3H).

### Example 23: LCMS Analysis of Drug Release by Tetrazines

A solution of each tetrazine (20 μM final concentration) in PBS is mixed with TCO-MMAE (10 μM final concentration). The solution is mixed thoroughly and an aliquot removed at the appropriate time for analysis by LCMS. The results are reported in the table below.

| | % TCO- | % MMAE |
|---|---|---|
| | MMAE | released |
| Compound | consumed (1 h) | (24 h) |
| 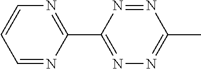 | 100 | 29 |
| 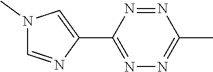 | 74 | 40 |
| 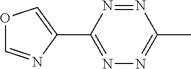 | 42 | 62 |
| 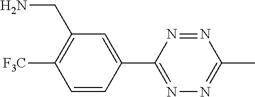 [SMILES: Cc1nnc(-c2ccc(C(F)(F)F)c(CN)c2)nn1] | 100 | 46 |
| 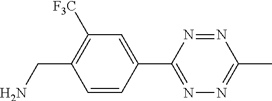 [SMILES: Cc1nnc(-c2ccc(CN)c(C(F)(F)F)c2)nn1] | 100 | 51 |
| 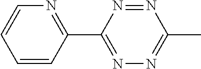 | 100 | 28 |
| 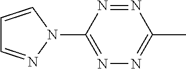 | 81 | 35 |

Based on at least the above data, it is contemplated that the drug release profile can be modified, enhanced, or attenuated by adjusting the structure of the bicyclic tetrazine or dihydrotetrazine portion targeting moiety.

### Example 24: Trastuzumab Fab-Me-Tet-PEG9 Conjugate Preparation and FACS Analysis

Fab of trastuzumab was synthesized by plasmid construction, HEK293 cell expression and purification. The Fab-tetrazine conjugate (ADC) was prepared by reacting Me-Tet-PEG9-NHS (structure shown below, purchased from SiChem; catalog No. SC-8808) to primary amines on the Fab to form stable amide bonds. The ADC was tested by flow cytometry (FACS) to compare binding to HER2 positive cells in comparison with un-conjugated Fab.

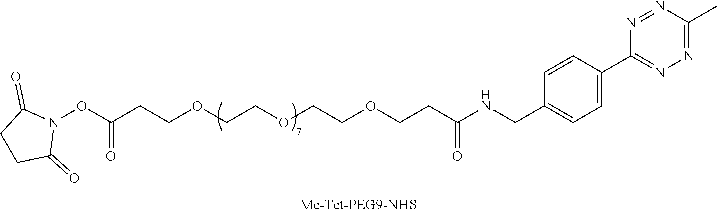

### Synthesis of Fab of Trastuzumab

Vector construction: Coding sequences (listed below) was synthesized and subcloned into expression vector. Constructed plasmids were transformed to E. coli for propagation. NucleoBond Xtra Maxi Plus EF kit was used for large scale plasmid generation. Purified plasmids were checked by agarose gel and confirmed by sequencing.

Trastuzumab-Fab HC sequence:
(SEQ ID NO. 3)
EVQLVESGGGLVQPGGSLRLSCAASGFNIKDTYIHWVRQAPGKGLEWVA
RIYPTNGYTRYADSVKGRFTISADTSKNTAYLQMNSLRAEDTAVYYCSR
WGGDGFYAMDYWGQGTLVTVSSASTKGPSVFPLAPSSKSTSGGTAALGC
LVKDYFPEPVTVSWNSGALTSGVHTFPAVLQSSGLYSLSSVVTVPSSSL
GTQTYICNVNHKPSNTKVDKKV.
Trastuzumab-Fab LC sequence:
(SEQ ID NO. 4)
DIQMTQSPSSLSASVGDRVTITCRASQDVNTAVAWYQQKPGKAPKLLIY
SASFLYSGVPSRFSGSRSGTDFTLTISSLQPEDFATYYCQQHYTTPPTF
GQGTKVEIKRTVAAPSVFIFPPSDEQLKSGTASVVCLLNNFYPREAKVQ
WKVDNALQSGNSQESVTEQDSKDSTYSLSSTLTLSKADYEKHKVYACEV
THQGLSSPVTKSFNRGEC.

Protein expression: The constructs containing heavy chain and light chain of the Fab were co-transfected into HEK293 cells with PEI. The culture medium was harvested at 6-7 days post transfection.

Protein purification: Conditional medium expressing target Fab was harvested by centrifugation and filtration, then loaded onto KappaSelect affinity column (Mabselect Prism). The loading buffer was 25 mM Tris containing 150 mM NaCl, pH 8.0, washed with 25 mM Tris buffer containing 150 mM NaCl, 0.2% Triton X-100/114, pH 8.0, and eluted with 100 mM Sodium-citrate buffer containing 150 mM NaCl, pH 2.5. The collected solution was neutralized with 1M arginine, 400 mM succinic acid buffer, pH 9.0. The affinity purified protein was further purified by gel filtration with Superdex S-200 column chromatography. Purified Fab was analyzed by SDS-PAGE, SEC-HPLC and endotoxin measurement.

Conjugate preparation: 120 mg Fab protein was dialyzed against PBS, pH 7.4 overnight with one buffer exchange at about 4 hours from the start. 10 mM Me-Tet-PEG9-NHS was prepared in DMSO. The two components were reacted at 3:1 drug to protein molar ratio at 25° C. for 2 hours before it was dialyzed against PBS, pH 7.4 to remove excess Me-Tet-PEG9-NHS compound from the protein component.

FACS binding assay of trastuzumab Fab, Fab-Me-Tet-PEG9-NHS conjugate against HER2 positive cell line: Cells (NCI-N87 cell line) cultured in RPMI1640+10% FBS were collected by centrifugation, re-suspended with FACS buffer (PBS containing 2% FBS, pH 7.4) and adjusted to density of 2E6/mL. 100 μL (2E5) of cells was seeded into a 96 well plate, centrifuged at 400 g for 5 minutes, discarded the supernatant and resuspended with serially diluted Fab solution at 1:10 ratio from a 20 μL/mL stock solution. The Ab and cells were incubated at 4° C. for 1 hour before the cells were treated by centrifugation at 400 g for 5 minutes to remove the supernatant and washed 3 times with FACS buffer (200 μL/well). The cells was resuspended in 100 μL alexa 488 labeled anti-human IgG (Life Technologies, Catalog No. 11013, 2 μg/mL), and incubated at 4° C. for 1 hour in the dark before the supernatant was removed and the cells were washed twice with 100 μL PBS by centrifugation at 400g for 5 minutes each time.

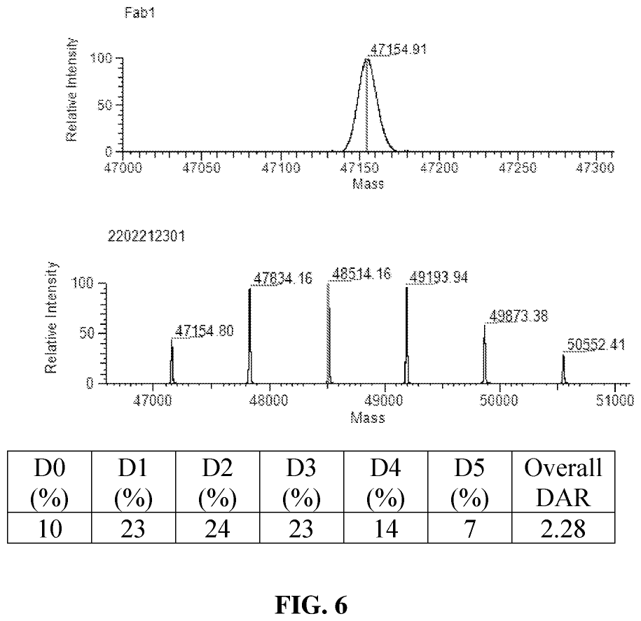

Results: 290 mg protein was obtained from the first step affinity purification of 6L culture medium with Mabselect Prism. 215 mg of Fab protein was obtained after purification with Superdex S-200 column chromatography. 112 mg conjugate was obtained after the conjugation and dialysis procedure. The drug to Ab ratio (DAR) determined with LC-MS method was 2.28 (FIG. 6).

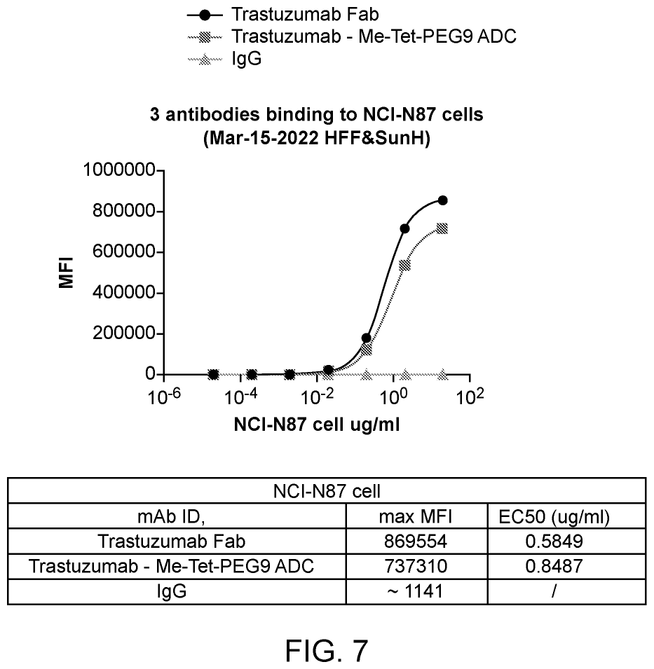

FACS binding assay of trastuzumab Fab, Trastuzumab-Me-Tet-PEG9 ADC against HER2 positive cell line: The binding profile (FIG. 7) shows comparable or slightly reduced binding activity of Me-Tet-PEG9 conjugated trastuzumab Fab in comparison to un-conjugated Fab.

### Example 25: Efficacy Study of Trastuzumab Fab-Tz (from Example 24) with Compound A in the Treatment of Subcutaneous NCI-N87 Xenograft Model in CB17 SCID Mice

In this study, the antitumor efficacy of MMAE, Tz-Fab, Tz-Fab+Compound A, Compound A, SQL70+Compound A and T-DM1 in the treatment of NCI-N87 xenograft model was evaluated. The objective was to evaluate the ability of a Fab-tetrazine conjugate to localize a partner TCO-MMAE protodrug in an efficacy study for the treatment of subcutaneous NCI-N87 xenograft model in female CB17 SCID mice.

### LIST OF ABBREVIATIONS

| | |
|---|---|
| ATCC | American Type Culture Collection |
| RT | Room temperature |
| QW | Once per week |
| QD | Once per day |
| BW | Body weight |
| RCBW | Relative change of body weight |
| TV | Tumor volume |
| IV | Injection Of Vein |
| IT | injected intratumorally |
| SEM | Standard error of the mean |
| N | Number of animals |
| mpk | mg/kg |
| PG | Post grouping |

Methods: 110 CB17 SCID female mice (Mus musculus; age: 6-8 weeks; body weight: 18-22 g; Shanghai LingChang Biotech Co., LTD) were inoculated subcutaneously at right flank with NCI-N87 cells (3×10⁶ cells was suspended in 0.2 mL mixture of 1640 with (1640: Matrigel=1:1) BD Matrigel for tumor development). After 7 days inoculation, 70 animals were divided into 8 groups with 10 animals in group 1, 2, 5, 6, 7 and 8, and with 5 animals in group 3 and 4 using stratified random grouping based on their body weight and tumor volume. The treatments were started from the mean tumor size reached to 158 mm³ (106-221 mm³) for these 70 mice. Group 1: Vehicle, i.v., QD×3×2 weeks. Group 2: MMAE, 0.25 mpk, i.v., QW×2 weeks. Group 3: Tz-Fab, 50 mpk, i.v., QW×2 weeks. Group 4: Compound A, 20 mpk, i.v., QD×3×2 weeks. Group 5: Tz-Fab, 50 mpk, i.v., QW×2 weeks+Compound A, 20 mpk, i.v., QD×3×2 weeks. Group 6: Tz-Fab, 50 mpk, i.v., QW×2 weeks+Compound A, 5 mpk, i.v., QD×3×2 weeks. Group 7: SQL70, 100 μL, i.t.×1 dose+Compound A, 1.9 mpk, i.v., QD×5. Group 8: T-DM1, 3 mpk, i.v., QW×2 weeks. The tumor sizes and body weight were measured twice a week during the treatment. The dosing schedule was finished on day PG-D10. The entire study was terminated on PG-D37 and collected all the tumor samples for group 5 and 6, and 3 tumors/group for group 1 and 2.

TABLE 2
Instruments

| Name | Vendor | Type |
|---|---|---|
| Class II Type A2 Biological safety | Thermo | 1389 |
| Cabinet | | |
| Vernier caliper | Mitutoyo | 500-172-20 |
| Analytical balance | METTLER TOLEDO | XP205 |
| Electronic scale | Changshu Shuangjie | JJ300Y |
| | Instrument Co., Ltd | |

TABLE 3
Reagents

| Item | Vendor | Catalog No. | Lot No. |
|---|---|---|---|
| PBS | HyClone | SH30256.01 | AC10237763 |
| RPMI-1640 | Gibco | 11875-093 | 2379429 |
| FBS | BI | 04-002-1A | 1841929 |
| PS | Gibco | 15140-122 | 2321139 |
| 0.25% Trypsin-EDTA | Gibco | 25200-072 | 2152925 |
| saline | Shandong Hualu | — | SD19121806 |
| | Pharmaceutical Co.,Ltd | | |
| HPCD | Accela | SY004863 | R1804474 |

Experimental Methods and Procedures: Cell preparation and implantation: The NCI-N87 (Gastric carcinoma, ATCC® CRL-5822™, Lot No. 7686255) cells were maintained in vitro as a monolayer culture in RPMI-1640 medium supplemented with 10% heat inactivated fetal bovine serum, 100 U/mL penicillin and 100 μg/mL streptomycin at 37° C. in an atmosphere of 5% CO₂ in air. The tumor cells were routinely sub-cultured per 5 days by trypsin-EDTA treatment. The cultured NCI-N₈₇ cells were harvested, re-suspended in base medium at a density of 1.5×10⁷ cells/mL with viability >90%. Each mouse was inoculated subcutaneously at the right flank with 3×10⁶ in 0.2 mL mixture of 1640 with (1640: Matrigel=1:1) BD Matrigel for tumor development.

Randomization and administration: The treatments were started from the mice were grouped based on their mean tumor size reached 158 mm³ (106-221 mm³) for group 1-8. Each group consisted of 10 or 5 mice. The test article was administrated to the mice according to the regimen as shown in the experimental design table (Table 4).

TABLE 4
Groups and Treatments

| | | | Dose | Dosing Volume | Dosing | |
|---|---|---|---|---|---|---|
| Group | n | Treatment | (mg/kg) | (mL/kg) | Route | Schedule |
| 1 | 10 | Vehicle | — | 10 | IV | QD 3 × 2 weeks |
| 2 | 10 | MMAE | 0.25 | 10 | IV | QW × 2 |
| 3 | 5 | Tz-Fab | 50 | 10 | IV | QW × 2 |
| 4 | 5 | Compound A | 20 | 10 | IV | QD 3 × 2 weeks |
| 5* | 10 | Tz-Fab + | 50 + 20 | 10 | IV + IV | QW × 2, QD 3 × 2 |
| | | Compound A | | | | weeks |
| 6* | 10 | Tz-Fab + | 50 + 5 | 10 | IV + IV | QW × 2, QD 3 × 2 |
| | | Compound A | | | | weeks |
| 7** | 10 | SQL70 + | 100 μL + 1.9 | 10 | IT + IV | QD × 5 |
| | | Compound A | | | | |
| 8 | 10 | T-DM1(Kadcyla) | 3 | 10 | IV | QW × 2 |

n: animal number.
Dosing volume: adjust dosing volume based on body weight 10 mL/kg.
Treatment schedule may be adjusted if body weight loss >15%.
*day 1 - 4 h post Tz-Fab dosing, day 2, day 3, day 8 - 4 h post Tz-Fab dosing, day 9, day 10
**The SQL70 (Srinivasan et al. Adv. Therap. 2021; formulated for injection) was injected intratumorally by the following scheme (20 μL in center of tumor and 20 μL at each pole of the tumor). Dose Compound A daily for five days. The first Compound A dose was given approximately 1 hour after SQL70 injection. Group 7 was only receiving a single cycle of dosing.

Formulation preparation is shown in Table 5.

TABLE 5
Formulation Preparation

| | | | Conc. | |
|---|---|---|---|---|
| Compound | Package | Preparation | (mg/mL) | Storage |
| Vehicle | — | Prepared 10% HPCD (Accela), used within 1 | — | 4° C. |
| | | week | | |
| | | Dissolved 5 g HPCD powder in 50 mL of sterile | | |
| | | deionized water. | | |
| | | Stirred to make HPCD dissolved as a clear | | |
| | | solution with a stir-bar. | | |
| | | Filter the clear solution with 0.22 μm sterile | | |
| | | filter and stored the filtrate at 4° C. | | |
| MMAE | | Prepared 0.025 mg/mL MMAE, freshly prepared | 0.025 | 4° C. |
| | | To a 50 mL vial, accurately weight 1 mg | | |
| | | MMAE, added 40 mL Saline, dissolved directly | | |
| | | into the saline to yield a clear solution. | | |
| | | Filter the solution with 0.22 μm sterile filter. | | |
| SQL-70 | | Formulated for injection | NA | −20° C. |
| Tz-Fab | 5.9 | Prepared 5 mg/mL Tz-Fab, fresh prepared | 5 | Fresh |
| | mg/mL | To a 15 mL vial, accurately pipette 6.7 mL of | | prepared |
| | | 5.9 mg/mL Tz-Fab, added 1.2 mL filtrated PBS | | |
| | | to the tube and mix well. | | |
| Compound | | Prepared 2 mg/mL Compound A, weekly | 2 | 4° C. |
| A | | prepared | | (Protected |
| | | To a 50 mL vial, accurately dispense 38 mg | | from light) |
| | | Compound A, added 19 mL 10% HPCD to wet | | |
| | | the compound. | | |
| | | Filter the solution with 0.22 μm sterile filter and | | |
| | | stored the filtrate at 4° C. | | |
| Compound | | Prepared 0.5 mg/mL Compound A, weekly | 0.5 | |
| A | | prepared | | |
| | | To a 15 mL vial, accurately pipette 2.5mL of | | |
| | | 2 mg/mL Compound A, added 7.5 mL 10% | | |
| | | HPCD to the tube and mix well. | | |
| | | Filter the solution with 0.22 μm sterile filter and | | |
| | | stored the filtrate at 4° C. | | |
| Compound | | Prepared 0.33 mg/mL Compound A, weekly | 0.19 | |
| A | | prepared | | |
| | | To a 50 mL vial, accurately pipette 2.5 mL of 2 | | |
| | | mg/mL Compound A, added 12.65 mL 10% | | |
| | | HPCD to the tube and mix well. | | |
| | | Filter the solution with 0.22 μm sterile filter and | | |
| | | stored the filtrate at 4° C. | | |
| T-DM1 | | Prepared 0.3 mg/mL T-DM1, freshly prepared | 0.3 | Freshly |
| | | To a 15 mL vial, accurately weight 1.8 mg T- | | prepared |
| | | DM1, added 6 mL PBS, dissolved directly into | | |
| | | the saline to yield a clear solution | | |
| | | Filter the solution with 0.22 μm sterile filter. | | |

Note:
Ensure that formulation is homogenous immediately before use by gentle pipetting.

Tumor Measurements and Endpoints: The major endpoint is to see if the tumor growth can be delayed or mice can be cured. Tumor sizes were measured in two dimensions using a caliper, and the volume was expressed in mm³ using the formula: V=0.5 a×b² where a and b are the long and short diameters of the tumor, respectively. The tumor sizes were then used for the calculations of both tumor growth inhibition (TGI) values.

TGI was calculated for each group using the formula: TGI (%)=[1−(TV_{Treatment_DayN}−TV_{Treatment_Day0})/(TV_{Vehicle_DayN}−TV_{Vehicle_Day0})]×100%; TV_{Treatment_DayN} is the average tumor volume of a treatment group on a given day, TV_{Treatment_Day0} is the average tumor volume of the treatment group on the first day of treatment, TV_{Vehicle_DayN} is the average tumor volume of the vehicle control group on a given day, and TV_{Vehicle_Day0} is the average tumor volume of the vehicle group on the first day of treatment.

The T/C value (in percent) is an indication of antitumor effectiveness, T/C (%)=RTV_{Treatment}/RTV_{Control}×100% (RTV_{Treatment}: the mean RTV of the treatment group; RTV_{Control}: the mean RTV of the vehicle treated group). RTV (relative tumor volume)=TV_{DayN}/TV_{Day0}. TV_{DayN} and TV_{Day0} is the tumor volume on day N and Day 0 respectively. T/C (%)≤42% is considered as significant antitumor activity and <10% is considered as highly significant antitumor activity by the United States National Cancer Institute criteria.

Relative change of body weight (RCBW) of each mouse was calculated according to the following formula: RCBW (%)=(BW_{Treatment_DayN}−BW_{Treatment_Day0})/BW_{Treatment_Day0}×100%.

Statistical Analysis: The tumor volume between different groups was analyzed by two-way repeated measures ANOVA followed by the Tukey's post hoc test. All data were analyzed using GraphPad Prism 6.0. P<0.05 is considered to be statistically significant.

### Results

Body weight change: Animal body weight was monitored as an indirect measure of toxicity. After grouping, all treatments were well tolerated. No significant change in body weight was observed. No other obvious abnormality was observed in these mice.

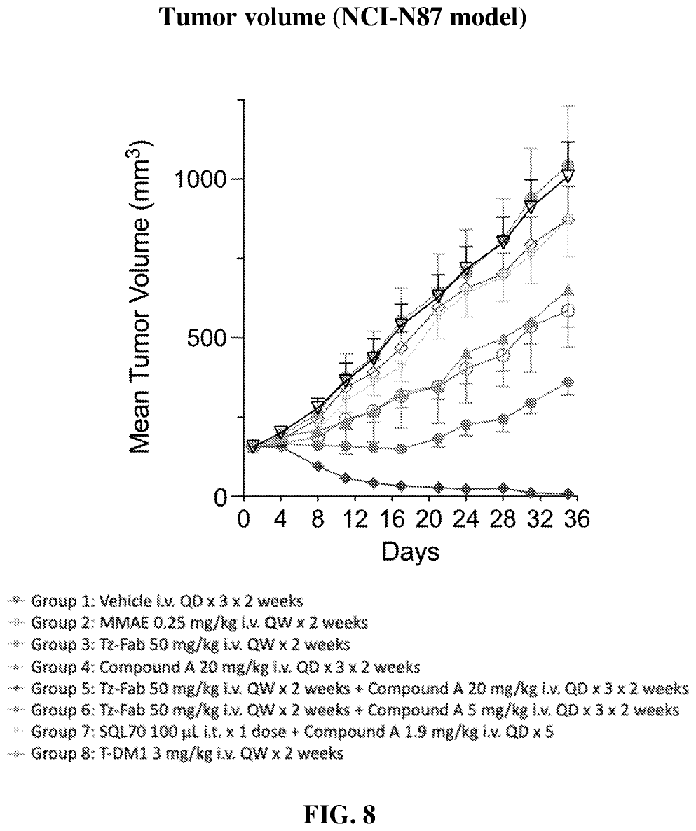

Tumor Volume and Tumor Growth Curve: Tumor growth curves (shown as mean tumor volumes) over time in NCI-N87 tumor bearing female CB17 SCID mice dosed with MMAE, Tz-Fab, Tz-Fab+Compound A, Compound A, SQL70+Compound A and T-DM1 is shown in FIG. 8 (data points represent group means, and error bars represent standard errors of the mean (SEM)).

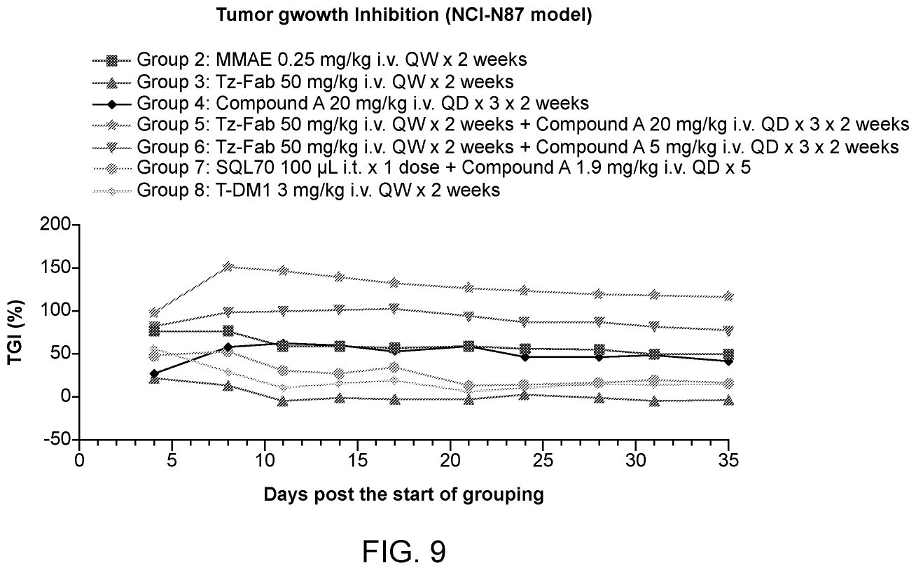

Tumor growth inhibition curves was shown in FIG. 9.

The results of tumor sizes in different groups at different time points are shown in FIG. 3. The mean tumor size of the vehicle control mice reached 1009 mm³ (Group1) on day 35 after grouping. The mean tumor size of each treatment groups on day 35 were as follows: 586 mm³ (Group 2), 1043 mm³ (Group 3), 653 mm³ (Group 4), 8 mm³ (Group 5), 361 mm³ (Group 6), 871 mm³ (Group 7), 873 mm³ (Group 8). Compared with the vehicle control group (Group 1), the treatments group 2, 4, 5 and 6 showed statistically significant difference (P<0.05, the TGI were 49.68%, 41.74%, 117.52%, and 76.30%, respectively; the T/C were 57.50%, 62.12%, 0.82% and 35.44%, respectively).

On day 35, five out of 10 animals in Group 5 were tumor-free (complete response). No other group had animals that were tumor-free on day 35. All treatments were well tolerated by the NCI-N87 tumor-bearing female CB17 SCID mice. And no other serious adverse reactions were found.

### Example 26: Antibody Fragment Moieties

Further targeting moieties can be prepared, as in e.g., Example 6, using the sequences shown below.

### a) Synthesis of Fab of L19-Binding to FN-1 (Gene ID 2335)

Vector construction: Coding sequences (listed below) are synthesized and subcloned into expression vector. Constructed plasmids are transformed to E. coli for propagation. NucleoBond Xtra Maxi Plus EF kit are used for large scale plasmid generation. Purified plasmids are checked by agarose gel and confirmed by sequencing.

L19-Fab HC sequence:
(SEQ ID NO. 5)
EVQLLESGGGLVQPGGSLRLSCAASGFTFSSFSMSWVRQAPGKGLEWVS
SISGSSGTTYYADSVKGRFTISRDNSKNTLYLQMNSLRAEDTAVYYCAK
PFPYFDYWGQGTLVTVSSASTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSSDKTHT.
L19-Fab LC sequence:
(SEQ ID NO. 6)
EIVLTQSPGTLSLSPGERATLSCRASQSVSSSFLAWYQQKPGQAPRLLI
YYASSRATGIPDRFSGSGSGTDFTLTISRLEPEDFAVYYCQQTGRIPPT
FGQGTKVEIKRTVAAPSVFIFPPSDEQLKSGTASVVCLLNNFYPREAKV
QWKVDNALQSGNSQESVTEQDSKDSTYSLSSTLTLSKADYEKHKVYACE
VTHQGLSSPCTKSFNRGEC

Protein expression: The constructs containing heavy chain and light chain of the Fab are co-transfected into HEK293 cells with PEI. The culture medium is harvested at 6-7 days post transfection.

Protein purification: Conditional medium expressing target Fab is harvested by centrifugation and filtration, and can then be loaded onto KappaSelect affinity column (Mabselect Prism). The loading buffer is 25 mM Tris containing 150 mM NaCl, pH 8.0; the wash buffer is 25 mM Tris buffer containing 150 mM NaCl, 0.2% Triton X-100/114, pH 8.0; the elution buffer is 100 mM Sodium-citrate buffer containing 150 mM NaCl, pH 2.5. The collected solution is neutralized with 1M arginine, 400 mM succinic acid buffer, pH 9.0. The affinity purified protein is further purified by gel filtration with Superdex S-200 column chromatography. Purified Fab is analyzed by SDS-PAGE, SEC-HPLC and endotoxin measurement.

Conjugate preparation: 120 mg Fab protein is dialyzed against PBS, pH 7.4 overnight with one buffer exchange at about 4 hours from the start. 10 mM Me-Tet-PEG9-NHS is prepared in DMSO. The two components are reacted at 3:1 drug to protein molar ratio at 25° C. for 2 hours; the reaction mixture is dialyzed against PBS, pH 7.4 to remove excess Me-Tet-PEG9-NHS compound from the protein component.

### b) Synthesis of Fab of F16-Binding to TNC (Gene ID 3371)

Vector construction: Coding sequences (listed below) are synthesized and subcloned into expression vector. Constructed plasmids are transformed to E. coli for propagation. NucleoBond Xtra Maxi Plus EF kit are used for large scale plasmid generation. Purified plasmids are checked by agarose gel and confirmed by sequencing.

F16-Fab HC sequence:
(SEQ ID NO. 7)
EVQLLESGGGLVQPGGSLRLSCAASGFTFSRYGMSWVRQAPGKGLEWVS
AISGSGGSTYYADSVKGRFTISRDNSKNTLYLQMNSLRAEDTAVYYCAK
AHNAFDYWGQGTLVTVSSCSTKGPSVFPLAPSSKSTSGGTAALGCLVKD
YFPEPVTVSWNSGALTSGVHTFPAVLQSSGLYSLSSVVTVPSSSLGTQT
YICNVNHKPSNTKVDKKVEPKSSDKTHT.
F16-Fab LC sequence:
(SEQ ID NO. 8)
SSELTQDPAVSVALGQTVRITCQGDSLRSYYASWYQQKPGQAPVLVIYG
KNNRPSGIPDRFSGSSSGNTASLTITGAQAEDEADYYCNSSVYTMPPVV
FGGGTKLTVLGQPKAAPSVTLFPPSSEELQANKATLVCLISDFYPGAVT
VAWKADSSPVKAGVETTTPSKQSNNKYAASSYLSLTPEQWKSHKSYSCQ
VTHEGSTVEKTVAPTECS.

Protein expression: The constructs containing heavy chain and light chain of the Fab are co-transfected into HEK293 cells with PEI. The culture medium is harvested at 6-7 days post transfection.

Protein purification: Conditional medium expressing target Fab is harvested by centrifugation and filtration, and can then be loaded onto KappaSelect affinity column (Mabselect Prism). The loading buffer is 25 mM Tris containing 150 mM NaCl, pH 8.0; the wash buffer is 25 mM Tris buffer containing 150 mM NaCl, 0.2% Triton X-100/114, pH 8.0; the elution buffer is 100 mM Sodium-citrate buffer containing 150 mM NaCl, pH 2.5. The collected solution is neutralized with 1M arginine, 400 mM succinic acid buffer, pH 9.0. The affinity purified protein is further purified by gel filtration with Superdex S-200 column chromatography. Purified Fab is analyzed by SDS-PAGE, SEC-HPLC and endotoxin measurement.

Conjugate preparation: 120 mg Fab protein is dialyzed against PBS, pH 7.4 overnight with one buffer exchange at about 4 hours from the start. 10 mM Me-Tet-PEG9-NHS is prepared in DMSO. The two components are reacted at 3:1 drug to protein molar ratio at 25° C. for 2 hours; the reaction mixture is dialyzed against PBS, pH 7.4 to remove excess Me-Tet-PEG9-NHS compound from the protein component.

### c) Synthesis of Constructs of NJB2 (ECM-Targeting Sequences)

Vector construction: Coding sequences (listed below) are synthesized and subcloned into expression vector. Constructed plasmids are transformed to E. coli for propagation. NucleoBond Xtra Maxi Plus EF kit are used for large scale plasmid generation. Purified plasmids are checked by agarose gel and confirmed by sequencing.

NJB2 construct sequences:

Monovalent 1-VHH Fibronectin-His6
QVQLVETGGGLVQAGGSLRLSCAASGSTFSHNAGGWYRQAPEKQRELVAGISSDGNIN
YADSVKDRFTISRDNASNTMYLQMNLKPEDTAVYVCNIRGSYGNTYYSRWGQGTQVTVSSGG
LPETGGHHHHHH (SEQ ID NO. 9).
Monovalent 2-VHH Fibronectin-His6-Cys
QVQLVETGGGLVQAGGSLRLSCAASGSTFSHNAGGWYRQAPEKQRELVAGISSDGNIN
YADSVKDRFTISRDNASNTMYLQMNLKPEDTAVYVCNIRGSYGNTYYSRWGQGTQVTVSSGG
LPETGGHHHHHHC (SEQ ID NO. 10)
Bivalent 1-VHH-6GS-VHH-His6
QVQLVETGGGLVQAGGSLRLSCAASGSTFSHNAGGWYRQAPEKQRELVAGISSDGNIN
YADSVKDRFTISRDNASNTMYLQMNLKPEDTAVYVCNIRGSYGNTYYSRWGQGTQVTVSSGG
LPET
GGGSGGGSGGGSGGGSGGGSGGGSQVQLVETGGGLVQAGGSLRLSCAASGSTFSHNAGGWYR
QAPEKQRELVAGISSDGNINYADSVKDRFTISRDNASNTMYLQMNLKPEDTAVYVCNIRGSYGN
TYYSRWGQGTQVTVSSGGLPETGGHHHHHH (SEQ ID NO. 11)
Bivalent 2-VHH-His6-cys-6GS-VHH
QVQLVETGGGLVQAGGSLRLSCAASGSTFSHNAGGWYRQAPEKQRELVAGISSDGNIN
YADSVKDRFTISRDNASNTMYLQMNLKPEDTAVYVCNIRGSYGNTYYSRWGQGTQVTVSSGG
LPETGGHHHHHHCGGGSGGGSGGGSGGGSGGGSGGGSQVQLVETGGGLVQAGGSLRLSCAAS
GSTFSHNAGGWYRQAPEKQRELVAGISSDGNINYADSVKDRFTISRDNASNTMYLQMNLKPED
TAVYVCNIRGSYGNTYYSRWGQGTQVTVSSGGLPETGG (SEQ ID NO. 12)
Bivalent 3-VHH-6GS-cys-His6-VHH
QVQLVETGGGLVQAGGSLRLSCAASGSTFSHNAGGWYRQAPEKQRELVAGISSDGNIN
YADSVKDRFTISRDNASNTMYLQMNLKPEDTAVYVCNIRGSYGNTYYSRWGQGTQVTVSSGG
LPETGGGGGSGGGSGGGSGGGSGGGSGGGSCHHHHHHQVQLVETGGGLVQAGGSLRLSCAAS
GSTFSHNAGGWYRQAPEKQRELVAGISSDGNINYADSVKDRFTISRDNASNTMYLQMNLKPED
TAVYVCNIRGSYGNTYYSRWGQGTQVTVSSGGLPETGG (SEQ ID NO. 13)
Bivalent 4-VHH-30GS-VHH-His6
QVQLVETGGGLVQAGGSLRLSCAASGSTFSHNAGGWYRQAPEKQRELVAGISSDGNIN
YADSVKDRFTISRDNASNTMYLQMNLKPEDTAVYVCNIRGSYGNTYYSRWGQGTQVTVSSGG
LPETGSGSGSGSGSGSGSGSGSGSGSGSGSGSGSGSGSGSGSGSGSGSGSGSGSGSGSGSGSGSQV
QLVETGGGLVQAGGSLRLSCAASGSTFSHNAGGWYRQAPEKQRELVAGISSDGNINYADSVKD
RFTISRDNASNTMYLQMNLKPEDTAVYVCNIRGSYGNTYYSRWGQGTQVTVSSGGLPETGGHH
HHHH (SEQ ID NO. 14)
Bivalent 5-VHH-His6-cys-30GS-VHH
QVQLVETGGGLVQAGGSLRLSCAASGSTFSHNAGGWYRQAPEKQRELVAGISSDGNIN
YADSVKDRFTISRDNASNTMYLQMNLKPEDTAVYVCNIRGSYGNTYYSRWGQGTQVTVSSGG
LPETGGHHHHHHCGSGSGSGSGSGSGSGSGSGSGSGSGSGSGSGSGSGSGSGSGSGSGSGSGSGS
GSGSGSGSQVQLVETGGGLVQAGGSLRLSCAASGSTFSHNAGGWYRQAPEKQRELVAGISSDG
NINYADSVKDRFTISRDNASNTMYLQMNLKPEDTAVYVCNIRGSYGNTYYSRWGQGTQVTVSS
GGLPETGG (SEQ ID NO. 15)
Bivalent 6-VHH-30GS-cys-His6-VHH
QVQLVETGGGLVQAGGSLRLSCAASGSTFSHNAGGWYRQAPEKQRELVAGISSDGNIN
YADSVKDRFTISRDNASNTMYLQMNLKPEDTAVYVCNIRGSYGNTYYSRWGQGTQVTVSSGG
LPETGSGSGSGSGSGSGSGSGSGSGSGSGSGSGSGSGSGSGSGSGSGSGSGSGSGSGSGSGSGSCH
HHHHHQVQLVETGGGLVQAGGSLRLSCAASGSTFSHNAGGWYRQAPEKQRELVAGISSDGNIN
YADSVKDRFTISRDNASNTMYLQMNLKPEDTAVYVCNIRGSYGNTYYSRWGQGTQVTVSSGG
LPETGG (SEQ ID NO. 16)

Protein expression: For each construct, the expression vector containing the gene target protein is transfected into HEK293 cells with PEI. The culture medium is harvested at 6-7 days post transfection.

Protein purification: Conditional medium expressing target protein is harvested by centrifugation and filtration, and can then be loaded onto an IMAC column (GE). The loading buffer is 25 mM Tris containing 150 mM NaCl, 10 mM histidine, pH 8.0; the wash buffer is 25 mM Tris buffer containing 150 mM NaCl, 20 mM histidine, 0.2% Triton X-100/114, pH 8.0; the elution buffer is 25 mM Tris buffer containing 150 mM NaCl, 1 M histidine, pH 8.0. The affinity purified protein is further purified by gel filtration with Superdex S-200 column chromatography. Purified protein is analyzed by SDS-PAGE, SEC-HPLC and endotoxin measurement.

Conjugate preparation: 120 mg protein is dialyzed against PBS, pH 7.4 overnight with one buffer exchange at about 4 hours from the start. 10 mM Me-Tet-PEG9-NHS is prepared in DMSO. The two components are reacted at 3:1 drug to protein molar ratio at 25° C. for 2 hours; the reaction mixture is dialyzed against PBS, pH 7.4 to remove excess Me-Tet-PEG9-NHS compound from the protein component.

Alternate Conjugate preparation: 120 mg protein is dialyzed against Tris, pH 8.0 overnight with one buffer exchange at about 4 hours from the start. 10 mM Me-Tet-PEG9-maleimide is prepared in DMSO. The two components are reacted at 3:1 drug to protein molar ratio at 25° C. for 2 hours; the reaction mixture is dialyzed against PBS, pH 7.4 to remove excess Me-Tet-PEG9-maleimide compound from the protein component.

### Example 27: Efficacy Study of Ab-Tz (Prepared as in Example 6) with Compound B in the Treatment of Subcutaneous NCI-N87 Xenograft Model in CB17 SCID Mice

In this study, the antitumor efficacy of MMAE, HER2 Tz-Nanofitin, HER2 Tz-Nanofitin+Compound B, isotype Tz-Nanofitin+Compound B, Compound B, and trastuzumab emtansine were tested in the NCI-N87 xenograft model of gastric cancer in SCID mice.

Animal welfare for this study complies with the U.S. Department of Agriculture's Animal Welfare Act (9 CFR Parts 1, 2 and 3) as applicable.

Materials and Methods: Female CB-17/SCID mice (6-9 weeks old, ˜20 g) were injected in the rear flank with 5 million viable cells suspended in serum-free media and Cultrex ECM to inoculate tumors. Groups were randomized when the average tumor size reached ˜100 mm³.

Treatments were performed as below.

| | | | | | | Dose |
|---|---|---|---|---|---|---|
| | | | Dose | Dosing Frequency | Dose Level | Volume |
| Group | N | Treatment | Route | & Duration | (mg/kg) | (mL/kg) |
| 1 | 8 | Vehicle (10% HPCD) | IV | Q3D × 3 Doses | N/A | 5 |
| 2 | 8 | MMAE | IV | Q3D × 3 Doses | 0.25 | 5 |
| 3 | 4 | HER2 Tz-Nanofitin | IV | Q3D × 3 Doses | 10 | 5 |
| 4 | 4 | Compound B | IV | Q3D × 3 Doses | 20 | 5 |
| 5 | 8 | HER2 Tz-Nanofitin | IV | Q3D × 3 Doses | 10 | 5 |
| | | Compound B (90 Min After | IV | Q3D × 3 Doses | 5 | 5 |
| | | Nanofitin) | | | | |
| 6 | 8 | HER2 Tz-Nanofitin | IV | Q3D × 3 Doses | 10 | 5 |
| | | Compound B (90 Min After | IV | Q3D × 3 Doses | 20 | 5 |
| | | Nanofitin) | | | | |
| 7 | 8 | Isotype Tz-Nanofitin | IV | Q3D × 3 Doses | 10 | 5 |
| | | Compound B (90 Min After | IV | Q3D × 3 Doses | 20 | 5 |
| | | Nanofitin) | | | | |
| 8 | 8 | T-DM1 | IV | Q3D × 3 Doses | 3 | 5 |

HER2 Tz-Nanofitin: produced by Example 6.

Animals were monitored weekly for palpable tumors, or any changes in appearance or behavior. Once tumors became palpable, tumors were measured at least once a week using calipers. Tumor volume will be calculated using the following equation: (longest diameter*shortest diameter²)/2. Once tumors became of appropriate size to begin the study, tumors and body weights were measured at least 2 times per week for the duration of the study. One individual was responsible for tumor measurements for the duration of the study.

Body weight was measured at least 2 times a week following randomization and initiation of treatment. Hydrogel/DietGel and/or dosing holidays may be given to animals due to body weight loss; body weight loss was calculated based on the body weight (BW) of the mouse on the first day of treatment.

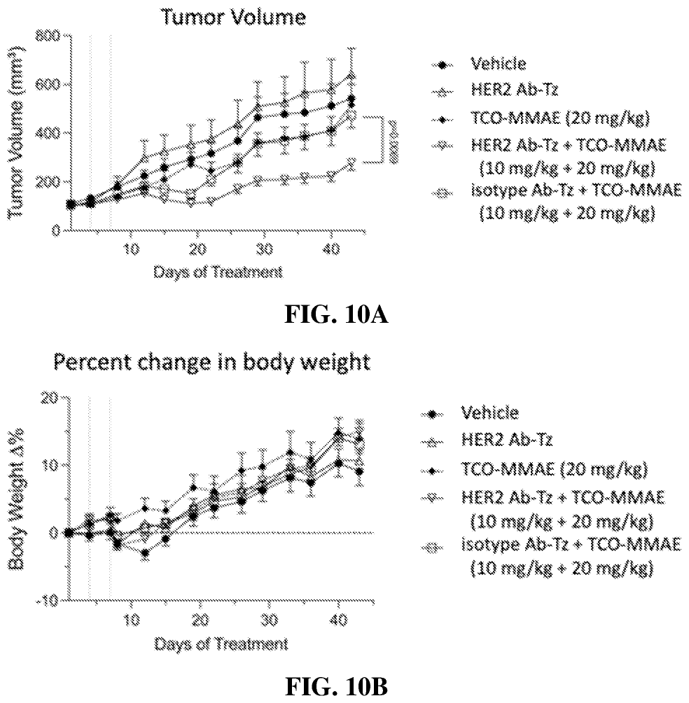

Clinical observations were performed at least 2 times a week at the time of tumor and body weight measurements. The results of tumor volume measurements and body weights are shown in FIG. 10A and FIG. 10B, respectively.

Unless otherwise defined, all technical and scientific terms used herein have the same meaning as commonly understood by one of ordinary skill in the art to which this disclosure belongs.

The embodiments illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claims.

All publications, patent applications, patents, and other references mentioned herein are expressly incorporated by reference in their entirety, to the same extent as if each were incorporated by reference individually. In case of conflict, the present specification, including definitions, will control.

It is to be understood that while the disclosure has been described in conjunction with the above embodiments, that the foregoing description and examples are intended to illustrate and not limit the scope of the disclosure. Other aspects, advantages and modifications within the scope of the disclosure will be apparent to those skilled in the art to which the disclosure pertains.

What is claimed is:

1. A targeting moiety of Formula I, Formula II, or Formula V: 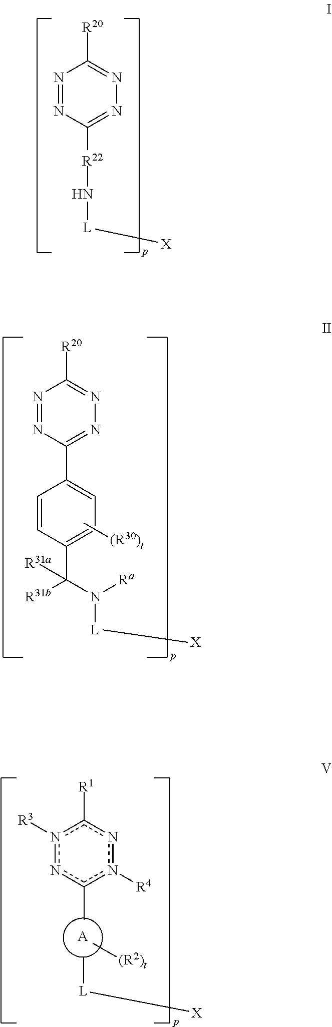
wherein:
ring A is aryl, cycloalkyl, heterocyclyl, or heteroaryl;
the dotted lines represent additional bonds to form a tetrazine when R³ and R⁴ are both absent, or a dihydrotetrazine when R³ and R⁴ are both present; provided that when ring A is aryl, then R³ and R⁴ are both present;
X is a biocompatible support, antibody, or antibody fragment moiety; provided that for Formula I and Formula II, X is not a biocompatible support;
p is 1-150;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or R³ and R⁴ are each independently hydrogen or a group capable of being removed after a triggering event;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R²², at each occurrence, is independently a linker of 1 to 100 linking atoms optionally comprising one or more ethylene-oxy, amine, ester, amide, carbamate, carbonate, or ketone functional group;
R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl;
R^{a}, R^{31a}, and R^{31b} are each independently hydrogen, C₁-C₆-alkyl, or C₁-C₆-haloalkyl;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl; and
t, at each occurrence, is independently is 0, 1, 2, 3, or 4.

2. The targeting moiety of claim 1, wherein p is 1-16, or 1-8, or 1-7, or 1-6, or 1-5, or 1-4, or 1-3, or 1-2.

3. A targeting moiety of Formula I or II: 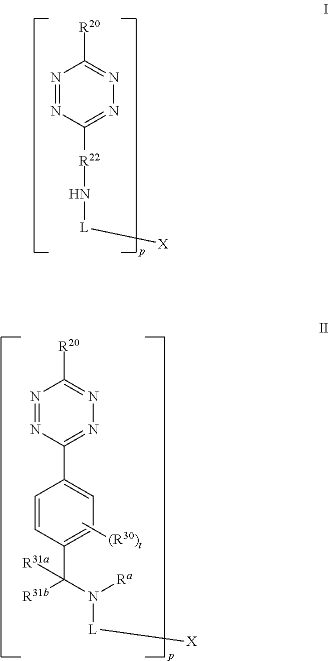
wherein:
X is an antibody or antibody fragment moiety;
p is 1-16;
L, at each occurrence, is independently a linker;
R²⁰, at each occurrence, is independently selected from the group consisting of hydrogen, halogen, cyano, nitro, alkyl, alkenyl, alkynyl, heteroalkyl, aryl, heteroaryl, heterocyclyl, cycloalkyl, cycloalkenyl, CF₃, CF₂—R′, NO₂, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R²², at each occurrence, is independently a linker of 1 to 100 linking atoms optionally comprising one or more ethylene-oxy, amine, ester, amide, carbamate, carbonate, or ketone functional group;
R³⁰, at each occurrence, is independently halogen, cyano, nitro, hydroxy, alkyl, haloalkyl, alkenyl, alkynyl, alkoxy, haloalkoxy, heteroalkyl, aryl, heteroaryl, heterocyclyl, cycloalkyl, or cycloalkenyl;
R^{a}, R^{31a} and R^{31b} are each independently hydrogen, C₁-C₆-alkyl, or C₁-C₆-haloalkyl;
R′ and R″, at each occurrence, are independently selected from hydrogen, aryl, and alkyl; R′″ at each occurrence is independently selected from aryl and alkyl; and
t, at each occurrence, is independently is 0, 1, 2, 3, or 4.

4. The targeting moiety of claim 3, represented by Formula IIA:

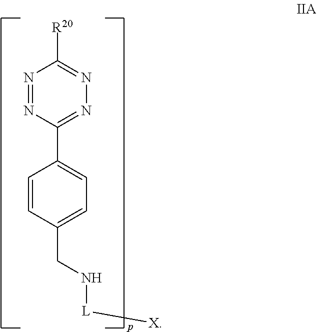

5. The targeting moiety of claim 3, represented by Formula IIB or IIC:

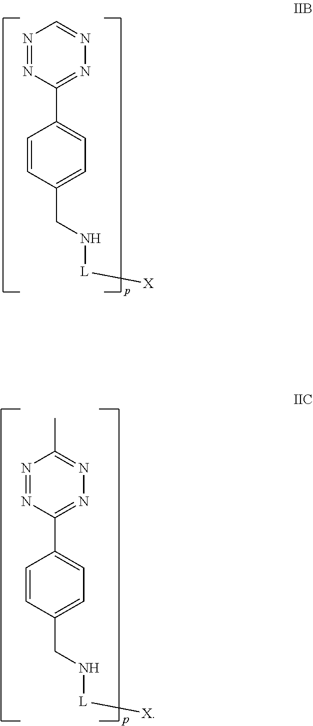

6. A targeting moiety of Formula V: 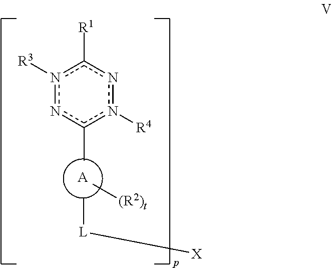
wherein:
ring A is aryl, cycloalkyl, heterocyclyl, or heteroaryl;
the dotted lines represent additional bonds to form a tetrazine when R³ and R⁴ are both absent, or a dihydrotetrazine when R³ and R⁴ are both present; provided that when ring A is aryl, then R³ and R⁴ are both present;
X is a biocompatible support, antibody, or antibody fragment moiety;
p is 1-150;
L, at each occurrence, is independently a linker;
R¹, at each occurrence, is independently selected from the group consisting of hydrogen, halo, cyano, nitro, alkyl, alkenyl, alkynyl, haloalkyl, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)OR′, C(═S)SR′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″; wherein each alkyl, alkenyl, alkynyl, haloalkyl, heteroalkyl, aryl, heteroaryl, heterocyclyl, or cycloalkyl is optionally substituted with one to three Z¹;
R², at each occurrence, is independently halo, cyano, nitro, hydroxy, alkyl, haloalkyl, alkenyl, alkynyl, alkoxy, haloalkoxy, heteroalkyl, aryl, heteroaryl, heterocyclyl, cycloalkyl, —C(═O)-alkyl, —C(═O)-haloalkyl, —C(═O)-alkenyl, —C(═O)-alkynyl, —C(═O)-alkoxy, —C(═O)-haloalkoxy, —C(═O)-heteroalkyl, —C(═O)-aryl, —C(═O)-heteroaryl, —C(═O)-heterocyclyl, or —C(═O)-cycloalkyl; wherein each alkyl, haloalkyl, alkenyl, alkynyl, alkoxy, haloalkoxy, heteroalkyl, aryl, heteroaryl, heterocyclyl, or cycloalkyl is optionally substituted with one to three Z¹;
R³ and R⁴ are both absent; or R³ and R⁴ are each independently hydrogen or a group capable of being removed after a triggering event;
each Z¹ is independently selected from halo, oxo, cyano, nitro, hydroxy, alkyl, haloalkyl, alkenyl, alkynyl, alkoxy, haloalkoxy, heteroalkyl, aryl, heteroaryl, heterocyclyl, cycloalkyl, OR′, SR′, C(═O)R′, C(═S)R′, OC(═O)R′″, SC(═O)R′″, OC(═S)R′″, SC(═S)R′″, S(═O)R′, S(═O)₂R′″, S(═O)₂NR′R″, C(═O)O—R′, C(═O)S—R′, C(═S)O—R′, C(═S)S—R′, C(═O)NR′R″, C(═S)NR′R″, NR′R″, NR′C(═O)R″, NR′C(═S)R″, NR′C(═O)OR″, NR′C(═S)OR″, NR′C(═O)SR″, NR′C(═S)SR″, OC(═O)NR′R″, SC(═O)NR′R″, OC(═S)R′R′″, SC(═S)R′R″, NR′C(═O)NR″R″, and NR′C(═S)NR″R″;
R′ and R″, at each occurrence, are independently selected from hydrogen, aryl, and alkyl;
R′″, at each occurrence, is independently selected from aryl and alkyl; and
t, at each occurrence, is independently is 0, 1, 2, 3, or 4.

7-14. (canceled)

15. The targeting moiety of claim 6, wherein at least one of the moiety: 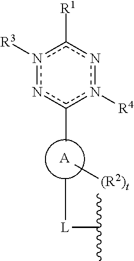
is represented by a formula selected from:

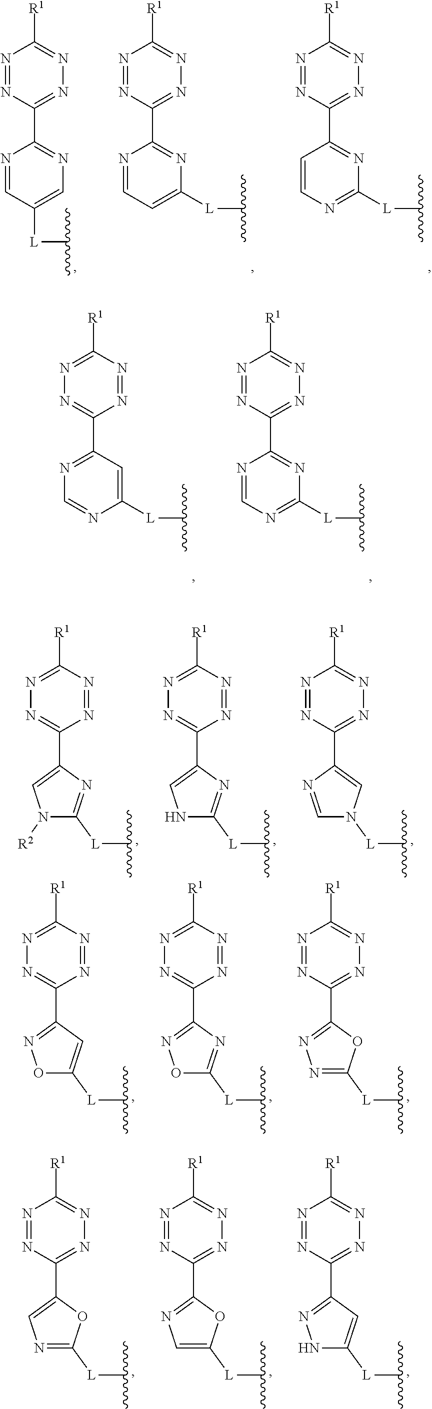

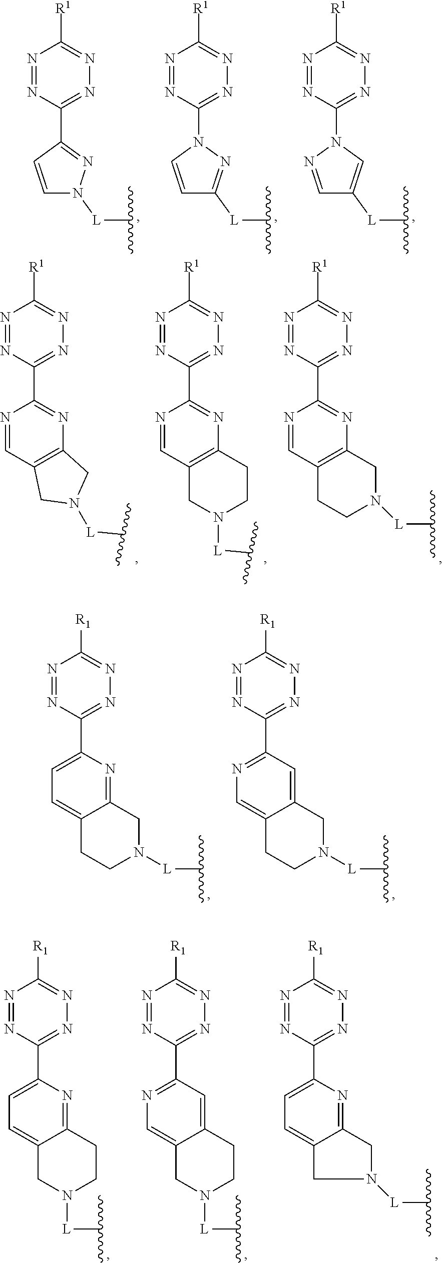

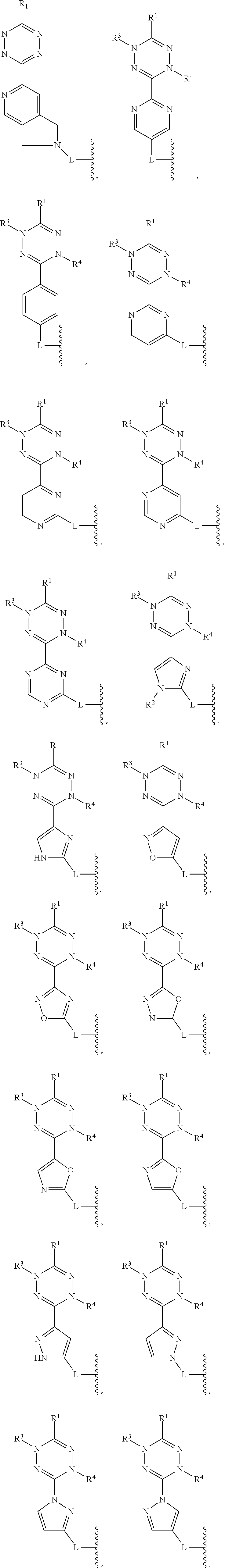

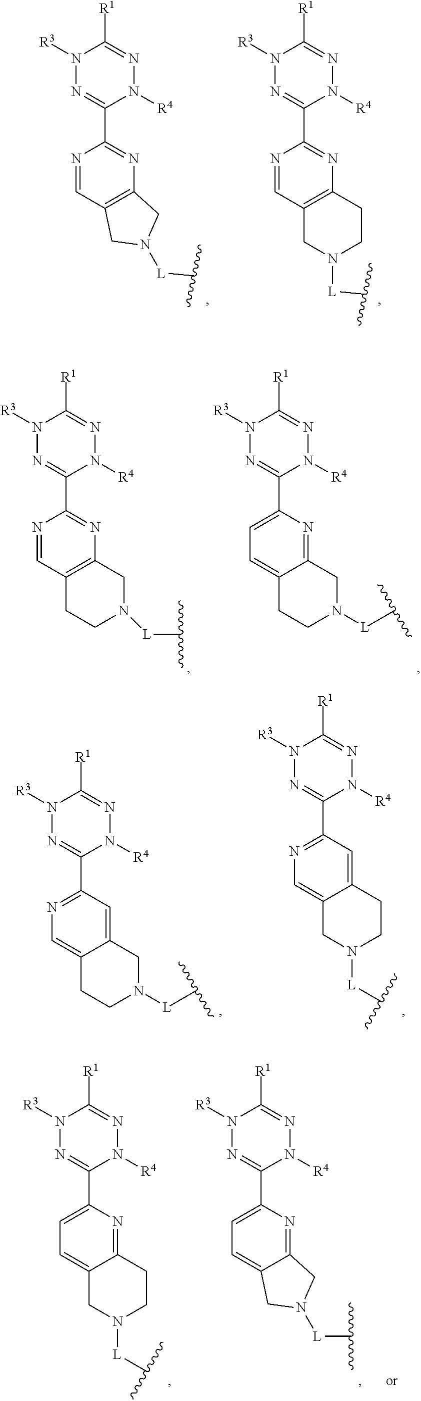

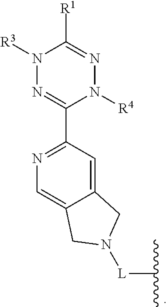

16-30. (canceled)

31. The targeting moiety of claim 6, wherein X further comprises an imaging contrast agent.

32-42. (canceled)

43. The targeting moiety of claim 1, wherein L is of the formula:
—Y¹⁰—(CHR¹³⁰)_{n′}—Y²⁰—(CHR¹⁴⁰)_{n″}—Y³⁰—(CHR¹⁵⁰)_{m″}—Y⁴⁰—
wherein:
each of Y¹⁰, Y²⁰, Y³⁰, and Y⁴⁰ are independently a bond, —NR¹¹⁰—, —O—, —S(O)₀₋₂—, —NR¹¹⁰C(O)—, —C(O)NR¹¹⁰, —NR¹¹⁰S(O)₂—, —S(O)₂NR¹¹⁰—, —CR¹²⁰═N—NR¹¹⁰—, —NR¹¹⁰—N═CR¹²⁰—, —C(O)—, —OC(O)—, —OC(O)O—, (CH₂CH₂O)₁₋₅—, —C(O)O—, alkylene, alkenylene, alkynylene, arylene, or heteroarylene; wherein each alkylene, alkenylene, alkynylene, arylene, or heteroarylene is independently optionally substituted with one to five substituents independently selected from oxo, halo, C₁₋₄ alkyl, C₁₋₄ alkoxy, and C₁₋₄ haloalkyl;
each R¹¹⁰ is independently hydrogen, C₁₋₄ alkyl, C₁₋₄ haloalkyl, aryl, heteroaryl, cycloalkyl, or heterocyclyl;
each R¹²⁰ is independently hydrogen, C₁₋₄ alkyl, C₁₋₄ haloalkyl, aryl, heteroaryl, cycloalkyl, or heterocyclyl;
each R¹³⁰ is independently hydrogen, C₁₋₄ alkyl, C₁₋₄ haloalkyl, aryl, heteroaryl, cycloalkyl, heterocyclyl, or an amino acid side chain;
each R¹⁴⁰ is independently hydrogen, C₁₋₄ alkyl, C₁₋₄ haloalkyl, aryl, heteroaryl, cycloalkyl, heterocyclyl, or an amino acid side chain;
each R¹⁵⁰ is independently hydrogen, C₁₋₄ alkyl, C₁₋₄ haloalkyl, aryl, heteroaryl, cycloalkyl, heterocyclyl, or an amino acid side chain; and
n′, n″, and m″ are each independently 0, 1, 2, 3, 4, 5, 6, 7, or 8.

44. The targeting moiety of claim 1, wherein L is of the formula:
—Y¹⁰—(CH₂)_{n′}—Y²⁰—(CH₂)_{m″}—Y³⁰—
wherein:
each of Y¹⁰, Y²⁰, and Y³⁰ are independently a bond, —NR¹¹⁰—, —O—, —S(O)₀₋₂—, —NR¹¹⁰C(O)—, —C(O)NR¹¹⁰—, —NR¹¹⁰S(O)₂—, —S(O)₂NR¹¹⁰—, —CR¹²⁰═N—NR¹¹⁰—, —NR¹¹⁰—N═CR¹²⁰—, —C(O)—, —OC(O)—, —OC(O)O—, alkylene, alkenylene, alkynylene, arylene, heteroarylene, cycloalkylene, or heterocycloalkylene; wherein each alkylene, alkenylene, alkynylene, arylene, heteroarylene, cycloalkylene, or heterocycloalkylene is independently optionally substituted with one to five substituents independently selected from oxo, halo, C₁₋₄ alkyl, C₁₋₄ alkoxy, and C₁₋₄ haloalkyl;
each R¹¹⁰ is independently hydrogen, C₁₋₄ alkyl, C₁₋₄ haloalkyl, aryl, heteroaryl, cycloalkyl or heterocyclyl;
each R¹²⁰ is independently hydrogen, C₁₋₄ alkyl, C₁₋₄ haloalkyl, aryl, heteroaryl, cycloalkyl or heterocyclyl; and
n′ and m″ are each independently 0, 1, 2, 3, 4, 5, 6, 7, or 8.

45. The targeting moiety of claim 1, wherein L is:

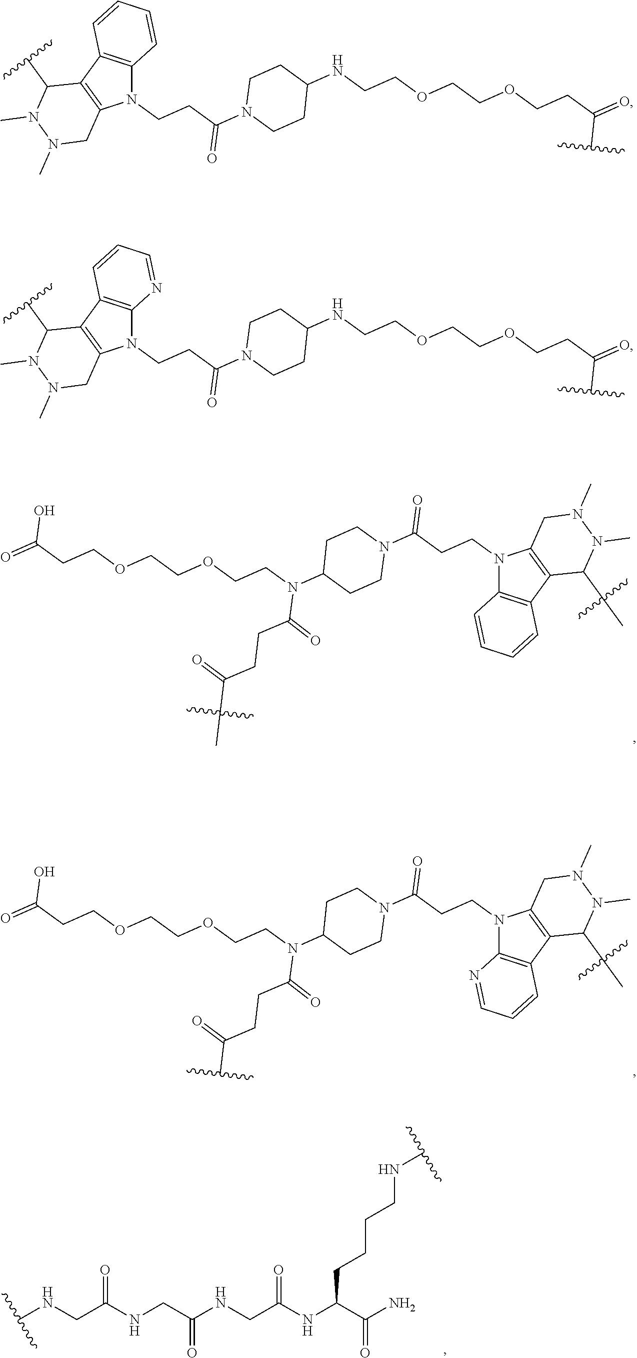 [SMILES: *C(=O)CCOCCOCCNC1CCN(C(=O)CCn2c3c(c4ccccc42)C(*)N(C)N(C)C3)CC1 ; *C(=O)CCOCCOCCNC1CCN(C(=O)CCn2c3c(c4cccnc42)C(*)N(C)N(C)C3)CC1 ; *C(=O)CCC(=O)N(CCOCCOCCC(=O)O)C1CCN(C(=O)CCn2c3c(c4ccccc42)C(*)N(C)N(C)C3)CC1 ; *C(=O)CCC(=O)N(CCOCCOCCC(=O)O)C1CCN(C(=O)CCn2c3c(c4cccnc42)C(*)N(C)N(C)C3)CC1 ; *NCCCC[C@H](NC(=O)CNC(=O)CNC(=O)CN*)C(N)=O]

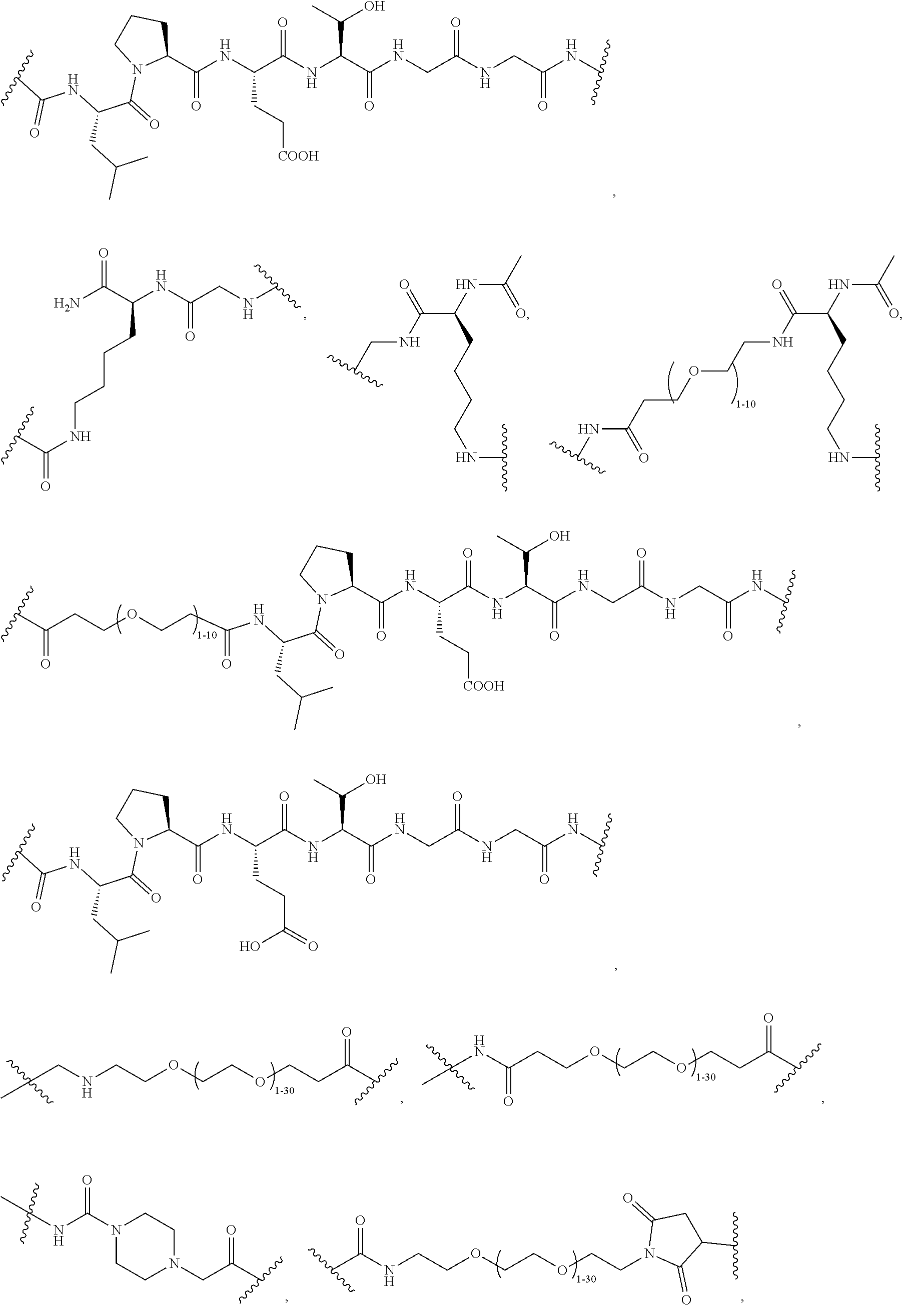

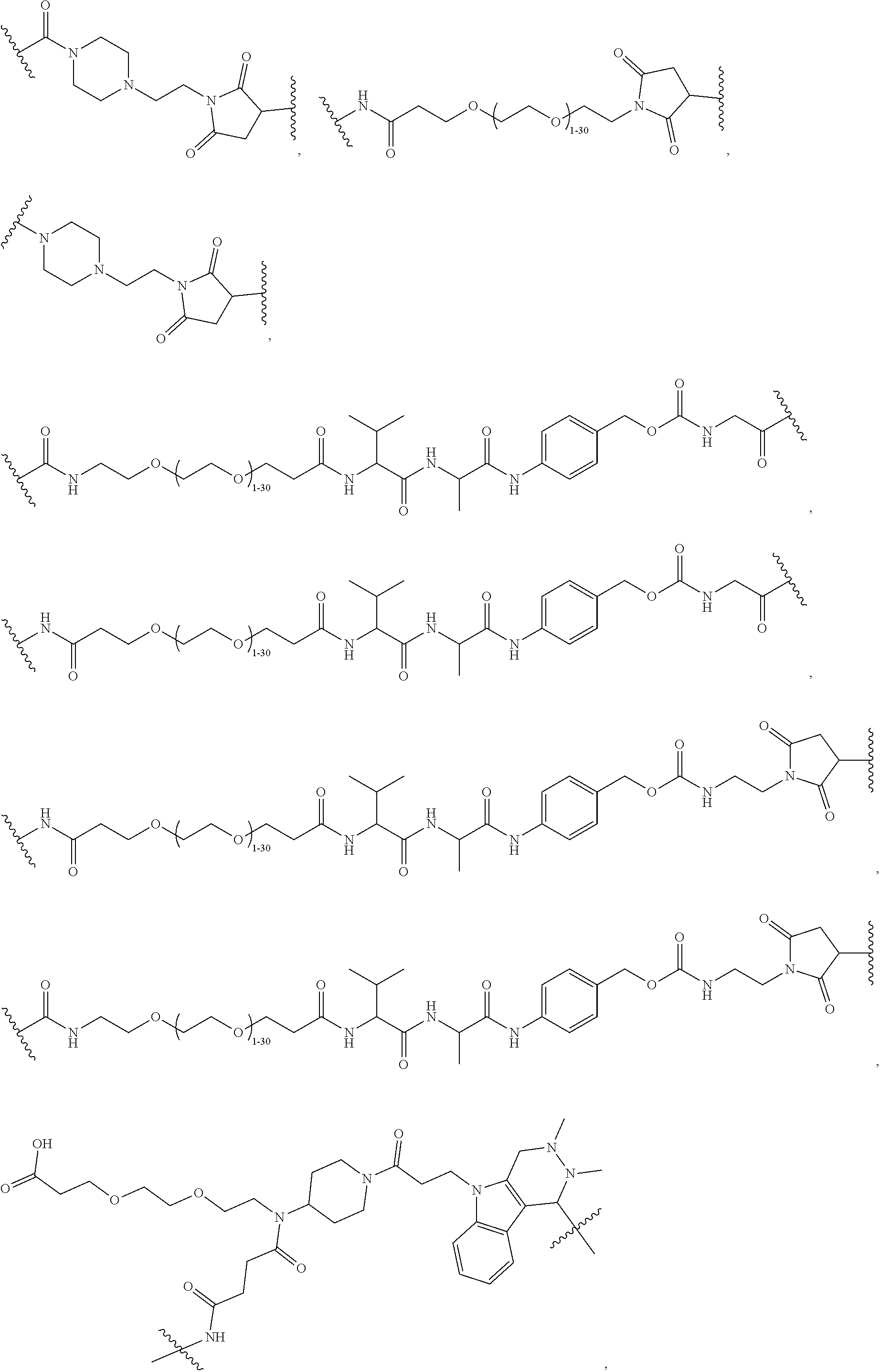

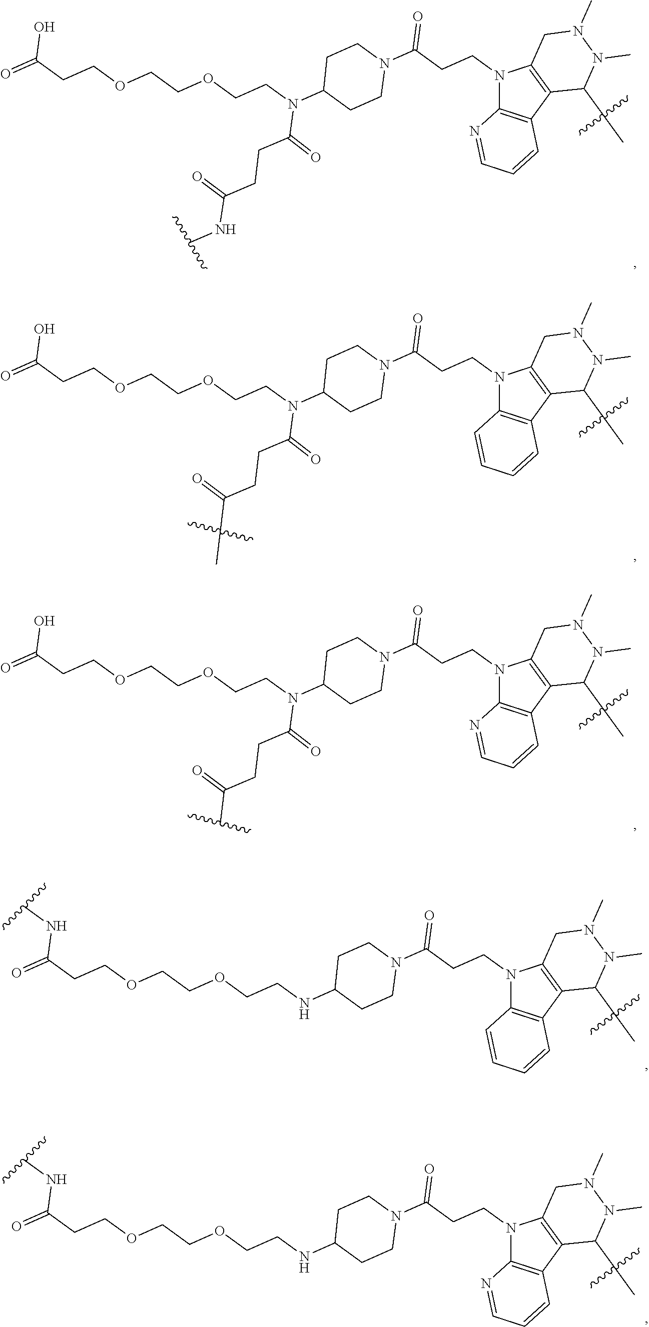 [SMILES: *NC(=O)CCC(=O)N(CCOCCOCCC(=O)O)C1CCN(C(=O)CCn2c3c(c4cccnc42)C(*)N(C)N(C)C3)CC1 ; *C(=O)CCC(=O)N(CCOCCOCCC(=O)O)C1CCN(C(=O)CCn2c3c(c4ccccc42)C(*)N(C)N(C)C3)CC1 ; *C(=O)CCC(=O)N(CCOCCOCCC(=O)O)C1CCN(C(=O)CCn2c3c(c4cccnc42)C(*)N(C)N(C)C3)CC1 ; *NC(=O)CCOCCOCCNC1CCN(C(=O)CCn2c3c(c4ccccc42)C(*)N(C)N(C)C3)CC1 ; *NC(=O)CCOCCOCCNC1CCN(C(=O)CCn2c3c(c4cccnc42)C(*)N(C)N(C)C3)CC1]

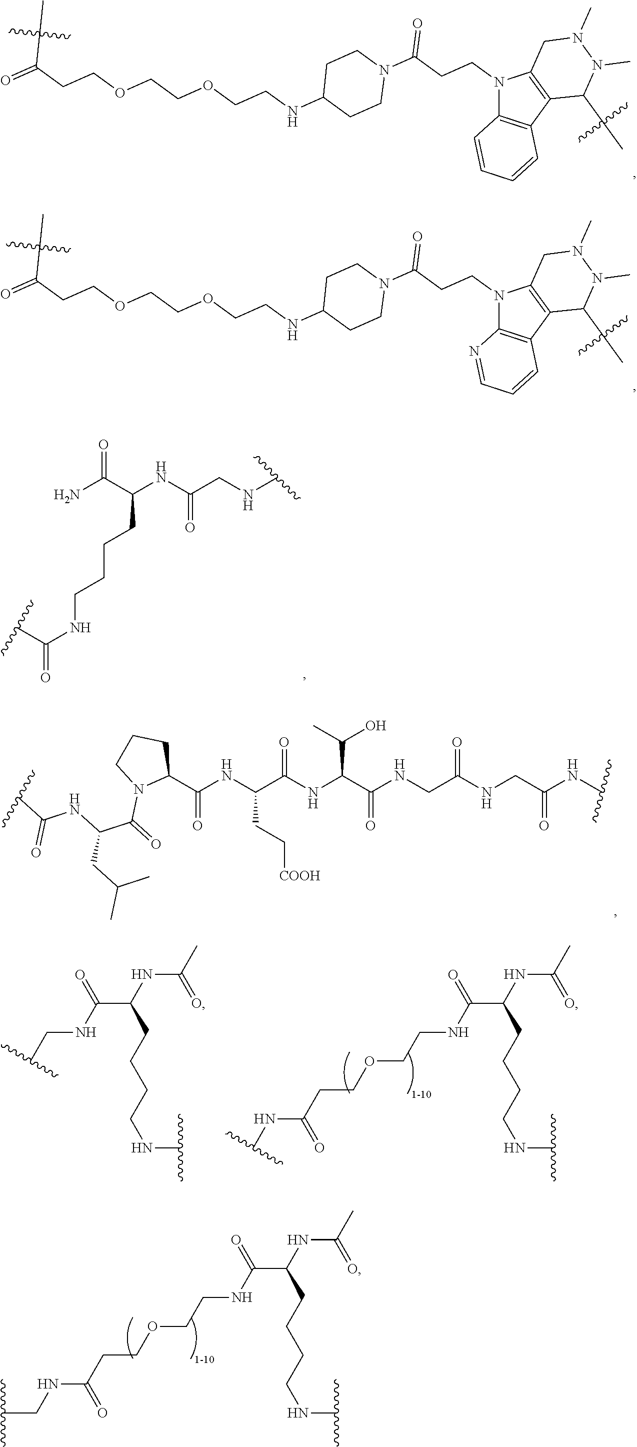

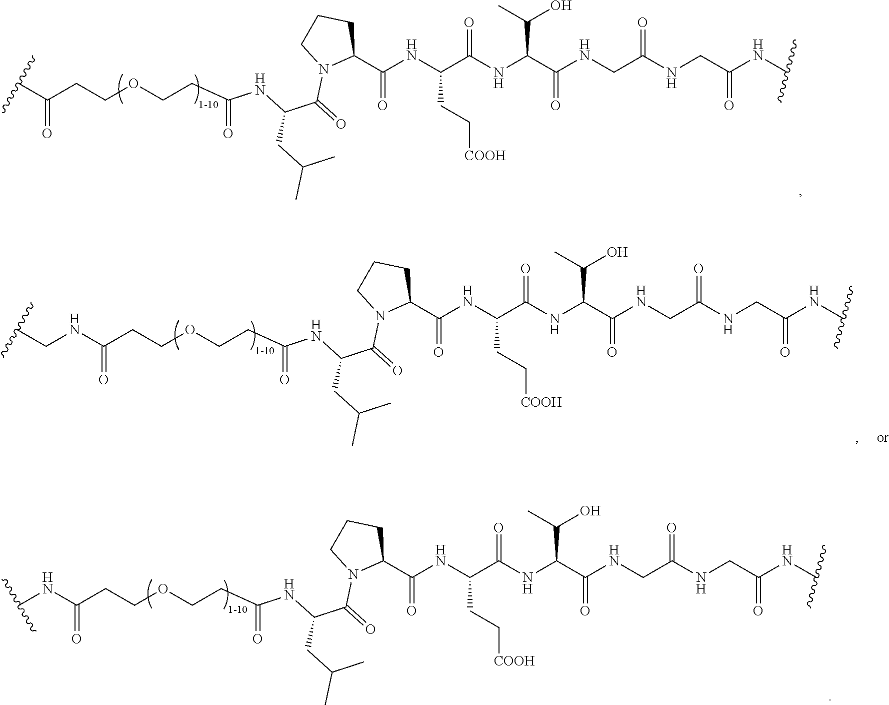

46. (canceled)

47. The targeting moiety of claim 1, wherein X is an antibody or antibody fragment moiety that targets HER2, TROP2, Nectin-4, Claudin-18.2, MMP9, mesothelin, FN1, FAP, TNC, or ECM, EPCAM, CEA, or CEACAM5; and
L, at each occurrence, is independently a linker selected from the group consisting of:

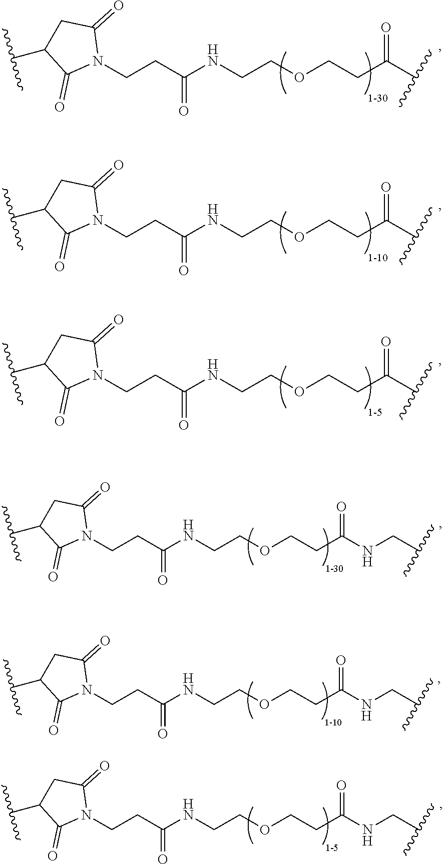

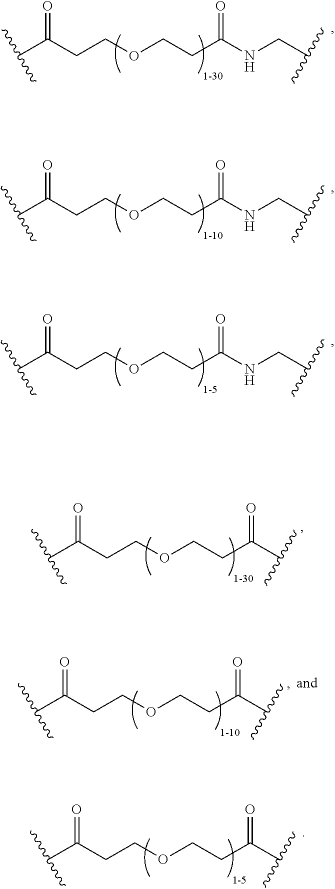

48. (canceled)

49. The targeting moiety of claim 1, wherein the targeting moiety is of Formula IID:

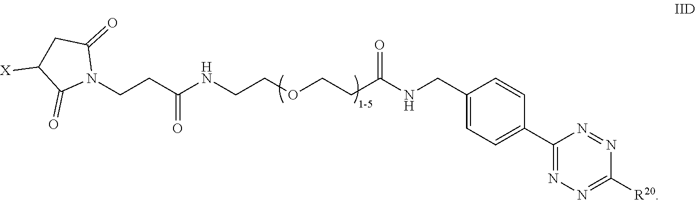

50. (canceled)

51. The targeting moiety of claim 1, wherein the targeting moiety is of Formula IIE: 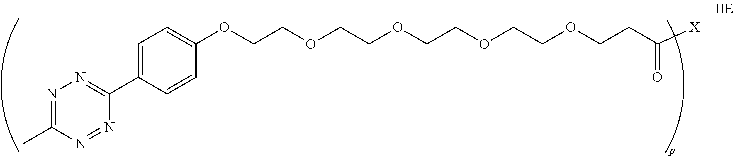
wherein p is 1-20; and X is an antibody or antibody fragment moiety.

52. (canceled)

53. A pharmaceutical composition comprising the targeting moiety of claim 1, or a pharmaceutically acceptable salt thereof, and a pharmaceutically acceptable carrier.

54. A method of treating cancer or enhancing or eliciting an immune response, the method comprising administering to a subject in need thereof, a therapeutically effective amount of the targeting moiety of claim 1, or a pharmaceutically acceptable salt thereof, or the pharmaceutical composition of claim 1, and a conjugate comprising payload linked to one or more trans-cyclooctene moieties, or a pharmaceutically acceptable salt thereof.

55. The method of claim 54, wherein the conjugate is of Formula X, or a pharmaceutically acceptable salt thereof: 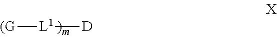
wherein:
G, at each occurrence, is independently 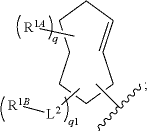
L¹, at each occurrence, is independently a linker;
m is an integer from 1-150;
D is a payload;
R^{1A}, at each occurrence, is independently selected from the group consisting of C₁₋₄alkyl, C₁₋₄haloalkyl, and C₁₋₄alkoxy;
q is 0, 1, or 2;
q1 is 0 or 1;
R^{1B}, at each occurrence, is independently selected from the group consisting of G¹, OH, —NR^{1c}—C₁₋₄alkylene-G¹, —NR^{1c}—C₁₋₄alkylene-N(R^{1d})₂, —NR^{1c}—C₁₋₆alkylene-N(C₁₋₄alkyl)₃⁺, —N(R^{1c})CHR^{1e}CO₂H, —N(R^{1c})—C₁₋₆alkylene-CO₂H, —N(R^{1f})—C₂₋₄alkylene-(N(C₁₋₄alkylene-CO₂H)—C₂₋₄alkylene)ₙ-N(C₁₋₄alkylene-CO₂H)₂, —N(R^{1c})CHR^{1e}C(O)OC₁₋₆alkyl, —N(R^{1c})—C₁₋₆alkylene-C(O)OC₁₋₆alkyl, —N(R^{1f})—C₂₋₄alkylene-(N(C₁₋₄alkylene-C(O)OC₁₋₆alkyl)-C₂₋₄alkylene)ₙ-N(C₁₋₄alkylene-C(O)OC₁₋₆alkyl)₂, —N(R^{1c})—C₁₋₆alkylene-SO₃H, —N(R^{1c})—(CH₂CH₂O)₁₋₃—CH₂CH₂N ((CH₂CH₂O)₁₋₃—C₁₋₆alkylene-CO₂H)₂, and —N(R^{1c})—CH(CH₂O—(CH₂CH₂O)₀₋₂—C₁₋₆alkylene-CO₂H)₂;
R^{1c} and R^{1d}, at each occurrence, are independently hydrogen or C₁₋₄alkyl;
R^{1e}, at each occurrence, is independently —C₁₋₄alkylene-CO₂H, —C₁₋₄alkylene-CONH₂, or —C₁₋₄alkylene-OH;
R^{1f}, at each occurrence, is independently hydrogen, C₁₋₆alkyl, or C₁₋₄alkylene-CO₂H;
n, at each occurrence, is independently 0, 1, 2, or 3;
L², at each occurrence, is independently selected from the group consisting of —C(O)— and C₁₋₃alkylene; and
G¹, at each occurrence, is independently an optionally substituted heterocyclyl.

56-64. (canceled)

65. The method of claim 54, wherein the method is a method of enhancing or eliciting an immune response.

66. The method of claim 65, wherein the immune response is an increase in one or more of leukocytes, lymphocytes, monocytes, and eosinophils.

67. The method of claim 54, further comprising administering a therapeutically effective amount of an additional therapeutic agent selected from the group consisting of an anticancer agent, an immunomodulatory agent, or a trans-cyclooctene prodrug thereof.

68-69. (canceled)
